(19) United States
(12) Patent Application Publication
(10) Pub. No.: US 2022/0339208 A1
(43) Pub. Date: Oct. 27, 2022

(54) COMPOSITIONS AND METHODS OF TREATING PSORIASIS AND ATOPIC DERMATITIS USING PREVOTELLA HISTICOLA

(71) Applicant: Evelo Biosciences, Inc., Cambridge, MA (US)

(72) Inventors: S. M. Abel, Barham Kent (GB); Kristie Barth, Cambridge, MA (US); Mark Bodmer, Boston, MA (US); Taylor A. Cormack, Dedham, MA (US); Tanmoy Ganguly, Cambridge, MA (US); Humphrey Gardner, Marblehead, MA (US); Andrea Itano, Arlington, MA (US); Duncan McHale, Kent (GB); Mustafa Noor, Middleton, MA (US); Holly Ponichtera, Watertown, MA (US); Kritika Ramani, Cambridge, MA (US); Peter Sandy, Revere, MA (US)

(21) Appl. No.: 17/633,105

(22) PCT Filed: Aug. 4, 2020
(86) PCT No.: PCT/US20/44851

§ 371 Date: Feb. 4, 2022

Related U.S. Application Data
(60) Provisional application No. 63/030,581, filed on May 27, 2020.
(60) Provisional application No. 63/023,559, filed on May 12, 2020.
(60) Provisional application No. 62/940,005, filed on Nov. 25, 2019.
(60) Provisional application No. 62/930,370, filed on Nov. 4, 2019.
(60) Provisional application No. 62/883,943, filed on Aug. 7, 2019.
(60) Provisional application No. 62/883,085, filed on Aug. 5, 2019.

(51) Int. Cl. A61K 35/74; A61K 9/00; A61P 17/00; A61P 17/06

(52) U.S. Cl. CPC A61K 35/74; A61K 9/0053; A61P 17/00; A61P 17/06; A61K 2035/11

(57) ABSTRACT

Provided herein are methods and compositions related to Prevotella bacteria useful as therapeutic agents, e.g., for the treatment of psoriasis or atopic dermatitis.

### RELATED APPLICATIONS

This application claims the benefit of priority to U.S. Provisional Patent Applications having Ser. Nos. 62/883,085, filed Aug. 5, 2019, 62/883,943, filed Aug. 7, 2019, and 62/930,370, filed Nov. 4, 2019, 62/940,005, filed Nov. 25, 2019, 63/023,559, filed May 12, 2020, and 63/030,581, filed May 27, 2020, the contents of each are hereby incorporated by reference in their entirety.

### SUMMARY

In certain aspects, provided herein are bacterial compositions (e.g., pharmaceutical compositions) comprising Prevotella histicola useful for the treatment and/or prevention of psoriasis (e.g., mild to moderate psoriasis) and/or atopic dermatitis (e.g., mild to moderate atopic dermatitis) (e.g., in a subject, e.g., a human subject) and methods of using such bacterial compositions (e.g., for the treatment of psoriasis, for the treatment of atopic dermatitis, for the reduction of Lesion Severity Scores (LSS), for the reduction of Psoriasis Area Severity Index (PASI) scores). In some embodiments, the bacterial compositions comprise whole Prevotella histicola bacteria (e.g., live bacteria, killed bacteria, attenuated bacteria).

In some embodiments, the Prevotella histicola is Prevotella Strain B 50329 (NRRL accession number B 50329; Strain B). In some embodiments, the Prevotella strain is a strain comprising at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the bacterial composition comprises one strain of bacteria, wherein the one strain of bacteria is a strain comprising at least 99.9% sequence identity to the nucleotide sequence of the Prevotella histicola Strain B 50329 (NRRL accession number B 50329). In some embodiments, the bacterial composition comprises one strain of bacteria, wherein the one strain of bacteria is the Prevotella histicola Strain B 50329 (NRRL accession number B 50329).

In some embodiments, the bacterial composition comprises at least 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola, e.g., of Prevotella Strain B 50329.

In some embodiments, the bacterial composition comprises at most 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola, e.g., of Prevotella Strain B 50329.

In some embodiments, the bacterial composition comprises about 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola, e.g., of Prevotella Strain B 50329.

In some embodiments, the bacterial composition comprises about 50 mg to about 600 mg of Prevotella histicola, e.g., of Prevotella Strain B 50329.

In some embodiments, the bacterial composition comprises about 600 mg to about 3 g of Prevotella histicola, e.g., of Prevotella Strain B 50329.

In some embodiments, the bacterial composition comprises about 55 mg of Prevotella histicola, e.g., of Prevotella Strain B 50329.

In some embodiments, the bacterial composition comprises about 550 mg of Prevotella histicola, e.g., of Prevotella Strain B 50329.

In some embodiments, the bacterial composition comprises about 2.76 g of Prevotella histicola, e.g., of Prevotella Strain B 50329.

In some embodiments, the bacterial composition comprises about 1.6×10¹⁰ total cells of Prevotella histicola, e.g., of Prevotella Strain B 50329.

In some embodiments, the bacterial composition comprises about 8×10¹⁰ total cells of Prevotella histicola, e.g., of Prevotella Strain B 50329.

In some embodiments, the bacterial composition comprises about 1.6×10¹¹ total cells of Prevotella histicola, e.g., of Prevotella Strain B 50329.

In some embodiments, the bacterial composition comprises about 3.2×10¹¹ total cells of Prevotella histicola, e.g., of Prevotella Strain B 50329.

In some embodiments, the bacterial composition comprises about 8×10¹¹ total cells of Prevotella histicola, e.g., of Prevotella Strain B 50329.

In some embodiments, the bacterial composition comprises about 1.6×10¹⁰ to about 8×10¹¹ total cells of Prevotella histicola, e.g., of Prevotella Strain B 50329.

In some embodiments, the bacterial composition comprises about 1.6×10¹⁰ to about 1.6×10¹¹ total cells of Prevotella histicola, e.g., of Prevotella Strain B 50329.

In some embodiments, the bacterial composition comprises about 1.6×10¹¹ to about 8×10¹¹ total cells of Prevotella histicola, e.g., of Prevotella Strain B 50329.

In some embodiments, the bacterial composition comprises about 8×10¹⁰ to about 8×10¹¹ total cells of Prevotella histicola, e.g., of Prevotella Strain B 50329.

In certain embodiments, the pharmaceutical composition (e.g., composition of the total dose administered, e.g., once or twice daily) comprises at least 1×10¹⁰ total cells (e.g., at least 1×10¹⁰ total cells, at least 2×10¹⁰ total cells, at least 3×10¹⁰ total cells, at least 4×10¹⁰ total cells, at least 5×10¹⁰ total cells, at least 6×10¹⁰ total cells, at least 7×10¹⁰ total cells, at least 8×10¹⁰ total cells, at least 9×10¹⁰ total cells, at least 1×10¹¹ total cells of the Prevotella bacteria. In some embodiments, the pharmaceutical composition comprises no more than 9×10¹¹ total cells (e.g., no more than 1×10¹⁰ total cells, no more than 2×10¹⁰ total cells, no more than 3×10¹⁰ total cells, no more than 4×10¹⁰ total cells, no more than 5×10¹⁰ total cells, no more than 6×10¹⁰ total cells, no more than 7×10¹⁰ total cells, no more than 8×10¹⁰ total cells, no more than 9×10¹⁰ total cells, no more than 1×10¹¹ total cells, no more than 2×10¹¹ total cells, no more than 3×10¹¹ total cells, no more than 4×10¹¹ total cells, no more than 5×10¹¹ total cells, no more than 6×10¹¹ total cells, no more than 7×10¹¹ total cells, no more than 8×10¹¹ total cells) of the Prevotella bacteria. In some embodiments, the pharmaceutical composition comprises about 6×10⁹ total cells of the Prevotella bacteria. In some embodiments, the pharmaceutical composition comprises about 1.6×10¹⁰ total cells of the Prevotella bacteria. In some embodiments, the pharmaceutical composition comprises about 8×10¹⁰ total cells of the Prevotella bacteria. In some embodiments, the pharmaceutical composition comprises about 1.6×10¹¹ total cells the Prevotella bacteria. In some embodiments, the pharmaceutical composition comprises about 3.2×10¹¹ total cells the Prevotella bacteria. In some embodiments, the pharmaceutical composition comprises about 8×10¹¹ total cells of the Prevotella bacteria. In some embodiments, the pharmaceutical composition comprises about 1.6×10¹⁰ to about 8×10¹¹ total cells of the Prevotella bacteria. In some embodiments, the pharmaceutical composition comprises about 1.6×10¹⁰ to about 1.6×10¹¹ total cells of the Prevotella bacteria. In some embodiments, the pharmaceutical composition comprises about 8×10¹⁰ to about 8×10¹¹ total cells of the Prevotella bacteria. In some embodiments, the pharmaceutical composition comprises about 1.6×10¹¹ to about 8×10¹¹ total cells of the Prevotella bacteria.

In certain embodiments, provided herein are solid dosage forms comprising the Prevotella bacteria. In some embodiments, the solid dosage form comprises an enteric coating (e.g., HPMC coat). In some embodiments, the solid dosage form is a capsule, e.g., an enteric coated capsule (e.g., HPMC coat). In some embodiments, each capsule comprises about 8×10¹⁰ total cells of the Prevotella bacteria. In some embodiments, 1, 2, 3, 4, 5, 6, 7, 8, 9, or 10 capsules are administered, e.g., once or twice daily to a subject. In some embodiments, 1 capsule (e.g., comprising about 8×10¹⁰ total cells) is administered, e.g., once or twice daily to a subject. In some embodiments, 2 capsules (e.g., each comprising about 8×10¹⁰ total cells) are administered, e.g., once or twice daily to a subject. In some embodiments, 4 capsules (e.g., each comprising about 8×10¹⁰ total cells) are administered, e.g., once or twice daily to a subject. In some embodiments, 10 capsules (e.g., each comprising about 8×10¹⁰ total cells) are administered, e.g., once or twice daily to a subject. In some embodiments, 1 capsule (e.g., comprising about 1.6×10¹¹ total cells) is administered, e.g., once or twice daily to a subject. In some embodiments, 2 capsules (e.g., each comprising about 1.6×10¹¹ total cells) are administered, e.g., once or twice daily to a subject. In some embodiments, 5 capsules (e.g., each comprising about 1.6×10¹¹ total cells) are administered, e.g., once or twice daily to a subject. In some embodiments, the Prevotella bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a capsule. In some embodiments, the capsule is an enteric coated capsule (e.g., HPMC coated). In some embodiments, the capsule comprises about 8×10¹⁰ total cells of the Prevotella bacteria (e.g., total dose of a capsule or plurality of capsules). In some embodiments, the capsule comprises about 1.6×10¹¹ total cells of the Prevotella bacteria (e.g., total dose of a capsule or plurality of capsules). In some embodiments, the capsule comprises about 3.2×10¹¹ total cells of the Prevotella bacteria (e.g., total dose of a capsule or plurality of capsules). In some embodiments, the capsule comprises about 8×10¹¹ total cells of the Prevotella bacteria (e.g., total dose of a capsule or plurality of capsules). In some embodiments, the Prevotella bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a tablet. In some embodiments, the tablet is an enteric coated tablet. In some embodiments, the enteric coated tablet is from 5 mm to 17 mm in diameter. In some embodiments, the tablet comprises about 8×10¹⁰ total cells of the Prevotella bacteria (e.g., total dose of a tablet or plurality of tablets). In some embodiments, the tablet comprises about 1.6×10¹¹ total cells of the Prevotella bacteria (e.g., total dose of a tablet or plurality of tablets). In some embodiments, the tablet comprises about 3.2×10¹¹ total cells of the Prevotella bacteria (e.g., total dose of a tablet or plurality of tablets). In some embodiments, the tablet comprises about 8×10¹¹ total cells of the Prevotella bacteria (e.g., total dose of a tablet or plurality of tablets). In some embodiments, the Prevotella bacteria in the tablet are lyophilized.

In certain embodiments, provided herein are solid dosage forms comprising the Prevotella bacteria. In some embodiments, the solid dosage form is a tablet, e.g., an enteric coated tablet. In some embodiments, the enteric coating comprises HPMC. In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P). In some embodiments, each tablet comprises about 8×10¹⁰ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 8×10¹⁰ total cells) is administered, e.g., once or twice daily to a subject. In some embodiments, 2 tablets (e.g., each comprising about 8×10¹⁰ total cells) are administered, e.g., once or twice daily to a subject. In some embodiments, 4 tablets (e.g., each comprising about 8×10¹⁰ total cells) are administered, e.g., once or twice daily to a subject. In some embodiments, 10 tablets (e.g., each comprising about 8×10¹⁰ total cells) are administered, e.g., once or twice daily to a subject. In some embodiments, each tablet comprises about 1.6×10¹¹ total cells of the Prevotella bacteria. In some embodiments, 1, 2, 3, 4, 5, 6, 7, 8, 9, or 10 tablets are administered, e.g., once or twice daily to a subject. In some embodiments, 1 tablet (e.g., comprising about 1.6×10¹¹ total cells) is administered, e.g., once or twice daily to a subject. In some embodiments, 2 tablets (e.g., each comprising about 1.6×10¹¹ total cells) are administered, e.g., once or twice daily to a subject. In some embodiments, 5 tablets (e.g., each comprising about 1.6×10¹¹ total cells) are administered, e.g., once or twice daily to a subject. In some embodiments, the Prevotella bacteria in the tablet are lyophilized (e.g., in a powder). In some embodiments, the Prevotella bacteria in the tablet are lyophilized in a powder, and the powder further comprises mannitol, magnesium stearate, and/or colloidal silicon dioxide.

In some embodiments, the solid dosage form comprises a mini-tablet. In some embodiments, the mini-tablet is enteric coated. In some embodiments, the mini-tablet is from 1 mm to 4 mm in diameter. In some embodiments, the mini-tablet (e.g., enteric coated mini-tablet) is a 1 mm mini-tablet, 1.5 mm mini-tablet, 2 mm mini-tablet, 3 mm mini-tablet, or 4 mm mini-tablet. In some embodiments, the solid dosage form comprises mini-tablets that comprise about 8×10¹⁰ total cells of the Prevotella bacteria (e.g., total dose of a plurality of mini-tablets). In some embodiments, the solid dosage form comprises mini-tablets that comprise about 1.6×10¹¹ total cells of the Prevotella bacteria (e.g., total dose of a plurality of mini-tablets). In some embodiments, the solid dosage form comprises mini-tablets that comprise about 3.2×10¹¹ total cells of the Prevotella bacteria (e.g., total dose of a plurality of mini-tablets). In some embodiments, the solid dosage form comprises mini-tablets that comprise about 8×10¹¹ total cells of the Prevotella bacteria (e.g., total dose of a plurality of mini-tablets). In some embodiments, the Prevotella bacteria in the mini-tablets are lyophilized.

In some embodiments, the mini-tablets (e.g., enteric coated mini-tablets) are contained in a capsule. In some embodiments, the capsule is a size 00, size 0, size 1, size 2, size 3, size 4, or size 5 capsule. In some embodiments, the capsule comprises a non-enteric coating (e.g., gelatin) (e.g., is coated with a non-enteric coating). In some embodiments, the capsule comprises a non-enteric coating. In some embodiments, the capsule comprises gelatin. In some embodiments, the mini-tablets (e.g., enteric coated mini-tablets) that comprise about 8×10¹¹ total cells of the Prevotella bacteria are contained in a capsule(s), wherein optionally the capsule comprises gelatin.

In some embodiments, the pharmaceutical composition comprising Prevotella bacteria is prepared as a powder (e.g., for resuspension or for use in a solid dose form (such as a capsule)) or as a solid dose form, such as a tablet, a mini-tablet, a capsule, a pill, or a powder; or a combination of these forms (e.g., mini-tablets comprised in a capsule). The powder can comprise lyophilized bacteria. In some embodiments, the powder further comprises mannitol, magnesium stearate, and/or colloidal silicon dioxide.

In some embodiments, the bacterial composition is administered orally. In some embodiments, the administration to the subject once daily. In some embodiments, the bacterial composition is administered in 2 or more doses (e.g., 3 or more, 4 or more or 5 or more doses). In some embodiments, the administration to the subject of the two or more doses are separated by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days.

In some embodiments, the bacterial composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days. In some embodiments, the bacterial composition is administered once daily for 1, 2, 3, 4, 5, 6, 7, 8, 9, 10, 11, 12, 13, 14, 15, or 16 weeks. In some embodiments, the bacterial composition is administered once daily for at least 1, 2, 3, 4, 5, 6, 7, 8, 9, 10, 11, 12, 13, 14, 15, or 16 weeks.

In some embodiments, the pharmaceutical composition comprises lyophilized Prevotella bacteria. In certain embodiments, the lyophilized Prevotella bacteria is formulated into a solid dose form, such as a tablet, a mini-tablet, a capsule, a pill, or a powder. In some embodiments, the lyophilized Prevotella bacteria is contained in a capsule. In some embodiments, the powder further comprises mannitol, magnesium stearate, and/or colloidal silicon dioxide. In some embodiments, the lyophilized Prevotella bacteria is resuspended in a solution.

In some embodiments, the bacterial composition is formulated as a capsule or a tablet. In some embodiments, the bacterial formulation (e.g., composition) comprises an enteric coating or micro encapsulation. In some embodiments, the capsule is an enteric coated capsule. In some embodiments, the enteric coating allows the bacterial composition to be released in the upper small intestine, e.g., duodenum. In some embodiments, the enteric coating comprises HPMC.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla or a chimpanzee).

In certain embodiments, provided herein are methods of treating a subject who has psoriasis (e.g., mild to moderate psoriasis) and/or atopic dermatitis (e.g., mild to moderate atopic dermatitis) comprising administering to the subject a bacterial composition described herein.

In certain embodiments, provided herein are methods of decreasing Lesion Severity Score (LSS) (e.g., mean LSS) (e.g., as compared to baseline or placebo control) in a subject (e.g., a subject with psoriasis) comprising administering to the subject a bacterial composition described herein. In certain embodiments, the LSS in the subject is reduced by at least 10%, 15%, 20%, 25%, 30%, 35%, 40%, 45%, 50%, 55%, 60%, 65%, 70%, 75%, 80%, or more (e.g., by day 7, 14, 21, 28, or 35 of treatment). In certain embodiments, the LSS in the subject is reduced by at least 10%, 15%, 20%, 25%, 30%, 35%, 40%, 45%, 50%, 55%, 60%, 65%, 70%, 75%, 80%, or more after dosing is stopped (e.g., 14 days after treatment has stopped).

In certain embodiments, provided herein are methods of decreasing Psoriasis Area and Severity Index (PASI) score (e.g., mean PASI score) (e.g., as compared to baseline or placebo control) in a subject (e.g., a subject with psoriasis) comprising administering to the subject a bacterial composition described herein. In certain embodiments, the PASI score in the subject is reduced by at least 10%, 15%, 20%, 25%, 30%, 35%, 40%, 45%, 50%, 55%, 60%, 65%, 70%, 75%, 80%, or more (e.g., by day 7, 14, 21, 28, or 35 of treatment). In certain embodiments, the PASI score in the subject is reduced by at least 10%, 15%, 20%, 25%, 30%, 35%, 40%, 45%, 50%, 55%, 60%, 65%, 70%, 75%, 80%, or more after dosing is stopped (e.g., 14 days after treatment has stopped).

In certain embodiments, provided herein are methods of increasing a sustained clinical effect (e.g., continued reductions from baseline (or placebo) in mean LSS and/or PASI, e.g., 1, 2, 3, 4, 5, 6 or more weeks after completion of dosing) in a subject (e.g., a subject with psoriasis) comprising administering to the subject a bacterial composition described herein. In certain embodiments, the LSS and/or PASI score in the subject is reduced by at least 10%, 15%, 20%, 25%, 30%, 35%, 40%, 45%, 50%, 55%, 60%, 65%, 70%, 75%, 80%, or more (e.g., by day 7, 14, 21, 28, or 35 of treatment).

In certain embodiments, provided herein are methods of enhancing anti-inflammatory cytokine production (e.g., increasing as compared to amount produced (e.g., mRNA and/or protein) in the absence of the bacterial composition) in a subject, the method comprising administering a bacterial composition described herein. In some embodiments, the anti-inflammatory cytokine is IL-10, IL-27, and/or IL1RA. In certain embodiments, the anti-inflammatory cytokine is expressed by M1-type APCs. In some embodiments, enhancing anti-inflammatory cytokine production comprises an increase in anti-inflammatory cytokine (e.g., IL-10, IL-27, and/or IL1RA) mRNA levels (e.g., in skin biopsies). In some embodiments, enhancing anti-inflammatory cytokine production comprises an increase in anti-inflammatory cytokine (e.g., IL-10, IL-27, and/or IL1RA) protein levels (e.g., in blood samples).

In certain embodiments, provided herein are methods of inhibiting pro-inflammatory cytokine production (e.g., decreasing as compared to amount produced (e.g., mRNA and/or protein) in the absence of the bacterial composition) in a subject, the method comprising administering a bacterial composition described herein. In some embodiments, the pro-inflammatory cytokine is GM-CSF, IL-17A, and/or IL-13. In some embodiments, the pro-inflammatory cytokine is IL-6, TNF, and/or IL-12p70. In some embodiments, the pro-inflammatory cytokine is IL23p40, IL17, IL-6, TNF, and/or IL-13. In some embodiments, inhibiting pro-inflammatory cytokine production comprises inhibiting pro-inflammatory cytokine production in a draining lymph node (e.g., cervical lymph node). In some embodiments, inhibiting pro-inflammatory cytokine production comprises inhibiting pro-inflammatory cytokine production in the spleen. In some embodiments, inhibiting pro-inflammatory cytokine production comprises a decrease in pro-inflammatory cytokine (e.g., Il17a) mRNA levels (e.g., in skin biopsies). In some embodiments, inhibiting pro-inflammatory cytokine production comprises a decrease in pro-inflammatory cytokine (e.g., IL-17A) protein levels (e.g., in blood samples).

In certain embodiments, provided herein are methods of inhibiting pro-inflammatory chemokines production (e.g., decreasing as compared to amount produced (e.g., mRNA and/or protein) in the absence of the bacterial composition) in a subject, the method comprising administering a bacterial composition described herein. In some embodiments, the pro-inflammatory chemokine is keratinocyte chemoattractant (KC).

In certain embodiments, provided herein are methods of altering cytokine production or chemokine production (e.g., altering as compared to amount produced (e.g., mRNA and/or protein) in the absence of the bacterial composition) in a subject, the method comprising administering a bacterial composition described herein. In some embodiments, blood samples from the subject are stimulated ex vivo and analyzed for levels of cytokines and/or chemokines. In some embodiments, the level of IL-1 beta, IL-2, IL-4, IL-6, IL-8, IL-10, IL-12p40, IL-17A, TNFα, and/or IFNγ is analyzed.

In some embodiments, the human subject is at least 18 years old. In some embodiments, the human subject is no more than 60 years old. In certain embodiments, the human subject has a body mass index of at least 18 kg/m². In some embodiments, the human subject has a body mass index of no more than 35 kg/m². In some embodiments, the human subject has not received live attenuated vaccination within 10 weeks prior to dosing. In some embodiments, the human subject does not require treatment with an anti-inflammatory drug. In some embodiments, the human subject does not have an active infection. In some embodiments, the human subject has not had an infection requiring antibiotic treatment within 6 weeks prior to dosing. In some embodiments, the human subject does not have renal or liver impairment. In some embodiments, the human subject does not have neoplastic disease or a history of neoplastic disease within 5 years prior to dosing. In some embodiments, the human subject has not had a major surgery within 4 weeks prior to dosing. In some embodiments, the human subject does not have impaired cardiac function or clinically significant cardiac diseases. In some embodiments, the human subject does not have a known history of human immunodeficiency virus (HIV), active hepatitis A, hepatitis B, or hepatitis C, and/or is not known to be positive for HCV ribonucleic acid and/or HBV surface antigen. In some embodiments, the human subject does not have an active central nervous system (CNS) malignancy. In some embodiments, the human subject does not have GI tract disease. In some embodiments, the human subject does not have a history of hypersensitivity or allergies to Prevotella (or Prevotella-containing probiotics) including, e.g., any associated excipients. In some embodiments, the human subject does not have a history of hypersensitivity or allergies to placebo capsule (magnesium stearate and cellulose) and/or to the hard capsule shells (hydroxyl propyl methyl cellulose and titanium dioxide). In some embodiments, the human subject does not have a significant history of drug abuse or regular use of illicit drugs or a history of alcohol abuse within 1 year prior to dosing. In some embodiments, the human subject does not have a clinically significant illness other than the immunoinflammatory disorder.

In embodiments, provided herein is a method of treating psoriasis comprising administering (e.g., orally administering) to a human subject a strain of a Prevotella histicola and/or a composition (e.g., a pharmaceutical composition and/or a solid dosage form) comprising a strain of a Prevotella histicola provided herein. In some embodiments, the human subject has a confirmed diagnosis of mild to moderate plaque-type psoriasis for at least 6 months involving no more than 10% of body surface area (BSA) (excluding the scalp). In some embodiments, the human subject has a minimum of 2 psoriatic lesions. In some embodiments, the subject has not received systemic non-biologic psoriasis therapy (methotrexate [MTX], steroids, cyclophosphamide) or psoralen plus ultraviolet A (PUVA)/ultraviolet A (UVA) phototherapy within 4 weeks prior to dosing. In some embodiments, subject has not received treatment with biologic agents within 12 months prior to first dose. In some embodiments, the subject is not continuing use of topical or oral pharmacologically active agents 2 weeks prior to the start of dosing. In some embodiments, the human subject has a documented diagnosis of plaque psoriasis for ≥6 months.

In some embodiments, the human subject has had mild to moderate plaque psoriasis with plaque covering BSA of ≥3% and ≤10% and meet both of the following additional criteria: (i) PASI score of ≥6 and ≤15, and (ii) PGA score of 2 or 3.

In some embodiments, the method decreases the PASI (Psoriasis Area and Severity Index) score in the subject, e.g., after 16 weeks of treatment (e.g., as compared to the subject's PASI score prior to the commencement of treatment).

In some embodiments, the method increases a PASI percentage response rate (e.g., PASI-50, PASI-75, PASI-90, or PASI-100), e.g., as described herein. For example, the percentage of subjects who achieve a 75% or greater reduction in PASI score from baseline is represented by the PASI-75 value, e.g., after 16 weeks of treatment.

In some embodiments, the method decreases the LSS (Lesion Severity Score) in the subject, e.g., after 16 weeks of treatment (e.g., as compared to the subject's LSS prior to the commencement of treatment), e.g., as described herein.

In some embodiments, the method decreases the PGA (Physician's Global Assessment) score in the subject, e.g., after 16 weeks of treatment (e.g., as compared to the subject's PGA score prior to the commencement of treatment), e.g., as described herein.

In some embodiments, the method decreases the percent of BSA (Body Surface Area) involvement in the subject, e.g., after 16 weeks of treatment (e.g., as compared to the subject's percent involvement prior to the commencement of treatment), e.g., as described herein.

In some embodiments, the method decreases the mNAPSI (Modified Nail Psoriasis Severity Index) score in the subject, e.g., after 16 weeks of treatment (e.g., as compared to the subject's mNAPSI score prior to the commencement of treatment), e.g., as described herein.

In some embodiments, the method improves the DLQI (Dermatology Life Quality Index) score in the subject, e.g., after 16 weeks of treatment (e.g., as compared to the subject's DLQI score prior to the commencement of treatment), e.g., as described herein.

In some embodiments, the method improves the PSI (Psoriasis Symptom Inventory) score in the subject, e.g., after 16 weeks of treatment (e.g., as compared to the subject's PSI score prior to the commencement of treatment), e.g., as described herein.

In some embodiments, the method decreases pain in the subject, e.g., after 16 weeks of treatment (e.g., as compared to the subject's pain prior to the commencement of treatment), e.g., as described herein. For example, pain can be assessed by the SF-36 Bodily Pain Scale (SF-36 BPS) or the VAS Pain.

In some embodiments, the method decreases fatigue in the subject, e.g., after 16 weeks of treatment (e.g., as compared to the subject's fatigue prior to the commencement of treatment), e.g., as described herein.

In embodiments, provided herein is a method of treating atopic dermatitis comprising administering (e.g., orally administering) to a human subject a strain of a Prevotella histicola and/or a composition (e.g., a pharmaceutical composition and/or a solid dosage form) comprising a strain of a Prevotella histicola. In some embodiments, the human subject has a confirmed diagnosis of mild to moderate atopic dermatitis for at least 6 months involving a minimum of 3% to a maximum of 15% body surface area. In some embodiments, the subject has had a confirmed diagnosis of mild to moderate atopic dermatitis with an IGA score of 2 or 3. In some embodiments, the subject has at least 2 atopic dermatitis lesions with at least 1 in a site suitable for biopsy. In some embodiments, the subject is not receiving systemic non-biologic atopic dermatitis therapy (methotrexate (MTX), steroids, cyclophosphamide) or has received therapy within 4 weeks prior to dosing. In some embodiments, wherein the human subject is not receiving treatment with biologic agents within 12 months prior to first dose. In some embodiments, wherein the human subject is not continuing to use topical or oral pharmacologically active agents 2 weeks prior to the start of dosing.

In some embodiments, the method decreases the EAST (Eczema Area and Severity Index) score in the subject, e.g., after 16 weeks of treatment (e.g., as compared to the subject's EAST score prior to the commencement of treatment). In some embodiments, the method decreases the IGA (Investigator's Global Assessment) score in the subject, e.g., after 16 weeks of treatment (e.g., as compared to the subject's IGA score prior to the commencement of treatment). In some embodiments, the method decreases the SCORAD (SCORing Atopic Dermatitis) score in the subject, e.g., after 16 weeks of treatment (e.g., as compared to the subject's SCORAD score prior to the commencement of treatment).

In some aspects, the disclosure provides a bacterial composition described herein (e.g., in an amount described herein) for use in treating psoriasis (e.g., mild to moderate psoriasis) and/or atopic dermatitis (e.g., mild to moderate atopic dermatitis).

In some aspects, the disclosure provides use of a bacterial composition described herein (e.g., in an amount described herein) for the preparation of a medicament for the treatment of psoriasis (e.g., mild to moderate psoriasis) and/or atopic dermatitis (e.g., mild to moderate atopic dermatitis).

In certain embodiments, provided herein are methods of fortifying an intestinal epithelial barrier in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition fortifies the intestinal epithelial barrier in the subject.

In some embodiments, administration of the bacterial composition to an in vitro model of intestinal epithelial barrier integrity (e.g., an intestinal epithelial co-culture transwell culture model) fortifies the intestinal epithelial barrier (e.g., as assessed in a transepithelial electrical resistance (TEER)) in the in vitro model.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition increases IL-10R signaling in the subject.

In some embodiments, administration of the bacterial composition to an in vivo model of inflammation (e.g., a DTH model) decreases inflammation in the in vivo model, and blocking IL-10R (e.g., by administration of an IL-10R blocking antibody) in the in vivo model decreases the effect of the bacterial composition on decreasing inflammation.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition increases TLR2 signaling in the subject.

In some embodiments, administration of the bacterial composition to an in vivo model of inflammation (e.g., a DTH model) decreases inflammation in the in vivo model, and blocking TLR2 (e.g., by administration of a TLR2 blocking antibody) in the in vivo model decreases the effect of the bacterial composition on decreasing inflammation.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein administration of the bacterial composition results in increased efficacy after 30 days of dosing in the subject (e.g., as compared to the level of efficacy after 15 days of dosing). Efficacy can be determined by the decrease in the level of inflammation being greater after 30 days of dosing than the level of inflammation after 15 days of dosing.

In some embodiments, administration of the bacterial composition to an in vivo model of inflammation (e.g., a DTH model) decreases inflammation in the in vivo model, and the level of inflammation in the in vivo model after 30 days of dosing with the bacterial composition is less than the level of inflammation at after 15 days of dosing with the bacterial composition.

In certain embodiments, provided herein are methods of decreasing inflammation in a subject (e.g., a subject who has psoriasis (e.g., mild to moderate psoriasis) and/or atopic dermatitis (e.g., mild to moderate atopic dermatitis)) comprising administering to the subject a bacterial composition described herein, wherein the effects on inflammation of the administration of the bacterial composition persist for at least 14 days after last dosing the subject (e.g., the level of inflammation is lower 14 days after last dosing the subject, as compared to the level of inflammation prior to commencement of dosing the subject). Persistence can be determined by the decrease in the level of inflammation being greater at 14 days after last dosing the subject than the level of inflammation prior to commencement of dosing the subject.

In some embodiments, administration of the bacterial composition to an in vivo model of inflammation (e.g., a DTH model) decreases inflammation in the in vivo model, and the level of inflammation in the in vivo model at 14 days after last dosing with the bacterial composition is less than the level of inflammation prior to commencement of dosing.

### BRIEF DESCRIPTION OF THE DRAWINGS

FIGS. 1A-1B show Prevotella histicola strain B is efficacious in reducing lesion severity score. FIG. 1A is a graph showing that patients dosed daily for 28 days with 550 mg of the enteric capsule formulation of Prevotella histicola Strain B (strain B) showed a statistically significant (p≤0.05) reduction in mean LSS at 28 days of 2 points, compared to a mean increase of 0.25 points in patients who received placebo. FIG. 1B is a graph showing mean percent changes in Lesion Severity Scores (LSS) over the course of the study.

FIG. 2 is a graph showing that patients dosed with Prevotella histicola Strain B showed a reduction in LSS over the dosing period ranging from 0 to 67 percent.

FIG. 3 is a graph showing a mean reduction of 2.25 cells/mm² in patients who received Prevotella histicola Strain B (strain B) compared to no change in patients receiving placebo.

FIG. 4 is a graph showing that the Prevotella histicola Strain B (strain B) dosed patient group showed a reduction in cytokine production indicative of a systemic anti-inflammatory response, compared to no reduction in the placebo group.

FIG. 5 is a graph showing mean LSS reduction of 15% at 28 days, which continued to 24% at day 42 for patients dosed with a high dose (2.76 g) of Prevotella histicola Strain B (strain B).

FIG. 6 is a graph showing LSS reduction consistent between high (2.76 g) and low (550 mg) doses of Prevotella histicola Strain B (strain B) over 28 days; the reduction in high dose continued to day 42.

FIG. 7 is a graph showing reduction in LSS of up to 80% at day 42 at high dose of Prevotella histicola Strain B (strain B).

FIG. 8 is a graph showing high dose mean PAST reduction consistent with LSS; the PAST reduction continued to improve after end of dosing of Prevotella histicola Strain B (strain B).

FIG. 9 is a graph showing reduction in PAST of up to 62% at day 42 at high dose of Prevotella histicola Strain B (strain B).

FIG. 10 is a graph showing that Prevotella histicola strain B enhances IL-10 and IL-27 cytokine production by human inflammatory M1-type APCs.

FIGS. 11A-11B show that Prevotella histicola strain B is efficacious in a model of peripheral T cell-mediated inflammation (KLH DTH). FIG. 11A shows delayed-type hypersensitivity (DTH) response to Keyhole Limpet Hemocyanin (KLH). C57Bl/6 mice were immunized with KLH and CFA and challenged intradermally in the ear 9 days later with KLH. Mice were treated from the day after immunization through ear challenge with placebo, dexamethasone (1 mg/kg IP QD), or Prevotella histicola strain B (1.8 mg PO QD). Ear inflammation was measured on day 9. FIG. 11B shows ex vivo stimulation of draining lymph node or spleen cells with KLH. At the end of the DTH study, mice were sacrificed and total cells from ear draining lymph nodes and spleens were incubated with KLH for 2 days. Cytokines from supernatants were measured by MSD. (Diamonds: placebo; squares: dexamethasone (Dex); stars: Prevotella histicola strain B)

FIGS. 12A-12C show that oral treatment of Prevotella histicola strain B is efficacious in a Type 17-driven model of skin inflammation (Imiquimod driven psoriasis). FIG. 12A shows skin scores for BALB/c mice that were topically treated with 5% imiquimod, a TLR7 and TLR8 agonist for 7 days on the back skin and ear. Mice were treated daily from day 1 through 7 with placebo, dexamethasone (1 mg/kg IP) or Prevotella histicola strain B (10 mg PO). Back scores were recorded daily to measure erythema and scaling associated with psoriasis. FIG. 12B shows that Il17a mRNA transcripts from the psoriatic skin of the mice were measured by RT-qPCR. FIG. 12C shows ex vivo stimulation of splenocytes. At termination of the study, mice were sacrificed and splenocytes were stimulated with PMA/Ionomycin for 48 hrs. IL-17A was measured from supernatants by MSD.

FIGS. 13A-13F show delayed-type hypersensitivity (DTH) response to Keyhole Limpet Hemocyanin (KLH). C57Bl/6 mice were immunized with KLH and CFA and challenged intradermally in the ear 9 days later with KLH. Mice were treated from the day after immunization through ear challenge with placebo, dexamethasone (1 mg/kg IP QD), or Prevotella histicola strain B (1.8 mg PO QD). Ear inflammation was measured on day 9. In all experiments, Prevotella histicola strain B, anaerobic sucrose and other mentioned strains was given orally daily starting on day 1 for 9 days Dexamethasone was given as daily i.p. injections. FIG. 13A shows change in ear thickness (n=5 mice/group) and (B) FIG. 13B shows dose response for Prevotella histicola strain B in modulating change in ear thickness as a measure of inflammation. ****p≤0.001, ns: not significant, as determined by unpaired Student's t-test.

FIG. 13C shows ex vivo stimulation of draining lymph node or spleen cells with KLH. At the end of the DTH study, tissues were harvested and total cells from mesenteric lymph nodes, spleen and ear draining lymph nodes were stimulated with LPS or KLH for 48-72 h. Cytokines from supernatants were measured by MSD. Data are representative from 2 to 4 experiments with n=5/group. All data show mean: SEM/SD. **p≤0.001, ***p≤0.0005, ****p≤0.0001, ns: not significant, as determined by unpaired Student's t-test. FIG. 13D shows Prevotella histicola strain B acts through IL-10 to reduce ear inflammation. Mice were treated with IL-10R blocking antibody on days 2, 4, and 6. Representative figure from n=2 experiments with 5 mice/group in each experiment; **p≤0.01, ****p≤0.0001, ns: not significant as determined by unpaired Student's t-test. FIG. 13E shows passive transfer of cells from mice treated with Prevotella histicola strain B. Mice with DTH were treated with Prevotella histicola strain B for 8 days and then different sets of cells from these treated animals were passively transferred into a second set of immunized animals that were not dosed with Prevotella histicola strain B. Representative figure from n=2 experiments with 5 mice/group in each experiment; **p≤0.01, ****p≤0.0001, ns: not significant as determined by Ordinary one-way ANOVA. FIG. 13F shows therapeutic dosing with Prevotella histicola strain B. KLH-DTH was induced in mice and they were treated with Prevotella histicola strain B for 8, 3 or 1 days as indicated above each panel in the figure. Data are representative of two independent experiments with 5 mice/group in each experiment; *p≤0.05 **p≤0.01, ****p≤0.0001, ns: not significant as determined by unpaired Student's t-test.

FIGS. 14A-14C show adoptive transfer of DO11 TCR Tg cells in delayed type hypersensitivity. BALB/c mice (n=5 per group) were adoptively transferred with 4×106 DO11 TCR Tg cells 24 h prior to induction of DTH with OVA+CFA subcutaneously. At day 8, mice were challenged in the ear intradermally with 20 μg OVA. 24 h post challenge, ear measurements were recorded. Spleens and ear draining lymph nodes cells were re-stimulated ex vivo for 72 h with OVA323-339 peptide to assess antigen specific cytokine responses. FIG. 14A shows that the bars represent the mean±SEM of the change in ear inflammation 24 h post ear challenge.

FIG. 14B shows pro-inflammatory cytokine levels and FIG. 14C shows type 3 cytokines in the supernatants from ear draining cervical lymph node cells. Data are representative of two independent experiments with 5 mice/group in each experiment. ***p≤0.01, ****p≤0.0001, ns: not significant as determined by one-way ANOVA.

FIGS. 15A-15D show that Imiquimod driven psoriasis mouse model BALB/c mice were topically treated with 5% imiquimod, a TLR7 and TLR8 agonist for 7 days on the back skin and ear. Mice were treated daily from day 1 through 7 with placebo, dexamethasone (1 mg/kg IP) or Prevotella histicola strain B (10 mg PO). FIG. 15A shows phenotypic presentation of mouse back skin after 7 days of imiquimod application. Back scores were recorded daily to measure erythema and scaling associated with psoriasis. **p≤0.01 ****p≤0.001, ns: not significant, as determined by one-way ANOVA. FIG. 15B shows that IMQ application alters proliferation of keratinocytes and infiltration of immune cells. H&E staining of back skin reveals increased nuclei in the stratum corneum of skin represented as hyperkeratosis and thickening of the epidermis shown as acanthosis which were scored by a pathologist. *p≤0.05 **p≤0.01, as determined by one-way ANOVA. FIG. 15C shows that at termination of the study, splenocytes were ex vivo re-stimulated with PMA/Ionomycin for 48 hrs. Additionally, ears were homogenized and protein levels of IL-17A was measured from supernatants by MSD. FIG. 15D shows that Prevotella histicola strain B was equally efficacious as anti-IL-17A in reducing ear inflammation after IMQ application over the course of 7 days. **p≤0.01 ****p≤0.001, as determined by one-way ANOVA.

FIGS. 16A-16C show that Prevotella histicola strain B suppressed neuroinflammation in a relapsing remitting model of EAE. EAE was induced in SJL mice by immunization with PLP91-110 in CFA and (pertussis toxin (PTX) was administered on days 1, 3 and 7 post EAE induction). Prevotella histicola strain B was orally dosed daily for 41 days. Mice in the control group (vehicle) dosed with anaerobic sucrose. FIG. 16A shows that cumulative EAE scores of mice treated with vehicle, Prevotella histicola strain B or fingolimod as a positive control. The data are representative of 3 independent experiments (n=15 mice per group). Clinical scores were assessed daily for the duration of the experiment. ***p≤0.0005 and ****p≤0.00005. FIG. 16B shows that treatment with Prevotella histicola strain B decreased inflammation and infiltrating inflammatory cells in the spinal cord of EAE mice. Representative hematoxylin and eosin (H&E)-stained images of the brain of spinal cords of mice treated with Prevotella histicola strain B, Vehicle or Fingolimod. Spinal cord sections are enlarged at 289× magnification to show regions with inflammation and inflammatory loci. Data are representative of 2 independent experiments (n=15 mice per group). FIG. 16C shows transcriptional profiling of Tregs related genes in the duodenum vs colon of EAE mice at termination of study at day 42 by qPCR. *p≤0.05 by unpaired Student's. Data are representative of 2 independent experiments (n=5 mice per group). Prevotella histicola strain B induces duodenal expression of Treg genes in EAE treatment.

FIGS. 17A-17B show in vitro activity of Prevotella histicola strain B. FIG. 17A shows that Prevotella histicola strain B is more potent and induces increased amounts of IL-10 secretion compared to P. jejuni. FIG. 17B shows that Prevotella histicola strain B consistently fortifies the intestinal epithelial barrier in a dose-dependent manner. Intestinal epithelial co-culture transwell cultures (60% Caco-2 and 40% HT-29 cell lines) were incubated with sucrose vehicle or varying concentrations of Prevotella histicola strain B for 24 hours. Before and after incubation with microbes, the epithelial barrier was assessed via transepithelial electrical resistance (TEER). Representative data from three independent experiments each with three technical replicates is shown. The barrier integrity is calculated as fold change from time zero and is reported as percent sucrose vehicle.

FIGS. 18A-18D show murine models of Th2 driven atopic dermatitis. Murine models of Th2 driven atopic dermatitis. In FIGS. 18A-18B, BALB/c mice were topically sensitized with 0.5% FITC, on day 1 and 2 and 6 days later challenged with 0.5% FITC on the ear. Mice were treated daily with vehicle, dexamethasone (1 mg/kg IP) or Prevotella histicola strain B (10 mg PO). Ear inflammation was measured, 24 h post ear challenge on day 7. FIG. 18A shows change in ear thickness (n=5 mice/group). FIG. 18B shows that upon termination of study, ear draining lymph nodes were harvested and single cell suspensions were ex vivo stimulated with PMA/Ionomycin for 48 hrs. Protein levels of IL-4 and KC were measured from supernatants by MSD. In FIGS. 18C-18D, BALB/c mice were topically sensitized daily on the ear with 45 nM MC903 from day 1 to 14. Mice were treated daily with vehicle or Prevotella histicola strain B (10 mg PO). Ear inflammation was measured on day 14. FIG. 18C shows change in ear thickness (n=5 mice/group). FIG. 18D shows that qPCR was done from ear tissue to determine Th2 related gene expression. Data are representative of two separate experiments. ***p≤0.01, ****p≤0.0001, ns: not significant as determined by Ordinary one-way ANOVA.

FIG. 19 shows that non-replicating forms of Prevotella histicola strain B protect against KLH-DTH. KLH-DTH was set up as previously described. Change in ear thickness in groups treated with a non-replicating gamma irradiated form of Prevotella histicola strain B (n=5 mice/group). ****p≤0.001, ns: not significant, as determined by unpaired Student's t-test.

FIG. 20 is a graph showing 24-hour ear measurements in a KLH DTH model after treatment with vehicle, dexamethasone, or with Prevotella histicola strain B, anti-TLR2 antibody, IgG1 isotype control, or the combinations as shown.

FIG. 21 is a graph showing 24-hour ear measurements in a KLH DTH model after treatment with vehicle, dexamethasone, or with Prevotella histicola strain B, anti-TLR2 antibody, IgG1 isotype control, or the combinations as shown.

FIGS. 22A and 22B are graphs showing 24-hour ear measurements in a KLH DTH model after treatment with vehicle, dexamethasone, or with Prevotella histicola strain B at day 15 (FIG. 22A) and day 30 (FIG. 22B) after dosing.

FIGS. 23A and 23B are graphs showing 24-hour ear measurements in a KLH DTH model after treatment with vehicle, dexamethasone, or with five separate powder preparations of Prevotella histicola strain B at day 15 (FIG. 23A) and day 30 (FIG. 23B) after dosing.

FIGS. 24A and 24B are graphs showing 24-hour ear measurements in a KLH DTH model after treatment with vehicle, dexamethasone, or with Prevotella histicola strain B in powder or solid dose form at day 8 (FIG. 24A) and day 23 (FIG. 24B) after dosing.

FIG. 25 shows study schema.

### DETAILED DESCRIPTION

In one aspect, described herein is an orally dosed, non-colonizing strain of Prevotella histicola, Prevotella histicola strain B, which modulated the small intestinal axis to suppress systemic inflammation in murine models of type 1 (TH1), type 2 (TH2) and type 3 (TH17) inflammation. Oral therapy with Prevotella histicola strain B in mouse models of disease including delayed type hypersensitivity (type 1, type 2 and 3 inflammation), psoriasis (type 3 inflammation) and EAE (type 3 inflammation) resulted in significant reduction in inflammation and immunopathology. Ex vivo cytokine analyses revealed that Prevotella histicola strain B treatment diminished production of pro-inflammatory cytokines including IL-6, TNF and IL-12p70, and downregulated chemokines including keratinocyte chemoattractant (KC) that are involved in inflammatory cascades. Prevotella histicola strain B treatment also induced IL-10, which plays a role in downregulating inflammation. Prevotella histicola strain B fortifies barrier integrity of gut epithelial cells in vitro. Finally, a non-replicating form of Prevotella histicola strain B was equally efficacious in suppressing inflammation demonstrating that colonization viability is not required for the pharmacological activity of the drug. It does not persist or colonize in the gut and does not modify the background microbiome. These results demonstrate that the small intestinal axis has a central role in controlling systemic inflammation that has not been previously appreciated. It can be modulated by orally-administered, gut-restricted drugs which act directly on host cells in the intestinal mucosa. Prevotella histicola strain B is in clinical development as the first of a next new generation of oral and potent anti-inflammatory drugs with the potential to treat a wide range of inflammatory diseases.

In certain aspects, described herein is an oral therapy with Prevotella histicola strain B, an anti-inflammatory microbe. Prevotella histicola strain B may be a pharmaceutical preparation of a single strain of Prevotella histicola isolated from the duodenum of a human donor. This strain was shown to reduce inflammation in transgenic murine disease models when orally administered. Here the range of anti-inflammatory effects and mechanisms of Prevotella histicola strain B are disclosed and discussed in terms of inflammation resolution mediated by the small intestinal axis.

As reported herein, colonization of the intestine by Prevotella histicola strain B is not required for its pharmacological activity. Thus, in certain embodiments there is no modification of the microbiome. The efficacy of the non-replicating gamma-irradiated form of Prevotella histicola strain B is evidence that its action is dependent on direct interactions with host cells, consistent with effects seen in in vitro assays. This is distinct from reports of live bacterial therapeutics altering the ecology of colonic microbiota. All experiments were done in specific pathogen-free animals with intact intestinal microbiota. The dose-dependent effects of Prevotella histicola strain B were superimposed on this microbial background.

The role of IL-10 in the efficacy of Prevotella histicola strain B in the delayed-type hypersensitivity model indicates the differences between the effects mediated by the small intestinal axis and established therapeutics based on suppressing pro-inflammatory mediators. The pharmacological demonstration of the role that IL-10 plays in the activity of Prevotella histicola strain B shows an alternative way to harness its anti-inflammatory effects.

Thus, in certain embodiments provided herein is a therapeutic approach for common inflammatory diseases suffered by millions of patients. Resolution of inflammation by a non-absorbed oral agent acting via the small intestinal axis has the potential to create a new class of effective, safe, oral medicines, which can be manufactured at reasonable cost for the treatment of inflammatory diseases suffered by millions of patients around the globe.

### Definitions

“Adjuvant” or “Adjuvant therapy” broadly refers to an agent that affects an immunological or physiological response in a patient or subject. For example, an adjuvant might increase the presence of an antigen over time or help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition to a subject. Examples of routes of administration include oral administration, rectal administration, topical administration, inhalation (nasal) or injection. Administration by injection includes intravenous (IV), intramuscular (IM), and subcutaneous (SC) administration. The bacterial compositions described herein can be administered in any form by any effective route, including but not limited to oral, parenteral, enteral, intravenous, intraperitoneal, topical, transdermal (e.g., using any standard patch), intradermal, ophthalmic, (intra)nasally, local, non-oral, such as aerosol, inhalation, subcutaneous, intramuscular, buccal, sublingual, (trans)rectal, vaginal, intra-arterial, and intrathecal, transmucosal (e.g., sublingual, lingual, (trans)buccal, (trans)urethral, vaginal (e.g., trans- and perivaginally), implanted, intravesical, intrapulmonary, intraduodenal, intragastrical, and intrabronchial. In preferred embodiments, the bacterial compositions described herein are administered orally, rectally, topically, intravesically, by injection into or adjacent to a draining lymph node, intravenously, by inhalation or aerosol, or subcutaneously. In some preferred embodiments, the bacterial compositions described herein are administered orally.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)2, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity. “Operational taxonomic units,” “OTU” (or plural, “OTUs”) refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. In 16S embodiments, OTUs that share ≥97% average nucleotide identity across the entire 16S or some variable region of the 16S are considered the same OTU (see e.g. Claesson M J, Wang Q, O'Sullivan O, Greene-Diniz R, Cole J R, Ros R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940). In embodiments involving the complete genome, MLSTs, specific genes, or sets of genes OTUs that share ≥95% average nucleotide identity are considered the same OTU (see e.g. Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940). OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Such characterization employs, e.g., WGS data or a whole genome sequence.

A “combination” of two or more monoclonal microbial strains includes the physical co-existence of the two monoclonal microbial strains, either in the same material or product or in physically connected products, as well as the temporal co-administration or co-localization of the monoclonal microbial strains.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size (e.g., in an animal tumor model)).

As used herein, “engineered bacteria” are any bacteria that have been genetically altered from their natural state by human intervention and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., Lupus, Scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease) and includes, for example, checkpoint inhibitors, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include the number of immune cells, bacterial cells, stromal cells, myeloid derived suppressor cells, fibroblasts, metabolites; the level of a cytokine; or another physical parameter (such as ear thickness (e.g., in a DTH animal model) or tumor size (e.g., in an animal tumor model).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. Immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM(PS)2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLRlO and TLRI 1. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The term “isolated” or “enriched” encompasses a microbe, bacteria or other entity or substance that has been (1) separated from at least some of the components with which it was associated when initially produced (whether in nature or in an experimental setting), and/or (2) produced, prepared, purified, and/or manufactured by the hand of man. Isolated microbes may be separated from at least about 10%, about 20%, about 30%, about 40%, about 50%, about 60%, about 70%, about 80%, about 90%, or more of the other components with which they were initially associated. In some embodiments, isolated microbes are more than about 80%, about 85%, about 90%, about 910%, about 92%, about 93%, about 94%, about 95%, about 96%, about 97%, about 98%, about 99%, or more than about 99% pure, e.g, substantially free of other components. The terms “purify,” “purifying” and “purified” refer to a microbe or other material that has been separated from at least some of the components with which it was associated either when initially produced or generated (e.g., whether in nature or in an experimental setting), or during any time after its initial production. A microbe or a microbial population may be considered purified if it is isolated at or after production, such as from a material or environment containing the microbe or microbial population, and a purified microbe or microbial population may contain other materials up to about 10%, about 20%, about 30%, about 40%, about 50%, about 60%, about 70%, about 80%, about 90%, or above about 90% and still be considered “isolated.” In some embodiments, purified microbes or microbial population are more than about 80%, about 85%, about 90%, about 91%, about 92%, about 93%, about 94%, about 95%, about 96%, about 97%, about 98%, about 99%, or more than about 99% pure. In the instance of microbial compositions provided herein, the one or more microbial types present in the composition can be independently purified from one or more other microbes produced and/or present in the material or environment containing the microbial type. Microbial compositions and the microbial components thereof are generally purified from residual habitat products.

“Metabolite” as used herein refers to any and all molecular compounds, compositions, molecules, ions, co-factors, catalysts or nutrients used as substrates in any cellular or microbial metabolic reaction or resulting as product compounds, compositions, molecules, ions, co-factors, catalysts or nutrients from any cellular or microbial metabolic reaction.

“Microbe” refers to any natural or engineered organism characterized as a bacterium, fungus, microscopic alga, protozoan, and the stages of development or life cycle stages (e.g., vegetative, spore (including sporulation, dormancy, and germination), latent, biofilm) associated with the organism.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state microbiome. The microbiome may be native to the subject or patient, or components of the microbiome may be modulated, introduced, or depleted due to changes in health state or treatment conditions (e.g., antibiotic treatment, exposure to different microbes). In some aspects, the microbiome occurs at a mucosal surface. In some aspects, the microbiome is a gut microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present in a sample. In some embodiments, the microbiome profile indicates the relative or absolute amount of each bacterial strain detected in the sample.

“Modified” in reference to a bacteria broadly refers to a bacteria that has undergone a change from its wild-type form. Examples of bacterial modifications include genetic modification, gene expression, phenotype modification, formulation, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium that increase or decrease virulence.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g. Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361: 1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6: 431-440. Konstantinidis K T, Ramette A, and Tiedje J M. 2006. The bacterial species definition in the genomic era. Philos Trans R Soc Lond B Biol Sci 361: 1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a substance is “pure” if it is substantially free of other components. The terms “purify,” “purifying” and “purified” refer to a microbe or other material that has been separated from at least some of the components with which it was associated either when initially produced or generated (e.g., whether in nature or in an experimental setting), or during any time after its initial production. A microbe may be considered purified if it is isolated at or after production, such as from one or more other bacterial components, and a purified microbe or microbial population may contain other materials up to about 10%, about 20%, about 30%, about 40%, about 50%, about 60%, about 70%, about 80%, about 90%, or above about 90% and still be considered “purified.” In some embodiments, purified microbes are more than about 80%, about 85%, about 90%, about 91%, about 92%, about 93%, about 94%, about 95%, about 96%, about 97%, about 98%, about 99%, or more than about 99% pure. Bacterial compositions and the microbial components thereof are, e.g., purified from residual habitat products.

“Residual habitat products” refers to material derived from the habitat for microbiota within or on a subject. For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a human or animal and that only microbial cells are detectable. In one embodiment, substantially free of residual habitat products may also mean that the microbial composition contains no detectable viral (including microbial viruses (e.g., phage)), fungal, mycoplasmal contaminants. In another embodiment, it means that fewer than 1×10-2%, 1×10-3%, 1×10-4%, 1×10-5%, 1×10-6%, 1×10-7%, 1×10-8% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10-8 or 10-9), such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), serology and antigen analysis, enzymatic and metabolic analysis, and methods using instrumentation such as flow cytometry with reagents that distinguish desired constituents from contaminants.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

The terms “subject” or “patient” refers to any animal. A subject or a patient described as “in need thereof” refers to one in need of a treatment for a disease. Mammals (i.e., mammalian animals) include humans, laboratory animals (e.g., primates, rats, mice), livestock (e.g., cows, sheep, goats, pigs), and household pets (e.g., dogs, cats, rodents). For example, the subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

As used herein, the term “treating” a disease in a subject or “treating” a subject having or suspected of having a disease refers to subjecting the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof.

### Bacteria

In certain aspects, provided herein are bacterial compositions (e.g., pharmaceutical compositions) comprising Prevotella histicola useful for the treatment and/or prevention of psoriasis (e.g., mild to moderate psoriasis) and/or atopic dermatitis (e.g., mild to moderate atopic dermatitis) and methods of using such bacterial compositions (e.g., for the treatment of psoriasis, for the treatment of atopic dermatitis), e.g., in a subject, e.g., in a human subject. In some embodiments, the bacterial compositions comprise whole Prevotella histicola bacteria (e.g., live bacteria, killed bacteria, attenuated bacteria). In some embodiments, the bacterial composition (e.g., pharmaceutical composition) comprises only one strain of bacteria, e.g., Prevotella histicola.

In some embodiments, the Prevotella histicola is Prevotella Strain B 50329 (NRRL accession number B 50329) (also referred to as “Prevotella histicola Strain B” or “Prevotella Strain B”). In some embodiments, the Prevotella strain is a strain comprising at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

Prevotella histicola Strain B can be cultured according to methods known in the art. For example, Prevotella histicola can be grown in ATCC Medium 2722, ATCC Medium 1490, or other medium using methods disclosed, for example in Caballero et al., 2017. “Cooperating Commensals Restore Colonization Resistance to Vancomycin-Resistant Enterococcus faecium” Cell Host & Microbe 21:592-602, which is hereby incorporated by reference in its entirety.

In some embodiments, the bacterial compositions comprise whole Prevotella histicola bacteria (e.g., live bacteria, killed bacteria, attenuated bacteria).

In some embodiments, the bacterial composition comprises about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², and/or 1×10¹³ total cells of Prevotella histicola.

In some embodiments, the bacterial composition comprises at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², or 1×10¹³ total cells of Prevotella histicola.

In some embodiments, the bacterial composition comprises at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², or 1×10¹³ total cells of Prevotella histicola.

In some embodiments, the bacterial composition comprises from about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ to about 8×10¹¹ total cells of Prevotella histicola.

In some embodiments, the bacterial composition comprises from about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ to about 1×10¹² total cells of Prevotella histicola.

In some embodiments, the bacterial composition comprises about 1.6×10¹⁰ total cells of Prevotella histicola, e.g., of Prevotella Strain B 50329.

In some embodiments, the bacterial composition comprises about 8×10¹⁰ total cells of Prevotella histicola, e.g., of Prevotella Strain B 50329.

In some embodiments, the bacterial composition comprises about 1.6×10¹¹ total cells of Prevotella histicola, e.g., of Prevotella Strain B 50329.

In some embodiments, the bacterial composition comprises about 3.2×10¹¹ total cells of Prevotella histicola, e.g., of Prevotella Strain B 50329.

In some embodiments, the bacterial composition comprises about 8×10¹¹ total cells of Prevotella histicola, e.g., of Prevotella Strain B 50329.

In some embodiments, the bacterial composition comprises about 1.6×10¹⁰ to about 8×10¹¹ total cells of Prevotella histicola, e.g., of Prevotella Strain B 50329.

In some embodiments, the bacterial composition comprises about 1.6×10¹⁰ to about 1.6×10¹¹ total cells of Prevotella histicola, e.g., of Prevotella Strain B 50329.

In some embodiments, the bacterial composition comprises about 1.6×10¹¹ to about 8×10¹¹ total cells of Prevotella histicola, e.g., of Prevotella Strain B 50329.

In some embodiments, the bacterial composition comprises about 8×10¹⁰ to about 8×10¹¹ total cells of Prevotella histicola, e.g., of Prevotella Strain B 50329.

In some embodiments, the Prevotella bacteria may be quantified based on total cells, e.g., total cell count (TCC) (e.g., determined by Coulter counter).

In some embodiments, the bacterial composition comprises at least 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises at most 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises about 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises about 1 g, 2 g, 2.5 g, 2.6 g, 2.61 g, 2.62 g, 2.63 g, 2.64 g, 2.65 g, 2.66 g, 2.67 g, 2.68 g, 2.69 g, 2.70 g, 2.71 g, 2.72 g, 2.73 g, 2.74 g, 2.75 g, 2.76 g, 2.77 g, 2.78 g, 2.79 g, 2.80, 2.81 g, 2.82 g, 2.83 g, 2.84 g, 2.85 g, 2.86 g, 2.87 g, 2.88 g, 2.89 g, 2.90 g, 3 g, 4 g, 5 g, 10 g, 20 g, 30 g, 40 g, or 50 g of Prevotella histicola.

In some embodiments, the bacterial composition is administered orally. In some embodiments, the administration to the subject once daily. In some embodiments, the bacterial composition is administered in 2 or more doses (e.g., 3 or more, 4 or more or 5 or more doses). In some embodiments, the administration to the subject of the two or more doses are separated by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days.

In some embodiments, the bacterial composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the bacterial composition is administered once daily for 1, 2, 3, 4, 5, 6, 7, 8, 9, 10, 11, 12, 13, 14, 15, or 16 weeks. In some embodiments, the bacterial composition is administered once daily for at least 1, 2, 3, 4, 5, 6, 7, 8, 9, 10, 11, 12, 13, 14, 15, or 16 weeks.

In some embodiments, the bacterial composition is formulated as a capsule or a tablet. In some embodiments, the bacterial formulation (e.g., composition) comprises an enteric coating or micro encapsulation. In some embodiments, the capsule is an enteric coated capsule. In some embodiments, the enteric coating allows release of the bacterial composition in the small intestine, e.g., in the upper small intestine, e.g., in the duodenum. In some embodiments, the enteric coating comprises HPMC.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla or a chimpanzee).

In some embodiments, the Prevotella histicola bacteria is a strain of Prevotella bacteria comprising one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) proteins listed in Table 1 and/or one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more) genes encoding proteins listed in Table 1. In some embodiments, the Prevotella bacteria comprises all of the proteins listed in Table 1 and/or all of the genes encoding the proteins listed in Table 1.

TABLE 1
Exemplary Prevotella proteins

| Seq. ID. No. | Name | Uniprot ID | Amino Acid Sequence |
|---|---|---|---|
| 1 | Cluster: | G6ADE1 | MNLKTFTKTVLCFALFAVSAITAKAADHLAIVGE |
| | Uncharacterized | | AVWGGWDLVKATAMVKSPNNPDVFMATVHLNAGK |
| | protein | | GFKFLTEREWGKLEYRSGASDVVLKSGIRYKLYA |
| | | | SIGASEDGKFKVSESANYEIICDLARKTVEVKKV |
| | | | AYQAKEIRYAALWMIGDATAGDWDYNNGVLLSQD |
| | | | SGNPTCYTATVELKEGEFKFTTNKQWGYDHSVYI |
| | | | ERDVNDQNKIVEGGEDNKWRITEDGMYNVTVDVP |
| | | | TKTISIKQIDDPAGHKPQFGNDVILVGDATIAGW |
| | | | NLDNAIYLEHTGQAGRVFKTTTYLEAGKGFKFLS |
| | | | MLSYDDIDYRPANNTVLNPGVPGTFVPSLPSSTD |
| | | | TKFSVERSGNYDIVCNMNNRTVVVTLSENQVLVN |
| | | | YPALWLIGSATSAGWNPGKAVELKRSEADPAVYT |
| | | | ARVQLKKGEFKILTSKNVGFDQPTYYRDSTNEHR |
| | | | IVFGVDGDEVAKKDCKWTLSENAEGTYDVTVDIE |
| | | | AMTIFCDKVNMDEPSVESTDKELILIGDATYSAW |
| | | | DLPKSIVMTPVGPTTFKAVTHLEAGKEFKFLTEL |
| | | | AWKRYEYRAESLRKELQEGSMSMLVPYRYTNDKD |
| | | | DKDHDFKFVVKESGNYEIVCDLYIPALIIRKVRY |
| | | | QDTPVTYSSLWIVGSATPGGWTIERGIKMTQDEN |
| | | | YPTKFTAKANLVPGELKFATNKFADFTQDFFFRG |
| | | | KDDYTAVLGGNDNKWNITEAGTYSVTIDVASKRV |
| | | | TITKPARNAPTGISTVDSSDEAPAEYFTLNGIKV |
| | | | TTPSSGIYIKRQGGRTTKVVMK |
| 2 | Nicotinamide_ | P24520 | MDTYQILDIIGCIVGLIYIYQEYKASIWLWMTGI |
| | riboside_ | | IMPVIYMEVYYEAGLYADEGMQIYYTLAAIYGYL |
| | transporter_PnuC | | YWKLGKKKGTEDKEIPITHFPRRYIIPAIIVFFV |
| | | | LWIALYYILICFTNSTVPVLDSEGNALSFIGLWA |
| | | | LAKKYLEQWWIWIVVDAELSALYIYKGIPFTAML |
| | | | YALYTVIAVAGYFKWRRYIKQQK |
| 3 | Pectate_ | Q8GCB2 | MRVRLYKNILLFLFLWVNTLACVSADTSRTVESQ |
| | trusaccgarude- | | PIENGLIITESKGWLETIYAKWKPVAEADGYYVY |
| | lyase | | VKGGQYADYSKVDSELIRVYNGYVRVDIPGLKAG |
| | | | TYSLKIVAVKGGKETQSSEVTGLKVLNYVREGFA |
| | | | HKNYSGVGAYNDDGTLKSGAVVIYVNKDNAKTVS |
| | | | AHLGKTTFIGLQAILNAYQKGNITTPLSVRILGL |
| | | | LRNGDTDTFGSSTEGIQIKGKQADSEMNITIEGI |
| | | | GEDASIYGFGFLVRNAKSVEFRNLGIMRAMDDGV |
| | | | SLDTNNSNIWIHHMDLFYGKASGGDHIKGDGSID |
| | | | VKTDSKYVTIDNCHFWDTGKTSMCGMKKETGPNY |
| | | | ITYHHNWFDHSDSRHARVRTMSVHLWNNYYDGCA |
| | | | KYGIGATMGCSVFSENNYFRATKNPILISKQGSD |
| | | | AKGTGKFSGEPGGMVKEYGSLFTEKGAESTYTPI |
| | | | SYADNNSSFDFYHAISRNEKVPASVKTLNGGNIY |
| | | | NNFDTDAALMYSYTPDATALVPSQVTGFYGAGRL |
| | | | NHGSLQFKFNNAVEDTNSTPIPALEALIDAYSGK |
| 4 | Glycosyltranserase_ | Q9AET5 | MKYNIAYCIEGFYNHGGMERILSVCANLLSDIYS |
| | Gtf1 | | ITIIVANQRGREHAYNLAQNVNVVDLGVSCKNYK |
| | | | EEYKKSLTRYLQDHQFSVVISLAGLELFFLPQIK |
| | | | DGSKKVMWFHFAFDVSKMFLSERFHGWKLNLLYY |
| | | | IHTIRRIYFAKKFDTIVVLSKSDCDSWSRFCNNV |
| | | | KYIYNPITIDRKVISNLSEESVIAVGRLGWQKGF |
| | | | DFLIDSWVLVDDKHPDWHLDIFGEGPDRLELQHQ |
| | | | IDRKGLHDKVRLCGVTKQIEEEYGKHSIYVMSSR |
| | | | AEGFPLALLEASSCGLPMISFNCHQGPNEIIQEG |
| | | | ENGFLVDKVGDIYTLSDRICKLIEDNNLRNMMGK |
| | | | KALDSSFRFEGEVIKKDWISLLKQLI |
| 5 | Cluster: | A0A096B759 | MKRLFFMFLFLGTITMNSLAQEEKPIKYETKNFS |
| | Protein | | LPDKMPLYPGGDGALRAFLSLNLHYPEKAQAFGV |
| | TonB | | EGRSLMKFCVSSDGSIKDISAVDCKITNYNRTEF |
| | | | NKLPLSKQESLKKECAKAFAKEAARVIRLMPKWE |
| | | | PAELNGKKMNVYYSLPFTFKLR |
| 6 | Cluster: | G6AEN6 | MNYPLFIARKIYNGGDRTRKVSKPAIRIATIGVA |
| | Uncharacterized | | IGLAVMIISVGVVLGFKHTIRNKVVGFGSDTTVA |
| | protein | | NFLTLQSSEQYPIQITDSLVKSLQITPGIKHVQR |
| | | | YDYTQGILKTDNDFLGVLLKGVGPDFDSTFTHEN |
| | | | MVEGSLPHFHDNESQQKIVISKTIADKLNLKVGQ |
| | | | RIFAYFINKQGVRTRKFTITGIYATNMKQFDSQT |
| | | | CFTDIYTTNKLNGWEPDQYSGAELQVDNFSQLTP |
| | | | ISMRVLNKVKNTVDHYGGTYSSENIIEQNPQIFS |
| | | | WLDLMDMNVWIILALMISVAGVTMISGLLIIILE |
| | | | RTQMIGILKALGSRNRQIRHIFLWFATFIIGKGL |
| | | | LWGNIIGLGCILFQSWTGLVKLDPQTYYVNTVPV |
| | | | EINIPLIIALNMVTMLVCLVILIAPSYLISHIHP |
| | | | AKSMHYE |
| 7 | Bifunctional_ | P9WHG9 | MEDKFIYTDKERKLSYQILDELKDTLDKSFLEND |
| | (p)ppGpp_ | | LPMLQVQLKDSVAKNTIHRNVFGLNPILCSLQTA |
| | synthase/ | | AIAVKDIGLKRDSVIAILLHQSVQDGYITLEDID |
| | hydrolase_ | | NRFGKSVAKIIHGLIRIQTLYQKNPIIESENFRN |
| | RelA | | LLLSFAEDMRVILIMIADRVNLMRQIRDAEDKEA |
| | | | QHKVAEEASYLYAPLAHKLGLYQLKRELEDLSLK |
| | | | YLEHDAYYLIKDKLNATKASRDAYINQFIAPVRE |
| | | | RLTAGGLRFHIKGRTKSIHSIWQKMKKQKCGFEG |
| | | | IYDLFAIRIILDAPLEKEKIQCWQAYSIVTDMYQ |
| | | | PNPKRLRDWLSVPKSNGYECLHITVLGPEKKWVE |
| | | | VQIRTERMDEIAEHGLAAHWRYKGIKEEGGLDDW |
| | | | LASIRAALEAGDNLEVMDQFKSDLYEKEIYVFTP |
| | | | KGDLLKFPKGATILDFAYHIHSKVGNQCVGGKIN |
| | | | AKNVSLRTELHSGDTVEILTSATQKPKAEWLKIV |
| | | | KSSRAKAKIRLALKETQIKDGLYAKELLERRFKN |
| | | | KKIEIEESTMGHLLRKLGFKEVSEFYKQVADEKL |
| | | | DPNYIIEEYQKVYNHDHNLNQPKETESAENFEFE |
| | | | NPTNEFLKKNDDVLVIDKNLKGLDFSLAKCCHPI |
| | | | YGDPVFGFVTVNGGIKIHRTDCPNAPEMRKRFGY |
| | | | RIVKARWSGKGSSQYAITLRVIGNDDIGIVSNIT |
| | | | NVISKDEKIVMRSINIDSHDGLFSGNLVVLLDDN |
| | | | SKLNMLIKKLRTVKGVKQVTRI |
| 8 | Vitamin_B12_ | P06609 | MKRRIFLFVALSVSIVILFGLNLIIGSVHIPLSD |
| | import_system_ | | ILTILSGSFTGKESWRFIIWDSRLPQALTAMLCG |
| | permase_ | | SSLAVCGLMLQTAFRNPLAGPDVFGISSGASLGV |
| | protein_BtuC | | ALVMLLLGGTVETSMFTASGFLAILIVAFAGAIL |
| | | | VTAFILFLSSVVRNSVLLLIVGIMVGYVASSAVT |
| | | | LLNFFSSEDGVKGYIVWGMGNFGGVSMSHIPLFA |
| | | | FLCLAGIIASFLLVKPLNILLLGPQYAESLGISI |
| | | | RRIRNILLVVVGILTAVTTAFCGPISFIGLAAPH |
| | | | VARLLFRTENHQKLLPGTLLVGTVVALLCNLICF |
| | | | LPRESGMIPLNAVTPLIGAPIIIYVIMKRH |
| 9 | NADH- | P33599 | MKLENKEFGFDSFATEMARLKNEKHFDYLVTVVG |
| | quinone_ | | EDFGTEEGLGCIYILENTSTHERCSVKQLAKKVG |
| | oxidoreductase_ | | EEFVIPSVIKLWADADLLEREVYDFYGIKFLGHP |
| | subunit_C/D | | DMRRLFLRNDFKGYPLRKDYDMDPAKNMYTTEDD |
| | | | VELDTTTEWNLDKNGELVGTQHALFTDDNFVVNT |
| | | | GPQHPSTHGVLRLQTVLDGETVTNTYPHLGYIHR |
| | | | GIEKLCEQFTYPQTLALTDRMNYLSAMMNRHALV |
| | | | GVIEEGMGIELSERILYIRTIMDELQRIDNHLLY |
| | | | TACCAQDLGALTAFLYGMRDREHVLNVMEETTGG |
| | | | RLIQNYYRIGGLQADIDPNFVSNVKELCKYLRPM |
| | | | IQEYVDVFGDNVITHQRFEGVGVMDEKDCISYGV |
| | | | TGPAGRASGWKNDVRKYHPYAMYDKVNFEEITLT |
| | | | NGDSMDRYFCHIKEIYQSLNIIEQLIDNIPEGEF |
| | | | YIKQKPIIKVPEGQWYFSVEGASGEFGAYLDSRG |
| | | | DKTAYRLKFRPMGLTLVGAMDKMLRGQKIADLVT |
| | | | TGAALDFVIPDIDR |
| 10 | FKBP- | P45523 | MRTSTQSKDMGKKQEYKLRNEEFLHNISKKDSIK |
| | type_pentidyl- | | TLPHGIFYEIIKEGSGEGTVQPRSIVICNYRGSL |
| | prolyl_cis- | | ISGQVFDDSWQKPTPEAFRLNELITGLQIALCAM |
| | trans_isomerase | | HKGDSWRIYIPYQEGYGSKRNADIPAFSTLIFDI |
| | | ELINIA | |
| 11 | Putative- | P9WKJ3 | MADNKIAKESVKREVIAGERLYTLLVYSENVAGV |
| | acetolactate_ | | LNQIAAVFTRRQVNIESLNVSASSIEGIHKYTIT |
| | synthase_ | | AWSDAATIEKITKQVEKKIDVIKADYYEDSDLFI |
| | small_subunit | | HEVGLYKIATPILLENAEVSRAIRKRNARMMEVN |
| | | | PTYSTVLLAGMTDEVTALYHDLKNFDCLLQYSRS |
| | | | GRVAVTRGFSEPVSDFLKSEEESSVL |
| 12 | Serine/ | P0AGE4 | MKKKVKIGLLPRVIIAILLGIFFGYFMPTPLARV |
| | threonine_ | | FLTFNGIFSQFLGFMIPLIIIGLVTPAIADIGKG |
| | transporter_ | | AGKLLLVTVIIAYVDTVVAGGLAYGTGLCLFPSM |
| | SstT | | IASTGGAMPHIDKATELAPYFSINIPAMADVMSG |
| | | | LVFSFMLGLGIAYGGLTATKNIFNEFKYVIEKVI |
| | | | AKAIIPLLPLYIFGVFLNMAHNGQAQQILLVFSQ |
| | | | IIIVILVLHVFILVYQFCIAGAIIRRNPFRLLWN |
| | | | MMPAYLTALGTSSSAATTPVTLEQTMKNGVGKEI |
| | | | AGFVVPLCATIHLSGSAMKITACALTICLLVGLP |
| | | | HDPALFIYFILMLSIIMVAAPGVPGGAIMAALAP |
| | | | LASILGFNSEAQALMIALYIAMDSFGTACNVTGD |
| | | | GAIALVVNKMFGKKER |
| 13 | Cluster: | G6AJ07 | MKKLLLLVCAAVMSLSASAQAGDKALGAQLVFGS |
| | uncharacterized | | ETNSLGFGVKGQYYFTDHIRGEGSFDYFLKNKGI |
| | protein | | SMWDINANVHYLFDVADKFKVYPLAGLGYTNWSY |
| | | | KYEYAGAPVVEGSDGRLAVNLGGGVEYELTKNLN |
| | | | VNAEAKYQIISNYNQLVLGVGVAYKF |
| 14 | Heterocyst_ | P22638 | MHFYCTKSSLDTMSERYVKRMIAKLASQGKTVIS |
| | differentiation_ | | IAHRFSTIMDAKHIILLAKGKVVAEGTHQELLKT |
| | ATP-binding | | SEDYRKLWSDQNDEID |
| | protein | | |
| 15 | UDP-2,3- | Q9I2V0 | MKNVYFLSDAHLGSLAIAHRRTQERRLVRFLDSI |
| | diacylglucosamine_ | | KHKASAVYLLGDMFDFWDEYKYVVPKGFTRFLGK |
| | hydrolase | | VSELTDMGVEVHFFTGNHDLWTYGYLEEECGVIL |
| | | | HRKPVTMEIYGKVFYLAHGDGLGDPDPMFQFLRK |
| | | | VFHNRVCQRLLNFFHPWWGMQLGLNWAKKSRLKR |
| | | | ADGKEMPYLGEDKEYLVRYTKDYMRSHKDIDYYI |
| | | | YCHRHIELDLTLSGKVRMLILGDWIWQFTYAVFD |
| | | | GEHMFLEEYIEGESKP |
| 16 | Anacrobic_ | P0A9C0 | MNSKQNDNYDVIIIGGGITGAGTARDCALRGLKV |
| | glycerol-3- | | LLVEKFDFTNGATGRNHGLLHSGARYAVTDPESA |
| | phosphate_ | | TECIKENMVLRRIAKHCIEETDGLFITLPEDDIN |
| | dehydrogenase | | YQKTFVEACARAGISANIISPEEALRLDPSVNPD |
| | | | LLGAVRVPDASVDPFHLTTANVLDARQHGADVLT |
| | | | YHEVVAILTSNGRVEGVRLRNNHTGEEIEKHAVL |
| | | | VINAAGIWGHDIAKMADIKINMFPAKGTLLVFGH |
| | | | RVNKMVINRCRKPANADILVPDDAVCVIGTTSDR |
| | | | VPYDTVDNLKITSEEVDTLIREGEKLAPSLATTR |
| | | | ILRAYAGVRPLVAADNDPTGRSISRGIVCLDHEK |
| | | | RDGLTGMITITGGKMMTYRLMAEQATDLACKKLG |
| | | | INKTCETATTPLPGTAGKDSDNPHHTYSTAHKAA |
| | | | KGRQGNRVKEIDERTEDDRALICECEEVSVGEAK |
| | | | YAIEELHVHDLLNLRRRTRVGMGTCQGELCACRA |
| | | | AGVMCENGVKVDKAMTDLTKFINERWKGMRPVAW |
| | | | GSTLDEAQLTTIIYQGLCGLGI |
| 17 | Anaerobic_ | P13033 | MRYDTIIIGGGLSGLTAGITLAKAGQKVCIVSAG |
| | glycerol-3- | | QSSLHFHSGSFDLLGYDADGEVVTHPLQAIADLK |
| | phosphate_ | | AEHPYSKIGISNIEHLASQAKTLLCEAGISVMGN |
| | dehydrogenase | | YEQNHYRVTPLGTLKPAWLTTEGYAMIDDPEILP |
| | | | WKKVELLNIQGFMDFPTQFIAENLRMMGVECQIK |
| | | | TFTTDELSTARQSPTEMRATNIAKVLANKDALSK |
| | | | VSERINAISGDPDALLLPAVLGFSNAESLDEMKQ |
| | | | WIKKPVQYIATLPPSVSGVRTTILLKRLFAQAGG |
| | | | TLLIGDSATTGQFSGNHLVSITTDHLPDEKLYAD |
| | | | HFILASGSFMSHGIRSNYAGVYEPVFKLDVDAAE |
| | | | KRDDWSVTNAFEAQPYMEFGVHTDKDFHATKDGK |
| | | | NIENLYAIGSVLSGHNSIKHADGTGVSLLTALYV |
| | | | AKKITGKG |
| 18 | Anaerobic_ | P0A996 | MAEGIQLKNISGNNLEQCLKCSICTAYCPVSAVE |
| | glycerol-3- | | PKYPGPKQSGPDQERYRLKDSKFFDEALKMCLNC |
| | phosphate_ | | KRCEVACPSGVRIADIIQASRITYSTHRPIPRDI |
| | dehydrogenase | | MLANTDFVGTMANMVAPTVNATLGLKPVKAVLHG |
| | | | VMGIDKHRTFPAYSSQKFETWYKRMAAKKQDSYS |
| | | | KHVSYFHGCYVNYNFPQLGKDLVKIMNAVGYGVH |
| | | | LLEKEKCCGVALIANGLSGQARRQGKVNIRSIRK |
| | | | AAEQNRIVLTTSSTCTFTMRDEYEHLLDTKTDDV |
| | | | RENITLATRFLYRLIEKGDIKLAFRKDFKMRTAY |
| | | | HSACHMEKMGWIIYSTELLKMIPGLELIMLDSQC |
| | | | CGIAGTYGFKKENYQRSQEIGEGLFKQIKELNPD |
| | | | CVSTDCETCKWQIEMSTGYEVKNPISILADALDV |
| | | | EETIKLNQ |
| 19 | Glycerol_ | P18156 | MMIKNIVLSIPISLIIYLNHLIMEYSMTTQFLME |
| | uptake_ | | LIGTLILVLFGDGVCACVTLNKSKGQKAGWVVIT |
| | facilitator_ | | IAWGLAVCMGVLVAGPYTGAHLNPAVSIGLAVAG |
| | protein | | MFPWSSVPYYIVAQMIGGFLGGLLVWFFYKDHYD |
| | | | ATDDEAAKLGTFCTSPAIRNYKMNFLSEVIATLV |
| | | | LVFIIISFSVDGNTGDAEHFKFGLAALGPIPVTL |
| | | | LIIALGMSLGGTTGYAMNPARDLSPRLAHAVCMK |
| | | | GDNDWSYSWIPVLGPIIGAIIAGFCGAALLLV |
| 20 | Serine/threonine- | Q97PA9 | MSEKIIPSNEPAQAASEPIKASYTEYTVIPSQGY |
| | protein_kinase_ | | CQFVKCKKGDQPVVLKGLKEAYRERVLLRNALKR |
| | StkP | | EFKQCQRLNHPGIVRYQGLVDVEGYGLCIEEEYV |
| | | | DGRTLQAYLKESHTDDEKITIVNQIADALRYAHQ |
| | | | QGVAHRNLKPSNILITKQGDHVKLIDFNVLSLDD |
| | | | VKPTADTTRFMAPELKDETMTADGTADIYSLGTI |
| | | | MKVMGLTLAYSEVIKRCCAFKRSDRYSDIDEFLA |
| | | | DFNHDGSSFSMPKIGKGTVVIGFIAVVVIALAAL |
| | | | AYNYGGALVDQVGKIDVTSIFKSDAETAPEDSAM |
| | | | VKSVEQNNNDSVADEAPATGKLAFMNTMKPALYK |
| | | | DLDRLFAKHSDDRAKLNRAIKVYYRGLIQANDTL |
| | | | DNEQRAELDRVFGNYVKQKKAALK |
| 21 | Cluster: | G6AHI1 | MLVAQLFVGVLQAQKPVQNRRQAVGQSMERQGLV |
| | D-alanyl- | | NVKAVVPSIKVALMYARTDNFCHRMALS |
| | D-alanine | | |
| | dipeptidase | | |
| 22 | Anaerobic_C4- | P0ABN5 | MITGLVIIQLLIVLALIFIGARVGGIGLGIYGMI |
| | C4-dicarboxylate_ | | GVFILVYGFGLAPGSAPIDVMMIIVAVITAASAL |
| | transporter_ | | QASGGLEYLVGVAAKFLQKHPDHITYFGPITCWL |
| | DcuA | | FCVVAGTAHTSYSLMPIIAEIAQTNKIRPERPLS |
| | | | LSVIAASLGITCSPVSAATAALISQDLLGAKGIE |
| | | | LGTVLMICIPTAFISILVAAFVENHIGKELEDDP |
| | | | EYKRRVAAGLINPEAACEEVQKAENEHDPSAKHA |
| | | | VWAFLFGVALVILFGFLPQLRPEGVSMSQTIEMI |
| | | | MMSDAALILLVGKGKVGDAVNGNIFKAGTVINAV |
| | | | VAIFGIAWMGNTFYVGNEKILDAALSSMISSTPI |
| | | | LFAVALFLLSIMLFSQAATVTTLYPVGIALGTNP |
| | | | LLLIAMFPACNGYFFLPNYPTEVAAIDFDRTGTT |
| | | | RVGKYVINHSFQIPGFITTIVSTLLGVLMVQFFR |
| 23 | L-asparaginase_2 | P00805 | MRILKITFVTVLALVMSTVVFAQKPKIRIIATGG |
| | | | TIAGVSASATSSAYGAGQVGVQTLIDAVPQIKDI |
| | | | ADVSGEQLVNIGSQDMNDEVWLKLAKRINDLLNK |
| | | | EGYDGVLITHGTDTMEETAYFLSLTVHTDKPVVM |
| | | | VGSMRPSTAISADGPANLYNGICTLVDPSSKGHG |
| | | | VMVCMNNELFEAKSVIKTHTTDVSTFKGGLYGEM |
| | | | GYVYNGKPYFLHKPVAKQGLTSEFNVDNLTSLPK |
| | | | VGIVYGYANCSPLPIQAFVNAKFDGIVLAGVGDG |
| | | | NFYKDVFDVALKAQNSGIQIVRSSRVPFGPTNLN |
| | | | GEVDDAKYHFVASLNLNPQKARVLLMLALTKTKD |
| | | | WQKIQQYFNEY |
| 24 | Trehalose_ | P9WQ19 | MALACAMTMSASAQMGTNPKWLGDAIFYQIYPSS |
| | synthase/ | | YMDTDGNGIGDLPGITQKLDYIKSLGVNAIWLNP |
| | amylase_TreS | | VFESGWFDGGYDVIDFYKIDPRFGTNTDMVNLVK |
| | | | EAHKRGIKVCLDLVAGHTSTKCPWFKESANGDRN |
| | | | SRYSDYFIWTDSISEADKKEIAERHKEANPASST |
| | | | HGRYVEMNAKRGKYYEKNFFECQPALNYGFAKPD |
| | | | PNQPWEQPVTAPGPQAVRREMRNIMAFWFDKGVD |
| | | | GFRVDMASSLVKNDWGKKEVSKLWNEMREWKDKN |
| | | | YPECVLISEWSDPAVAIPAGFNIDFMIHFGIKGY |
| | | | PSLFFDRNTPWGKPWPGQDISKDYKFCYFDKAGK |
| | | | GEVKEFVDNFSEAYNATKNLGYIAIPSANHDYQR |
| | | | PNIGTRNTPEQLKVAMTFFLTMPGVPFIYYGDEI |
| | | | GMKYQMDLPSKEGSNERAGTRTPMQWTSGPTAGF |
| | | | STCNPSQLYFPVDTEKGKLTVEAQQNDPRSLLNY |
| | | | TRELTRLRHSQPALRGNGEWILVSKESQPYPMVY |
| | | | KRTSGGETVVVAINPSDKKVSANIAHLGKAKSLI |
| | | | MTGKASYKTGKTEDAVELNGVSAAVFKIAE |
| 25 | Ribitol-5- | Q720Y7 | MNIAVIFAGGSGLRMHTKSRPKQFLDLNGKPIII |
| | phosphate_ | | YTLELFDNHPGIDAIVVACIESWIPFLEKQLRKF |
| | cytidyl | | EINKVVKIVPGGESGQASIYNGLCAAEAYIKSKN |
| | yltransferase | | VASEDTTVLIHDGVRPLITEETITDNINKVAEVG |
| | | | SCITCIPATETLVVKQHDGSLEIPSRADSLIARA |
| | | | PQSFLLSDILTAHRRAIDEKKNDFIDSCTMMSHY |
| | | | GYRLGTIIGPMENIKITTPTDFFVLRAMVKVHED |
| | | | QQIFGL |
| 26 | UDP-Glc: | B5L3F2 | MTEKKSVSIVLCTYNGTKYLQEQLDSILAQTYPL |
| | alpha-D- | | HEIIIQDDGSTDNTWQILEKYEEKYPLIHIYHNE |
| | GlcNAc- | | GTHGVNANFLSAMHRTTGDFIAIADQDDIWETDK |
| | diphosphoundecap | | IANQMTTIGNKLLCSGLTRPFSSDGSFAYFDNRP |
| | renol | | RNVSIFRMMFLGLPGHTMLFRRELLRMMPPVTHS |
| | | | FFNVSLYDAALSILAASHDSIAFCNKVLVNFRRH |
| | | | ADATTYNDYSRSLPSWQNGLYELLWGLRHYHQAR |
| | | | SIALPIYRGKLALMEGITTNYHDFIEAKAIMRLE |
| | | | TQKGLWAFLRLQYLLTKNHQRLFQTSGGSFIKMI |
| | | | RAWLYPVMQLYMYHHALRRCK |
| 27 | UDP-N- | P33038 | MESFIIEGGHRLSGTIAPQGAKNEALEVICATLL |
| | acetylglucosamine | | TTEEVIIRNIPNILDVNNLIKLLQDIGVKVKKLG |
| | | | ANDFSFQADEVKLDYLESIDFVKKCSSLRGSVLM |
| | | | IGPLLGRFGKATIAKPGGDKIGRRRLDTHFLGFK |
| | | | NLGARFVRIEDRDVYEIQADKLVGDYMLLDEASV |
| | | | TGTANIIMSAVMAEGTTTIYNAACEPYIQQLCHL |
| | | | LNAMGAKITGIASNLITIEGVTSLHGAEHRILPD |
| | | | MIEVGSFIGMAAMVGDGVRIKDVSIPNLGLILDT |
| | | | FRRLGVQIIEDEDDLIIPRQDHYVIDSFIDGTIM |
| | | | TISDAPWPGLTPDLISVLLVVATQAQGSVLFHQK |
| | | | MFESRLFFVDKLIDMGAQIILCDPHRAVVVGHDH |
| | | | AKKLRAGRMSSPDIRAGIALLIAALTAEGTSRID |
| | | | NIAQIDRGYENIEGRLNALGAKVQRVEIC |
| 28 | Sensor_protein_ | P30855 | MERSGNFYKAIRLGYILISILIGCMAYNSLYEWQ |
| | EvgS | | EIEALELGNKKIDELRKEINNINIQMIKFSLLGE |
| | | | TILEWNDKDIEHYHARRMAMDSMLCRFKATYPAE |
| | | | RIDSVRHLLEDKERQMCQIVQILEQQQAINDKIT |
| | | | SQVPVIVQKSVQEQPKKSKRKGFLGIFGKKEEAK |
| | | | PTVTTTMHRSFNRNMRTEQQAQSRRLSVHADSLA |
| | | | ARNAELNRQLQGLVVQIDGKVQTDLQKREAEITA |
| | | | MRERSFIQIGGLTGFVILLLVISYIIIHRNANRI |
| | | | KRYKQETADLIERLQQMAKRNEALITSRKKAVHT |
| | | | ITHELRTPLTAITGYAGLIQKNFNADKTGMYIRN |
| | | | IQQSSDRMREMLNTLLSFFRLDDGKEQPNFSTCR |
| | | | ISSIAHTLESEFMPIAINKGLALTVTNHTDAVVL |
| | | | TDKERILQIGNNLLSNAIKFTENGAVSLTMGYDN |
| | | | GMLKLIVKDTGSGMTEEEQQRVFGAFERLSNAAA |
| | | | KDGFGLGLSIVQRIVTMLGGTIQLKSEKGKGSRF |
| | | | TVEIPMQSAEELPERINKTQIHHNRTLHDIVAID |
| | | | NDKVLLLMLKEMYAQEGTHCDTCTNAAELMEMIR |
| | | | RKEYSLLLTDLNMPDINGFELLELLRTSNVGNSR |
| | | | IIPIIVTTASGSCNREELLERGFSDCLLKPFSIS |
| | | | ELMEVSDKCAMKGKQNEKPDFSSLLSYGNESVML |
| | | | DKLIAETEKEMQSVRDGEQRKDFQELDALTHHLR |
| | | | SSWEILRADQPLRELYKQLHGSAVPDYEALNNAV |
| | | | TAVLDKGSEIIRLAKEERRKYENG |
| 29 | Phosphate- | Q7A5Q2 | MKRSRFYITVGLILSLTLLMSACGQKKAKDGRTD |
| | binding_protein_ | | TPTSGTIKFASDESFSPTVEELLQNYQFRYPQAH |
| | PstS | | LLPIYTDDNTGMKLLLDQKVNLFITSHAMTKGED |
| | | | AILRGKGPTPEVFPIGYDGIAFIVNRSNPDSCIT |
| | | | VDDVKKILQGKIAKWNQLNPKNNRGSIEVVFDNK |
| | | | ASATLHYVVDSTLGGKNIKSENTVAAKNSKSVID |
| | | | YVNKTPNAIGVIGSNWLNDHRDTTNTTFKKDVTV |
| | | | ASISKATVASPSNSWQPYQAYLLDGRYPFVRTIY |
| | | | ALLADPHKALPYAFANYIANPIGQMIIFKAGLLP |
| | | | YRGNINIREVEVKNQ |
| 30 | Bifunctional_ | P9WHM7 | MAGTKRIKTALISVFHKDGLDDLLKKLDEEGVQF |
| | purine_ | | LSTGGTQQFIESLGYECQKVEDVTSYPSILGGRV |
| | biosynthesis_ | | KTLHPKIFGGILARRDNEEDQKQMVEYTIPAIDL |
| | protein_PurH | | VIVDLYPFEQTVASGASAQDIIEKIDIGGISLIR |
| | | | AGAKNFKDVVIVPSKAEYPVLLQLLNTKGAETEI |
| | | | EDRKMFAERAFGVSSHYDTAIHSWFAAE |
| 31 | Multidrug_ | P0AE06 | MEEEKGGRIGQRPYILKIITERNYIIIIDMKKAK |
| | efflux_pump_ | | ILLFVTALVAVLTSCGGGQKGLPTSDEYPVITIG |
| | subunit_AcrA | | ASNAQLKTTYPATIKGVQDVEVRPKVSGFITKLN |
| | | | IHEGEYVHAGQVLFVIDNSTYQAAVRQAQAQVNS |
| | | | AQSAVAQAKANVVQANASLNSANAQAATSRLTYN |
| | | | NSQNLYNNKVIGDYELQSAKNTYETAQASVRQAQ |
| | | | SGIASAQAAVKQAEAGVRQAQAMLSTAKDNLGFC |
| | | | YVKSPASGYVGSLPFKEDALVSASSAQPVTTISN |
| | | | TSTIEVYFSMTEADVLKLSRTDDGLSNAIKKFPA |
| | | | VSLLLADGSTYNHEGAIVKTSGMTDATTGTINVI |
| | | | ARFPNPEHLLKSGGSGKIVIAKNNNRALLIPQEA |
| | | | VTQVQNKMFVYKVDAKDKVHYSEITVDPQNDGIN |
| | | | YIVTSGLKMGERIVSKGVSSLEDGAKIKALTPAE |
| | | | YEEAIKKAEKLGENQSSASGFLKTMKGDSK |
| 32 | Cell_division_ | Q81X30 | MAKRRNKARSHHSLQVVTLCISTAMVLILIGMVV |
| | protein_FtsX | | LTVFTSRNLSSYVKENLTVTMILQPDMSTEESAA |
| | | | LCQRIRSLHYINSLNFISKEQALKEGTRELGANP |
| | | | AEFAGQNPFTGEIELQLKANYANNDSTKNIEREL |
| | | | RTYRGVSDITYPQNLVESVNHTLGKISLVLLVIA |
| | | | ILLTIVSFSLMNNTIRLSIYARRFSIHTMKLVGA |
| | | | SWGFIRAPFLRRAVMEGLVSALLAIAVLGVGLCL |
| | | | LYDYEPDITKVLSWDVLVITAGVMLAFGVLIATF |
| | | | CSWLSVNKFLRMKAGDLYKI |
| 33 | Fe(2+)_ | Q9PMQ9 | MKLSDLKTGETGVIVKVLGHGGFRKRIIEMGFIQ |
| | transporter_ | | GKQVEVLLNAPLRDPVKYKIMGYEVSLRHSEADQ |
| | FeoB | | IEVISAEEARQLEQAKADNEPQQGALSNNIPDES |
| | | | DHALTPFELTDAANRKSKVINVALVGNPNCGKTS |
| | | | LFNFASGAHERVGNYSGVTVDAKVGRANYEGYEF |
| | | | HLVDLPGTYSLSAYSPEELYVRKQLVEKTPDVVI |
| | | | NVIDASNLERNLYLTTQLIDMHVRMVCALNMFDE |
| | | | TEQRGDNIDYQKISELFGIPMVPTVFTNGRGVKE |
| | | | LFHQVIAVYEGKEDETSQFRHIHINHGHELEGGI |
| | | | KNIQEHLRAYPDICQRYSTRYLAIKLLEHDKDVE |
| | | | ELIKPLKDSDEIFKHRDIAAQRVKEETGNESETA |
| | | | IMDAKYGFIHGALEEADYSTGQKKDTYQTTHFID |
| | | | QILTNKYFGFPIFFLILFIMFTATFVIGQYPMDW |
| | | | IDGGVSWLGDFISSNMPDGPVKDMLVDGIIGGVG |
| | | | AVIVFLPQILILYFFISYMEDSGYMARAAFIMDK |
| | | | LMHKMGLHGKSFIPLIMGFGCNVPAVMATRTIES |
| | | | RRSRLVTMLILPLMSCSARLPIYVMITGSFFALK |
| | | | YRSLAMLSLYVIGILMSVIMSRVFSRFLVKGEDT |
| | | | PFVMELPPYRFPTWKAIGRHTWEKGKQYLKKMGG |
| | | | IILVASIIVWALGYFPLPDKPDMGQQERQEHSFI |
| | | | GQIGHAVEPVFRPQGFNWKLDVGLLAGVGAKEIV |
| | | | ASTMGVLYSNDDSFKDDNSFSSEGGKYVKLHKQI |
| | | | TQDVANLHGVSYNEAEPIATLTAFCFLLFVLLYF |
| | | | PCIATIAAIKGETGSWGWALFAAGYTTLLAWVVS |
| | | | AIVFQVGMLFIG |
| 34 | Pneumolysin | Q04IN8 | MKKNLLKAVLPASLALFAVTFGSCSQDGQLTGTK |
| | | | EDTGERVLDNTREIQNYLRTLPLAPMMSRASDPV |
| | | | PSDDGTTVPVDEGTSKTEEKGVLNGIPGSWVKTT |
| | | | RRYKMTQAFDESFLFDPTSDIVYPGCVLKGGTIA |
| | | | NGTYAIITSHETGDVTFSINLSPANPQEARETSA |
| | | | TVHNIRKSEYQEVWNKWANMQWKESPITTIESVE |
| | | | KINSQEELATKLGVAVNSPVANGSLNFGFNFNKK |
| | | | KNHILARLIQKYFSVSTDAPKKGNIFESIDKEAL |
| | | | DGYQPVYISNINYGRIIYLSVESDEDEKVVDEAI |
| | | | NFAMNQIKGVDVSVSADQSLHYRKVLANCDIRIT |
| | | | VLGGGQTIQKEVLKGDIDSFQRFLNADIPMEQMS |
| | | | PISFSLRYAVDNSQARVVTSNEFTVTQRDFVPEF |
| | | | KKVRMQLQVLGFSGTNTGPFPNLDREAGLWGSIS |
| | | | LSLNGQDNELVKISQSNPFFFNYREKKETMHPIG |
| | | | FGGIVTVEFDKDPNESLEDFVDHQKMTFVSDLHS |
| | | | TRSTYNYNFGRTTFTHTLGTLYTKYKGDDPIFVL |
| | | | ESNNKNVKIHTYVKVLDMKFFN |
| 35 | Cluster: | G6AG77 | MTKFIYAMSLFLLAAISIKAQPIQKTSGCLLHGS |
| | Uncharacterized | | VVSSTDATAIAGATVRLYQLKKLVGGTVSDASGN |
| | protein | | FDVKCPSSGSLQLRITAVGFKEVDTTLNVPTVTP |
| | | | LSIYMRAGKHAMDEVTVTASEKRGMTSTTVTGQT |
| | | | AMEHLQPSSFADLLALLPGGMTKIPALGSANVIT |
| | | | LREAGPPSSQYATSSLGTKFVIDGQAIGTDANMQ |
| | | | YIAGSFQGDADNSRNHVSYGVDMREIPTDNIEKV |
| | | | EVVRGIPSVKYGELTSGLINITRKRSQSPLLLRL |
| | | | KADEYGKLVSVGKGFLLSGKWNLNVDGGLLDARK |
| | | | EPRNRFETYRRLTFSARLRRKWNLGERYVLEWSG |
| | | | ATDYSLNIDNVKTDPEIQIHREDSYRSSYLKMGM |
| | | | NHRLLLRRKALVGLQSVSLAYSASLASDRIHQTE |
| | | | AVALQRDYVVPLAYEGGEYDGLFLPMQYLCDYRV |
| | | | EGKPFYSTLRGETEWLARTSFISHHITAGGEFLL |
| | | | NKNYGRGQIFDITKPLHASTARRPRSYKDIPATD |
| | | | ILSFYAEDKATMPIGKHQLTVMAGLRTTQMLNIP |
| | | | ASYAVHGKLFTDTRVNVQWDFPSFLGFKSFVSGG |
| | | | LGMMTKMPTVLDLYPDYVYKDITEMNYWDIRPAY |
| | | | KRIHIRTYKLNQVNPDLRPARNKKWEIRLGMDKG |
| | | | AHHFSVTYFHEDMKDGFRSTTTMRPFIYKRYDTS |
| | | | VINPSALTGPPSLASLPVVTDTLLDGYGRTENGS |
| | | | RITKQGIEFQYSSPRIPVIQTRITVNGAWFRTLY |
| | | | ENSIPLFRSAPNVVVGTVAIADRYAGYYMSTDKY |
| | | | DKQIFTSNFIFDSYVDKLGLILSATAECFWMSNT |
| | | | KRPATSSTPMGYMDITGTVHPYVEADQSDPYLRW |
| | | | LVLTGTAGQDMDYRERSYMLVNFKATKRFGRHLS |
| | | | LSFFADRVFYVAPDYEVNGFIVRRTFSPYFGMEI |
| | | | GLKI |
| 36 | Cell_division_ | P0A9R7 | MLIDFKKVNIYQDERLILKDIDFQATEGEFIYLI |
| | ATP-binding_ | | GRVGSGKSSLLKTFYGELDIDQEDAEKAEVLGES |
| | protein_FtsE | | VLDIKQKRIPALRRQMGIIFQDFQLLHDRSVAKN |
| | | | LKFVLQATGWKDKEKIKQRIKEVLEQVGMIDKAA |
| | | | KMPSELSGGEQQRTAIARAFLNNPKIILADEPTG |
| | | | NLDPETASNIVSILKDTCKNGTTVIMSTHNINLL |
| | | | SQFPGKVYRCMEQALVPVTNEAQTKDLEEDSTSV |
| | | | EPLIEPVLEEEAQAEDSKE |
| 37 | Di- | P0C2U3 | MFENQPKALYALALANTGERFGYYTMTAVFALFL |
| | /tripeptide_ | | RANFGLEPGTAGLIYSIFLGLVYFLPLIGGIMAD |
| | transporter | | KFGYGKMVTTGIIVMFAGYLFLSVPLGGGTVAFG |
| | | | AMLAALLLISFGTGLFKGNLQVMVGNLYDTPELA |
| | | | SKRDSAFSIFYMAINIGALFAPTAAVKTKEWAET |
| | | | SLGYAGNDAYHFSFAVACVSLIVSMGIYYAFRST |
| | | | FKHVEGGIKKTEKAAAAAVEELTPQQTKERIVAL |
| | | | CLVFAVVIFFWMAFHQNGLTLTYFADEFVSPTST |
| | | | GVQSMAFDVVNLVMIVFIVYSIMALFQSKTTKAK |
| | | | GIACAVILAAIAVLAYKYMNVNGQVEVSAPIFQQ |
| | | | FNPFYVVALTPTSMAIFGSLAAKGKEPSAPRKIA |
| | | | YGMIVAGCAYLLMVLASQGLLTPHEQKLAKAAGE |
| | | | TVPFASANWLIGTYLVLTFGELLLSPMGISFVSK |
| | | | VAPPKYKGAMMGGWFVATAIGNILVSVGGYLWGD |
| | | | LSLTVVWTVFTVLCLVSASFMFLMMKRLEKVA |
| 38 | Calcium: | Q47910 | MKKILIFVAGLCMSLAASAQIQRPKLVVGLVVDQ |
| | transporting_ | | MRWDYLYYYYNEYGTDGLRRLVDNGFSFENTHIN |
| | ATPase | | YAPTVTAIGHSSVYTGSVPAITGIAGNYFFQDDK |
| | | | NVYCCEDPNVKSVGSDSKEGQMSPHRLLASTIGD |
| | | | ELQISNDFRSKVIGVALKDRASILPAGHAADAAY |
| | | | WWDTSAGHFVTSTFYTDHLPQWVIDFNEKNHTAP |
| | | | NFNIKTSTQGVTMTFKMAEAALKNENLGKGKETD |
| | | | MLAVSISSTDAIGHVYSTRGKENHDVYMQLDKDL |
| | | | AHFLKTLDEQVGKGNYLLFLTADHGAAHNYNYMK |
| | | | EHRIPAGGWDYRQSVKDLNGYLQGKFGIAPVMAE |
| | | | DDYQFFLNDSLIAASGLKKQQIIDESVEYLKKDP |
| | | | RYLYVFDEERISEVTMPQWIKERMINGYFRGRSG |
| | | | EIGVVTRPQVFGAKDSPTYKGTQHGQPFPYDTHI |
| | | | PFLLYGWNVKHGATTQQTYIVDIAPTVCAMLHIQ |
| | | | MPNGCIGTARNMALGN |
| 39 | Poly-beta-1,6-N- | Q5HKQ0 | MDRQVFQTDSRQRWNRFKWTLRVLITIAILLGVV |
| | acetyl-D- | | FVAMFALEGSPQMPFRHDYRSVVSASEPLLKDNK |
| | glucosamine_ | | RAEVYKSFRDFFKEQKMHSNYAKVAARQHRFVGH |
| | synthase | | TDNVTQKYIKEWTDPRMGIRSAWYVNWDKHAYIS |
| | | | LKNNLKNLNMVLPEWYFINPKTDRIEARIDQRAL |
| | | | KLMRRAHIPVLPMLTNNYNSAFRPEAIGRIMRDS |
| | | | TKRMGMINELVAACKHNGFAGINLDLEELNINDN |
| | | | ALLVTLVKDFARVFHANGLYVTQAVAPFNEDYDM |
| | | | QELAKYDDYLFLMAYDEYNAGSQAGPVSSQRWVE |
| | | | KATDWAAKNVPNDKIVLGMATYGYNWAQGQGGTT |
| | | | MSFDQTMATALNAGAKVNFNDDTYNLNFSYQDED |
| | | | DGTLHQVFFPDAVTTFNIMRFGATYHLAGFGLWR |
| | | | LGTEDSRIWKYYGKDLSWESAARMPIAKIMQLSG |
| | | | TDDVNFVGSGEVLNVTSEPHAGRIGIVLDKDNQL |
| | | | IIEERYLSLPATYTVQRLGKCKEKQLVLTFDDGP |
| | | | DSRWTPKVLSILKHYKVPAAFFMVGLQIEKNIPI |
| | | | VKDVFNQGCTIGNHTFTHHNMIENSDRRSFAELK |
| | | | LTRMLIESITGQSTILFRAPYNADADPTDHEEIW |
| | | | PMIIASRRNYLFVGESIDPNDWQQGVTADQIYKR |
| | | | VLDGVHQEYGHIILLHDAGGDTREPTVTALPRII |
| | | | ETLQREGYQFISLEKYLGMSRQTLMPPIKKGKEY |
| | | | YAMQANLSLAELIYHISDFLTALFLVFLVLGFMR |
| | | | LVFMYVLMIREKRAENRRNYAPIDPLTAPAVSII |
| | | | VPAYNEEVNIVRTISNLKEQDYPSLKIYLVDDGS |
| | | | KDNTLQRVREVFENDDKVVIISKKNGGKASALNY |
| | | | GIAACSTDYIVCVDADTQLYKDAVSKLMKHFIAD |
| | | | KTGKLGAVAGNVKVGNQRNMLTYWQAIEYTTSQN |
| | | | FDRMAYSNINAITVIPGAIGAFRKDVLEAVGGFT |
| | | | TDTLAEDCDLTMSINEHGYLIENENYAVAMTEAP |
| | | | ESLRQFIKQRIRWCFGVMQTFWKHRASLFAPSKG |
| | | | GFGMWAMPNMLIFQYIIPTFSPIADVLMLFGLFS |
| | | | GNASQIFIYYLIFLLVDASVSIMAYIFEHESLWV |
| | | | LLWIIPQRFFYRWIMYYVLFKSYLKAIKGELQTW |
| | | | GVLKRTGHVKGAQTIS |
| 40 | ATP_synthase_ | P29707 | MSQINGRISQIIGPVIDVYFDTKGENPEKVLPNI |
| | subunit_beta_ | | YDALRVKKADGQDLIIEVQQQIGEDTVRCVAMDN |
| | sodium_ion_ | | TDGLQRGLEVVPTGSPIVMPAGEQIKGRMMNVIG |
| | specific | | QPIDGMSALQMEGAYPIHREAPKFEDLSTHKEML |
| | | | QTGIKVIDLLEPYMKGGKIGLFGGAGVGKTVLIM |
| | | | ELINNIAKGHNGYSVFAGVGERTREGNDLIRDML |
| | | | ESGVIRYGEKFRKAMDEGKWDLSLVDSEELQKSQ |
| | | | ATLVYGQMNEPPGARASVALSGLTVAEEFRDHGG |
| | | | KNGEAADIMFFIDNIFRFTQAGSEVSALLGRMPS |
| | | | AVGYQPTLASEMGAMQERITSTKHGSITSVQAVY |
| | | | VPADDLTDPAPATTFTHLDATTELSRKITELGIY |
| | | | PAVDPLGSTSRILDPLIVGKEHYDCAQRVKQLLQ |
| | | | KYNELQDIIAILGMDELSDDDKLVVNRARRVQRF |
| | | | LSQPFTVAEQFTGVKGVMVPIEETIKGFNAILNG |
| | | | EVDDLPEQAFLNVGTIEDVKEKAKQLLEATKA |
| 41 | Cluster: | G6AGX5 | MNPIYKIITSILFCVLSINTMAQDLTGHVTSKAD |
| | Uncharacterized | | DKPIAYATVTLKENRLYAFTDEKGNYTIKNVPKG |
| | protein | | KYTVVFSCMGYASQTVVVMVNAGGATQNVRLAED |
| | | | NLQLDEVQVVAHRKKDEITTSYTIDRKTLDNQQT |
| | | | MTLSDIAQLLPGGKSVNPSLMNDSKLTLRSGTLE |
| | | | RGNASFGTAVEVDGIRLSNNAAMGETAGVSTRSV |
| | | | SASNIESVEVVPGIASVEYGDLTNGVVKVKTRRG |
| | | | SSPFIVEGSINQHTRQIALHKGVDLGGNVGLLNF |
| | | | SIEHARSFLDAASPYTAYQRNVLSLRYMNVFMKK |
| | | | SLPLTLEVGLNGSTGGYNSKADPDRSLDDYNKVK |
| | | | DNNVGGNIHLGWLLNKRWITNVDLTAAFTYADRL |
| | | | SESYTNESSNATQPYIHTLTEGYNIAEDYDRNPS |
| | | | ANIILGPTGYWYLRGFNDSKPLNYSLKMKANWSK |
| | | | AFGKFRNRLLVGGEWTSSMNRGRGTYYADMRYAP |
| | | | SWREYRYDALPSLNNIAIYAEDKLSMDVNERQNA |
| | | | ELTAGIREDITSIPGSEYGSVGSFSPRIVINARY |
| | | | VFRFGQNSWLNSMTLHAGWGRSVKIPSFQVLYPS |
| | | | PSYRDMLAFASTSDADNRSYYAYYTYPSMARYNA |
| | | | NLKWQRADQWDLGVEWRTKIADVSLSFFRSKVSN |
| | | | PYMATDVYTPFTYKYTSPAMLQRSGTAVADRRFS |
| | | | IDPQTGIVTVSDASGVKSPVTLGYEERNTYVTNT |
| | | | RYVNADALQRYGLEWIVDFKQIKTLRTQVRLDGK |
| | | | YYHYKAQDETLFADVPVGLNTRQSDGRLYQYVGY |
| | | | YRGGAATTTNYTANASASNGSVSGQVDLNATITT |
| | | | HIPKIRLIVALRLESSLYAFSRATSSRGYVVSSG |
| | | | NEYFGVPYDDKTENQTVIVYPEYYSTWDAPDVLI |
| | | | PFAEKLRWAETNDRGLFNDLAQLVVRTNYPYTLN |
| | | | PNRLSAYWSANLSVTKEIGRHVSVSFYANNFFNT |
| | | | LSQVHSTQTGLETSLFGSGYVPSFYYGLSLRLKI |

In some embodiments, the Prevotella bacteria is a strain of Prevotella bacteria free or substantially free of one or more (e.g., 1, 2, 3, 4, 5, 6, 7, 8, 9, 10, 11, 12, 13, 14, 15, 16, 17, 18, 19, 20, 21, 22, 23, 24, 25 or more) proteins listed in Table 2 and/or one or more (e.g., 1, 2, 3, 4, 5, 6, 7, 8, 9, 10, 11, 12, 13, 14, 15, 16, 17, 18, 19, 20, 21, 22, 23, 24, 25 or more) genes encoding proteins listed in Table 2. In some embodiments, Prevotella bacteria is free of all of the proteins listed in Table 2 and/or all of the genes encoding the proteins listed in Table 2.

TABLE 2
Other Prevotella proteins

| Seq. ID. No. | Name | Uniprot ID | Amino Acid Sequence |
|---|---|---|---|
| 42 | UDP-Gal: alpha-D- | Q03084 | MERIDISVLMAVYKKDNPAFLRESLESIFSQTVEA |
| | GlcNAc- | | AEVVLLEDGPLTDALYDVIKSYEAIYSTLKVVSYP |
| | diphosphoundecap | | ENRGLGKTLNDGLLLCKYNLVARMDADDICKPNRL |
| | renol | | EMEYNWLKSHEDYDVIGSWVDEFTDNKTRVKSIRK |
| | | | VPEAYDEIKNYAQYRCPINHPTAMYRKAAVLAVGG |
| | | | YLTEYFPEDYFLWLRMLNNGSKFYNIQESLLWFRY |
| | | | SEETVAKRGGWAYACDEVRILVRMLKMGYIPFHVF |
| | | | CQSVVIRFTTRVMPLPIRQRLYNLIRKT |
| 43 | ATP_synthase_ | A1B8P0 | MSQINGRISQIIGPVIDVYFDTKGENPEKVLPKIH |
| | subunit_beta | | DALRVKRANGQDLIIEVQQHIGEDTVRCVAMDNTD |
| | | | GLQRNLEVVPTGSPIVMPAGDQIKGRMMNVIGQPI |
| | | | DGMEALSMEGAYPIHREAPKFEDLSTHKEMLQTGI |
| | | | KVIDLLEPYMKGGKIGLFGGAGVGKTVLIMELINN |
| | | | IAKGHNGYSVFAGVGERTREGNDLIRDMLESGVIR |
| | | | YGEKFRKAMDEGKWDLSLVDQEELQKSQATLVYGQ |
| | | | MNEPPGARASVALSGLTVAEEFRDHGGKNGEAADI |
| | | | MFFIDNIFRFTQAGSEVSALLGRMPSAVGYQPTLA |
| | | | SEMGTMQERITSTKHGSITSVQAVYVPADDLTDPA |
| | | | PATTFTHLDATTELSRKITELGIYPAVDPLGSTSR |
| | | | ILDPLIVGKDHYECAQRVKQLLQHYNELQDIIAIL |
| | | | GMDELSDEDKLVVNRARRVQRFLSQPFTVAEQFTG |
| | | | VKGVMVPIEETIKGFNAILNGEVDDLPEQAFLNVG |
| | | | TIEDVKEKAKRLLEATK |
| 44 | Cell_division_ | O05779 | MPIGNGQKYQLTIINHTEIIMLIDYKKVNIYQDER |
| | ATP-binding_ | | LILKDVDFQAETGEFIYLIGRVGSGKSSLLKTIYG |
| | protein_FtsE | | ELDIDSEDAEKAVVLDESMPNIKRSRIPALRKQMG |
| | | | IIFQDFQLLHDRSVAKNLKFVLQATGWTSKQKIER |
| | | | RIEEVLAQVGMTDKKNKMPSELSGGEQQRIAIARA |
| | | | LLNTPKIIIADEPTGNLDPETAANIVSILKDSCQA |
| | | | GTTVIMSTHNINLIDQFPGKVYRCHEGELHQLTDK |
| | | | KEVSELAEETAPVETIDEPEQND |
| 45 | Hemin_transport_ | Q56992 | MKRNILLFICLATSILLLFGLNLTTGSVQIPFADI |
| | system_ | | LDILCGRFIGKESWEYIILENRLPQTLTAILCGAS |
| | permease_ | | LSVCGLMLQTAFRNPLAGPDVFGISSGAGLGVALV |
| | protein_HumU | | MLLLGGTVSTSIFTVSGFLAILTAAFVGAIAVTAL |
| | | | ILFLSTLVRNSVLLLIVGIMVGYVSSSAVSLLNFF |
| | | | ASEEGVKSYMVWGMGNEGAVSMNHIPLESILCLIG |
| | | | IIASFLLVKPLNILLLGPQYAESLGISTRQIRNIL |
| | | | LVVVGLLTAITTAFCGPISFIGLAIPHIARLLFRT |
| | | | ENHQILLPGIVLSGAAIALLCNFICYLPGESGIIP |
| | | | LNAVTPLIGAPIIIYVIIQRR |
| 46 | Hexuronate_ | P9WN45 | MKKYYPWVLVALLWFVALLNYMDRQMLSTMQEAMK |
| | transporter | | VDIAELNHAEAFGALMAVFLWIYGIVSPFAGIIAD |
| | | | RVNRKWLVVGSIFVWSAVTYLMGYAESFDQLYWLR |
| | | | AFMGISEALYIPAALSLIADWHEGKSRSLAIGIHM |
| | | | TGLYVGQAVGGFGATLAAMFSWHAAFHWFGIIGIV |
| | | | YSLVLLLFLKENPKHGQKSVLQGETKPSKNPFRGL |
| | | | SIVESTWAFWVILFYFAVPSLPGWATKNWLPTLFA |
| | | | NSLDIPMSSAGPMSTITIAVSSFIGVIMGGVISDR |
| | | | WVQRNLRGRVYTSATGLGLTVPALMLLGFGHSLVS |
| | | | VVGAGLCFGIGYGMFDANNMPILCQFISSKYRSTA |
| | | | YGIMNMTGVFAGAAVTQVLGKWTDGGNLGNGFAIL |
| | | | GGIVVLALVLQLSCLKPTTDNME |
| 47 | 1,4-alpha- | P9WN45 | MVTKKTTTKKAPVKKTSAKTTKVKEPSHIGLVKND |
| | glucan_ | | AYLAPYEDAIRGRHEHALWKMNQLTQNGKLTLSDF |
| | branching_ | | ANGHNYYGLHQTADGWVFREWAPNATEIYLVGDFN |
| | enzyme_GlgB | | GWNEQEAYQCHRIEGTGNWELTLPHDAMQHGQYYK |
| | | | MRVHWEGGEGERIPAWTQRVVQDEASKIFSAQVWA |
| | | | PAEPYVWEKKTFKPQTSPLLIYECHIGMAQDEEKV |
| | | | GTYNEFREKVLPRIIKDGYNAIQIMAIQEHPYYGS |
| | | | FGYHVSSFFAASSRFGTPEELKALIDEAHKNGIAV |
| | | | IMDIVHSHAVKNEVEGLGNLAGDPNQYFYPGERHE |
| | | | HPAWDSLCFDYGKDEVLHFLLSNCKYWLEEYHFDG |
| | | | FRFDGVTSMLYYSHGLGEAFCNYADYFNGHQDDNA |
| | | | ICYLTLANCLIHEVNKNAVTIAEEVSGMPGLAAKF |
| | | | KDGGYGFDYRMAMNIPDYWIKTIKELPDEAWKPSS |
| | | | IFWEIKNRRSDEKTISYCESHDQALVGDKTIIFRL |
| | | | VDADMYWHFRKGDETEMTHRGIALHKMIRLATIAA |
| | | | INGGYLNFMGNEFGHPEWIDFPREGNGWSHKYARR |
| | | | QWNLVDNEELCYHLLGDFDRKMLEVITSEKKFNET |
| | | | PIQEIWHNDGDQILAFSRGELVFVFNFSPSHSYSD |
| | | | YGFLVPEGSYNVVLNTDAREFGGFGFADDTVEHFT |
| | | | NSDPLYEKDHKGWLKLYIPARSAVVLRKK |
| 48 | Cluster: | D9RW24 | MKIDIERIKYFLTVGMFMKTEHSSKRRNMLIRQFQ |
| | YihY family | | KFYLTVKFFFVRDHAASTAQLSFSTIMAIVPIASM |
| | protein | | IFAIANGFGFGQFLEKQFREMLSAQPEAATWLLKL |
| | | | TQSYLVHAKTGLFIGIGLMIMLYSVFSLIRTVETT |
| | | | FDNIWQVKDSRPISRIVIDYTALMFLVPISIIILS |
| | | | GLSIYFYSFVENLNGLRFLGTIASFSLRYLVPWAI |
| | | | LTLMFIVLYVFMPNAKVKITKTVAPAMIASIAMLC |
| | | | LQAVYIHGQIFLTSYNAIYGSFAALPLFMLWILAS |
| | | | WYICLFCAELCYFNQNLEYYECLIDTEDICHNDLL |
| | | | ILCATVLSHICQRFANDQKPQTALQIKTETHIPIR |
| | | | VMTDILYRLKEVNLISENFSPTSDEVTYTPTHDTN |
| | | | NITVGEMIARLESTPASDFALLGFSPKKAWNHDIY |
| | | | DRVGSTREIYLNELKSINIKELISYSEN |
| 49 | Capsule_ | P19579 | MMKRPSIARVVKVIICLLTPILLSFSGIGDNDIDK |
| | biosynthesis_ | | KKSTSKEVDDTLRIVITGDLLLDRGVRQKTDMAGV |
| | protein_CapA | | DALFSPTIDSLFHSSNYVIANLECPVTKIRERVFK |
| | | | RFIFRGEPEWLPTLRRHGITHLNLANNHSIDQGRN |
| | | | GLLDTQEQIKKAGMIPIGAGKNMEEAAEPVLISTS |
| | | | PRHVWVISSLRLPLENFLYLPQKPCVSQESIDSLI |
| | | | MRVKRLRATDKNCYILLILHWGWEHHFRATPQQRE |
| | | | DAHKLIDAGADAIVGHHSHTLQTIETYRGKPIYYG |
| | | | IGNFIFDQRKPMNSRACLVELSITAEKCKAKALPI |
| | | | EIKNCTPYLSK |
| 50 | Peptidoglycan_ | B5ZA76 | MILLSFDTEEFDVPREHGVDFSLEEGMKVSIEGTN |
| | deacetylase | | RILDILKANNVCATFFCTGNFAELAPEVMERIKNE |
| | | | GHEVACHGVDHWQPKPEDVFRSKEIIERVTGVKVA |
| | | | GYRQPRMFPVSDEDIEKAGYLYNSSLNPAFIPGRY |
| | | | MHLTTSRTWFMQGKVMQIPASVSPHLRIPLFWLSM |
| | | | HNFPEWFYLRLVRQVLRHDGYFVTYFHPWEFYDLK |
| | | | SHPEFKMPFIIKNHSGHELEQRLDRFIKAMKADKQ |
| | | | EFITYVDFVNRQKK |
| 51 | Fumarate_ | P0AC47 | MAKNISFTIKYWKQNGPQDQGHFDTHEMKNIPDDT |
| | reductase_ | | SFLEMLDILNEELIAAGDEPFVFDHDCREGICGMC |
| | iron-sulfur_ | | SLYINGTPHGKTERGATTCQLYMRRFNDGDVITVE |
| | subunit | | PWRSAGFPVIKDCMVDRTAFDKIIQAGGYTTIRTG |
| | | | QAQDANAILISKDNADEAMDCATCIGCGACVAACK |
| | | | NGSAMLFVSSKVSQLALLPQGKPEAAKRAKAMVAK |
| | | | MDEVGFGNCTNTRACEAVCPKNEKIANIARLNREF |
| | | | IKAKFAD |
| 52 | Serine/threonine- | P9WI71 | MSENKLSTNEQAQTADAPVKASYTEYKVIPSQGYC |
| | protein_kinase_ | | MIVKCRKGDQTVVLKTLKEEYRERVLLRNALKREF |
| | PknH | | KQCQRLNHSGIVRYQGLVEVDGYGLCIEEEYVEGR |
| | | | TLQAYLKENHTDDEKIAIINQIADALRYAHQQGVI |
| | | | HRNLKPSNVLVTTQGDYVKLIDFSVLSPEDVKPTA |
| | | | ETTRFMAPEMKDETLTADATADIYSLGTIMKVMGL |
| | | | TLAYSEVIKRCCAFKRSDRYSNVDELLADLNNEGS |
| | | | SFSMPKIGKGTVVLGLIIAVVIGIGALLYNYGGAL |
| | | | IDQVGKIDVSSVFSSDAETAPEDTVKVNTAEQSDS |
| | | | LSTEAEAPAIGKLAFMNRMKPALYKDLDNTFEKNS |
| | | | ADKAKLTKAIKTYYRGLIQANDTLDNEQRAEVDRV |
| | | | FGDYVKQKKAALN |
| 53 | Carboxy- | O34666 | MRKYICLLLFYLFTFLPLSAQQGNDSPLRKLQLAE |
| | terminal_ | | MAIKNFYVDSVNEQKLVEDGIRGMLEKLDPHSTYT |
| | processing_ | | DAKETKAMNEPLQGDFEGIGVQFNMTEDTLVVIQP |
| | protease_CtpA | | VVNGPSQKVGILAGDRIVSVNDSTIAGVKMARIDI |
| | | | MKMLRGKKGTKVKLGVVRRGVKGVLTFVVTRAKIP |
| | | | VHTINASYMIRPNVGYIRIESFGMKTHDEFMSAVD |
| | | | SLKKKGMKTLLLDLQDNGGGYLQSAVQISNEFLKN |
| | | | NDMIVYTEGRRARRQNFKAIGNGRLQDVKVYVLVN |
| | | | ELSASAAEIVTGAIQDNDRGTVVGRRTFGKGLVQR |
| | | | PFDLPDGSMIRLTIAHYYTPSGRCIQKPYTKGDLK |
| | | | DYEMDIEKRFKHGELTNPDSIQFSDSLKYYTIRKH |
| | | | RVVYGGGGIMPDNFVPLDTTKFTRYHRMLAAKSII |
| | | | INAYLKYADANRQALKAQYSSFDAFNKGYVVPQSL |
| | | | LDEIVAEGKKEKIEPKDAAELKATLPNIALQIKAL |
| | | | TARDIWDMNEYFRVWNTQSDIVNKAVALATGK |
| 54 | Cluster: | D9RRG3 | MKLTEQRSSMLHGVLLITLFACAAFYIGDMGWVKA |
| | Uncharacterized | | LSLSPMVVGIILGMLYANSLRNNLPDTWVPGIAFC |
| | protein | | GKRVLRFGIILYGFRLTFQDVVAVGFPAIIVDAII |
| | | | VSGTILLGVLVGRLLKMDRSIALLTACGSGICGAA |
| | | | AVLGVDGAIRPKPYKTAVAVATVVIFGTLSMFLYP |
| | | | ILYRAGIFDLSPDAMGIFAGSTIHEVAHVVGAGNA |
| | | | MGAAVSNSAIIVKMIRVMMLVPVLLVIAFFVAKNV |
| | | | AERDDEAGGSRKINIPWFAILFLVVIGFNSLNLLP |
| | | | KELVDFINTLDTFLLTMAMSALGAETSIDKFKKAG |
| | | | FKPFLLAAILWCWLIGGGYCLAKYLVPVLGVAC |
| 55 | Cluster: | X6Q2J4 | MNKQFLLAALWLSPLGLYAHKANGIGAVTWKNEAP |
| | Cna protein | | KERMIRGIDEDKTHQRFTLSGYVKDRNGEPLINAT |
| | B-type domain | | IYDLTTRQGTMTNAYGHFSLTLGEGQHEIRCSYVG |
| | protein | | YKTLIETIDLSANQNHDIILQNEAQLDEVVVTTDL |
| | | | NSPLLKTQTGKLSLSQKDIKTEYALLSSPDVIKTL |
| | | | QRTSGVADGMELASGLYVHGGNGDENLFLLDGTPL |
| | | | YHTNHSLGLFSSFNADVVKNVDFYKSGFPARYGGR |
| | | | LSSVIDVRTADGDLYKTHGSYRIGLLDGAFHIGGP |
| | | | IRKGKTSYNFGLRRSWMDLLTRPAFAIMNHKSDNE |
| | | | DKLSMSYFFHDLNFKLTNIFNERSRMSLSVYSGED |
| | | | RLDAKDEWHSNNSSGYNDVDIYVNRFHWGNFNAAL |
| | | | DWNYQFSPKLFANFTAVYTHNRSTVSSSDEWRFTR |
| | | | PGEKEQLTLTSHGYRSSIDDIGYRAAFDFRPSPRH |
| | | | HIRFGQDYTYHRFQPQTYNRFDNYQTNSEAKADTI |
| | | | ATHSYNKNVAHQLTFYAEDEMTLNEKWSLNGGVNA |
| | | | DVFHISGKTFATLSPRLSMKFQPTERLSLKASYTL |
| | | | MSQFVHKIANSFLDLPTDYWVPTTARLHPMRSWQV |
| | | | AAGAYMKPNKHWLLSLEAYYKRSSHILQYSSWAGL |
| | | | EPPAANWDYMVMEGDGRSYGVELDADYNVSNLTLH |
| | | | GSYTLSWTQKKFDDFYDGWYYDKFDNRHKLTLTGR |
| | | | WNITKKIAAFAAWTFRTGNRMTIPTQYIGLPDVPA |
| | | | QEQGGLTFNSSDDNTLNFAYEKPNNVILPAYHRLD |
| | | | IGFDFHHTTKKGHERIWNLSFYNAYCHLNSLWVRV |
| | | | KIDSNNQMKIRNIAFIPVIPSFSYTFKF |
| 56 | Poly-beta-1,6-N- | P75905 | MSKQVFQTDSRQRWSYFKWTLRVILTILSLLGIVF |
| | acetyl-D- | | LAMFALEGSPQMPFRHDYRNAVTAASPYTKDNKTA |
| | glucosamine_ | | KLYKSFRDFFKEKKMHNNYAKATIKKQRFIGKADS |
| | synthase | | VTQKYFREWDDPRIGVRSAWYVNWDKHAYISLKNN |
| | | | IKHLNMVLPEWFFINPKTDKVEYRIDKQALRLMRR |
| | | | TGIPVLPMLTNNYNSDFHPEAIGRIMRDEKKRMAL |
| | | | INEMVRTCRHYGFAGINLDLEELNIQDNDLLVELL |
| | | | KDFSRVFHANGLYVTQAVAPFNEDYNMQELAKYND |
| | | | YLFLMAYDEHNIESQPGAVSSQRWVEKATDWAAKN |
| | | | VPNDKIVLGMATYGYDWANGEGGTTVSFDQTMAIA |
| | | | QDADAKVKFDDDTYNVNFSYQNTDDGKIHHVFFTD |
| | | | AATTFNIMRFGAEYHLAGYGLWRLGTEDKRIWRFY |
| | | | GKDMSWENVARMSVAKLMQLNGTDDVNFVGSGEVL |
| | | | EVTTEPHPGDISIRIDKDNRLISEEYYRALPSTYT |
| | | | IQRLGKCKDKQLVITFDDGPDSRWTPTVLSTLKKY |
| | | | NVPAAFFMVGLQMEKNLPLVKQVYEDGHTIGNHTF |
| | | | THHNMIENSDRRSYAELKLTRMLIESVTGHSTILF |
| | | | RAPYNADADPTEHEEIWPMIVASRRNYLFVGESID |
| | | | PNDWEPNVTSDQIYQRVIDGVHHEDGHIILLHDAG |
| | | | GSSRKPTLDALPRIIETLQHEGYQFISLEQYLGMG |
| | | | KQTLMPEINKGKAYYAMQTNLWLAEMIYHVSDFLT |
| | | | ALFLVFLALGMMRLIFMYVLMIREKRAENRRNYAP |
| | | | IDAATAPAVSIIVPGYNEEVNIVRTITTLKQQDYP |
| | | | NLHIYFVDDGSKDHTLERVHEAFDNDDTVTILAKK |
| | | | NGGKASALNYGIAACRSEYVVCIDADTQLKNDAVS |
| | | | RLMKHFIADTEKRVGAVAGNVKVGNQRNMLTYWQA |
| | | | IEYTSSQNFDRMAYSNINAITVVPGAIGAFRKEVI |
| | | | EAVGGFTTDTLAEDCDLTMSINEFIGYIIENENYA |
| | | | VALTEAPETLRQFVKQRIRWCFGVMQAFWKHRSSL |
| | | | FAPSKKGFGLWAMPNMLIFQYTIPTFSPLADVLML |
| | | | IGLFTGNALQIFFYYLIFLVIDASVSIMAYIFEGE |
| | | | RLWVLLWVIPQRFFYRWIMYYVLFKSYLKAIKGEL |
| | | | QTWGVLKRTGHVKG |
| 57 | Cell_division_ | O34876 | MAKKRNKARSRHSLQVVTLCISTAMVLMLIGIVVL |
| | protein_FtsX | | TGFTSRNLSSYVKENLTITMILQPDMNTEESAALC |
| | | | ERIRTLHYINSLNFISKEQALKDGTKELGANPAEF |
| | | | AGENPFTGEIEVQLKANYANNDSIRNIVQQLRTYR |
| | | | GVSDITYPQSLVESVNQTLGKISLVLLVIAVLLTI |
| | | | ISFSLINNTIRLSIYAHRFSIHTMKLVGGSWSFIR |
| | | | APFLRRAVLEGLVSALLAIAVLGIGICLLYEKEPE |
| | | | ITKLLSWDALIITAIVMLAFGVIIATFCAWLSVNK |
| | | | FLRMKAGDLYKI |
| 58 | UDP-2,3- | P44046 | MKNIYFLSDAHLGSLAIDHRRTHERRLVRFLDSIK |
| | diacylglucosamine_ | | HKAAAVYLLGDMFDFWNEYKYVVPKGFTRFLGKIS |
| | hydrolase | | ELTDMGVEVHFFTGNHDLWTYGYLEKECGVILHRK |
| | | | PITTEIYDKVFYLAHGDGLGDPDPMFRFLRKVFHN |
| | | | RFCQRLLNFFHPWWGMQLGLNWAKRSRLKRKDGKE |
| | | | VPYLGEDKEYLVQYTKEYMSTHKDIDYYIYGHRHI |
| | | | ELDLTLSRKARLLILGDWIWQFTYAVFDGEHMFLE |
| | | | EYVEGESKP |
| 59 | Poly-beta-1,6-N- | P75905 | MVGLDVLCYFIHAKGREKECYFERIIYQITCHSRT |
| | acetyl-D- | | KCYLCNIMKYSIIVPVFNRPDEVEELLESLLSQEE |
| | glucosamine_ | | KDFEVVIVEDGSQIPCKEVCDKYADKLDLHYYSKE |
| | synthase | | NSGPGQSRNYGAERAKGEYLLILDSDVVLPKGYIC |
| | | | AVSEELKREPADAFGGPDCAHESFTDTQKAISYSM |
| | | | TSFFTTGGIRGGKKKLDKFYPRSFNMGIRRDVYQE |
| | | | LGGFSKMRFGEDIDFSIRIFKAGKRCRLFPEAWVW |
| | | | HKRRTDFRKFWKQVYNSGIARINLYKKYPESLKLV |
| | | | HLLPMVFTVGTALLVLMILFGLFLQLFPIINVFGS |
| | | | VFIMMGLMPLVLYSVIICVDSTMQNNSLNIGLLSI |
| | | | EAAFIQLTGYGCGFISAWWKRCVCGMDEFAAYEKN |
| | | | FYK |
| 60 | Enolase | Q8DTS9 | MKIEKVHAREIMDSRGNPTVEVEVTLENGVMGRAS |
| | | | VPSGASTGENEALELRDGDKNRFLGKGVLKAVENV |
| | | | NNLIAPALKGDCVLNQRAIDYKMLELDGTPTKSKL |
| | | | GANAILGVSLAVAQAAAKALNIPLYRYIGGANTYV |
| | | | LPVPMMNIINGGAHSDAPIAFQEFMIRPVGAPSEK |
| | | | EGIRMGAEVFHALAKLLKKRGLSTAVGDEGGFAPK |
| | | | FDGIEDALDSIIQAIKDAGYEPGKDVKIAMDCAAS |
| | | | EFAVCEDGKWFYDYRQLKNGMPKDPNGKKLSADEQ |
| | | | IAYLEHLITKYPIDSIEDGLDENDWENWVKLTSAI |
| | | | GDRCQLVGDDLFVTNVKFLEKGIKMGAANSILIKV |
| | | | NQIGSLTETLEAIEMAHRHGYTTVTSHRSGETEDT |
| | | | TIADIAVATNSGQIKTGSMSRTDRMAKYNQLIRIE |
| | | | EELGACAKYGYAKLK |
| 61 | Outer_membrane_ | Q9G0Y6 | MKKLFTIAMLLGVTLGIHAQEVYSLQKCRELALQN |
| | efflux_protein_ | | NRQLKVSRMTVDVAENTRKAAKTKYLPRVDALAGY |
| | BepC | | QHFSREISLLSDDQKNAFSNLGTNTFGQLGGQIGQ |
| | | | NLTSLAQQGILSPQMAQQLGQLFSNVATPLTQVGN |
| | | | NIGQSINDAFRSNTKNVYAGGIVVNQPIYMGGAIK |
| | | | AANDMAAIGEQVAQNNISLKRQLVLYGVDNAYWLA |
| | | | ISLKKKEALAIRYRDLAQKLNEDVKKMIREGVATR |
| | | | ADGLKVEVAVNTADMQTARIQSGVSLAKMALCELC |
| | | | GLELNGDIPLSDEGDADLPPTPSTQFDNYTVSSSD |
| | | | TTGLNEARPELRLLQNAVDLSIQNTKLIRSLYMPH |
| | | | VLLTAGYSVSNPNLFNGFQKRFTDLWNIGITVQVP |
| | | | VWNWGENKYKVRASKTATTIAQLEMDDVRKKIDLE |
| | | | IEQNRLRLKDANKQLATSQKNMAAAEENLRCANVG |
| | | | FKEGVMTVTEVMAAQTAWQTSRMAIIDAEISVKLA |
| | | | QTGLQKALGGL |
| 62 | Phosphoethanolamine_ | Q7CPC0 | MKRTFVTKMVKPIEENSLFFMFMLLVGAFTNVSHR |
| | transferase_ | | NVFGYIELIADVYIICFLLSLCQRTIRQGLVIMLS |
| | CptA | | SVIYVVAIIDTCCKTLFDTPITPTMLLLAQETTGR |
| | | | EATEFFLQYLNLKLFFSAADIILFLAFCHIVMAVK |
| | | | KMKFSTSYLKQPFVAFVLMFTIFVGMALSIYDKVQ |
| | | | LYTVKNLSGLEVAVTNGFAHLYHPVERIVYGLYSN |
| | | | HLIAKQVDGVIMANQQIKVDSCSFTSPTIVLVIGE |
| | | | SANRHHSQLYGYPLPTTPYQLAMKNGKDSLAVFTN |
| | | | VVSPWNLTSKVFKQIFSLQSVDEKGDWSKYVLFPA |
| | | | VFKKAGYHVSFLSNQFPYGINYTPDWTNNLVGGFF |
| | | | LNHPQLNKQMFDYRNVTIHNYDEDLLNDYKEIISY |
| | | | KKPQLIIFHLLGQHFQYSLRCKSNMKKFGIKDYKR |
| | | | MDLTDKEKQTIADYDNATLYNDFVLNKIVEQFRNK |
| | | | DAIIVYLSDHGEDCYGKDVNMAGRLTEVEQINLKK |
| | | | YHEEFEIPFWIWCSPIYKQRHRKIFTETLMARNNK |
| | | | FMTDDLPHLLLYLAGIKTKDYCEERNVISPSFNNN |
| | | | RRRLVLKTIDYDKALYQ |
| 63 | Dipeptide_and_ | P36837 | MFKNHPKGLLQAAFSNMGERFGYYIMNAVLALFLC |
| | tripeptide_ | | SKFGLSDETSGLIASLFLAAIYVMSLVGGVIADRT |
| | prermease_B | | QNYQRTIESGLVVMALGYVALSIPVLATPENNSYL |
| | | | LAFTIFALVLIAVGNGLFKGNLQAIVGQMYDDFET |
| | | | EAAKVSPERLKWAQGQRDAGFQIFYVFINLGALAA |
| | | | PFIAPVLRSWWLGRNGLTYDAALPQLCHKYINGTI |
| | | | GDNLGNLQELATKVGGNSADLASFCPHYLDVFNTG |
| | | | VHYSFIASVVTMLISLIIFMSSKKLFPMPGKKEQI |
| | | | VNVEYTDEEKASMAKEIKQRMYALFAVLGISVFFW |
| | | | FSFHQNGQSLSFFARDFVNTDSVAPEIWQAVNPFF |
| | | | VISLTPLIMWVFAYFTKKGKPISTPRKIAYGMGIA |
| | | | GFAYLFLMGFSLVHNYPSAEQFTSLEPAVRATMKA |
| | | | GPMILILTYFFLTVAELFISPLGLSFVSKVAPKNL |
| | | | QGLCQGLWLGATAVGNGFLWTGPLMYNKWSIWTCW |
| | | | LVFAIVCFISMVVMFGMVKWLERVTKS |
| 64 | C4- | Q9I4F5 | MQKKIKIGLLPRVIIAILLGLFLGYYLPDPAVRVF |
| | dicarboxylate_ | | LTFNSIFSQFLGFMIPLIIIGLVTPAIAGIGKGAG |
| | transport_ | | KLLLATVAIAYVDTIVAGGLSYGTGTWLFPSMIAS |
| | proptein_2 | | TGGAIPHIDKATELTPYFTINIPAMVDVMSSLVFS |
| | | | FIAGLGIAYGGLRTMENLFNEFKTVIEKVIEKAII |
| | | | PLLPLYIFGVFLSMTHNGQARQVLLVFSQIIIVIL |
| | | | VLHVLILIYEFCIAGAIVKHNPFRLLWNMLPAYLT |
| | | | ALGTSSSAATIPVTLKQTVKNGVSEEVAGFVVPLC |
| | | | ATIHLSGSAMKITACALTICMLTDLPHDPGLFIYF |
| | | | ILMLAIIMVAAPGVPGGAIMAALAPLSSILGFNEE |
| | | | AQALMIALYIAMDSFGTACNVTGDGAIALAVNKFF |
| | | | GKKKETSILS |
| 65 | Inner_membrane_ | P76090 | MISVYSIKPQFQRVLTPILELLHRAKVTANQITLW |
| | protein_YnbA | | ACVLSLVIGILFWFAGDVGTWLYLCLPVGLLIRMA |
| | | | LNALDGMMARRYNQITRKGELLNEVGDVVSDTIIY |
| | | | FPLLKYHPESLYFIVAFIALSIINEYAGVMGKVLS |
| | | | AERRYDGPMGKSDRAFVLGLYGVVCLFGINLSGYS |
| | | | VYIFGVIDLLLVLSTWIRIKKTLKVTRNSQTPE |
| 66 | 2′,3′-cyclic- | P08331 | MKLSTILLSIMLGLSSSTMAQQKDVTIKLIETTDV |
| | nucleotide | | HGSFFPYDFITRKPKSGSMARVYTLVEELRKKDGK |
| | | | DNVYLLDNGDILQGQPISYYYNYVAPEKTNIAASV |
| | | | LNYMGYDVATVGNHDIETGHKVYDKWFKELKFPIL |
| | | | GANIIDTKTNKPYILPYYTIKKKNGIKVCVIGMLT |
| | | | PAIPNWLKESIWSGLRFEEMVSCAKRTMAEVKTQE |
| | | | KPDVIVGLFFISGWDGGIKTPEYDEDASKKVAKEV |
| | | | PGFDIVFFGHDHTPHSSIEKNIVGKDVICLDPANN |
| | | | AQRVAIATLTLRPKTVKGKRQYTVTKATGELVDVK |
| | | | ELKADDAFIQHFQPEIDAVKAWSDQVIGRFENTTY |
| | | | SKDSYFGNSAFNDLILNLELETTKADTAFNAPLLE |
| | | | NASIKAGPITVADMFNLYKYENNLCTMRLTGKETR |
| | | | KHLEMSYDLWCNTMKSPEDHLLLLSSTQNDAQRLG |
| | | | FKNFSFNFDSAAGIDYEVDVTKPDGQKVRTLRMSN |
| | | | GEPFDENKWYTVAVNSYRANGGGELLTKGAGIPRD |
| | | | SLKSRIIWESPKDQRHYLMEEIKKAGVMNPQPNHN |
| | | | WKFTPETWTVPAAARDRKLLFGE |
| 67 | Fe(2+)_ | P33650 | KLSELKTGETGVIVKVSGHGGFRKRIIEMGFIKG |
| | transporter_FcoB | | KTVEVLLNAPLQDPVKYKIMGYEVSLRHSEADQTE |
| | | | VLSDVKTHSVGNEEEQEDNQLEMDSTTYDSTDKEL |
| | | | TPEKQSDAVRRKNHTINVALVGNPNCGKTSLFNFA |
| | | | SGAHERVGNYSGVTVDAKVGRAEFDGYVFNLVDLP |
| | | | GTYSLSAYSPEELYVRKQLVDKTPDVVINVIDSSN |
| | | | LERNLYLTTQLIDMHIRMVCALNMFDETEQRGDHI |
| | | | DAQKLSELFGVPMIPTVFTNGRGVKELFRQIIAVY |
| | | | EGKEDESLQFRHIHINHGHEIENGIKEMQEHLKKY |
| | | | PELCHRYSTRYLAIKLLEHDKDVEQLVSPLGDSIE |
| | | | IFNHRDTAAARVKEETGNDSETAIMDAKYGFINGA |
| | | | LKEANFSTGDKKDTYQTTHVIDHVLTNKYFGFPIF |
| | | | FLVLLVMFTATFVIGQYPMDWIEAGVGWLGEFISK |
| | | | NMPAGPVKDMIVDGIIGGVGAVIVFLPQILILYFF |
| | | | ISYMEDCGYMSRAAFIMDRLMHKMGLHGKSFIPLI |
| | | | MGFGCNVPAVMATRTIESRRSRLITMLILPLMSCS |
| | | | ARLPIYVMITGSFFALKYRSLAMLSLYIIGVLMAV |
| | | | AMSRLFSAFVVKGEDTPFVMELPPYRFPTWKAIGR |
| | | | HTWEKGKQYLKKMGGIILVASIIVWALGYFPLPDD |
| | | | PNMDNQARQEQSYIGRIGKAVEPVFRPQGFNWKLD |
| | | | VGLLSGMGAKEIVASTMGVLYSNDGSFSDDNGYSS |
| | | | ETGKYSKLHNLITKDVATMHHISYEEAEPIATLTA |
| | | | FSFLLFVLLYFPCVATIAAIKGETGSWGWALFAAG |
| | | | YTTALAWIVSAVVFQVGMLFM |
| 68 | UDP-N- | P9WJM1 | MESFIIEGGHQLSGTIAPQGAKNEALEVICATLLT |
| | acetylglucosamine | | SEEVIIRNVPDILDVNNLIKLLQDIGVKVKKLAPN |
| | | | EFSFQADEVNLDYLESSDFVKKCSSLRGSVLMIGP |
| | | | LLGRFGKATIAKPGGDKIGRRRLDTHFLGFKNLGA |
| | | | HFGRVEDRDVYEIQADKLVGTYMLLDEASITGTAN |
| | | | IIMAAVLAEGTTTIYNAACEPYIQQLCKMLNAMGA |
| | | | KISGIASNLITIEGVKELHSADHRILPDMIEVGSF |
| | | | IGIAAMIGDGVRIKDVSVPNLGLILDTFHRLGVQI |
| | | | IVDNDDLIIPRQDHYVIDSFIDGTIMTISDAPWPG |
| | | | LTPDLISVLLVVATQAQGSVLFHQKMFESRLFFVD |
| | | | KLIDMGAQIILCDPHRAVVVGHDNAKKLRAGRMSS |
| | | | PDIRAGIALLIAALTAQGTSRIDNIVQIDRGYENI |
| | | | EGRLNALGAKIQRAEVC |
| 69 | Ribitol-5- | Q8RKI9 | MNIAVIFAGGSGLRMHTKSRPKQFLDLNGKPIIIY |
| | phosphate_citidyl | | TLELFDNHPNTDAIVVACIESWIPFLEKQLRKFEI |
| | yltransferase | | NKVVKIIPGGKSGQESIYKGLCAAEEYAQSKGVSN |
| | | | EETTVLIHDGVRPLITEETITDNIKKVEEVGSCIT |
| | | | CIPATETLIVKQADDALEIPSRADSFIARAPQSFR |
| | | | LIDIITAHRRSLAEGKADFTDSCTMMSHYGYKLGT |
| | | | IIGPMENIKITTPTDFFVLRAMVKVHEDQQIFGL |

In some embodiments, the Prevotella bacteria are from a strain of Prevotella bacteria comprising one or more of the proteins listed in Table 1 and that is free or substantially free of one or more proteins listed in Table 2. In some embodiments, the Prevotella bacteria are from a strain of Prevotella bacteria that comprises all of the proteins listed in Table 1 and/or all of the genes encoding the proteins listed in Table 1 and that is free of all of the proteins listed in Table 2 and/or all of the genes encoding the proteins listed in Table 2.

In some embodiments, the engineered Prevotella bacteria described herein are modified to improve Prevotella bacterial (e.g., higher oxygen tolerance, stability, improved freeze-thaw tolerance, shorter generation times). For example, in some embodiments, the engineered Prevotella bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may results in the overexpression and/or underexpression of one or more genes. The engineered microbe(s) may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

In some embodiments, the Prevotella bacteria described herein are modified such that they comprise, are linked to, and/or are bound by a therapeutic moiety.

### Bacterial Compositions

In certain embodiments, the methods provided herein comprise use of bacterial compositions (e.g., pharmaceutical compositions) comprising Prevotella bacteria provided herein.

In some embodiments, the bacterial compositions comprise whole Prevotella histicola bacteria (e.g., live bacteria, killed bacteria, attenuated bacteria). In some embodiments, the Prevotella histicola bacteria is non-viable. In some embodiments, the Prevotella histicola bacteria has been gamma irradiated (e.g., according to a method described herein). In some embodiments, the Prevotella histicola bacteria is live.

In some embodiments, the bacterial composition (e.g., pharmaceutical composition) comprises only one strain of bacteria, e.g., Prevotella histicola.

In some embodiments, the Prevotella histicola is Prevotella Strain B 50329 (NRRL accession number B 50329). In some embodiments, the Prevotella strain is a strain comprising at least at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella Strain B 50329.

In some embodiments, the bacterial compositions comprise whole Prevotella histicola bacteria (e.g., live bacteria, killed bacteria, attenuated bacteria).

In some embodiments, the bacterial composition comprises about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², and/or 1×10¹³ total cells of Prevotella histicola.

In some embodiments, the bacterial composition comprises at least about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², or 1×10¹³ total cells of Prevotella histicola.

In some embodiments, the bacterial composition comprises at most about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ 5×10¹¹, 6×10¹¹, 7×10¹¹, 8×10¹¹, 9×10¹¹, 1×10¹², 2×10¹², 3×10¹², 4×10¹¹, 5×10¹¹, 6×10¹², 7×10¹¹, 8×10¹¹, 9×10¹², or 1×10¹³ total cells of Prevotella histicola.

In some embodiments, the bacterial composition comprises from about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ to about 8×10¹¹ total cells of Prevotella histicola.

In some embodiments, the bacterial composition comprises from about 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2×10¹⁰, 2.1×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4×10¹⁰, 5×10¹⁰, 6×10¹⁰, 7×10¹⁰, 8×10¹⁰, 9×10¹⁰, 1×10¹¹, 1.1×10¹¹, 1.2×10¹¹, 1.3×10¹¹, 1.4×10¹¹, 1.5×10¹¹, 1.6×10¹¹, 1.7×10¹¹, 1.8×10¹¹, 1.9×10¹¹, 2×10¹¹, 2.1×10¹¹, 2.2×10¹¹, 2.3×10¹¹, 2.4×10¹¹, 2.5×10¹¹, 2.6×10¹¹, 2.7×10¹¹, 2.8×10¹¹, 2.9×10¹¹, 3×10¹¹, 3.1×10¹¹, 3.2×10¹¹, 3.3×10¹¹, 3.4×10¹¹, 3.5×10¹¹, 3.6×10¹¹, 3.7×10¹¹, 3.8×10¹¹, 3.9×10¹¹, 4×10¹¹ to about 1×10¹² total cells of Prevotella histicola.

In some embodiments, the bacterial composition comprises about 1.6×10¹⁰ total cells of Prevotella histicola, e.g., of Prevotella Strain B 50329.

In some embodiments, the bacterial composition comprises about 8×10¹⁰ total cells of Prevotella histicola, e.g., of Prevotella Strain B 50329.

In some embodiments, the bacterial composition comprises about 1.6×10¹¹ total cells of Prevotella histicola, e.g., of Prevotella Strain B 50329.

In some embodiments, the bacterial composition comprises about 3.2×10¹¹ total cells of Prevotella histicola, e.g., of Prevotella Strain B 50329.

In some embodiments, the bacterial composition comprises about 8×10¹¹ total cells of Prevotella histicola, e.g., of Prevotella Strain B 50329.

In some embodiments, the bacterial composition comprises about 1.6×10¹⁰ to about 8×10¹¹ total cells of Prevotella histicola, e.g., of Prevotella Strain B 50329.

In some embodiments, the bacterial composition comprises about 1.6×10¹⁰ to about 1.6×10¹¹ total cells of Prevotella histicola, e.g., of Prevotella Strain B 50329.

In some embodiments, the bacterial composition comprises about 1.6×10¹¹ to about 8×10¹¹ total cells of Prevotella histicola, e.g., of Prevotella Strain B 50329.

In some embodiments, the bacterial composition comprises about 8×10¹⁰ to about 8×10¹¹ total cells of Prevotella histicola, e.g., of Prevotella Strain B 50329.

In some embodiments, the Prevotella bacteria may be quantified based on total cells, e.g., total cell count (TCC) (e.g., determined by Coulter counter).

In some embodiments, the bacterial composition comprises at least 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises at most 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition comprises about 2.76 mg, 5 mg, 10 mg, 20 mg, 30 mg, 40 mg, 50 mg, 55 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, or 2.76 g of Prevotella histicola.

In some embodiments, the bacterial composition is administered orally. In some embodiments, the administration to the subject once daily. In some embodiments, the bacterial composition is administered in 2 or more doses (e.g., 3 or more, 4 or more or 5 or more doses). In some embodiments, the administration to the subject of the two or more doses are separated by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days.

In some embodiments, the bacterial composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the bacterial composition is administered once daily for 1, 2, 3, 4, 5, 6, 7, 8, 9, 10, 11, 12, 13, 14, 15, or 16 weeks. In some embodiments, the bacterial composition is administered once daily for at least 1, 2, 3, 4, 5, 6, 7, 8, 9, 10, 11, 12, 13, 14, 15, or 16 weeks.

In some embodiments, the bacterial composition is formulated as a capsule or a tablet. In some embodiments, the bacterial formulation (e.g., composition) comprises an enteric coating or micro encapsulation. In some embodiments, the capsule is an enteric coated capsule (e.g., HPMC coated). In some embodiments, the enteric coating allows release of the bacterial composition in the small intestine, e.g., in the upper small intestine, e.g., in the duodenum. In some embodiments, the enteric coating comprises HPMC.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla or a chimpanzee).

In some embodiments, to quantify the numbers of Prevotella histicola bacteria present in a bacterial sample, electron microscopy (e.g., EM of ultrathin frozen sections) can be used to visualize the bacteria and count their relative numbers. Alternatively, combinations of nanoparticle tracking analysis (NTA), Coulter counting, and dynamic light scattering (DLS) or a combination of these techniques can be used. NTA and the Coulter counter count particles and show their sizes. DLS gives the size distribution of particles, but not the concentration. Bacteria frequently have diameters of 1-2 um. The full range is 0.2-20 um. Combined results from Coulter counting and NTA can reveal the numbers of bacteria in a given sample. Coulter counting reveals the numbers of particles with diameters of 0.7-10 um. NTA reveals the numbers of particles with diameters of 50-1400 nm. For most bacterial samples, the Coulter counter alone can reveal the number of bacteria in a sample.

In some embodiments, the bacterial composition comprises an enteric coating or micro encapsulation. In certain embodiments, the enteric coating or micro encapsulation improves targeting to a desired region of the gastrointestinal tract. For example, in certain embodiments, the bacterial composition comprises an enteric coating and/or microcapsules that dissolves at a pH associated with a particular region of the gastrointestinal tract. In some embodiments, the enteric coating and/or microcapsules dissolve at a pH of about 5.5-6.2 to release in the duodenum, at a pH value of about 7.2-7.5 to release in the ileum, and/or at a pH value of about 5.6-6.2 to release in the colon. Exemplary enteric coatings and microcapsules are described, for example, in U.S. Pat. Pub. No. 2016/0022592, which is hereby incorporated by reference in its entirety. In some embodiments, the enteric coating comprises HPMC.

In certain aspects, provided are bacterial compositions for administration subjects. In some embodiments, the bacterial compositions are combined with additional active and/or inactive materials in order to produce a final product, which may be in single dosage unit or in a multi-dose format. In some embodiments, the bacterial compositions is combined with an adjuvant such as an immuno-adjuvant (e.g., STING agonists, TLR agonists, NOD agonists).

In some embodiments the composition comprises at least one carbohydrate. A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

In some embodiments the composition comprises at least one lipid. As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans). In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0). In some embodiments the composition comprises at least one modified lipid, for example a lipid that has been modified by cooking.

In some embodiments the composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments the composition comprises at least one supplemental vitamin. The at least one vitamin can be fat-soluble or water-soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments the composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments the excipient is a buffering agent. Non-limiting examples of suitable buffering agents include sodium citrate, magnesium carbonate, magnesium bicarbonate, calcium carbonate, and calcium bicarbonate.

In some embodiments the excipient comprises a preservative. Non-limiting examples of suitable preservatives include antioxidants, such as alpha-tocopherol and ascorbate, and antimicrobials, such as parabens, chlorobutanol, and phenol.

In some embodiments the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments the composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isoamorphous silicate, and microcrystalline cellulose as high HLB emulsifier surfactants.

In some embodiments the composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products, including biscuits, cookies, and the like, candies, chewing gums, gummies, cold desserts including jellies, cream caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies.

In some embodiments the composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Dose Forms

Dose forms comprising Prevotella histicola bacteria are also provided herein, e.g., for use in methods to treat or prevent inflammation (such as atopic dermatitis and/or psoriasis) in a subject (e.g., a human subject). A bacterial composition (e.g., pharmaceutical composition) comprising Prevotella histicola bacteria can be formulated as a solid dose form, e.g., for oral administration. The solid dose form can comprise one or more excipients, e.g., pharmaceutically acceptable excipients. The Prevotella histicola bacteria in the solid dose form can be isolated Prevotella histicola bacteria. Optionally, the Prevotella histicola bacteria in the solid dose form can be lyophilized. Optionally, the Prevotella histicola bacteria in the solid dose form are live. Optionally, the Prevotella histicola bacteria in the solid dose form are gamma irradiated. The solid dose form can comprise a tablet, a minitablet, a capsule, a pill, or a powder; or a combination of these forms (e.g., minitablets comprised in a capsule).

The Prevotella histicola bacteria in the solid dose form can be in a powder (e.g., the powder comprises lyophilized Prevotella histicola bacteria). In some embodiments, the powder further comprises mannitol, magnesium stearate, and/or colloidal silicon dioxide. In some embodiments, the powder further comprises mannitol, magnesium stearate, and colloidal silicon dioxide.

In some embodiments, the lyophilized Prevotella bacteria is resuspended in a solution.

In certain embodiments, the bacterial composition (e.g., pharmaceutical composition) provided herein is prepared as a solid dosage form comprising Prevotella histicola bacteria and a pharmaceutically acceptable carrier.

In some embodiments, the solid dosage form comprises a capsule. The capsule can comprise an enteric coating. The capsule can be a size 00, size 0, size 1, size 2, size 3, size 4, or size 5 capsule. The capsule can comprise Prevotella histicola bacteria powder (e.g., lyophilized Prevotella histicola bacteria). In some embodiments, the powder further comprises mannitol, magnesium stearate, and/or colloidal silicon dioxide. In some embodiments, the powder further comprises mannitol, magnesium stearate, and colloidal silicon dioxide.

In some embodiments, the solid dosage form described herein can be, e.g., a tablet or a mini-tablet. In some embodiments, a plurality of mini-tablets can be in (e.g., loaded into) a capsule.

In some embodiments, the solid dosage form comprises a tablet (>4 mm) (e.g., 5 mm-17 mm). For example, the tablet is a 5 mm, 6 mm, 7 mm, 8 mm, 9 mm, 10 mm, 11 mm, 12 mm, 13 mm, 14 mm, 15 mm, 16 mm or 17 mm tablet. The size refers to the diameter of the tablet, as is known in the art. As used herein, the size of the tablet refers to the size of the tablet prior to application of an enteric coating.

In some embodiments, the solid dosage form comprises a mini-tablet. The mini-tablet can be in the size range of 1 mm-4 mm range. E.g., the mini-tablet can be a 1 mm mini-tablet, 1.5 mm mini-tablet, 2 mm mini-tablet, 3 mm mini-tablet, or 4 mm mini-tablet. The size refers to the diameter of the mini-tablet, as is known in the art. As used herein, the size of the minitablet refers to the size of the mini-tablet prior to application of an enteric coating.

The mini-tablets can be in a capsule. The capsule can be a size 00, size 0, size 1, size 2, size 3, size 4, or size 5 capsule. The capsule that contains the mini-tablets can comprise a single layer coating, e.g., a non-enteric coating such as gelatin. The mini-tablets can be inside a capsule: the number of mini-tablets inside a capsule will depend on the size of the capsule and the size of the mini-tablets. As an example, a size 0 capsule can contain 31-35 (an average of 33) mini-tablets that are 3 mm mini-tablets.

The solid dosage form (e.g., tablet or mini-tablet or capsule) described herein can be enterically coated. In some embodiments, the enteric coating comprises HPMC (hydroxyl propyl methyl cellulose). In some embodiments, the enteric coating comprises a polymethacrylate-based copolymer. In some embodiments, the enteric coating comprises a methacrylic acid ethyl acrylate (MAE) copolymer (1:1). In some embodiments, the enteric coating comprises methacrylic acid ethyl acrylate (MAE) copolymer (1:1) (such as Kollicoat MAE 100P).

The solid dose form can comprise a coating. The solid dose form can comprise a single layer coating, e.g., enteric coating, e.g., a Eudragit-based coating, e.g., EUDRAGIT L30 D-55, triethylcitrate, and talc. The solid dose form can comprise two layers of coating. For example, an inner coating can comprise, e.g., EUDRAGIT L30 D-55, triethylcitrate, talc, citric acid anhydrous, and sodium hydroxide, and an outer coating can comprise, e.g., EUDRAGIT L30 D-55, triethylcitrate, and talc. EUDRAGIT is the brand name for a diverse range of polymethacrylate-based copolymers. It includes anionic, cationic, and neutral copolymers based on methacrylic acid and methacrylic/acrylic esters or their derivatives. Eudragits are amorphous polymers having glass transition temperatures between 9 to >150° C. Eudragits are non-biodegradable, nonabsorbable, and nontoxic. Anionic Eudragit L dissolves at pH>6 and is used for enteric coating, while Eudragit S, soluble at pH>7 is used for colon targeting. Eudragit RL and RS, having quaternary ammonium groups, are water insoluble, but swellable/permeable polymers which are suitable for the sustained release film coating applications. Cationic Eudragit E, insoluble at pH≥5, can prevent drug release in saliva.

The solid dose form (e.g., a capsule) can comprise a single layer coating, e.g., a non-enteric coating such as gelatin.

A bacterial composition (e.g., pharmaceutical composition) comprising Prevotella histicola bacteria can be formulated as a suspension, e.g., for oral administration or for injection. Administration by injection includes intravenous (IV), intramuscular (IM), and subcutaneous (SC) administration. For a suspension, Prevotella histicola bacteria can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The Prevotella bacteria in the suspension can be isolated Prevotella histicola bacteria. Optionally, the Prevotella histicola bacteria in the suspension can be lyophilized. Optionally, the Prevotella histicola bacteria in the solid dose form are live. Optionally, the Prevotella histicola bacteria in the suspension can be gamma irradiated.

### Dosage

For oral administration to a human subject, the dose of Prevotella histicola bacteria can be, e.g., about 2×10⁶-about 2×10¹⁶ particles. The dose can be, e.g., about 1×10⁷-about 1×10¹⁵, about 1×10⁸-about 1×10¹⁴, about 1×10⁹-about 1×10¹³, about 1×10¹⁰-about 1×10¹⁴, or about 1×10⁸-about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 2×10¹⁴ particles. The dose can be, e.g., about 2×10¹² particles. The dose can be, e.g., about 2×10¹⁰ particles. The dose can be, e.g., about 1×10¹⁰ particles. Particle count can be determined, e.g., by NTA.

For oral administration to a human subject, the dose of Prevotella histicola bacteria can be, e.g., based on total protein. The dose can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. The dose can be, e.g., about 10 mg total protein. Total protein can be determined, e.g., by Bradford assay or by the BCA assay.

For administration by injection (e.g., intravenous administration) to a human subject, the dose of Prevotella histicola bacteria can be, e.g., about 1×10⁶-about 1×10¹⁶ particles. The dose can be, e.g., about 1×10⁷-about 1×10¹⁵, about 1×10⁸-about 1×10¹⁴, about 1×10⁹-about 1×10¹³, about 1×10¹⁰-about 1×10¹⁴, or about 1×10⁸-about 1×10¹² particles. The dose can be, e.g., about 2×10⁶, about 2×10⁷, about 2×10⁸, about 2×10⁹, about 1×10¹⁰, about 2×10¹⁰, about 2×10¹¹, about 2×10¹², about 2×10¹³, about 2×10¹⁴, or about 1×10¹⁵ particles. The dose can be, e.g., about 1×10¹⁵ particles. The dose can be, e.g., about 2×10¹⁴ particles. The dose can be, e.g., about 2×10¹³ particles. Particle count can be determined, e.g., by NTA.

For administration by injection (e.g., intravenous administration), the dose of Prevotella histicola bacteria can be, e.g., about 5 mg to about 900 mg total protein. The dose can be, e.g., about 20 mg to about 800 mg, about 50 mg to about 700 mg, about 75 mg to about 600 mg, about 100 mg to about 500 mg, about 250 mg to about 750 mg, or about 200 mg to about 500 mg total protein. The dose can be, e.g., about 10 mg, about 25 mg, about 50 mg, about 75 mg, about 100 mg, about 150 mg, about 200 mg, about 250 mg, about 300 mg, about 400 mg, about 500 mg, about 600 mg, or about 750 mg total protein. The dose can be, e.g., about 700 mg total protein. The dose can be, e.g., about 350 mg total protein. The dose can be, e.g., about 175 mg total protein. Total protein can be determined, e.g., by Bradford assay or by the BCA assay.

In certain embodiments, the bacterial composition (e.g., pharmaceutical composition) (e.g., composition of the total dose administered, e.g., once or twice daily) comprises at least 1×10¹⁰ total cells (e.g., at least 1×10¹⁰ total cells, at least 2×10¹⁰ total cells, at least 3×10¹⁰ total cells, at least 4×10¹⁰ total cells, at least 5×10¹⁰ total cells, at least 6×10¹⁰ total cells, at least 7×10¹⁰ total cells, at least 8×10¹⁰ total cells, at least 9×10¹⁰ total cells, at least 1×10¹¹ total cells of the Prevotella histicola bacteria. In some embodiments, the pharmaceutical composition comprises no more than 9×10¹¹ total cells (e.g., no more than 1×10¹⁰ total cells, no more than 2×10¹⁰ total cells, no more than 3×10¹⁰ total cells, no more than 4×10¹⁰ total cells, no more than 5×10¹⁰ total cells, no more than 6×10¹⁰ total cells, no more than 7×10¹⁰ total cells, no more than 8×10¹⁰ total cells, no more than 9×10¹⁰ total cells, no more than 1×10¹¹ total cells, no more than 2×10¹¹ total cells, no more than 3×10¹¹ total cells, no more than 4×10¹¹ total cells, no more than 5×10¹¹ total cells, no more than 6×10¹¹ total cells, no more than 7×10¹¹ total cells, no more than 8×10¹¹ total cells) of the Prevotella histicola bacteria. In some embodiments, the bacterial composition (e.g., pharmaceutical composition) comprises about 6×10⁹ total cells of the Prevotella histicola bacteria. In some embodiments, the bacterial composition (e.g., pharmaceutical composition) comprises about 1.6×10¹⁰ total cells of the Prevotella histicola bacteria. In some embodiments, the bacterial composition (e.g., pharmaceutical composition) comprises about 8×10¹⁰ total cells of the Prevotella histicola bacteria. In some embodiments, the bacterial composition (e.g., pharmaceutical composition) comprises about 1.6×10¹¹ total cells the Prevotella histicola bacteria. In some embodiments, the bacterial composition (e.g., pharmaceutical composition) comprises about 3.2×10¹¹ total cells the Prevotella histicola bacteria. In some embodiments, the bacterial composition (e.g., pharmaceutical composition) comprises about 8×10¹¹ total cells of the Prevotella histicola bacteria. In some embodiments, the bacterial composition (e.g., pharmaceutical composition) comprises about 1.6×10¹⁰ to about 8×10¹¹ total cells of the Prevotella histicola bacteria. In some embodiments, the bacterial composition (e.g., pharmaceutical composition) comprises about 1.6×10¹⁰ to about 1.6×10¹¹ total cells of the Prevotella histicola bacteria. In some embodiments, the bacterial composition (e.g., pharmaceutical composition) comprises about 8×10¹⁰ to about 8×10¹¹ total cells of the Prevotella histicola bacteria. In some embodiments, the bacterial composition (e.g., pharmaceutical composition) comprises about 1.6×10¹¹ to about 8×10¹¹ total cells of the Prevotella histicola bacteria.

In some embodiments, the Prevotella histicola bacteria may be quantified based on total cells, e.g., total cell count (TCC) (e.g., determined by Coulter counter).

In certain embodiments, provided herein are solid dosage forms comprising the Prevotella histicola bacteria. In some embodiments, the solid dosage form comprises an enteric coating. In some embodiments, the solid dosage form is a capsule, e.g., an enteric coated capsule. In some embodiments, each capsule comprises about 8×10¹⁰ total cells of the Prevotella histicola bacteria. In some embodiments, 1, 2, 3, 4, 5, 6, 7, 8, 9, or 10 capsules are administered, e.g., once or twice daily to a subject. In some embodiments, 1 capsule (e.g., comprising about 8×10¹⁰ total cells) is administered, e.g., once or twice daily to a subject. In some embodiments, 2 capsules (e.g., each comprising about 8×10¹⁰ total cells) are administered, e.g., once or twice daily to a subject. In some embodiments, 4 capsules (e.g., each comprising about 8×10¹⁰ total cells) are administered, e.g., once or twice daily to a subject. In some embodiments, 10 capsules (e.g., each comprising about 8×10¹⁰ total cells) are administered, e.g., once or twice daily to a subject. In some embodiments, the Prevotella histicola bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a capsule. In some embodiments, the capsule is an enteric coated capsule. In some embodiments, the capsule comprises about 8×10¹⁰ total cells of the Prevotella histicola bacteria (e.g., total dose of a capsule or plurality of capsules). In some embodiments, the capsule comprises about 1.6×10¹¹ total cells of the Prevotella histicola bacteria (e.g., total dose of a capsule or plurality of capsules). In some embodiments, the capsule comprises about 3.2×10¹¹ total cells of the Prevotella histicola bacteria (e.g., total dose of a capsule or plurality of capsules). In some embodiments, the capsule comprises about 8×10¹¹ total cells of the Prevotella histicola bacteria (e.g., total dose of a capsule or plurality of capsules). In some embodiments, the Prevotella histicola bacteria in the capsule are lyophilized (e.g., in a powder). In some embodiments, the Prevotella bacteria in the capsule are lyophilized in a powder, and the powder further comprises mannitol, magnesium stearate, and/or colloidal silicon dioxide.

In some embodiments, the solid dosage form comprises a tablet. In some embodiments, the tablet is an enteric coated tablet. In some embodiments, the enteric coated tablet is from 5 mm to 17 mm in diameter. In some embodiments, the tablet comprises about 8×10¹⁰ total cells of the Prevotella histicola bacteria (e.g., total dose of a tablet or plurality of tablets). In some embodiments, the tablet comprises about 1.6×10¹¹ total cells of the Prevotella histicola bacteria (e.g., total dose of a tablet or plurality of tablets). In some embodiments, the tablet comprises about 3.2×10¹¹ total cells of the Prevotella histicola bacteria (e.g., total dose of a tablet or plurality of tablets). In some embodiments, the tablet comprises about 8×10¹¹ total cells of the Prevotella histicola bacteria (e.g., total dose of a tablet or plurality of tablets). In some embodiments, the Prevotella histicola bacteria in the tablet are lyophilized (e.g., in a powder).

In some embodiments, the solid dosage form comprises a mini-tablet. In some embodiments, the mini-tablet is enteric coated. In some embodiments, the mini-tablet is from 1 mm to 4 mm in diameter. In some embodiments, the mini-tablet (e.g., enteric coated mini-tablet) is a 1 mm mini-tablet, 1.5 mm mini-tablet, 2 mm mini-tablet, 3 mm mini-tablet, or 4 mm mini-tablet. In some embodiments, the solid dosage form comprises mini-tablets that comprise about 8×10¹⁰ total cells of the Prevotella histicola bacteria (e.g., total dose of a plurality of mini-tablets). In some embodiments, the solid dosage form comprises mini-tablets that comprise about 1.6×10¹¹ total cells of the Prevotella histicola bacteria (e.g., total dose of a plurality of mini-tablets). In some embodiments, the solid dosage form comprises mini-tablets that comprise about 3.2×10¹¹ total cells of the Prevotella histicola bacteria (e.g., total dose of a plurality of mini-tablets). In some embodiments, the solid dosage form comprises mini-tablets that comprise about 8×10¹¹ total cells of the Prevotella histicola bacteria (e.g., total dose of a plurality of mini-tablets). In some embodiments, the Prevotella histicola bacteria in the mini-tablets are lyophilized (e.g., in a powder).

In some embodiments, the mini-tablets (e.g., enteric coated mini-tablets) are contained in a capsule. In some embodiments, the capsule is a size 00, size 0, size 1, size 2, size 3, size 4, or size 5 capsule. In some embodiments, the capsule comprises a non-enteric coating (e.g., gelatin) (e.g., is coated with a non-enteric coating). In some embodiments, the capsule comprises a non-enteric coating. In some embodiments, the capsule comprises gelatin. In some embodiments, the mini-tablets (e.g., enteric coated mini-tablets) that comprise about 8×10¹¹ total cells of the Prevotella histicola bacteria are contained in a capsule(s), wherein optionally the capsule comprises gelatin.

### Gamma-Irradiation

Powders (e.g., of Prevotella histicola bacteria) can be gamma-irradiated at 17.5 kGy radiation unit at ambient temperature.

Frozen biomasses (e.g., of Prevotella histicola bacteria) can be gamma-irradiated at 25 kGy radiation unit in the presence of dry ice.

### Therapeutic Agents

In certain aspects, the methods provided herein include the administration to a subject of a bacterial composition described herein either alone or in combination with an additional therapeutic. In some embodiments, the additional therapeutic is an immunosuppressant, or a steroid.

In some embodiments the Prevotella histicola bacteria is administered to the subject before the therapeutic is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments the Prevotella histicola bacteria is administered to the subject after the therapeutic is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the Prevotella histicola bacteria and the therapeutic are administered to the subject simultaneously or nearly simultaneously (e.g., administrations occur within an hour of each other). In some embodiments, the subject is administered an antibiotic before the Prevotella bacteria is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the subject is administered an antibiotic after the Prevotella bacteria is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the Prevotella bacteria and the antibiotic are administered to the subject simultaneously or nearly simultaneously (e.g., administrations occur within an hour of each other).

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacillin, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilimide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin P1, clarithromycin, erythromycins, furazolidone, fusidic acid, Na fusidate, gramicidin, imipenem, indolicidin, josamycin, magainan II, metronidazole, nitroimidazoles, mikamycin, mutacin B-Ny266, mutacin B-JHl 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic is an immunosuppressive agent, a DMARD, a pain-control drug, a steroid, a non-steroidal anti-inflammatory drug (NSAID), or a cytokine antagonist, and combinations thereof. Representative agents include, but are not limited to, cyclosporin, retinoids, corticosteroids, propionic acid derivative, acetic acid derivative, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966; rofecoxib, acetominophen, Celecoxib, Diclofenac, tramadol, piroxicam, meloxicam, tenoxicam, droxicam, lornoxicam, isoxicam, mefanamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists (e.g., TNF alpha antagonists or TNF alpha receptor antagonists), e.g., ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra/Actemra®), integrin antagonists (TYSABRI® (natalizumab)), IL-1 antagonists (ACZ885 (Ilaris)), Anakinra (Kineret®)), CD4 antagonists, IL-23 antagonists, IL-20 antagonists, IL-6 antagonists, BLyS antagonists (e.g., Atacicept, Benlysta®/LymphoStat-B® (belimumab)), p38 Inhibitors, CD20 antagonists (Ocrelizumab, Ofatumumab (Arzerra®)), interferon gamma antagonists (Fontolizumab), prednisolone, Prednisone, dexamethasone, Cortisol, cortisone, hydrocortisone, methylprednisolone, betamethasone, triamcinolone, beclometasome, fludrocortisone, deoxycorticosterone, aldosterone, Doxycycline, vancomycin, pioglitazone, SBI-087, SCIO-469, Cura-100, Oncoxin+Viusid, TwHF, Methoxsalen, Vitamin D—ergocalciferol, Milnacipran, Paclitaxel, rosig tazone, Tacrolimus (Prograf®), RADOOl, rapamune, rapamycin, fostamatinib, Fentanyl, XOMA 052, Fostamatinib disodium, rosightazone, Curcumin (Longvida™), Rosuvastatin, Maraviroc, ramipn1, Milnacipran, Cobiprostone, somatropin, tgAAC94 gene therapy vector, MK0359, GW856553, esomeprazole, everolimus, trastuzumab, JAK1 and JAK2 inhibitors, pan JAK inhibitors, e.g., tetracyclic pyridone 6 (P6), 325, PF-956980, denosumab, IL-6 antagonists, CD20 antagonistis, CTLA4 antagonists, IL-8 antagonists, IL-21 antagonists, IL-22 antagonist, integrin antagonists (Tysarbri® (natalizumab)), VGEF antagonsits, CXCL antagonists, MMP antagonists, defensin antagonists, IL-1 antagonists (including IL-1 beta antagonsits), and IL-23 antagonists (e.g., receptor decoys, antagonistic antibodies, etc.).

In some embodiments, the additional therapeutic is an oral PDE4 inhibitor (such as apremilast). In some embodiments, the additional therapeutic is apremilast, etanercept, infliximab, adalimumab, ustekinumab, or secukinumab.

In some embodiments, the agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

### Administration

In some embodiments, the bacterial composition is administered orally. In some embodiments, the administration to the subject once daily. In some embodiments, the bacterial composition is administered in 2 or more doses (e.g., 3 or more, 4 or more or 5 or more doses). In some embodiments, the administration to the subject of the two or more doses are separated by at least 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days or 21 days.

In some embodiments, the bacterial composition is administered once daily for 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, 35 days, 36 days, 37 days, 38 days, 39 days, 40 days, 41 days, or 42 days.

In some embodiments, the bacterial composition is formulated as a capsule or a tablet. In some embodiments, the bacterial formulation comprises an enteric coating or micro encapsulation. In some embodiments, the capsule is an enteric coated capsule.

In some embodiments, the subject is a mammal. In some embodiments, the subject is a human. In some embodiments, the subject is a non-human mammal (e.g., a dog, a cat, a cow, a horse, a pig, a donkey, a goat, a camel, a mouse, a rat, a guinea pig, a sheep, a llama, a monkey, a gorilla or a chimpanzee).

In some embodiments of the methods provided herein, the bacterial composition is administered in conjunction with the administration of an additional therapeutic. In some embodiments, the bacterial composition comprises Prevotella bacteria co-formulated with the additional therapeutic. In some embodiments, the bacterial composition is co-administered with the additional therapeutic. In some embodiments, the additional therapeutic is administered to the subject before administration of the bacterial composition (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic is administered to the subject after administration of the bacterial composition (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments the same mode of delivery are used to deliver both the bacterial composition and the additional therapeutic. In some embodiments different modes of delivery are used to administer the bacterial composition and the additional therapeutic. For example, in some embodiments the bacterial composition is administered orally while the additional therapeutic is administered via injection (e.g., an intravenous, and/or intramuscular injection).

In certain embodiments, the bacterial compositions, dosage forms, and kits described herein can be administered in conjunction with any other conventional treatment. These treatments may be applied as necessary and/or as indicated and may occur before, concurrent with or after administration of the bacterial compositions, dosage forms, and kits described herein.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, and other compounds such as drugs being administered concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of the bacterial compositions described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (e.g., autoimmune disease, inflammatory disease, metabolic disease), or treat disease, e.g., delay its onset, ameliorate one or more symptom of the disease, lessen the severity of the disease (or a symptom thereof), or slow or stop its progression. One skilled in the art will recognize that dosage will depend upon a variety of factors including the strength of the particular compound employed, as well as the age, species, condition, and body weight of the subject. The size of the dose will also be determined by the route, timing, and frequency of administration as well as the existence, nature, and extent of any adverse side-effects that might accompany the administration of a particular compound and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications (e.g., for treatment and/or prevention), the dosages of the active agents used in accordance with the invention vary depending on the active agent, the age, weight, and clinical condition of the recipient patient, and the experience and judgment of the clinician or practitioner administering the therapy, among other factors affecting the selected dosage.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a bacterial composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response and/or the time period for a subject to clear the bacteria from normal tissue. In one example, the time period can be a function of the time period for a subject to mount an immune response; for example, the time period can be more than the time period for a subject to mount an immune response, such as more than about one week, more than about ten days, more than about two weeks, or more than about a month; in another example, the time period can be less than the time period for a subject to mount an immune response, such as less than about one week, less than about ten days, less than about two weeks, or less than about a month. In another example, the time period can be a function of the time period for a subject to clear the bacteria from normal tissue; for example, the time period can be more than the time period for a subject to clear the bacteria from normal tissue, such as more than about a day, more than about two days, more than about three days, more than about five days, or more than about a week.

In some embodiments, the delivery of an additional therapeutic in combination with the bacterial composition described herein reduces the adverse effects and/or improves the efficacy of the additional therapeutic.

The effective dose of an additional therapeutic described herein is the amount of the therapeutic agent that is effective to achieve the desired therapeutic response for a particular patient, composition, and mode of administration, with the least toxicity to the patient. The effective dosage level can be identified using the methods described herein and will depend upon a variety of pharmacokinetic factors including the activity of the particular compositions administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. In general, an effective dose of an additional therapy will be the amount of the therapeutic agent which is the lowest dose effective to produce a therapeutic effect. Such an effective dose will generally depend upon the factors described above.

The toxicity of an additional therapy is the level of adverse effects experienced by the subject during and following treatment. Adverse events associated with additional therapy toxicity include, but are not limited to, abdominal pain, acid indigestion, acid reflux, allergic reactions, alopecia, anaphylaxis, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hyperchloremia, hyperglycemia, hyperkalemia, hyperlipasemia, hypermagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heart beat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Immune Disorders

In some embodiments, the methods and compositions described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the disease or disorder is psoriasis (e.g., mild to moderate psoriasis). In some embodiments, the disease or disorder is atopic dermatitis (e.g., mild to moderate atopic dermatitis).

The methods described herein can be used to treat any subject in need thereof. As used herein, a “subject in need thereof” includes any subject that has a disease or disorder associated with a pathological immune response (psoriasis (e.g., mild to moderate psoriasis) or atopic dermatitis (e.g., mild to moderate atopic dermatitis)), as well as any subject with an increased likelihood of acquiring a such a disease or disorder.

The compositions described herein can be used, for example, as a bacterial composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease; an allergic disease, such as a food allergy, pollenosis, or asthma; an infectious disease, such as an infection with Clostridium difficile; an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease); a bacterial composition for suppressing rejection in organ transplantation or other situations in which tissue rejection might occur; a supplement, food, or beverage for improving immune functions; or a reagent for suppressing the proliferation or function of immune cells.

In some embodiments, the methods provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below.

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, coeliac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and compositions described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, good pasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and compositions described herein may be used to treat T-cell mediated hypersensitivity diseases having an inflammatory component. Such conditions include, but are not limited to, contact hypersensitivity, contact dermatitis (including that due to poison ivy), uticaria, skin allergies, respiratory allergies (hay fever, allergic rhinitis, house dustmite allergy) and gluten-sensitive enteropathy (Celiac disease).

Other immune disorders which may be treated with the methods and compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autroimmine) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosis, psoriasis, chronic obstructive pulmonary disease, and inflammation accompanying infectious conditions (e.g., sepsis).

In some aspects, bacterial compositions for use of treating psoriasis and/or atopic dermatitis are disclosed. In some aspects, a bacterial composition comprising Prevotella histicola, wherein the Prevotella histicola is a strain comprising at least 85% sequence identity to the nucleotide sequence of the Prevotella histicola Strain B 50329 (NRRL accession number B 50329) for use in treating psoriasis is described herein. In other aspects, a bacterial composition comprising Prevotella histicola, wherein the Prevotella histicola is a strain comprising at least 85% sequence identity to the nucleotide sequence of the Prevotella histicola Strain B 50329 (NRRL accession number B 50329) for use in treating atopic dermatitis is described herein.

In some aspects, uses of a bacterial composition for the preparation of a medicament for treating psoriasis (e.g., mild to moderate psoriasis) and/or atopic dermatitis (e.g., mild to moderate atopic dermatitis) are disclosed. In some aspects, use of a bacterial composition for the preparation of a medicament for treating psoriasis wherein the bacterial composition comprises Prevotella histicola, wherein the Prevotella histicola is a strain comprising at least 85% sequence identity to the nucleotide sequence of the Prevotella histicola Strain B 50329 (NRRL accession number B 50329) is described herein. In other aspects, use of a bacterial composition for the preparation of a medicament for treating atopic dermatitis wherein the bacterial composition comprises Prevotella histicola, wherein the Prevotella histicola is a strain comprising at least 85% sequence identity to the nucleotide sequence of the Prevotella histicola Strain B 50329 (NRRL accession number B 50329) is described herein.

Numerous embodiments are further provided that can be applied to any aspect of the present invention described herein. For example, in some embodiments, the Prevotella histicola is a strain comprising at least 99.9% sequence identity to the nucleotide sequence of the Prevotella histicola Strain B 50329 (NRRL accession number B 50329). In some embodiments, the Prevotella histicola is the Prevotella histicola Strain B 50329 (NRRL accession number B 50329). In some embodiments, the bacterial composition is administered orally. In some embodiments, the bacterial composition is formulated as a capsule or a tablet. In some embodiments, the capsule is an enteric coated capsule. In some embodiments, the bacterial composition comprises about 1.6×10¹⁰ total cells of Prevotella histicola. In some embodiments, the bacterial composition comprises at most about 1.6×10¹⁰ total cells of Prevotella histicola. In some embodiments, the bacterial composition comprises about 1.6×10¹¹ total cells of Prevotella histicola. In some embodiments, the bacterial composition comprises at most about 1.6×10¹¹ total cells of Prevotella histicola. In some embodiments, the bacterial composition comprises about 8×10¹¹ total cells of Prevotella histicola. In some embodiments, the bacterial composition comprises at most about 8×10¹¹ total cells of Prevotella histicola. In some embodiments, the bacterial composition comprises from about 1.6×10¹⁰ to about 8×10¹¹ total cells of Prevotella histicola. In some embodiments, the bacterial composition comprises about 2.76 mg, about 55 mg, about 550 mg, or about 2.76 g of Prevotella histicola. In some embodiments, the bacterial composition is administered at least once daily. In some embodiments, the bacterial composition is administered once daily. In some embodiments, the bacterial composition is administered once daily for 15 continuous days. In some embodiments, the bacterial composition is administered once daily for 28 continuous days. In some embodiments, the bacterial composition is administered once daily for 29 continuous days. In some embodiments, the psoriasis is mild to moderate psoriasis. In some embodiments, the atopic dermatitis is mild to moderate atopic dermatitis.

### Additional Exemplary Embodiments

In exemplary embodiment 1, provided herein is a method of treating psoriasis in a subject comprising administering to the subject a bacterial composition comprising Prevotella histicola, wherein the Prevotella histicola is a strain comprising at least 85% sequence identity to the nucleotide sequence of the Prevotella histicola Strain B 50329 (NRRL accession number B 50329).

In exemplary embodiment 2, provided herein is a method of decreasing Lesion Severity Score (LSS) (e.g., mean LSS) (e.g., as compared to baseline or placebo control) in a subject (e.g., a subject with psoriasis) comprising administering to the subject a bacterial composition comprising Prevotella histicola, wherein the Prevotella histicola is a strain comprising at least 85% sequence identity to the nucleotide sequence of the Prevotella histicola Strain B 50329 (NRRL accession number B 50329).

In exemplary embodiment 3, provided herein is the method of embodiment 2, wherein the mean LSS is decreased in the subject.

In exemplary embodiment 4, provided herein is the method of embodiment 2 or embodiment 3, wherein the LSS is reduced as compared to baseline or placebo control.

In exemplary embodiment 5, provided herein is a method of decreasing Psoriasis Area and Severity Index (PASI) score (e.g., mean PASI score) (e.g., as compared to baseline or placebo control) in a subject (e.g., a subject with psoriasis) comprising administering to the subject a bacterial composition comprising Prevotella histicola, wherein the Prevotella histicola is a strain comprising at least 85% sequence identity to the nucleotide sequence of the Prevotella histicola Strain B 50329 (NRRL accession number B 50329).

In exemplary embodiment 6, provided herein is the method of embodiment 5, wherein the mean PASI score is decreased in the subject.

In exemplary embodiment 7, provided herein is the method of embodiment 5 or embodiment 6, wherein the PASI score is reduced as compared to baseline or placebo control.

In exemplary embodiment 8, provided herein is a method of increasing a sustained clinical effect (e.g., continued reductions from baseline (or placebo) in mean LSS and/or PASI, e.g., two weeks after completion of dosing) in a subject (e.g., a subject with psoriasis) comprising administering to the subject a bacterial composition comprising Prevotella histicola, wherein the Prevotella histicola is a strain comprising at least 85% sequence identity to the nucleotide sequence of the Prevotella histicola Strain B 50329 (NRRL accession number B 50329).

In exemplary embodiment 9, provided herein is the method of embodiment 8, wherein the sustained clinical effect comprises continued reductions from baseline or placebo in mean LSS and/or PASI after completion of dosing.

In exemplary embodiment 10, provided herein is the method of embodiment 9, wherein the reductions from baseline or placebo in mean LSS and/or PASI are continued for at least 2 weeks after dosing.

In exemplary embodiment 11, provided herein is the method of any one of embodiments 2-10, wherein the LSS and/or PASI score are reduced by at least 10%, 15%, 20%, 25%, 30%, 35%, 40% 50%, 60%, 70%, 80%, or 90% compared to baseline or placebo.

In exemplary embodiment 12, provided herein is the method of any one of embodiments 1-11, wherein the Prevotella histicola is a strain comprising at least 99.9% sequence identity to the nucleotide sequence of the Prevotella histicola Strain B 50329 (NRRL accession number B 50329).

In exemplary embodiment 13, provided herein is the method of any one of embodiments 1-11, wherein the Prevotella histicola is the Prevotella histicola Strain B 50329 (NRRL accession number B 50329).

In exemplary embodiment 14, provided herein is the method of any one of embodiments 1-13, wherein the bacterial composition is administered orally.

In exemplary embodiment 15, provided herein is the method of any one of embodiments 1-14, wherein the bacterial composition is formulated as a capsule or a tablet.

In exemplary embodiment 16, provided herein is the method of embodiment 15, wherein the capsule is an enteric coated capsule.

In exemplary embodiment 17, provided herein is the method of any one of embodiments 1-16, wherein the bacterial composition comprises at least about 1.6×10¹⁰ total cells of Prevotella histicola.

In exemplary embodiment 18, provided herein is the method of any one of embodiments 1-16, wherein the bacterial composition comprises about 1.6×10¹⁰ total cells of Prevotella histicola.

In exemplary embodiment 19, provided herein is the method of any one of embodiments 1-16, wherein the bacterial composition comprises about 1.6×10¹¹ total cells of Prevotella histicola.

In exemplary embodiment 20, provided herein is the method of any one of embodiments 1-16, wherein the bacterial composition comprises at most about 1.6×10¹¹ total cells of Prevotella histicola.

In exemplary embodiment 21, provided herein is the method of any one of embodiments 1-16, wherein the bacterial composition comprises about 8×10¹¹ total cells of Prevotella histicola.

In exemplary embodiment 22, provided herein is the method of any one of embodiments 1-16, wherein the bacterial composition comprises at most about 8×10¹¹ total cells of Prevotella histicola.

In exemplary embodiment 23, provided herein is the method of any one of embodiments 1-16, wherein the bacterial composition comprises from about 1.6×10¹⁰ to about 8×10¹¹ total cells of Prevotella histicola.

In exemplary embodiment 24, provided herein is the method of any one of embodiments 1-16, wherein the bacterial composition comprises at least about 2.76 g of Prevotella histicola.

In exemplary embodiment 25, provided herein is the method of any one of embodiments 1-16, wherein the bacterial composition comprises about 2.76 g of Prevotella histicola.

In exemplary embodiment 26, provided herein is the method of any one of embodiments 1-16, wherein the bacterial composition comprises about 55 mg of Prevotella histicola.

In exemplary embodiment 27, provided herein is the method of any one of embodiments 1-16, wherein the bacterial composition comprises about 550 mg of Prevotella histicola.

In exemplary embodiment 28, provided herein is the method of any one of embodiments 1-27, wherein the bacterial composition is administered at least once daily.

In exemplary embodiment 29, provided herein is the method of any one of embodiments 1-27, wherein the bacterial composition is administered once daily.

In exemplary embodiment 30, provided herein is the method of any one of embodiments 1-27, wherein the bacterial composition is administered once daily for 15 continuous days.

In exemplary embodiment 31, provided herein is the method of any one of embodiments 1-27, wherein the bacterial composition is administered once daily for 28 continuous days.

In exemplary embodiment 32, provided herein is the method of any one of embodiments 1-27, wherein the bacterial composition is administered once daily for 29 continuous days.

In exemplary embodiment 33, provided herein is the method of any one of embodiments 1-32, wherein the psoriasis is mild to moderate psoriasis.

In exemplary embodiment 34, provided herein is a method of treating atopic dermatitis in a subject comprising administering to the subject a bacterial composition comprising Prevotella histicola, wherein the Prevotella histicola is a strain comprising at least 85% sequence identity to the nucleotide sequence of the Prevotella histicola Strain B 50329 (NRRL accession number B 50329).

In exemplary embodiment 35, provided herein is the method of embodiment 34, wherein the Prevotella histicola is a strain comprising at least 99.9% sequence identity to the nucleotide sequence of the Prevotella histicola Strain B 50329 (NRRL accession number B 50329).

In exemplary embodiment 36, provided herein is the method of embodiment 34, wherein the Prevotella histicola is the Prevotella histicola Strain B 50329 (NRRL accession number B 50329).

In exemplary embodiment 37, provided herein is the method of any one of embodiments 34-36, wherein the bacterial composition is administered orally.

In exemplary embodiment 38, provided herein is the method of any one of embodiments 34-37, wherein the bacterial composition is formulated as a capsule or a tablet.

In exemplary embodiment 39, provided herein is the method of embodiment 38, wherein the capsule is an enteric coated capsule.

In exemplary embodiment 40, provided herein is the method of any one of embodiments 34-39, wherein the bacterial composition comprises at least about 1.6×10¹⁰ total cells of Prevotella histicola.

In exemplary embodiment 41, provided herein is the method of any one of embodiments 34-39, wherein the bacterial composition comprises about 1.6×10¹⁰ total cells of Prevotella histicola.

In exemplary embodiment 42, provided herein is the method of any one of embodiments 34-39, wherein the bacterial composition comprises about 1.6×10¹¹ total cells of Prevotella histicola.

In exemplary embodiment 43, provided herein is the method of any one of embodiments 34-39, wherein the bacterial composition comprises at most about 1.6×10¹¹ total cells of Prevotella histicola.

In exemplary embodiment 44, provided herein is the method of any one of embodiments 34-39, wherein the bacterial composition comprises about 8×10¹¹ total cells of Prevotella histicola.

In exemplary embodiment 45, provided herein is the method of any one of embodiments 34-39, wherein the bacterial composition comprises at most about 8×10¹¹ total cells of Prevotella histicola.

In exemplary embodiment 46, provided herein is the method of any one of embodiments 34-39, wherein the bacterial composition comprises from about 1.6×10¹⁰ to about 8×10¹¹ total cells of Prevotella histicola.

In exemplary embodiment 47, provided herein is the method of any one of embodiments 34-39, wherein the bacterial composition comprises at least about 2.76 g of Prevotella histicola.

In exemplary embodiment 48, provided herein is the method of any one of embodiments 34-39, wherein the bacterial composition comprises about 2.76 g of Prevotella histicola.

In exemplary embodiment 49, provided herein is the method of any one of embodiments 34-39, wherein the bacterial composition comprises about 55 mg of Prevotella histicola.

In exemplary embodiment 50, provided herein is the method of any one of embodiments 34-39, wherein the bacterial composition comprises about 550 mg of Prevotella histicola.

In exemplary embodiment 51, provided herein is the method of any one of embodiments 34-50, wherein the bacterial composition is administered at least once daily.

In exemplary embodiment 52, provided herein is the method of any one of embodiments 34-50, wherein the bacterial composition is administered once daily.

In exemplary embodiment 53, provided herein is the method of any one of embodiments 34-50, wherein the bacterial composition is administered once daily for 15 continuous days.

In exemplary embodiment 54, provided herein is the method of any one of embodiments 34-50, wherein the bacterial composition is administered once daily for 28 continuous days.

In exemplary embodiment 55, provided herein is the method of any one of embodiments 34-50, wherein the bacterial composition is administered once daily for 29 continuous days.

In exemplary embodiment 56, provided herein is the method of any one of embodiments 34-55, wherein the atopic dermatitis is mild to moderate atopic dermatitis.

In exemplary embodiment 57, provided herein is the method of any one of embodiments 1-56, wherein the bacterial composition comprises one strain of bacteria, wherein the one strain of bacteria is a strain comprising at least 99.9% sequence identity to the nucleotide sequence of the Prevotella histicola Strain B 50329 (NRRL accession number B 50329).

In exemplary embodiment 58, provided herein is the method of any one of embodiments 1-56, wherein the bacterial composition comprises one strain of bacteria, wherein the one strain of bacteria is the Prevotella histicola Strain B 50329 (NRRL accession number B 50329).

In exemplary embodiment 59, provided herein is a method of enhancing anti-inflammatory cytokine production comprising administering a bacterial composition (e.g., pharmaceutical composition) comprising Prevotella histicola, wherein the Prevotella histicola is a strain comprising at least 85% sequence identity to the nucleotide sequence of the Prevotella histicola Strain B 50329 (NRRL accession number B 50329).

In exemplary embodiment 60, provided herein is the method of embodiment 59, wherein the Prevotella histicola is the Prevotella histicola Strain B 50329 (NRRL accession number B 50329).

In exemplary embodiment 61, provided herein is the method of any one of embodiments 59-60, wherein the bacterial composition is administered orally.

In exemplary embodiment 62, provided herein is the method of any one of embodiments 59-61, wherein the bacterial composition is formulated as a capsule or a tablet.

In exemplary embodiment 63, provided herein is the method of embodiment 62, wherein the capsule is an enteric coated capsule.

In exemplary embodiment 64, provided herein is the method of any one of embodiments 59-63, wherein the bacterial composition comprises at least about 1.6×10¹⁰ total cells of Prevotella histicola.

In exemplary embodiment 65, provided herein is the method of any one of embodiments 59-63, wherein the bacterial composition comprises about 1.6×10¹⁰ total cells of Prevotella histicola.

In exemplary embodiment 66, provided herein is the method of any one of embodiments 59-63, wherein the bacterial composition comprises about 1.6×10¹¹ total cells of Prevotella histicola.

In exemplary embodiment 67, provided herein is the method of any one of embodiments 59-63, wherein the bacterial composition comprises at most about 1.6×10¹¹ total cells of Prevotella histicola.

In exemplary embodiment 68, provided herein is the method of any one of embodiments 59-63, wherein the bacterial composition comprises about 8×10¹¹ total cells of Prevotella histicola.

In exemplary embodiment 69, provided herein is the method of any one of embodiments 59-63, wherein the bacterial composition comprises at most about 8×10¹¹ total cells of Prevotella histicola.

In exemplary embodiment 70, provided herein is the method of any one of embodiments 59-63, wherein the bacterial composition comprises from about 1.6×10¹⁰ to about 8×10¹¹ total cells of Prevotella histicola.

In exemplary embodiment 71, provided herein is the method of any one of embodiments 59-63, wherein the bacterial composition comprises at least about 2.76 g of Prevotella histicola.

In exemplary embodiment 72, provided herein is the method of any one of embodiments 59-63, wherein the bacterial composition comprises about 2.76 g of Prevotella histicola.

In exemplary embodiment 73, provided herein is the method of any one of embodiments 59-63, wherein the bacterial composition comprises about 55 mg of Prevotella histicola.

In exemplary embodiment 74, provided herein is the method of any one of embodiments 59-63, wherein the bacterial composition comprises about 550 mg of Prevotella histicola.

In exemplary embodiment 75, provided herein is the method of any one of embodiments 59-74, wherein the bacterial composition is administered at least once daily.

In exemplary embodiment 76, provided herein is the method of any one of embodiments 59-74, wherein the bacterial composition is administered once daily.

In exemplary embodiment 77, provided herein is the method of any one of embodiments 59-74, wherein the bacterial composition is administered once daily for 15 continuous days.

In exemplary embodiment 78, provided herein is the method of any one of embodiments 59-74, wherein the bacterial composition is administered once daily for 28 continuous days.

In exemplary embodiment 79, provided herein is the method of any one of embodiments 59-74, wherein the bacterial composition is administered once daily for 29 continuous days.

In exemplary embodiment 80, provided herein is the method of any one of embodiments 59-79, wherein the anti-inflammatory cytokine is IL-10 and/or IL-27.

In exemplary embodiment 81, provided herein is the method of any one of embodiments 59-80, wherein the anti-inflammatory cytokine is expressed by M1-type APCs.

### EXAMPLES

### Example 1: Prevotella histicola Strain B in a Mouse Model of Delayed-Type Hypersensitivity (DTH)

Delayed-type hypersensitivity (DTH) is an animal model of atopic dermatitis (or allergic contact dermatitis), as reviewed by Petersen et al. (In vivo pharmacological disease models for psoriasis and atopic dermatitis in drug discovery. Basic & Clinical Pharm & Toxicology. 2006. 99(2): 104-115; see also Irving C. Allen (ed.) Mouse Models of Innate Immunity: Methods and Protocols, Methods in Molecular Biology, 2013. vol. 1031, DOI 10.1007/978-1-62703-481-4_13). It can be induced in a variety of mouse and rat strains using various haptens or antigens, for example an antigen emulsified with Complete Freund's Adjuvant, (CFA) or other adjuvant. DTH is characterized by sensitization as well as an antigen-specific T cell-mediated reaction that results in erythema, edema, and cellular infiltration—especially infiltration of antigen presenting cells (APCs), eosinophils, activated CD4+ T cells, and cytokine-expressing Th2 cells.

Generally, mice are primed with an antigen administered in the context of an adjuvant (e.g. Complete Freund's Adjuvant) in order to induce a secondary (or memory) immune response measured by swelling and antigen-specific antibody titer.

Prevotella histicola Strain B are tested for their efficacy in the mouse model of DTH, either alone or in combination, with or without the addition of other anti-inflammatory treatments. For example, 6-8 week old C57Bl/6 mice are obtained from Taconic (Germantown, N.Y.), or other vendor. Groups of mice are administered four subcutaneous (s.c.) injections at four sites on the back (upper and lower) of antigen (e.g., Keyhole limpet hemocyanin (KLH) or Ovalbumin (OVA)) in an effective dose (50 ul total volume per site). For a DTH response, animals may be injected intradermally (i.d.) in the ears using methods known in the art. Some mice serve as control animals. Some groups of mice may be challenged with 10 ul per ear (vehicle control (0.01% DMSO in saline) in the left ear and antigen (approximately 21.2 ug (12 nmol) in the right ear) on day 8. To measure ear inflammation, the ear thickness of manually restrained animals may be measured using a Mitutoyo micrometer. The ear thickness may be measured before intradermal challenge as the baseline level for each individual animal. Subsequently, the ear thickness may be measured two times after intradermal challenge, at approximately 24 hours and 48 hours (i.e. days 9 and 10). The corticosteroid, Dexamethasone, may be used for a positive control.

Treatment with bacteria is initiated at some point, either around the time of priming or around the time of DTH challenge. For example, bacteria may be administered at the same time as the subcutaneous injections (day 0), or they may be administered prior to, or upon, intradermal injection. Bacteria are administered at varied doses and at defined intervals. While some mice receive bacteria through i.v. injection, other mice may receive bacteria through intraperitoneal (i.p.) injection, subcutaneous (s.c.) injection, nasal route administration, oral gavage, topical administration, intradermal (i.d.) injection, or other means of administration. Some mice may receive bacteria every day (e.g. starting on day 0), while others may receive bacteria at alternative intervals (e.g. every other day, or once every three days). The bacterial cells may be live, dead, or weakened. The bacterial cells may be harvested fresh (or frozen) and administered, or they may be irradiated, lyophilized, or heat-killed prior to administration.

For example, some groups of mice may receive between 1×10⁴ and 5×10⁹ bacterial cells.

In other experiments, some groups of mice may be treated with anti-inflammatory agent(s) (e.g. anti-CD154, blockade of members of the TNF family, or other treatment), and/or an appropriate control (e.g. vehicle or control antibody) at various timepoints and at effective doses. Furthermore, some mice may be treated with antibiotics prior to treatment. For example, vancomycin (0.5 g/L), ampicillin (1.0 g/L), gentamicin (1.0 g/L) and amphotericin B (0.2 g/L) are added to the drinking water, and antibiotic treatment is halted at the time of treatment or a few days prior to treatment. Some immunized mice are treated without receiving antibiotics.

At various timepoints, serum samples are taken. Other groups of mice are sacrificed and lymph nodes, spleen, mesenteric lymph nodes (MLN), the small intestine, colon, and other tissues may be removed for histology studies, ex vivo histological, cytokine and/or flow cytometric analysis using methods known in the art. Some mice are exsanguinated from the orbital plexus under O2/CO2 anesthesia and ELISA assays performed.

Tissues may be dissociated using dissociation enzymes according to the manufacturer's instructions. Cells are stained for analysis by flow cytometry using techniques known in the art. Staining antibodies can include anti-CD11c (dendritic cells), anti-CD80, anti-CD86, anti-CD40, anti-MHCII, anti-CD8a, anti-CD4, and anti-CD103. Other markers that may be analyzed include pan-immune cell marker CD45, T cell markers (CD3, CD4, CD8, CD25, Foxp3, T-bet, Gata3, Roryt, Granzyme B, CD69, PD-1, CTLA-4), and macrophage/myeloid markers (CD11b, MHCII, CD206, CD40, CSF1R, PD-L1, Gr-1, F4/80). In addition to immunophenotyping, serum cytokines are analyzed including, but not limited to, TNFa, IL-17, IL-13, IL-12p70, IL12p40, IL-10, IL-6, IL-5, IL-4, IL-2, IL-1b, IFNy, GM-CSF, G-CSF, M-CSF, MIG, IP10, MIP1b, RANTES, and MCP-1. Cytokine analysis may be carried out on immune cells obtained from lymph nodes or other tissue, and/or on purified CD45+ infiltrated immune cells obtained ex vivo. Finally, immunohistochemistry is carried out on various tissue sections to measure T cells, macrophages, dendritic cells, and checkpoint molecule protein expression.

Mice were primed and challenged with KLH as described above and, following measurement of the ear swelling at 48 hours, mice were sacrificed.

Ears were removed from the sacrificed animals and placed in cold EDTA-free protease inhibitor cocktail (Roche). Ears were homogenized using bead disruption and supernatants analyzed for IL-1β by Luminex kit (EMD Millipore) as per manufacturer's instructions.

In order to examine the impact and longevity of DTH protection, rather than being sacrificed, some mice may be rechallenged with the challenging antigen. Mice are analyzed for susceptibility to DTH and severity of response at various timepoints.

### Example 2: Prevotella histicola Strain B in a Mouse Model of Psoriasis

Psoriasis is a T-cell-mediated chronic inflammatory skin disease. So-called “plaque-type” psoriasis is the most common form of psoriasis and is typified by dry scales, red plaques, and thickening of the skin due to infiltration of immune cells into the dermis and epidermis. Several animal models have contributed to the understanding of this disease, as reviewed by Gudjonsson et al. (Mouse models of psoriasis. J Invest Derm. 2007. 127: 1292-1308; see also van der Fits et al. Imiquimod-induced psoriasis-like skin inflammation in mice is mediated via the IL-23/IL-17 axis. J. Immunol. 2009 May 1. 182(9): 5836-45).

Psoriasis can be induced in a variety of mouse models, including those that use transgenic, knockout, or xenograft models, as well as topical application of imiquimod (IMQ), a TLR7/8 ligand.

Prevotella histicola strain B is tested for its efficacy in the mouse model of psoriasis, with or without the addition of other anti-inflammatory treatments. For example, 6-8 week old C57Bl/6 or Balb/c mice are obtained from Taconic (Germantown, N.Y.), or other vendor. Mice are shaved on the back and the right ear. Groups of mice receive a daily topical dose of 62.5 mg of commercially available IMQ cream (5%) (Aldara; 3M Pharmaceuticals). The dose is applied to the shaved areas for 5 or 6 consecutive days. At regular intervals, mice are scored for erythema, scaling, and thickening on a scale from 0 to 4, as described by van der Fits et al. (2009). Mice are monitored for ear thickness using a Mitutoyo micrometer.

Treatment with bacteria is initiated at some point, either around the time of the first application of IMQ, or something thereafter. For example, bacteria may be administered at the same time as the subcutaneous injections (day 0), or they may be administered prior to, or upon, application. Bacteria are administered at varied doses and at defined intervals. While some mice receive bacteria through i.v. injection, other mice may receive bacteria through intraperitoneal (i.p.) injection, nasal route administration, oral gavage, topical administration, intradermal (i.d.) injection, subcutaneous (s.c.) injection, or other means of administration. Some mice may receive bacteria every day (e.g. starting on day 0), while others may receive bacteria at alternative intervals (e.g. every other day, or once every three days). The bacterial cells may be live, dead, or weakened. The bacterial cells may be harvested fresh (or frozen) and administered, or they may be irradiated, lyophilized, or heat-killed prior to administration.

For example, some groups of mice may receive between 1×10⁴ and 5×10⁹ bacterial cells.

Some groups of mice may be treated with anti-inflammatory agent(s) (e.g. anti-CD154, blockade of members of the TNF family, or other treatment), and/or an appropriate control (e.g. vehicle or control antibody) at various timepoints and at effective doses.

In addition, some mice are treated with antibiotics prior to treatment. For example, vancomycin (0.5 g/L), ampicillin (1.0 g/L), gentamicin (1.0 g/L) and amphotericin B (0.2 g/L) are added to the drinking water, and antibiotic treatment is halted at the time of treatment or a few days prior to treatment. Some immunized mice are treated without receiving antibiotics.

At various timepoints, samples from back and ear skin are taken for cryosection staining analysis using methods known in the art. Other groups of mice are sacrificed and lymph nodes, spleen, mesenteric lymph nodes (MLN), the small intestine, colon, and other tissues may be removed for histology studies, ex vivo histological, cytokine and/or flow cytometric analysis using methods known in the art. Some tissues may be dissociated using dissociation enzymes according to the manufacturer's instructions. Cryosection samples, tissue samples, or cells obtained ex vivo are stained for analysis by flow cytometry using techniques known in the art. Staining antibodies can include anti-CD11c (dendritic cells), anti-CD80, anti-CD86, anti-CD40, anti-MHCII, anti-CD8a, anti-CD4, and anti-CD103. Other markers that may be analyzed include pan-immune cell marker CD45, T cell markers (CD3, CD4, CD8, CD25, Foxp3, T-bet, Gata3, Roryt, Granzyme B, CD69, PD-1, CTLA-4), and macrophage/myeloid markers (CD11b, MHCII, CD206, CD40, CSF1R, PD-L1, Gr-1, F4/80). In addition to immunophenotyping, serum cytokines are analyzed including, but not limited to, TNFa, IL-17, IL-13, IL-12p70, IL12p40, IL-10, IL-6, IL-5, IL-4, IL-2, IL-1b, IFNy, GM-CSF, G-CSF, M-CSF, MIG, IP10, MIP1b, RANTES, and MCP-1. Cytokine analysis may be carried out on immune cells obtained from lymph nodes or other tissue, and/or on purified CD45+ skin-infiltrated immune cells obtained ex vivo. Finally, immunohistochemistry is carried out on various tissue sections to measure T cells, macrophages, dendritic cells, and checkpoint molecule protein expression.

In order to examine the impact and longevity of psoriasis protection, rather than being sacrificed, some mice may be studied to assess recovery, or they may be rechallenged with IMQ. The groups of rechallenged mice is analyzed for susceptibility to psoriasis and severity of response.

### Example 3: Prevotella histicola Strain B in Healthy Participants and Participants with Mild to Moderate Psoriasis or Mild to Moderate Atopic Dermatitis

Rationale:

This first-in-human (FIH) study investigates the safety and tolerability of the monoclonal microbial Prevotella histicola Strain B in healthy volunteers, and in patients with mild to moderate psoriasis and patients with mild to moderate atopic dermatitis. Furthermore, the potential of Prevotella histicola Strain B to modify the immune system to provide benefit to these patient populations is also assessed. Therefore, this FIH study is designed to give the maximum information and understanding about the potential benefit of Prevotella histicola Strain B by investigating its pharmacodynamic effects in healthy volunteers and in patient cohorts with mild to moderate psoriasis and mild to moderate atopic dermatitis.

Prevotella histicola is a natural human commensal organism, commonly found on oral, nasopharyngeal, gastrointestinal (GI), and genito-urinary mucosal surfaces. Preclinical studies using Prevotella histicola Strain B have been carried out across a range of human and mouse primary cell in vitro assays, as well as in 5 key in vivo models, which all support the use of this agent in the treatment of immunoinflammatory diseases.

In vitro, Prevotella histicola Strain B has been found to stimulate secretion of anti-inflammatory cytokines such as interleukin (IL)-10, IL-27, and IL-1RA from human macrophages and dendritic cells, whilst inducing only minimal levels of pro-inflammatory cytokines such as IL-17, IL-12, and granulocyte-macrophage colony-stimulating factor (GM-CSF).

In vivo, Prevotella histicola Strain B has shown evidence of efficacy in delayed-type hypersensitivity (DTH), dextran sulphate sodium (DSS) colitis, fluorescein isothyocyanate (FITC) cutaneous hypersensitivity, collagen-induced arthritis (CIA) models of immunoinflammatory disease, and experimental allergic encephalomyelitis (EAE). No potentially related adverse effects were seen in the animals used in these experiments with daily dosing up to 21 days. These data suggest that treatment with this monoclonal microbial strain of Prevotella could provide benefit in a range of immunoinflammatory conditions, including psoriasis and atopic dermatitis.

Two cohorts of healthy volunteers followed by a cohort of participants with mild to moderate psoriasis are studied to provide evidence of safety and tolerability of the product. Following these first 3 cohorts, up to 4 more cohorts (2 in mild to moderate psoriasis and 2 in mild to moderate atopic dermatitis) are studied to assess the safety and tolerability of Prevotella histicola Strain B, in addition to investigating the evidence of potential beneficial changes in the tissue and systemic immune environment. These cohorts evaluate doses between 1 and 5 times the human equivalent dose based on allometric scaling. The order of the doses is based first on any safety or tolerability concerns observed in the first 3 cohorts and then, assuming no concerns, by the availability of drug supply, meaning these cohorts may be operationalised in a non-numeric order (i.e. Cohorts 6 and 7 may run before Cohorts 4 and 5). The expected doses to be studied are 1 times and 5 times the human equivalent dose (HED) based on allometric scaling from the mouse in vivo models.

### Objectives and Endpoints

| Objectives | Endpoints |
|---|---|
| Primary | |
| Safety and tolerability of | Serious adverse event (SAE) and |
| Prevotella histicola Strain B | adverse event (AE) incidents |
| | Clinical safety laboratory |
| | measurements |
| | Electrocardiogram (ECG) |
| | measurements |
| | Vital sign measurements |
| | Physical examination |
| | Bristol stool scale |
| | Specific markers of GI integrity |
| | Specific immune biomarkers |
| Secondary | |
| Clinical improvement in | Psoriasis Area and Severity Index |
| participants with mild to moderate | (PASI) |
| psoriasis | Eczema Area and Severity Index |
| Clinical improvement in | (EASI) |
| participants with mild to moderate | SCORing Atopic Dermatitis |
| atopic dermatitis | (SCORAD) |
| | Lesion Severity Score (LSS) |
| | Percentage of Body Surface Area |
| | (BSA) |
| | affected by disease in Cohorts 3, 4, |
| | 5, 6, and 7 |
| | Investigator's Global Assessment |
| | (IGA) |
| Exploratory | |
| Evidence of pharmacodynamic | Immunohistochemistry (IHC) on |
| effects on skin | skin biopsies |
| Evidence of systemic immune | Messenger ribonucleic acid (mRNA) |
| modulation | transcription analysis on skin |
| Effect of Prevotella histicola | biopsies |
| Strain B on gene and protein | IHC/immunofluorescence (IF) |
| expression, and explore the | of skin tissue looking at immune cell |
| relationship between genomic, | infiltrates |
| genetic, and proteomic biomarkers | Blood cytokine and chemokine levels |
| and disease biology, drug | Blood gene expression profiling |
| treatment and inflammatory and | Microbiome composition (in faeces) |
| immune responses | |

### Overall Design:

This is a single center, randomized placebo-controlled clinical study with dose escalations and dose expansions to assess preliminary safety, tolerability, and efficacy of Prevotella histicola Strain B in healthy participants and participants with either mild to moderate psoriasis or mild to moderate atopic dermatitis. The investigators and participants are blinded to study drug but the Sponsor is unblinded. The study consists of 2 cohorts of healthy volunteers, 3 cohorts of participants with mild to moderate psoriasis, and 2 cohorts of participants with mild to moderate atopic dermatitis. Escalating doses from 1/10^{th} of the estimated therapeutic dose to up to 5 times the estimated therapeutic dose versus placebo is evaluated. The primary aim of the study is to assess safety and tolerability of Prevotella histicola Strain B. Secondary and exploratory endpoints are designed to establish whether there are any effects on the systemic immune system and potential clinical benefit.

A within-cohort single and multiple dose regimen is used with an interval of at least 48 hours between the single dose and the start of the multiple dosing period for each individual. Prior to the multiple dosing period for each individual, an evaluation of that individual's safety data is performed by the Principal Investigator (or delegate) and the Medical Monitor.

Participants who are successfully screened are randomized to either to the active (Prevotella histicola Strain B) or placebo group on Day 1 and dosing is initiated. For each dose level of healthy volunteers, there is a sentinel group of 2 participants (1 active, 1 placebo). The remainder of the cohort is dosed following a review of their safety data after at least 3 days of multiple dosing.

All safety data is reviewed in an ongoing and cumulative manner by the Principal Investigator (or delegate), Medical Monitor and the safety review committee (SRC).

### Number of Participants:

The minimum number of participants (Cohorts 1 to 7) is 120 in total and the maximum number is 132 participants in total, although additional replacements may be enrolled if necessary.

Sufficient participants are screened to achieve 24 healthy volunteers randomly assigned to Prevotella histicola Strain B or placebo using a 2:1 randomization ratio: a total of 12 evaluable participants in Cohort 1 and Cohort 2.

Sufficient participants are screened to achieve up to 60 evaluable participants with mild to moderate psoriasis randomly assigned to Prevotella histicola Strain B or placebo using a 2:1 randomization ratio: a total of 12 evaluable participants in Cohort 3 and up to a total of 24 evaluable participants in Cohort 4 and up to a total of 24 evaluable participants in Cohort 6.

Sufficient participants are screened to achieve up to 48 evaluable participants with mild to moderate atopic dermatitis randomly assigned to Prevotella histicola Strain B or placebo using a 2:1 randomization ratio a total of 24 evaluable participants in Cohort 5 and up to a total of 24 evaluable participants in Cohort 7.

Dosing in Cohorts 4, 5, 6 and 7 can occur in parallel following a review of the safety data from Cohort 3. The sequencing of the cohorts can be adjusted to accommodate the available drug supply, e.g. Cohort 6 can be conducted before Cohort 4 and Cohort 7 can be conducted before Cohort 5. All safety data from previous lower doses cohorts are reviewed prior to dose escalation.

### Intervention Groups and Duration:

The design of the study allows a dose escalation in healthy participants to ensure Prevotella histicola Strain B is safe and well tolerated in humans (Cohorts 1 and 2). Prevotella histicola Strain B is then tested in participants with mild to moderate psoriasis for safety and tolerability and for an effect on the disease pathology (Cohort 3). Prevotella histicola Strain B is then tested in 2 more psoriasis cohorts (Cohort 4 and Cohort 6) and in 2 cohorts of participants with mild to moderate atopic dermatitis (Cohort 5 and Cohort 7) to investigate the potential for Prevotella histicola Strain B to treat Th2-driven immunoinflammatory disorders.

Cohort 1: 12 healthy participants are randomized into Cohort 1: 8 participants are randomized to the lowest dose of Prevotella histicola Strain B of approximately 1.6×10¹⁰ total cells which is 0.1× the allometric scaled preclinical efficacious dose level (Dose 1, approximately 1/10^{th} of the estimated therapeutic dose) and 4 participants are randomized to placebo. Sentinel dosing of the first pair (1 on active and 1 on placebo) happens on Day 1. The remainder of the cohort is dosed from Day 6. Following a 48-hour washout period, the participants then start a 14-day multiple dosing period.

Cohort 2: 12 healthy participants are randomized into Cohort 2: 8 participants are randomized to Prevotella histicola Strain B up to a maximum of approximately 1.6×10¹¹ total cells which is 1× the allometric scaled preclinical efficacious dose level (Dose 2, the estimated therapeutic dose) and 4 participants are randomized to placebo. Sentinel dosing of the first pair (1 on active and 1 on placebo) happens on Day 1. The remainder of the cohort is dosed from Day 6. Following a 48-hour washout period, the participants then start a 14-day multiple dosing period.

Cohort 3: 12 participants with mild to moderate psoriasis are randomized into Cohort 3: 8 participants are randomized to Prevotella histicola Strain B up to a maximum of approximately 1.6×10¹¹ total cells which is 1× the allometric scaled preclinical efficacious dose level (Dose 2, the estimated therapeutic dose) and 4 participants are randomized to placebo. Sentinel dosing of the first pair (1 on active and 1 on placebo) happens on Day 1. The remainder of the cohort is dosed from Day 6. Following a 48-hour washout period, the participants then start a 28-day multiple dosing period.

Cohort 4: Up to 24 participants with mild to moderate psoriasis are randomized into Cohort 4: 16 participants are randomized to Prevotella histicola Strain B up to a maximum of approximately 8.0×10¹¹ total cells which is 5× the allometric scaled preclinical efficacious dose level (Dose 3, 5 times the estimated therapeutic dose) and 8 participants are randomized to placebo. Sentinel dosing of the first pair (1 on active and 1 on placebo) happens on Day 1. The remainder of the cohort is dosed from Day 6. Following a 48-hour washout period, the participants then start a 28-day multiple dosing period.

Cohort 5: Up to 24 participants with mild to moderate atopic dermatitis are randomized into Cohort 5: 16 participants are randomized to Prevotella histicola Strain B up to a maximum of approximately 8.0×10¹¹ total cells which is 5× the allometric scaled preclinical efficacious dose level (Dose 3, 5 times the estimated therapeutic dose) and 8 participants are randomized to placebo. Sentinel dosing of the first pair (1 on active and 1 on placebo) happens on Day 1. The remainder of the cohort is dosed from Day 6. Following a 48-hour washout period, the participants then start a 28-day multiple dosing period.

Cohort 6: Up to 24 participants with mild to moderate psoriasis are randomized into Cohort 6: 16 participants are randomized to Prevotella histicola Strain B up to a maximum of approximately 8.0×10¹¹ total cells which is 5× the allometric scaled preclinical efficacious dose level (Dose 3, 5 times the estimated therapeutic dose) and 8 participants are randomized to placebo. All participants are dosed for 28 days.

Cohort 7: Up to 24 participants with mild to moderate atopic dermatitis are randomized into Cohort 7: 16 participants are randomized to Prevotella histicola Strain B up to a maximum of approximately 8.0×10¹¹ total cells which is 5× the allometric scaled preclinical efficacious dose level (Dose 3, 5 times the estimated therapeutic dose) and 8 participants are randomized to placebo. All participants are dosed for 28 days.

Table 3 below describes the starting dose and the anticipated and maximum dose levels that may be evaluated during the study for all parts of the study.

TABLE 3
Summary of Dose Levels

| | | Anticipated Dose | Maximum Dose | |
|---|---|---|---|---|
| | | Levels (once | Levels (once | Maximum |
| | | daily dosing of | daily dosing of | Number of |
| | | Prevotella | Prevotella | Participants |
| | | histicola Strain | histicola Strain | (active + |
| Cohort | Participants | B or placebo | B or placebo) | placebo) |
| 1 | Healthy | 1/10^{th} of HED | 1/10^{th} of HED | 8 + 4 |
| | volunteers | | | |
| 2 | Healthy | HED | ≤HED | 8 + 4 |
| | volunteers | | | |
| 3 | Mild to | HED | ≤HED | 8 + 4 |
| | moderate | | | |
| | psoriasis | | | |
| 4 | Mild to | 5 × HED | ≤5 × HED | 16 + 8 |
| | moderate | | | |
| | psoriasis | | | |
| 5 | Mild to | 5 × HED | ≤5 × HED | 16 + 8 |
| | moderate | | | |
| | atopic | | | |
| | dermatitis | | | |
| 6 | Mild to | 1 × HED | ≤5 × HED | 16 + 8 |
| | moderate | | | |
| | psoriasis | | | |
| 7 | Mild to | 1 × HED | ≤5 × HED | 16 + 8 |
| | moderate | | | |
| | atopic | | | |
| | dermatitis | | | |

HED = human equivalent dose

### Safety Review Committee:

An SRC consisting of the Principal Investigator (or delegate), Medical Monitor, Statistician and Sponsor's Clinical Lead review blinded safety data and provide governance over the study and dose escalation steps. The SRC will decide whether to proceed to the next dosing level at the end of each cohort, and they can decide to omit a cohort or dose escalation step if warranted. Dose escalation decisions will be made when at least 9 participants have completed the multiple dosing period of the stated dose level. To implement dose escalation decisions, the available adverse events (AEs) and laboratory test data will be evaluated at a dose decision meeting or teleconference. Drug administration at the next dose cohort will not proceed until the investigator receives written confirmation from Sponsor indicating that the results of the previous dose cohort were evaluated and that it is permissible to proceed to the next higher dose cohort. Ad hoc SRC meetings may be convened if deemed necessary by the Sponsor or the Principal Investigator (or delegate). A detailed description of the procedures will be outlined in a separate SRC charter. Following the successful completion of Cohort 3 and the SRC decision to continue, then Cohorts 4 to 7 can be run in parallel or in an order which optimizes the use of available drug supply. A review of the safety data will be performed after each cohort is finished, but it is not requirement to move from one cohort to the next in Cohorts 4 to 7.

Each safety review will be based on the following data, which shall all be checked and Quality Controlled (QC'd) as far is practically possible:

TABLE 4
Summary of Safety Reviews

| | | Approximate | | |
|---|---|---|---|---|
| | | Strength | | |
| | | Relative to | | |
| | | Minimum | | |
| | | Expected | Sentinel | |
| Cohort | Population | Clinical Dose | Pair | Safety Review |
| 1 | Healthy | ×0.1 | Yes | 1. Sentinel dosing used for first 2 |
| | volunteers | Placebo | | participants who will receive ≥3 |
| 2 | Healthy | ≤ × 1 | Yes | daily doses in the multiple |
| | volunteers | Placebo | | dosing period before enrolment |
| 3 | Mild to | ≤ × 1 | Yes | is opened to the remaining |
| | moderate | Placebo | | individuals in the cohort. |
| | psoriasis | | | 2. Principal Investigator (or |
| 4 | Mild to | ≤ × 5 | Yes | delegate) and Medical Monitor |
| | moderate | Placebo | | will review the transition within |
| | psoriasis | | | each cohort, from single dose to |
| 5 | Mild to | ≤ × 5 | Yes | multiple dose for each |
| | moderate | Placebo | | participant. |
| | atopic | | | 3. SRC (Principal Investigator or |
| | dermatitis | | | delegate, Medical Monitor, |
| | | | | Statistician & Sponsor's Clinical |
| | | | | Lead) will review blinded safety |
| | | | | data before dose escalation |
| | | | | decision. |
| 6 | Mild to | ≤ × 5 | No | 1. Principal Investigator (or |
| | moderate | Placebo | | delegate) and Medical Monitor |
| | psoriasis | | | will review the transition within |
| 7 | Mild to | ≤ × 5 | No | each cohort, from single dose to |
| | moderate | Placebo | | multiple dose for each |
| | atopic | | | participant. |
| | dermatitis | | | 2. SRC (Principal Investigator or |
| | | | | delegate, Medical Monitor, |
| | | | | Statistician & Sponsor's Clinical |
| | | | | Lead) will review blinded safety |
| | | | | data before dose escalation |
| | | | | decision. |

Doses specified as ≤ indicates a dose up to the maximum specified value

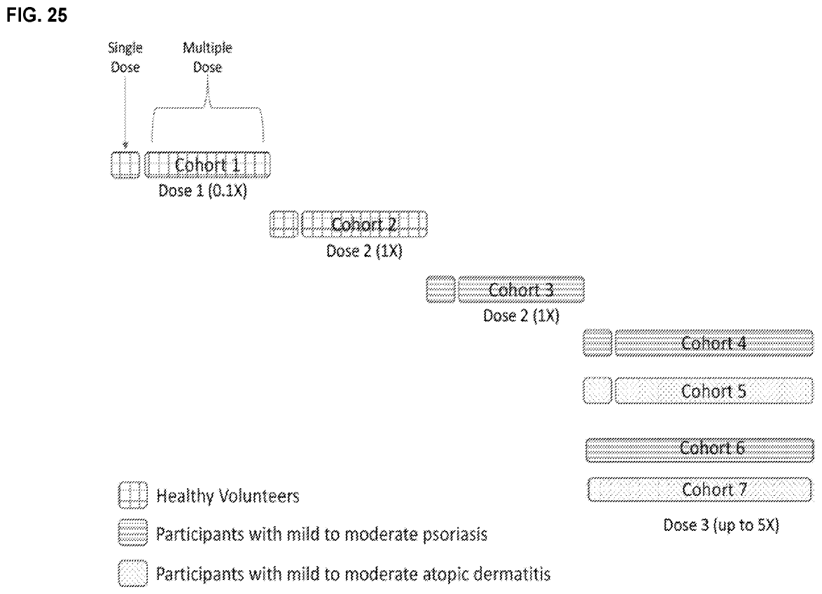

### 1.1. Schema (FIG. 25)

Note: progression to the next cohort for Cohorts 1 to 3 will be decided by the SRC. Cohorts 4 to 7 will be run according to availability of drug supply. Sentinel dosing of the first pair (1 on active and 1 on placebo) will happen on Day 1 of Cohorts 4, 5, 6 and 7 if dose escalation occurs.

### 1.2. Schedule of Activities (SoA)

| | Procedure | | | | | | | | | | | | | | |
|---|---|---|---|---|---|---|---|---|---|---|---|---|---|---|---|
| | Screening | Baseline | Intervention Period [Days] | | | | | | | | | | | | Follow-up ^{p} |
| | Day | | | | | | | | | | | | | | |
| | −28 | −1 | 1 | 2 | 3 ^{a} | 4 | 5 | 6 | 7-9 | 10 | 11-13 | 14 | 15 | 16 | 30 |
| | Visit number | | | | | | | | | | | | | | |
| | 1 | 2 | 3 | 4 | 5 | 6 | 7 | 8 | 9-11 | 12 | 13-15 | 16 | 17 | 18 | 19 |
| | Visit window | | | | | | | | | | | | | | |
| | +28 | 0 | 0 | 0 | +3 | 0 | 0 | 0 | 0 | 0 | 0 | 0 | 0 | 0 | +14 |
| Inpatient/overnight | | X | X | X | X | X | X | | | | | | | | |
| stay ^{b} | | | | | | | | | | | | | | | |
| Informed consent | X | | | | | | | | | | | | | | |
| Inclusion and | X | X | | | | | | | | | | | | | |
| exclusion criteria ^{c} | | | | | | | | | | | | | | | |
| Demography | X | | | | | | | | | | | | | | |
| Full physical | X | X | X | X | X | | | X | | | | X | | X | X |
| examination | | | | | | | | | | | | | | | |
| (including height | | | | | | | | | | | | | | | |
| and weight) ^{d} | | | | | | | | | | | | | | | |
| Medical history | X | X | | | | | | | | | | | | | |
| (includes | | | | | | | | | | | | | | | |
| substance usage) ^{e} | | | | | | | | | | | | | | | |
| Current medical | X | | | | | | | | | | | | | | |
| conditions | | | | | | | | | | | | | | | |
| Pregnancy test ^{f} | X | X | | | | | | | | | | | | | X |
| HBsAg, HCV and | X | | | | | | | | | | | | | | |
| HIV screening | | | | | | | | | | | | | | | |
| Laboratory | X | X | | X | X | X | X | | | X | | | | X | X |
| assessments | | | | | | | | | | | | | | | |
| (haematology, | | | | | | | | | | | | | | | |
| biochemistry, and | | | | | | | | | | | | | | | |
| urinalysis) ^{g} | | | | | | | | | | | | | | | |
| 12-lead ECG ^{h} | X | | X | X | | | X | | | | | | | X | X |
| Vital signs ⁱ | X | X | X | X | X | | X | X | | X | | X | | X | X |
| Randomisation | | | X | | | | | | | | | | | | |
| HLA sample ^{j} | | X | | | | | | | | | | | | | |
| Dosing ^{k,l} | | | X | | ============================= | | | | | | | | | | |
| | | | | | ====→ | | | | | | | | | | |
| Bristol stool scale ^{m} | X | X | ←================================ | | | | | | | | | | | | X |
| | | | ======→ | | | | | | | | | | | | |
| Samples for | X | X | | | X | | | | | | | | | X | X |
| microbiome | | | | | | | | | | | | | | | |
| investigation ⁿ | | | | | | | | | | | | | | | |
| Sample for | | X | | | X | | X | | | | | | | X | X |
| systemic levels of | | | | | | | | | | | | | | | |
| microbes ^{o} | | | | | | | | | | | | | | | |
| Samples for blood | | X | | | X | | | | | | | | | X | X |
| biomarkers ^{o} | | | | | | | | | | | | | | | |
| Sample for CRP ^{o} | X | X | | X | X | X | X | | | X | | | | | X |
| Sample for faecal | X | X | | X | X | X | X | | | X | | | | | X |
| calprotectin ^{o} | | | | | | | | | | | | | | | |

| | | | | |
|---|---|---|---|---|
| AE review ¹ | X | X | ←================================ | X |
| | | | ======→ | |
| SAE review ¹ | X | X | ←================================ | X |
| | | | ======→ | |
| Concomitant | | | ←================================ | |
| medication review ¹ | | X | ======→ | X |

Abbreviations:
AE = adverse event;
CRP = C-reactive protein;
ECG = electrocardiogram;
HBsAg = surface antigen of hepatitis B;
HCG = human chorionic gonadotrophin;
HCV = hepatitis C;
HIV = human immunodeficiency virus;
HLA = human leukocyte antigen;
SAE = serious adverse event
^{a} Start of the 14-day multiple dosing period (after a 48-hour washout period).
^{b} Inpatient stay only required for participants in Cohort 1. Inpatient requirement from Day -1 (at least 24 hours prior to the first dose) to 48 hours post first dose in multiple dosing period.
^{c} Recheck clinical status before first dose of study intervention.
^{d} Height at Screening only. Recheck full physical to ensure participant can move to multiple dosing period.
^{e} Substances: drugs urine test and alcohol breath test.
^{f} Women of child-bearing potential only. Serum HCG will be performed. Pregnancy testing will be performed whenever a menstrual cycle is missed or when pregnancy is otherwise suspected. Details of all pregnancies in female participants will be collected until 28 days after the last dose.
^{g} Laboratory samples taken at the specified visit and reviewed at the next visit prior to dosing (i.e. at each visit the laboratory results from the previous visit are reviewed). Fasting glucose at baseline and end of dosing only. Day 4 sample only collected for Cohort 1.
^{h} All ECGs to be measured in triplicate. All ECGs on dosing days to be conducted post-dosing and within 2 hours after the dose.
ⁱ Blood pressure, pulse, respiratory rate and oral temperature - check prior to dosing and/or any procedures.
^{j} Predose genetic sample.
^{k} Daily dosing starting on Visit 5 - to occur at approximately the same time ±2 hours. Refrain from consuming acidic drinks 1 hour either side of dosing and from eating 2 hours before dosing and 1 hour after dosing.
^{l} Visits will be daily during the multiple dosing period (Visits 5 to 18). Following Visit 5 and up to the follow-up Visit, daily review of AE, SAE and concomitant medications will be required.
^{m} 7 days before dosing, 7 days after final dosing, and daily (or at each bowel movement) throughout the dosing period.
ⁿ Predose sample need only be taken once at any time before Day 1. Samples after Day 1 should be taken on the specified day where possible and if this is not possible, then a sample should be collected as close to the planned timepoint as possible (i.e. within 48 hours).
^{o} Take predose. Samples should be taken on the specified day where possible and if this is not possible, then a sample should be collected as close to the planned timepoint as possible (i.e. within 48 hours). Day 4 sample only collected for Cohort 1.
^{p} Participants who withdraw from the study early should complete these assessments.

### Cohorts 3, 4, 5, 6 and 7 (Psoriasis and Atopic Dermatitis Participants)

| | Procedure | | | | | | | | | | |
|---|---|---|---|---|---|---|---|---|---|---|---|
| | Screening | Baseline | Intervention Period [Days] | | | | | | | | Follow-up ^{q} |
| | Day | | | | | | | | | | |
| | −28 | −1 | 1 | 2 | 3 ^{a} | 5 | 10 | 17 | 24 | 30 | 44 |
| | Visit number | | | | | | | | | | |
| | 1 | 2 | 3 | 4 | 5 | 6 | 7 | 8 | 9 | 10 | 11 |
| | Visit window | | | | | | | | | | |
| | +28 | 0 | 0 | 0 | +3 | 0 | 0 | 0 | 0 | 0 | +14 |
| Informed consent | X | | | | | | | | | | |
| Inclusion and exclusion | X | X | | | | | | | | | |
| criteria ^{b} | | | | | | | | | | | |
| Demography | X | | | | | | | | | | |
| Full physical examination | X | X | | X | | X | X | X | X | X | X |
| (including height and | | | | | | | | | | | |
| weight) ^{c} | | | | | | | | | | | |
| Medical history (includes | X | X | | | | | | | | | |
| substance usage) ^{d} | | | | | | | | | | | |
| Current medical conditions | X | | | | | | | | | | |
| Pregnancy test ^{e} | X | X | | | | | | | | | X |
| HBsAg, HCV and HIV | X | | | | | | | | | | |
| screening | | | | | | | | | | | |
| Laboratory assessments | X | X | | X | X | X | X | X | X | X | X |
| (haematology, | | | | | | | | | | | |
| biochemistry and | | | | | | | | | | | |
| urinalysis) ^{f} | | | | | | | | | | | |
| 12-lead ECG ^{g} | X | | X | X | | X | | | X | | X |
| Vital signs ^{h} | X | X | X | X | X | X | X | X | X | X | X |
| Randomisation | | | X | | | | | | | | |
| HLA sample ⁱ | | X | | | | | | | | | |
| Dosing ^{j} | | | X | ←================ | | | | | | | |
| | | | | =====→ | | | | | | | |
| Skin biopsy | | X | | | | | | | | X | |
| Bristol stool scale ^{k} | X | X | ←================== | | | | | | | | X |
| | | | =====→ | | | | | | | | |
| Samples for microbiome | X | X | | X | | | X | | | X | X |
| investigation ^{l} | | | | | | | | | | | |
| Sample for systemic levels | | X | | X | X | | X | | | X | X |
| of microbes ^{m} | | | | | | | | | | | |
| Samples for blood | | X | | X | | | X | | | X | X |
| biomarkers ^{m} | | | | | | | | | | | |
| Sample for CRP ^{m} | X | X | | X | X | X | X | X | | X | X |
| Sample for faecal | X | X | | X | X | X | X | X | | X | X |
| calprotectin ^{m} | | | | | | | | | | | |
| AE review | X | X | ←================= | | | | | | | | X |
| | | | =====→ | | | | | | | | |
| SAE review | X | X | ←================= | | | | | | | | X |
| | | | =====→ | | | | | | | | |
| Concomitant medication | | X | ←================= | | | | | | | | X |
| review | | | =====→ | | | | | | | | |
| PASP ⁿ | X | | | | X | | X | X | X | X | X |
| IGA | X | | | | X | | X | X | X | X | X |
| BSA affected by disease | X | | | | X | | X | X | X | X | X |
| (%) | | | | | | | | | | | |
| EASI ^{o} | X | | | | X | | X | X | X | X | X |
| SCORAD ^{o} | X | | | | X | | X | X | X | X | X |
| LSS | X | | | | X | | X | X | X | X | X |
| Photos of lesion sites ^{p} | X | | | | | | X | | | X | X |

Abbreviations:
AE = adverse event;
BSA = body surface area;
CRP = C-reactive protein;
EAST = Eczema Area and Severity Index;
ECG = electrocardiogram;
HBsAg = surface antigen of hepatitis B;
HCG = human chorionic gonadotrophin;
HCV = hepatitis C;
HIV = human immunodeficiency virus;
HLA = human leukocyte antigen;
IGA = Investigator's Global Assessment;
LSS = lesion severity score;
PAST = Psoriasis Area and Severity Index;
SAE = serious adverse event;
SCORAD = SCORing Atopic Dermatitis.
^{a} Start of the 28-day multiple dosing period (after a 48-hour washout period).
^{b} Recheck clinical status before first dose of study intervention.
^{c} Height at Screening only. Recheck full physical to ensure participant can move to multiple dosing period.
^{d} Substances: drugs urine test and alcohol breath test.
^{e} Women of child-bearing potential only. Serum HCG will be performed. Pregnancy testing will be performed whenever a menstrual cycle is missed or when pregnancy is otherwise suspected. Details of all pregnancies in female participants will be collected until 28 days after the last dose.
^{f} Laboratory samples taken at the specified visit and reviewed at the next visit prior to dosing (i.e. at each visit the laboratory results from the previous visit are reviewed). Fasting glucose at baseline and end of dosing only.
^{g} All ECGs to be measured in triplicate. All ECGs on dosing days to be conducted post-dosing and within 2 hours after the dose.
^{h} Blood pressure, pulse, respiratory rate and oral temperature - check prior to dosing and/or any procedures.
ⁱ Predose genetic sample.
^{j} Daily dosing starting on Visit 5 - to occur at approximately the same time ±2 hours. Refrain from consuming acidic drinks 1 hour either side of dosing and from eating 2 hours before dosing and 1 hour after dosing.
^{k} 7 days before dosing, 7 days after final dosing, and daily (or at each bowel movement) throughout the dosing period.
^{l} Predose sample need only be taken once at any time before Day 1. Samples after Day 1 should be taken on the specified day where possible and if this is not possible, then a sample should be collected as close to the planned timepoint as possible (i.e. within 48 hours).
^{m} Take predose. Samples should be taken on the specified day where possible and if this is not possible, then a sample should be collected as close to the planned timepoint as possible (i.e. within 48 hours).
ⁿ Psoriasis participants only.
^{o} Atopic dermatitis participants only.
^{p} Photos should be taken of up to 6 lesion sites that have a lesion area ≥2 × 2 cm at baseline.
^{q} Participants who withdraw from the study early should complete these assessments.

### Introduction

Prevotella histicola Strain B is a pure monoclonal microbial of Prevotella histicola, which, in in vitro mouse and human cell assays, increases secretion of anti-inflammatory cytokines such as interleukin (IL)-10, IL-27 and IL-1RA from human macrophages and dendritic cells, whilst inducing only minimal levels of pro-inflammatory cytokines such as IL-17, IL-12, and granulocyte-macrophage colony-stimulating factor (GM-CSF). Although the monoclonal microbials are delivered orally and exposure is restricted to the gastrointestinal (GI) tract, in vivo studies have shown that measurable effects on the immune system also occur beyond the GI tract, which suggests that host-microbe interactions in the gut can affect the immune response in peripheral tissues. Based on our own, as well as published studies [Mangalam, 2017; Marietta, 2016], evidence is building that orally administered Prevotella histicola has the potential to ameliorate systemic disease. The effects of chronic Prevotella histicola Strain B administration will be investigated in a range of immunoinflammatory disorders, e.g. psoriasis and atopic dermatitis, to understand its value in treating these conditions.

Preclinical studies using Prevotella histicola Strain B have been carried out across a range of human and mouse primary cell in vitro assays as well as in 5 key in vivo models: delayed-type hypersensitivity (DTH), dextran sulphate sodium (DSS) colitis, fluorescein isothyocyanate (FITC) cutaneous hypersensitivity, collagen-induced arthritis (CIA), and experimental allergic encephalomyelitis (EAE), which all support the use of this agent in the treatment of immunoinflammatory diseases. Evidence of a positive pharmacodynamic effect has been seen in all the in vivo models suggesting the potential for positive clinical benefit. No potentially related adverse effects were seen in the animals used in these experiments with daily dosing up to 21 days.

### Study Rationale

Prevotella histicola Strain B-101 is the first-in-human (FIH) study for Prevotella histicola Strain B, which is a specific pure strain of Prevotella histicola, a natural human commensal organism, commonly found on oral, nasopharyngeal, GI, and genito-urinary mucosal surfaces. Sponsor unpublished data indicate that the monoclonal microbials being evaluated for a therapeutic effect do not penetrate into the systemic circulation so the safety and tolerability is likely to be good. Prevotella histicola Strain B-101 has therefore been designed to confirm the safety and tolerability of Prevotella histicola Strain B in both healthy participants and participants with mild to moderate psoriasis or mild to moderate atopic dermatitis, as these are a relatively healthy group.

The healthy volunteer cohorts will establish safety and tolerability of escalating doses from 1/10^{th} of the estimated therapeutic dose to up to 5 times the estimated therapeutic dose of Prevotella histicola Strain B. The formulation being used in this study is an enteric coated capsule designed to release the microbes at the start of the duodenum based on a pH sensitive coating.

The potential of the product to modulate the systemic immune response in an immunoinflammatory condition will be established using individuals with mild to moderate psoriasis and mild to moderate atopic dermatitis. Both conditions are being used as they have different key immunological drivers and have the advantage of allowing biopsies of the disease tissue. These cohorts (3 to 7) are dosed for 28 days continuous dosing as this is the minimum time required to demonstrate a clinical response. Paired skin biopsies, taken pre-treatment and at Day 30, will be used to evaluate biomarker changes predictive of specific immunomodulation. The combination of participants with psoriasis and atopic dermatitis and healthy participants in this study will allow Sponsor to efficiently establish the safety and tolerability of Prevotella histicola Strain B while informing the potential indications of patient populations that could benefit from this product.

### 1.3. Background

Prevotella histicola is a gram-negative, non-sporulating, obligate anaerobe. It is a natural human commensal organism, and enrichment of the genus Prevotella has been associated with high-fiber, plant-based, non-Western diets [Wu, 2011]. Lower relative abundance of Prevotella in the gut microbiome is associated with obesity [Tagliabue, 2013] and in some diseases such as multiple sclerosis [Cosorich, 2017; Mangalam, 2017; Marietta, 2016; Jangi, 2016; Miyake, 2015], whereas higher abundance is associated with an exercise-rich lifestyle [Petersen, 2017] and maintenance of healthy weight [Hjorth, 2018]. The preclinical data generated in the Prevotella histicola Strain B program has highlighted that individual strains have different properties even within a single genus, demonstrating that strain choice is important. In addition, a dose-response curve has been observed suggesting that exposure levels of organisms are important to obtaining the required pharmacological properties. Sponsor intends to develop Prevotella histicola Strain B as a medicinal product for the treatment of a range of immunoinflammatory indications and patient populations. Further information may be found in the Investigator's Brochure (IB).

### Benefit/Risk Assessment

Prevotella histicola Strain B is a specific pure strain of Prevotella histicola, a natural human commensal organism, commonly found on oral, nasopharyngeal, GI, and genito-urinary mucosal surfaces. It is a gram-negative bacterium sensitive to the major classes of antibiotics, e.g. penicillins and cephalosporins.

Prevotella histicola Strain B is being investigated for its potential benefit in chronic immunoinflammatory disorders. The initial conditions being tested are mild to moderate psoriasis and mild to moderate atopic dermatitis. A well-tolerated oral therapy could offer significant benefit in both of these conditions and at present it is anticipated that Prevotella histicola Strain B would be used in established but early disease before the intervention of biologic therapies is required.

### Objectives and Endpoints

| Objectives | Endpoints |
|---|---|
| Primary | |
| • Safety and tolerability | • Serious adverse event (SAE) and |
| of Prevoiella | adverse event (AE) incidents |
| histicola Strain B | • Clinical safety laboratory |
| | measurements |
| | • Electrocardiogram (ECG) |
| | measurements |
| | • Vital sign measurements |
| | • Physical examination |
| | • Bristol stool scale |
| | • Specific markers of GI integrity |
| | • Specific immune biomarkers |
| Secondary | |
| • Clinical improvement | • Psoriasis Area and Severity Index |
| in participants | (PASI) |
| with mild to moderate | • Eczema Area and Severity Index |
| psoriasis | (EASI) |
| • Clinical improvement | • SCORing Atopic Dermatitis |
| in participants | (SCORAD) |
| with mild to moderate | • Lesion Severity Score (LSS) |
| atopic dermatitis | • Percentage of Body Surface Area |
| | (BSA) affected by disease in Cohorts |
| | 3, 4, 5, 6, and 7 |
| | • Investigator's Global Assessment |
| | (IGA) |
| Exploratory | |
| • Evidence of pharmacodynamic | • Immunohistochemistry (IHC) on |
| effects on skin | skin biopsies |
| • Evidence of systemic immune | • Messenger ribonucleic acid (mRNA) |
| modulation | transcription analysis on skin |
| • Effect of Prevotella histicola | biopsies |
| Strain B on gene and | • IHC/immunofluorescence (IF) of |
| protein expression, and | skin tissue looking at immune cell |
| explore the relationship | infiltrates |
| between genomic, genetic, | • Blood cytokine and chemokine |
| and proteomic biomarkers | levels |
| and disease biology, drug | • Blood gene expression profiling |
| treatment and inflammatory and | • Microbiome composition (in faeces) |
| immune responses | |

### Study Design

This is a FIH single center study that is being designed and conducted in accordance with the European Medicines Agency (EMA) FIH Guidance [EMA, 2017]. However, as Prevotella histicola Strain B is a naturally occurring organism commonly found on oral, nasopharyngeal, GI, and genito-urinary mucosal surfaces, the study development program has been modified accordingly. The study design is consistent with advice from the Health Authorities on other commensal-type organisms.

This study will use a within-cohort progression from the single to multiple dosing period of the study. Participants who are successfully screened will be randomized to either the active (Prevotella histicola Strain B) or placebo group on Day 1 and dosing will be initiated. For Cohorts 1 to 5, there will be a sentinel group of 2 participants (1 active, 1 placebo). The remainder of the cohort will be dosed following a review of the safety data from the sentinel group after at least 3 days of multiple dosing. Following single dosing and a 48-hour washout period, healthy participants will start the 14-day multiple dosing period of the protocol and participants with psoriasis or atopic dermatitis will start a 28-day multiple dosing period. For the first cohort only, all participants will remain as an inpatient in the clinical facility on Day −1 (at least 24 hours prior to the first dose) until 24 hours post the first dose in the multiple dosing period. Thereafter, if considered safe to do so following a safety review, all further dosing in that cohort and all other cohorts will be on an outpatient basis. All healthy volunteer participants will return to the clinical facility daily for each dosing and will be observed for at least 0.5 hours after each outpatient dosing. Participants with mild to moderate psoriasis or atopic dermatitis will return to the clinical facility at the scheduled outpatient visit for collection of their study intervention.

Potential adverse effects can be readily monitored in humans during the clinical study. Systemic adverse effects are unlikely, however indicators of potential disruption of intestinal epithelial junctions can be monitored by systemic measures such as C-reactive protein (CRP) and fecal calprotectin. These will be monitored by the safety review committee (SRC) and will be reviewed prior to dose escalation.

Indicators of local effects can be monitored by AEs, Bristol stool scale and fecal calprotectin. General safety can be monitored by routine safety blood and monitoring of vital signs. Safety will be continuously and cumulatively evaluated.

### Overall Design

The study is a single center, randomized, placebo-controlled clinical study with dose escalations and dose expansions in healthy volunteers and participants with either mild to moderate psoriasis or mild to moderate atopic dermatitis. The investigators and participants will be blinded to study drug but the Sponsor will be unblinded. The rationale for the Sponsor being unblinded is to enable the Sponsor to make strategic decisions about the program and plan for the next studies. In addition, the availability of the biomarker data will enable the planning of future studies with regard to the choice of indication and patient population.

The study consists of 2 cohorts of healthy volunteers, 3 cohorts of participants with mild to moderate psoriasis, and 2 cohorts of participants with mild to moderate atopic dermatitis, and will test escalating doses from approximately 1/10th of the estimated therapeutic dose to a maximum of approximately 5 times the estimated therapeutic dose versus placebo. The primary aim of the study is to assess safety and tolerability of Prevotella histicola Strain B. Secondary and exploratory endpoints are designed to establish whether there are any effects on the systemic immune system and potential clinical benefit. The description of the cohorts is detailed below and the rationale for each is described herein.

The design of the study allows a dose escalation in healthy participants to ensure Prevotella histicola Strain B is safe and well tolerated in humans (Cohorts 1 and 2). Prevotella histicola Strain B will then be tested in participants with mild to moderate psoriasis for safety and tolerability and for an effect on the disease pathology (Cohort 3). Prevotella histicola Strain B will then be tested in 2 more psoriasis cohorts (Cohorts 4 and 6) and in 2 cohorts of participants with mild to moderate atopic dermatitis (Cohorts 5 and 7) to investigate the potential for Prevotella histicola Strain B to treat Th2-driven immunoinflammatory disorders.

Cohort 1: 12 healthy participants will be randomized into Cohort 1: 8 participants will be randomized to the lowest dose of Prevotella histicola Strain B of approximately 1.6×10¹⁰ total cells which is 0.1× the allometric scaled preclinical efficacious dose level (Dose 1, approximately 1/10th of the estimated therapeutic dose) and 4 participants will be randomized to placebo. Sentinel dosing of the first pair (1 on active and 1 on placebo) will happen on Day 1. The remainder of the cohort will be dosed from Day 6. Following a 48-hour washout period, the participants will then start a 14-day multiple dosing period.

Cohort 2: 12 healthy participants will be randomized into Cohort 2: 8 participants will be randomized to Prevotella histicola Strain B up to a maximum of approximately 1.6×10¹⁰ total cells which is 1× the allometric scaled preclinical efficacious dose level (Dose 2, the estimated therapeutic dose) and 4 participants will be randomized to placebo. Sentinel dosing of the first pair (1 on active and 1 on placebo) will happen on Day 1. The remainder of the cohort will be dosed from Day 6. Following a 48-hour washout period, the participants will then start a 14-day multiple dosing period.

Cohort 3: 12 participants with mild to moderate psoriasis will be randomized into Cohort 3: 8 participants will be randomized to Prevotella histicola Strain B up to a maximum of approximately 1.6×10¹¹ total cells which is 1× the allometric scaled preclinical efficacious dose level (Dose 2, the estimated therapeutic dose) and 4 participants will be randomized to placebo. Sentinel dosing of the first pair (1 on active and 1 on placebo) will happen on Day 1. The remainder of the cohort will be dosed from Day 6. Following a 48-hour washout period, the participants will then start a 28-day multiple dosing period.

Cohort 4: Up to 24 participants with mild to moderate psoriasis will be randomized into Cohort 4 (in a 2:1 randomization ratio of Prevotella histicola Strain B and placebo):

16 participants will be randomized to Prevotella histicola Strain B up to a maximum of approximately 8.0×10¹¹ total cells which is 5× the allometric scaled preclinical efficacious dose level (Dose 3, 5 times the estimated therapeutic dose) and 8 participants will be randomized to placebo. Sentinel dosing of the first pair (1 on active and 1 on placebo) will happen on Day 1. The remainder of the cohort will be dosed from Day 6. Following a 48-hour washout period, the participants will then start a 28-day multiple dosing period.

Cohort 5: Up to 24 participants with mild to moderate atopic dermatitis will be randomized into Cohort 5 (in a 2:1 randomization ratio of Prevotella histicola Strain B and placebo): 16 participants will be randomized to Prevotella histicola Strain B up to a maximum of approximately 8.0×10¹¹ total cells which is 5× the allometric scaled preclinical efficacious dose level (Dose 3, 5 times the estimated therapeutic dose) and 8 participants will be randomized to placebo. Sentinel dosing of the first pair (1 on active and 1 on placebo) will happen on Day 1. The remainder of the cohort will be dosed from Day 6. Following a 48-hour washout period, the participants will then start a 28-day multiple dosing period.

Cohort 6: Up to 24 participants with mild to moderate psoriasis will be randomized into Cohort 6 (in a 2:1 randomization ratio of Prevotella histicola Strain B and placebo):

16 participants will be randomized to Prevotella histicola Strain B up to a maximum of approximately 8.0×10¹¹ total cells which is 5× the allometric scaled preclinical efficacious dose level (Dose 3, 5 times the estimated therapeutic dose) and 8 participants will be randomized to placebo. All participants will be dosed for 28 days.

Cohort 7: Up to 24 participants with mild to moderate atopic dermatitis will be randomized into Cohort 7 (in a 2:1 randomization ratio of Prevotella histicola Strain B and placebo): 16 participants will be randomized to Prevotella histicola Strain B up to a maximum of approximately 8.0×10¹¹ total cells which is 5× the allometric scaled preclinical efficacious dose level (Dose 3, 5 times the estimated therapeutic dose) and 8 participants will be randomized to placebo. All participants will be dosed for 28 days.

Dosing in Cohorts 4, 5, 6 and 7 can occur in parallel following a review of the safety data from Cohort 3. The sequencing of the cohorts can be adjusted to accommodate the available drug supply, e.g. Cohort 6 can be conducted before Cohort 4 and Cohort 7 can be conducted before Cohort 5. All safety data from previous lower doses cohorts will be reviewed prior to dose escalation.

### Scheme 1 Study Schema (FIG. 25)

Note: progression to the next cohort for Cohorts 1 to 3 will be decided by the SRC. Cohorts 4 to 7 will be run according to availability of drug supply. Sentinel dosing of the first pair (1 on active and 1 on placebo) will happen on Day 1 of Cohorts 4, 5, 6 and 7 if dose escalation occurs.

### Scientific Rationale for Study Design

The design of the study allows a dose escalation in healthy volunteers to ensure Prevotella histicola Strain B is tolerated in humans and this will be followed by the testing of 2 further hypotheses.

### Hypothesis 1:

Prevotella histicola Strain B is well tolerated in humans.

Endpoints: standard safety and tolerability endpoints will be measured including CRP, fecal calprotectin and Bristol stool scale. Particular attention will be given to GI AEs and potentially infectious AEs.

### Hypothesis 2:

Daily administration of Prevotella histicola Strain B improves mild to moderate psoriasis.

Endpoints:
1. Standard safety and tolerability endpoints
2. PASI score
3. IGA
4. LSS
5. Percentage of BSA affected by disease in Cohorts 3, 4, 5, 6, and 7
6. IHC and IHC/IF on paired biopsy samples
7. Tissue and circulating chemokine and cytokine measurements
8. Tissue transcription profiling

### Hypothesis 3:

Daily administration of Prevotella histicola Strain B improves mild to moderate atopic dermatitis.

Endpoints:
1. Standard safety and tolerability endpoints
2. EASI score
3. SCORAD
4. IGA
5. LSS
6. Percentage of BSA affected by disease in Cohorts 3, 4, 5, 6, and 7
7. IHC and IHC/TF on paired biopsy samples
8. Tissue and circulating chemokine and cytokine measurements
9. Tissue transcription profiling

### Justification for Dose

Dosing will occur in a sequential fashion, with the first cohort receiving the lowest dose level, and dosing only proceeding to the next dose level following review of the safety data from the previous cohort by the SRC. The first dose level (Cohort 1) will receive 1/10^{th} (×0.1) of the estimated therapeutic dose, and then Cohort 2 will receive up to the maximum of the 1× estimated therapeutic dose. This sequential dose regimen is designed to minimize risk to participants, with starting doses guided by the general experience in probiotic formulations and an understanding of the dose-response relationship as defined in the preclinical models. For considerations of scaling, the drug product was regarded as having predominantly local interaction with cells of the GI mucosa, with subsequent systemic effects on cells of the immune system. Such a mechanism is not consistent with the assumptions of traditional allometric scaling, therefore 2 other parameters were considered that might reflect the topical interaction in the GI tract, namely relative GI mucosal surface area and relative stool mass. While a complete analysis of relative GI mucosal surface area could not be identified in the literature, it has been estimated as a function of body mass to the ¾ power [Karasov, 2012]. Using stool mass ratio, standard allometric scaling, and GI mucosal surface area calculations, human:mouse dose ratios of approximately 100×, 300×, and 450×, respectively, were calculated. For the purpose of considering a likely efficacious dose, calculations on a 345× scale factor have been used.

The starting dose for the clinical study is based on the predicted therapeutic range based on preclinical in vitro and in vivo experiments. This expected range is based on the total cell count of microbes given by oral gavage to the mice in the preclinical animal model experiments. This has been adjusted using allometric scaling approaches and converted to a milligram equivalent dose providing an estimate of the likely therapeutic range.

### End of Study Definition

A participant is considered to have completed the study if he/she has completed treatment to the end of their assigned cohort and completed their final safety follow-up visit 14 days after their last dose.

The end of the study is defined as the date of the last visit of the last participant in the study or last scheduled procedure shown in the Schedule of Activities (SoA).

### Risk Mitigation and Management

This is a FIH study and will be conducted in a Medicines and Healthcare Products Regulatory Agency (MHRA) accredited clinical research facility that has experience in conducting FIH studies. The study will be conducted in accordance with the EMA FIH Guidance, the terms of the clinical trial authorization, and all relevant good practices.

The investigator will write a detailed risk management plan in accordance with local procedures prior to the start of the study and in accordance with the requirements of the MHRA accreditation scheme.

Prevotella histicola Strain B is a naturally occurring organism defined as a risk group 1 microbe and therefore, is very low risk to staff and environment. Prevotella histicola Strain B is not genetically modified. There are no specific biological safety requirements. No additional requirements for staff over and above normal clinical care and no specific containment procedures are required. Overall, although this is a FIH study for a novel form of therapeutic, the therapeutic agent is a common natural organism commonly found on oral, nasopharyngeal, GI, and genito-urinary mucosal surfaces, and is therefore expected to be generally well tolerated. Prevotella spp. can cause anaerobic infections in the respiratory tract (lungs, nose, throat, ear infections) and periodontal disease and abscesses can be associated with Prevotella spp. infections. A range of antibiotics can be used to treat these patients depending on the clinical setting and antibiotic sensitivity testing. Prevotella histicola Strain B is sensitive to standard antibiotics such as penicillins and cephalosporins which will be available as rescue therapy. If participants are allergic to these rescue therapies, then macrolides (e.g. clarithromycin or erythromycin) or tetracyclines (e.g. doxycycline) may be used as an alternative.

Using quantitative polymerase chain reaction (PCR) and strain-specific primers, a bio-distribution study was performed for Prevotella histicola Strain B during transit through the intestinal tract as well as the level of systemic distribution of following a single oral dose in mice. Total commensal bacterial load from the same samples was also measured by 16s analysis. Oral administration of Prevotella histicola Strain B led to a rise in the abundance of the organism, as measured by strain-specific PCR primers, but stayed well below the level of the total resident microbial load by approximately 6 logs. Importantly, Prevotella histicola Strain B was not detected outside of the GI tract at any timepoint and was only detected in the intestine for up to 8 hours post-treatment, suggesting that the bacteria do not establish long-term colonization in the intestinal tract after a single dose. These data demonstrate that Prevotella histicola Strain B is luminally restricted with undetectable systemic exposure following oral dosing. It is theoretically possible that Prevotella histicola Strain B may cause local gut inflammatory responses and/or disruption of the intestinal epithelial junctions. These effects can be monitored in humans by methods such as symptoms (AEs), CRP, change in bowel habits (Bristol stool scale) and fecal calprotectin.

### Study Population

This protocol contains healthy volunteers and participants with mild to moderate psoriasis and mild to moderate atopic dermatitis. Prospective approval of protocol deviations, also known as protocol waivers or exemptions, is not permitted.

### Inclusion Criteria

### All Participants

Participants are eligible to be included in the study only if all of the following criteria apply:
1. Capable of giving signed informed consent as described in Appendix 1 which includes compliance with the requirements and restrictions listed in the informed consent form (ICF) and in this protocol. Informed consent will be obtained prior to any screening procedures and in accordance with national, local, institutional guidelines.
2. Age≥18 years to 60 years, inclusive.
3. Participant has a body mass index of ≥18 kg/m² to ≤35 kg/m² at Screening.
4. Contraception:

Male participants:
A male participant must agree to use contraception as detailed in Appendix 4 of this protocol during their participation in this study and for a period of 90 days after the last dose and refrain from donating sperm during this period.
   Female participants:
A female participant is eligible to participate if she is not pregnant (see Appendix 4), not breastfeeding, and at least 1 of the following conditions applies:
   i. Not a woman of child-bearing potential (WOCBP) as defined in
   Appendix 4
OR
   ii. A WOCBP who agrees to follow the contraceptive guidance in Appendix 4 during their participation in this study, 28 days prior to the first dose and for at least 1 complete menstrual cycle (≥30 days) after the last dose.
5. The participant has clinical laboratory evaluations (including clinical chemistry, haematology, and complete urinalysis) within the reference range for the testing laboratory, unless the results are deemed not to be clinically significant by the investigator or Sponsor (1 repeat test is permitted).
6. CRP≤10 mg/L and fecal calprotectin≤110 mcg/g faeces (note a participant with a slightly elevated fecal calprotectin can be included if the results are deemed to be not clinically significant by the investigator).
7. Participants who are overtly healthy as determined by medical evaluation including medical history, physical examination, laboratory tests, and ECG monitoring at Screening and on Day 1.

Additional Inclusion Criteria for Participants With Mild to Moderate Psoriasis
1. Participant has had a confirmed diagnosis of mild to moderate plaque-type psoriasis for at least 6 months involving ≤10% of body surface area (BSA) (excluding the scalp).
2. Participant has a minimum of 2 psoriatic lesions with at least 1 plaque in a site suitable for biopsy.

Additional Inclusion Criteria for Participants With Mild to Moderate Atopic Dermatitis
1. Mild to moderate atopic dermatitis with a minimum of 3% to a maximum of 15% BSA involvement.
2. Participant has had a confirmed diagnosis of mild to moderate atopic dermatitis for at least 6 months (IGA score of 2 or 3).
3. Participant has a minimum of 2 atopic dermatitis lesions with at least 1 in a site suitable for biopsy.

### Exclusion Criteria

### All Participants

1. Female participant who is pregnant, or plans to become pregnant during the study, or breastfeeding, or sexually active with child-bearing potential who is not using a highly effective birth control method as indicated in Appendix 4.
2. Participant has received live attenuated vaccination within 6 weeks prior to Screening or intends to have such a vaccination during the course of the study.
3. Participant has received any investigational drug or experimental procedure within 90 days or 5 half-lives, whichever is longer, prior to study intervention administration.
4. Participant requires treatment with an anti-inflammatory drug during the study period. Paracetamol will be permitted for use as an antipyretic and/or analgesic (maximum of 4 grams/day in any 24-hour period).
5. Participant has an active infection (e.g. sepsis, pneumonia, abscess) or has had an infection requiring antibiotic treatment within 6 weeks prior to study intervention administration. When in doubt, the investigator should confer with the Sponsor study physician.
6. Participant has renal or liver impairment, defined as:
   a. For healthy volunteers:
   i. For women, serum creatinine level≥72 μmol/L; for men, ≥102 μmol/L, or
   ii. Alanine aminotransferase (ALT) and aspartate aminotransferase (AST)≥1.5× upper limit of normal (ULN), or
   iii. Alkaline phosphatase (ALP) and/or bilirubin>1.5×ULN
b. For participants with mild to moderate psoriasis or atopic dermatitis:
   i. For women, serum creatinine level≥72 μmol/L; for men, ≥102 μmol/L, or
   ii. ALT or AST>2×ULN and/or bilirubin>1.5×ULN
7. Participant has active neoplastic disease or history of neoplastic disease within 5 years of Screening (except for basal or squamous cell carcinoma of the skin or carcinoma in situ that has been definitively treated with standard of care).
8. Major surgery within the previous 4 weeks.
9. Impaired cardiac function or clinically significant cardiac diseases, including any of the following:
   a. Unstable angina or acute myocardial infarction≤3 months prior to Screening;
   b. Clinically significant heart disease (e.g. symptomatic congestive heart failure [e.g. New York Heart Association [NYHA]>Class 2]; uncontrolled arrhythmia, or hypertension; history of labile hypertension or poor compliance with an antihypertensive regimen).
10. Participant has a known history of human immunodeficiency virus (HIV); HIV testing is required as part of this study.
11. Known, active hepatitis A, hepatitis B (HBV), or hepatitis C (HCV) infection; or known to be positive for HCV ribonucleic acid (RNA) or hepatitis B surface antigen (HBsAg).
12. Participant has active central nervous system (CNS) malignancy. Participants who have only had prophylactic intrathecal or intravenous chemotherapy against CNS disease are eligible.
13. Participant has GI tract disease (e.g. short bowel syndrome, diarrhoea predominant irritable bowel syndrome [IBS]) that could interfere with the GI delivery and transit time of Prevotella histicola Strain B.
14. Serious psychiatric or medical conditions that, in the opinion of the investigator, could interfere with treatment, compliance, or the ability to give consent.
15. Participant has a history of hypersensitivity or allergies to Prevotella (or Prevotella-containing probiotics) including any associated excipients, or has a history of hypersensitivity or allergies to placebo capsule (magnesium stearate and cellulose) or to the hard capsule shells (hydroxyl propyl methyl cellulose and titanium dioxide).
16. The participant has taken any over-the-counter (OTC) or prescription medication including vitamins, herbal supplements and nutraceuticals (e.g. supplements including high doses of probiotics and prebiotics, as usually found in capsules/tablets/powders) but with the exception of paracetamol and anti-histamines, within 14 days prior to baseline (Day −1) or anticipates an inability to abstain from these products for the duration of the study period. Note that probiotic and prebiotic foods that contain low doses are allowed (e.g. yoghurt, kefir, kombucha).
17. The participant has a significant history of drug abuse or regular use of illicit drugs or a history of alcohol abuse within 1 year prior to Screening, or has tested positive for drugs of abuse or alcohol at Screening.
18. The participant intends to donate sperm during the course of this study and for a period of 90 days after the last dose.
19. The participant has donated more than 400 mL of blood or blood products within 90 days prior to baseline (Day −1) or plans to donate blood during the study.
20. The participant has had an acute, clinically significant illness within 30 days prior to the first dose of study intervention.

Additional Exclusion Criteria for Participants With Mild to Moderate Psoriasis
1. Participant has received systemic nonbiologic psoriasis therapy (methotrexate [MTX], steroids, cyclophosphamide) or psoralen plus ultraviolet A (PUVA)/ultraviolet A (UVA) phototherapy within 4 weeks prior to Screening.
2. Participant has received treatment with biologic agents within 12 months prior to first dose.
3. Participant is unwilling to comply with the protocol including required biopsies and sample collections required to measure disease.
4. Participant continues to use topical or oral pharmacologically active agents 2 weeks prior to the start of dosing. Emollients may be used if the participant was already using them as part of their care.

Additional Exclusion Criteria for Participants With Mild to Moderate Atopic Dermatitis 1. Participant is receiving systemic non-biologic atopic dermatitis therapy (MTX, steroids, cyclophosphamide) or has received therapy within 4 weeks prior to Screening.
2. Participant has received treatment with biologic agents within 12 months prior to first dose.
3. Participant is unwilling to comply with the protocol including required biopsies and sample collections required to measure disease.
4. Participant continues to use topical or oral pharmacologically active agents 2 weeks prior to the start of dosing. Emollients may be used if the participant was already using them as part of their care.

### 1.4. Lifestyle Restrictions

In Cohort 1, participants are asked to stay in the study unit for 5 nights, from Day −1 (24 hours prior to the first dose) until 48 hours post the first dose in the multiple dosing period. Participants must abstain from taking prescription or OTC drugs (including high doses of probiotics and prebiotics as usually found in capsules/tablets/powders, vitamins and dietary or herbal supplements), but with the exception of paracetamol and anti-histamines, for 14 days prior to the baseline visit (Day −1) and until completion of the follow-up visit, unless, in the opinion of the investigator and Sponsor, the medication will not interfere with the study. Note that probiotic and prebiotic foods that contain low doses are allowed (e.g. yoghurt, kefir, kombucha). Participants must refrain from consuming acidic drinks for 1 hour either side of dosing and from eating 2 hours before dosing and 1 hour after dosing. Apart from this, there are no lifestyle restrictions in this protocol.

### 1.5. Screen Failures

Screen failures are defined as participants who consent to participate in the clinical study but are not subsequently randomly assigned to study intervention/entered in the study. A minimal set of screen failure information is required to ensure transparent reporting of screen failure participants to meet the Consolidated Standards of Reporting Trials (CONSORT) publishing requirements and to respond to queries from regulatory authorities. Minimal information includes demography, screen failure details, eligibility criteria, and any SAEs.

Individuals who do not meet the criteria for participation in this study (screen failure) may be rescreened if they failed on inclusion criterion #5, exclusion criteria #2, 3, 5, 16, or 20 and/or additional exclusion criteria for patients with mild to moderate psoriasis or atopic dermatitis #1, 2 and 4. Rescreening of all participants should be agreed with the Medical Monitor prior to retest. Rescreened participants will be assigned a new participant number as for the initial screening.

Participants may also be rescreened if they initially pass the screening assessments but go beyond the 28-day screening period time limit.

### 2. STUDY INTERVENTIONS

Study intervention is defined as any investigational intervention(s), marketed product(s), placebo, or medical device(s) intended to be administered to a study participant according to the study protocol.

### 2.1. Study Interventions Administered

All study interventions in this study will be administered orally. Prevotella histicola Strain B capsules will be enteric coated to release the contents in the duodenum and will be supplied. Three dose levels of Prevotella histicola Strain B will be provided:

Dose level 1= 1/10^{th} estimated therapeutic dose

Dose level 2=up to 1× estimated therapeutic dose based on preclinical data

Dose level 3=up to 5× estimated therapeutic dose based on preclinical data

Matched placebo capsules will be supplied. Participants must refrain from consuming acidic drinks 1 hour either side of dosing and from eating 2 hours before dosing and 1 hour after dosing. See Table 5 below for a summary of the key information or alternatively, refer to the Pharmacy Manual for further details.

TABLE 5
Summary of Study Interventions

| | | | | Anticipated | Maximum | |
|---|---|---|---|---|---|---|
| | | | Maximum | Dose Levels | Dose Levels | |
| | | | sample | (once daily | (once daily | Total Cell |
| Cohort | Population | Formulation | size | dosing) | dosing) | Count |
| 1 | Healthy | Enteric | 8 | 1/10^{th} of | 1/10^{th} of KED | Approximately |
| | volunteers | coated | | HED | | 1.6 × 10¹⁰ |
| | | capsule | 4 | Placebo | Placebo | Placebo |
| 2 | Healthy | Enteric | 8 | ≤HED | ≤HED | Up to |
| | volunteers | coated | | | | approximately |
| | | capsule | | | | 1.6 × 10¹¹ |
| | | | 4 | Placebo | Placebo | Placebo |
| 3 | Mild to | Enteric | 8 | ≤HED | ≤HED | Up to |
| | moderate | coated | | | | approximately |
| | psoriasis | capsule | | | | 1.6 × 10¹¹ |
| | | | 4 | Placebo | Placebo | Placebo |
| 4 | Mild to | Enteric | 16 | ≤5 × HED | ≤5 × HED | Up to |
| | moderate | coated | | | | approximately |
| | psoriasis | capsule | | | | 8.0 × 10¹¹ |
| | | | 8 | Placebo | Placebo | Placebo |
| 5 | Mild to | Enteric | 16 | ≤5 × HED | ≤5 × HED | Up to |
| | moderate | coated | | | | approximately |
| | atopic | capsule | | | | 8.0 × 10¹¹ |
| | dermatitis | | 8 | Placebo | Placebo | Placebo |
| 6 | Mild to | Enteric | 16 | ≤1 × HED | ≤5 × HED | Up to |
| | moderate | coated | | | | approximately |
| | psoriasis | capsule | | | | 8.0 × 10¹¹ |
| | | | 8 | Placebo | Placebo | Placebo |
| 7 | Mild to | Enteric | 16 | ≤1 × HED | ≤5 × HED | Up to |
| | moderate | coated | | | | approximately |
| | atopic | capsule | | | | 8.0 × 10¹¹ |
| | dermatitis | | 8 | Placebo | Placebo | Placebo |

HED = human equivalent dose
Doses specified as ≤ indicates a dose up to the maximum specified value

### 2.2. Preparation/Handling/Storage/Accountability

All capsules will be supplied in blister packs and must be kept in controlled conditions of 2-8° C.
1. The investigator or designee must confirm appropriate temperature conditions have been maintained during storage and transit for all study interventions received and any excursions are reported and resolved before use of the study intervention.
2. Only participants enrolled in the study may receive the study intervention and only authorised site staff may supply or administer the study intervention. All study intervention must be stored in a secure, environmentally controlled, and monitored area (manual or automated with the ability to show minimum and maximum temperatures daily), in accordance with the labelled storage conditions with access limited to the investigator and authorised site staff.
3. The investigator is responsible for study intervention accountability, reconciliation, and record maintenance (i.e. receipt, reconciliation, and final disposition records).
4. Further guidance and information for the final disposition of unused study interventions are provided in the Pharmacy Manual.

### 2.3. Measures to Minimize Bias: Randomization and Blinding

This is a randomized controlled study and therefore, the treatment allocation within cohorts is random. Randomized treatment ensures minimization of selection bias, so that the individuals in the 2 treatment groups are not systematically different, other than the treatment that they receive. A paper randomization will be used to assign participants their study intervention.

The investigational drug blind shall not be broken by the investigator unless information concerning the study intervention is necessary for the medical treatment of the participant.

For unblinding a participant, the investigational drug blind can be obtained by opening the sealed envelope.

The Sponsor must be notified immediately (within 24 hours) if the investigational drug blind is broken. The date, time, and reason the blind was broken must be recorded on the appropriate Case Report Form (CRF).

### 2.4. Study Intervention Compliance

Drug supplies will be counted and reconciled at the study site before being returned. The investigator must maintain 100% accountability for all study intervention received and dispensed during his or her entire participation in the study. Proper drug accountability includes, but is not limited to:
Continuously monitoring expiration dates if expiry date or retest date is provided to the investigator.
Frequently verifying that actual inventory matches documented inventory.
Verifying that the log is completed for the drug lot used to prepare each dose.
Verifying that all containers and/or packs used are documented accurately on the log.
Verifying that required fields are completed accurately and legibly.

If any dispensing errors or discrepancies are discovered, the Sponsor must be notified immediately.

The investigator must maintain a current inventory (Drug Accountability Log) of all study intervention delivered to the site, inventory at the site, dispensing log, and participants' use records. This log must accurately reflect the drug accountability of the study intervention at all times. The following information will be recorded at a minimum: protocol number and title, name of investigator, site identifier and number, description of study intervention, Med ID numbers, expiry or retest date and amount dispensed, and the date and amount returned to the site by the participant, including the initials of the person dispensing and receiving the study intervention. The log should include all required information as a separate entry for each participant to whom study intervention is dispensed.

Prior to site closure or at appropriate intervals, a representative from the Sponsor or its designee will perform clinical study material accountability and reconciliation before clinical study materials are returned to the Sponsor or its designee for destruction. The investigator will retain the original documentation regarding clinical study material accountability, return, and/or destruction, and copies will be sent to the Sponsor.

The investigator will be notified of any change in expiry date or retest date of clinical study material during the study conduct. On expiry date notification from the Sponsor or designee, the site must complete all instructions outlined in the notification, including segregation of expired clinical study material for return to the Sponsor or its designee for destruction. In the event of expiry date extension of supplies already at the study site, supplies may be relabeled with the new expiry date at that site. In such cases, the Sponsor or its designee will prepare additional labels, certificates of analyses, and all necessary documentation for completion of the procedure at the sites.

### 2.5. Concomitant Therapy

Participants must abstain from taking prescription or OTC drugs (including high doses of probiotics and prebiotics as usually found in capsules/tablets/powders, vitamins and dietary or herbal supplements) within 14 days before the start of study intervention until completion of the follow-up visit, unless, in the opinion of the investigator and Sponsor, the medication will not interfere with the study. Note that probiotic and prebiotic foods that contain low doses are allowed (e.g. yoghurt, kefir, kombucha).

Investigational agents other than Prevotella histicola Strain B are not allowed during the study. Pharmacologically active treatments for psoriasis or atopic dermatitis, apart from emollients, are contraindicated and should be stopped at least 2 weeks prior to entry into the study.

The use of any concomitant medication, including OTC medications, deemed absolutely necessary for the care of the participant is permitted during the study provided they do not have a known effect on GI transit time or function. The use of any immunosuppressive agents must be discussed between the investigator and the Medical Monitor on a case-by-case basis. Hormonal contraceptives are permitted in WOCBP (hormonal contraceptives include any marketed contraceptive agent that includes an estrogen and/or a progestational agent).

Any medication or vaccine (including OTC or prescription medicines, probiotics, and/or herbal supplements) that the participant is receiving at the time of enrolment or receives during the study must be recorded along with:
Reason for use
Dates of administration including start and end dates
Dosage information including dose and frequency

Any diagnostic, therapeutic, or surgical procedure performed during the study period should be recorded, including the dates, description of the procedure(s), and any clinical findings, if applicable. The Medical Monitor should be contacted if there are any questions regarding concomitant or prior therapy.

Anti-histamines and paracetamol doses of s; 4 grams/day (in any 24-hour period) are permitted for use any time during the study. Other concomitant medication may be considered on a case-by-case basis by the investigator in consultation with the Medical Monitor if required. Participants with psoriasis or atopic dermatitis, who are willing to participate in this study, must not be on topical or oral pharmacologically active agents 2 weeks prior to first study intervention dose. Emollients may be used if the participants were already using them as part of their care.

Participants should be willing to stop their current medication due to intolerability or ineffectiveness of their medication rather than purely for the sake of participation in this study. This should be clearly documented in the participants' notes.

### 2.5.1. Rescue Medicine

The study site will supply an appropriate antibiotic if the clinical situation suggests this is required. The following rescue medications may be used:
1. Penicillin V
2. Amoxicillin
   Or if allergic to the above medications:
3. Macrolides (e.g. clarithromycin or erythromycin)
4. Tetracyclines (e.g. doxycycline) Use of further alternative antibiotics may be discussed with the Medical Monitor.

The date and time of rescue medication administration as well as the name and dosage regimen of the rescue medication must be recorded.

### 2.6. Dose Escalations and Transitions

The decision to permit transition from single dose to multiple dose for each participant will be made by the Principal Investigator (or delegate) and Medical Monitor.

Dose escalation decisions will be made when at least 9 participants have completed the multiple dosing period of the stated dose level. To implement dose escalation decisions, the available AEs and laboratory test data will be evaluated at a dose decision meeting or teleconference. Drug administration at the next dose cohort will not proceed until the investigator receives written confirmation from Sponsor indicating that the results of the previous dose cohort were evaluated and that it is permissible to proceed to the next higher dose cohort.

Dose escalation increments will be up to 10-fold, as described in Table 5 above. The rationale for this increment is based on the fact that Prevotella is a human commensal organism that can reach up to 50% of total microbial load in some populations [De Filippo, 2010], suggesting minimal risk to participants.

### 2.6.1. Stopping Rules

Safety data will be evaluated against these stopping rules on an ongoing basis. If either the investigator or Sponsor considers any of these events to be either moderate or severe and possibly related to study treatment, the treatment for that participant(s) will be unblinded to determine if they were receiving Prevotella histicola Strain B. If 1 or more of the following criteria are met for a participant(s) on active treatment, dose escalation will stop and no other participant will receive this or a higher dose. Prior to escalating the dose, an amended protocol will be submitted the Ethics Committee and the MHRA for review and approval.
One participant experiences an SAE that is related to study intervention
More than 1 participant experiences a non-diarrheal AE of severe intensity that is related to study intervention
Two participants in the same group experience the same AE of moderate intensity that is related to the study intervention
Two participants in the same group experience evidence of GI mucosal barrier disruption such as CRP≥20 mg/L or fecal calprotectin≥165 mcg/g, AND significant changes in bowel habits or other indicators of local intolerability
One participant's serum creatinine>1.5×ULN and the results are confirmed on a repeat taken within 24 hours of the initial sample
One participant meets the following criteria for drug-induced liver injury as defined by the US Food and Drug Administration (FDA) in Guidance for Industry Drug Induced Liver Injury: Premarketing Clinical Evaluation [FDA, 2009]
   Hepatocellular injury (≥3-fold elevations above ULN for AST or ALT); and
   Elevation of serum total bilirubin to >2×ULN, without initial findings of cholestasis (serum ALP activity>2×ULN); and
   No other reason can be found to explain the combination of increased transaminase and serum total bilirubin (such as viral hepatitis, pre-existing or acute liver disease, or another drug capable of causing the observed injury)
   Elevations of transaminases and bilirubin of clinical concern should be confirmed on a repeat sample 48 to 72 hours later.
Two participants experience either AST or ALT≥3×ULN or elevation of serum total bilirubin to >2×ULN
The Principal Investigator (or delegate) or Sponsor may decide to halt escalation for other reasons.

Participants experiencing any of the above will be followed up until the AE has resolved.

Formal documented safety review will be conducted as follows:
1. At least 46 hours after dosing in each sentinel group prior to progression to multiple dosing period in the sentinel group.
2. Following at least 3 doses of multiple dosing in sentinel group prior to dosing the remainder of that cohort.
3. Following completion of a cohort at each dose level prior to escalating to the following dose level.
4. Prior to progression into patients.

### Data Requirements for Documented Safety Review

Safety review will be based on the following data (Table 6), which shall all be checked and Quality Controlled (QC'd) as far is practically possible.

TABLE 6
Data Requirements for Documented Safety Review

| Review | | | | Data |
|---|---|---|---|---|
| Time- | | | Required | Quality |
| point | Purpose | Minimal Data Set | Attendees | Review |
| 1 | Cohorts 1-5: | AEs, 24-hour lab | Principal | No |
| | Sentinel pair to | safety, ECG, | Investigator | |
| | proceed to | 24-hour | (or delegate) | |
| | multiple dosing | post-dose CRP, | and Medical | |
| | (after single dose) | Bristol stool | Monitor | |
| | Cohort 1-5: | scale up | | |
| | Participants from | to 24 hours (if | | |
| | the main | available), and a | | |
| | cohort to proceed to | review of the | | |
| | multiple dosing | stopping rules | | |
| | (after single dose) | based on | | |
| | | available data | | |
| 2 | Cohorts 1-5: | AEs, 48-hour lab | Principal | No |
| | After sentinel | safety¹, ECG, 48- | Investigator | |
| | pair receives | hour post-dose | (or delegate) | |
| | 3 multiple doses, | CRP¹, Bristol stool | and Medical | |
| | approval | scale up to | Monitor | |
| | to expand to the | Day 5 (if | | |
| | main cohort | available), and a | | |
| | (non-sentinels) | review of the | | |
| | | stopping rules | | |
| | | based on | | |
| | | available data | | |
| 3 | Cohort 1 only: | AEs, Day 4 lab | Principal | No |
| | Discharge from | safety, Day 4 | Investigator | |
| | clinic on Day 5 | post-dose CRP, | (or delegate) | |
| | | Bristol stool scale | and Medical | |
| | | up to Day 5 (if | Monitor | |
| | | available), | | |
| | | stopping rules | | |
| 4 | Dose escalation: | All AEs, lab safety, | SRC | Yes |
| | ≥9 participants for | ECGs, CRP, fecal | (Principal | |
| | Cohort 1; | calprotectin, Bristol | Investigator | |
| | ≥9 participants for | stool scale up to 24 | or delegate, | |
| | Cohort 2; | hours after last dose | Medical | |
| | ≥9 participants | (if available) and | Monitor, | |
| | for Cohort | stopping rules | Statistician, | |
| | 3; to progress to | based on | Sponsor's | |
| | Cohorts 4, | available data. | Clinical | |
| | 5, 6 and 7 | | Lead) | |

¹The 48-hour timepoint refers to data from Day 5 (i.e. 48 hours after multiple dosing has started on Day 3), and not 48 hours after the third dose on Day 5.

Dose escalation increments may not exceed those proposed (i.e. 10-fold). However, lower dose increments, dose decrements and repeated dose levels are acceptable if required. The new dose level will be agreed with the Principal Investigator (or delegate) and the Medical Monitor.

### 2.7. Intervention after the End of the Study

No specific interventions are planned after the end of the study which is the safety visit 14 days after the last dose of the study intervention. A sample for microbiome analysis will be collected at the final safety visit 14 days after the last dose of the study intervention or as close as possible to this timepoint (i.e. within 48 hours).

### 3. DISCONTINUATION OF STUDY INTERVENTION AND PARTICIPANT DISCONTINUATION/WITHDRAWAL

### 3.1. Discontinuation of Study Intervention

Discontinuation of study intervention for abnormal liver function should be considered by the investigator when a participant meets the conditions outlined in Appendix 6 or if the investigator believes that it is in the best interest of the participant.

If a clinically significant finding is identified (including, but not limited to changes from baseline in QT interval corrected using Fridericia's formula [QTcF]) after enrolment, the investigator or qualified designee will determine if the participant can continue in the study and if any change in participant management is needed. This review of the ECG printed at the time of collection must be documented. Any new clinically relevant finding should be reported as an AE. See the SoA for data to be collected at the time of intervention discontinuation and follow-up and for any further evaluations that need to be completed.

### 3.1.1. Temporary Discontinuation

Dosing may be temporarily suspended at the investigator's discretion due to AE or intercurrent illness for a period of up to 48 hours, following which, the participant may continue with the remaining doses if the investigator considers it safe to do so. The participant should discontinue permanently if it occurs a second time.

### 3.1.2. Rechallenge

Rechallenge for participants who have discontinued for liver or cardiac effects is not permitted.

### 3.2. Participant Discontinuation/Withdrawal from the Study

A participant may withdraw from the study at any time at his/her own request, or may be withdrawn at any time at the discretion of the investigator for safety, behavioral, compliance, or administrative reasons. Any participant who withdraws from the study may be replaced so as to achieve a minimum of 120 evaluable participants.
If the participant withdraws consent for disclosure of future information, the Sponsor may retain and continue to use any data collected before such a withdrawal of consent.
If a participant withdraws from the study, he/she may request destruction of any samples taken and not tested, and the investigator must document this in the site study records.
Participants who withdraw from the study should complete the assessments for the follow-up visit, as detailed in the SoA.

### 3.3. Lost to Follow-Up

A participant will be considered lost to follow-up if he/she repeatedly fails to return for scheduled visits and is unable to be contacted by the study site. The follow-up visit should be at least 14 days and a maximum of 28 days after the last dose.

The following actions must be taken if a participant fails to return to the clinic for a required study visit:
The site must attempt to contact the participant and reschedule the missed visit as soon as possible. The participant will be reminded of the importance of attending every visit and if there are issues with attendance a discussion should be had to ascertain whether or not the participant wishes to and/or should continue in the study.
Before a participant is deemed lost to follow-up, the investigator or designee must make every effort to regain contact with the participant (where possible, 3 telephone calls and, if necessary, a certified letter to the participant's last known mailing address or local equivalent methods). These contact attempts should be documented in the participant's records.
Should the participant continue to be unreachable, he/she will be considered to have withdrawn from the study.

### 4. STUDY ASSESSMENTS AND PROCEDURES

Study procedures and their timing are summarized in the SoA. Protocol waivers or exemptions are not allowed.
Immediate safety concerns should be discussed with the Sponsor immediately upon occurrence or awareness to determine if the participant should continue or discontinue study intervention.
Adherence to the study design requirements, including those specified in the SoA, is essential and required for study conduct.
All screening evaluations must be completed and reviewed to confirm that potential participants meet all eligibility criteria. The investigator will maintain a screening log to record details of all participants screened and to confirm eligibility or record reasons for screening failure, as applicable.
Procedures conducted as part of the participant's routine clinical management (e.g. blood count) and obtained before signing of the ICF may be utilized for screening or baseline purposes provided the procedures met the protocol-specified criteria and were performed within the time frame defined in the SoA.
The maximum amount of blood collected from each participant over the duration of the study, including any extra assessments that may be required, will not exceed 400 mL. Repeat or unscheduled samples may be taken for safety reasons or for technical issues with the samples.
Repeat or unscheduled visits may be conducted at the investigator's discretion but all details must be recorded in the CRF.

### 4.1. Efficacy Assessments

For Cohorts 3, 4, 5, 6 and 7 only, the following efficacy measurements will be collected post-dose at planned timepoints as provided in the SoA:

PASI composite score (psoriasis participants only)
EASI composite score (atopic dermatitis participants only)

### LSS—Lesion Severity Score

### IGA—Investigator's Global Assessment

### SCORAD—SCORing Atopic Dermatitis

The percentage of BSA affected by psoriasis or atopic dermatitis will also be collected at the planned timepoints corresponding to the PASI and EASI measurements (recorded as % of the whole body).
In addition, photos should be taken of up to 6 lesion sites that have a lesion area≥2×2 cm at baseline. The same sites should be photographed at baseline, Day 10, Day 30 and at the follow-up visit.

### 4.2. Safety Assessments

Planned timepoints for all safety assessments are provided in the SoA.

### 4.2.1. Physical Examinations

A complete physical examination will include, at a minimum, assessments of the cardiovascular, respiratory, GI and neurological systems. Height (Screening only) and weight will also be measured and recorded.
Investigators should pay special attention to clinical signs related to previous serious illnesses.

### 4.2.2. Vital Signs

Blood pressure, pulse rate, respiratory rate, and oral temperature will be assessed.
Blood pressure and pulse measurements will be assessed in a sitting position with a completely automated device.
Blood pressure and pulse measurements should be preceded by at least 5 minutes of rest for the participant in a quiet setting without distractions (e.g. television, mobile phones).
Vital signs (to be checked prior to dosing and/or any procedures) will consist of 1 pulse and 3 blood pressure measurements (3 consecutive blood pressure readings will be recorded at intervals of at least 1 minute). The average of the 3 blood pressure readings will be recorded on the CRF.

### 4.2.3. Electrocardiograms

Single 12-lead ECGs will be obtained as outlined in the SoA (see herein) using an ECG machine that automatically calculates the heart rate and measures PR, QRS, QT, and the corrected QT (QTc) intervals. Refer to other section herein for QTc withdrawal criteria and any additional QTc readings that may be necessary.
At each timepoint a triplicate ECG is required, with 3 individual ECG tracings being obtained as closely as possible in succession, but no more than 2 minutes apart. The full set of triplicates should be completed in less than 4 minutes.
Post-dose ECGs should be conducted within 2 hours after the dose.

### 4.2.4. Clinical Safety Laboratory Assessments

See Appendix 2 for the list of clinical laboratory tests to be performed and the SoA for the timing and frequency.
The investigator must review the laboratory report, document this review, and record any clinically relevant changes occurring during the study in the AE section of the CRF. The laboratory reports must be filed with the source documents. Clinically significant abnormal laboratory findings are those which are not associated with the underlying disease, unless judged by the investigator to be more severe than expected for the participant's condition.
All laboratory tests with values considered clinically significantly abnormal during participation in the study or within 28 days after the last dose of study intervention should be repeated until the values return to normal or baseline or are no longer considered clinically significant by the investigator or Medical Monitor.
   If such values do not return to normal/baseline within a period of time judged reasonable by the investigator, the etiology should be identified and the Sponsor notified.
   All protocol-required laboratory assessments, as defined in Appendix 2, must be conducted in accordance with the laboratory manual and the SoA in addition to local procedures.
   If laboratory values from non-protocol specified laboratory assessments performed at the institution's local laboratory require a change in participant management or are considered clinically significant by the investigator (e.g. SAE or AE or dose modification), then the results must be recorded in the CRF.

### 4.3. Adverse Events and Serious Adverse Events

The definitions of an AE or SAE can be found in Appendix 3.

AEs will be reported by the participant (or, when appropriate, by a caregiver, surrogate, or the participant's legally authorized representative).

The investigator and any qualified designees are responsible for detecting, documenting, and recording events that meet the definition of an AE or SAE and remain responsible for following up AEs that are serious, considered related to the study intervention or study procedures, or that caused the participant to discontinue the study intervention (see herein).

### 4.3.1. Time Period and Frequency for Collecting AE and SAE Information

All AEs and SAEs will be collected from the signing of the ICF at Screening until the follow-up visit (14-28 days after last dose) at the timepoints specified in the SoA (herein).

Medical occurrences that begin before the start of study intervention but after obtaining informed consent will be recorded on the Medical History/Current Medical Conditions section of the CRF not the AE section.

All SAEs will be recorded and reported to the Sponsor or designee within 24 hours, as indicated in Appendix 3. The investigator will submit any updated SAE data to the Sponsor within 24 hours of it being available.

Investigators are not obligated to actively seek AEs or SAEs after conclusion of the study participation. However, if the investigator learns of any SAE, including a death, at any time after a participant has been discharged from the study, and he/she considers the event to be reasonably related to the study intervention or study participation, the investigator must promptly notify the Sponsor.

The method of recording, evaluating, and assessing causality of AEs and SAEs and the procedures for completing and transmitting SAE reports are provided in Appendix 3.

### 4.3.2. Method of Detecting AEs and SAEs

Care will be taken not to introduce bias when detecting AEs and/or SAEs. Open-ended and non-leading verbal questioning of the participant is the preferred method to inquire about AE occurrences.

### 4.3.3. Follow-Up of AEs and SAEs

After the initial AE/SAE report, the investigator is required to proactively follow each participant at subsequent visits/contacts. All SAEs will be followed until resolution, stabilization, the event is otherwise explained, or the participant is lost to follow-up (as defined herein). Further information on follow-up procedures is given in Appendix 3.

### 4.3.4. Regulatory Reporting Requirements for SAEs

Prompt notification by the investigator to the Sponsor of an SAE is essential so that legal obligations and ethical responsibilities towards the safety of participants and the safety of a study intervention under clinical investigation are met.
The Sponsor has a legal responsibility to notify both the local regulatory authority and other regulatory agencies about the safety of a study intervention under clinical investigation. The Sponsor will comply with country-specific regulatory requirements relating to safety reporting to the regulatory authority, Institutional Review Boards (IRB)/Independent Ethics Committees (IEC), and investigators.
Investigator safety reports must be prepared for suspected unexpected serious adverse reactions (SUSAR) according to local regulatory requirements and Sponsor policy and forwarded to investigators as necessary.
An investigator who receives an investigator safety report describing an SAE or other specific safety information (e.g. summary or listing of SAEs) from the Sponsor will review and then file it along with the IB and will notify the IRB/IEC, if appropriate according to local requirements.

### 4.3.5. Pregnancy

Details of all pregnancies in female participants will be collected after the start of study intervention and until the end of the pregnancy.
If a pregnancy is reported, the investigator should inform the Sponsor within 24 hours of learning of the pregnancy and should follow the procedures outlined in Appendix 4.
Abnormal pregnancy outcomes (e.g. spontaneous abortion, fetal death, stillbirth, congenital anomalies, ectopic pregnancy) are considered SAEs.

### 4.3.6. Cardiovascular and Death Events

All deaths regardless of the relatedness assessment will be urgently reviewed by the SRC before any more participants are dosed. All deaths and cardiovascular events will be reported to the MHRA regardless of the causality assessment.

4.3.7. Disease-Related Events and/or Disease-Related Outcomes not Qualifying as AEs or SAEs

There are no expected AEs based on the understanding of the mechanism or knowledge of the patient population.

### 4.4. Treatment of Overdose

For this study, any dose of Prevotella histicola Strain B taken which is more than the daily dose specified for that cohort within a 24-hour time period will be considered an overdose.

The Sponsor does not recommend specific treatment for an overdose unless there is evidence of infection and/or colitis. If the clinical situation warrants it, then the Sponsor would recommend the use of a penicillin-based antibiotic (e.g. Penicillin V) which may be used in case of overdose.

In the event of an overdose, the investigator should:
1. Contact the Medical Monitor immediately upon becoming aware of the overdose.
2. Closely monitor the participant for any AE/SAE and laboratory abnormalities for 72 hours or until they have resolved, whichever is the longer.
3. Document the quantity of the excess dose as well as the duration of the overdose in the CRF.

Decisions regarding dose interruptions or modifications will be made by the investigator in consultation with the Medical Monitor based on the clinical evaluation of the participant.

### 4.5. Pharmacokinetics

Specific pharmacokinetic (PK) parameters are not evaluated in this study due to the nature of the therapy. Prevotella histicola Strain B is orally administered and exposure is restricted to the gut and so systemic exposure is not expected. Samples will be taken to confirm the lack of systemic absorption through the study but specific PK parameters will not be derived. Microbiome samples will be analyzed during the study (see herein) to look for colonization, although this is not expected.

### 4.6. Pharmacodynamics

Venous blood samples not exceeding 400 mL will be collected for measurement of the assessments according to the SoA.

Skin biopsy samples are taken in the patient cohorts at baseline and Day 30 in line with the SoA. These will be 4 mm punch biopsies.

Fecal samples will be collected for measurement of microbiome diversity and Prevotella histicola Strain B at baseline (any time before day of dosing), at the end of the single and multiple dosing periods, and at 14-28 days following the last dose.

### 4.7. HLA Testing

Two blood samples for DNA isolation will be collected from participants. One sample will be analyzed for HLA status and the other for additional analyses that may be conducted.

Details on processes for collection and shipment and destruction of these samples can be found in the laboratory manual.

### 4.8. Biomarkers

Collection of samples for other biomarker research is also part of this study. The following samples for biomarker research are required and will be collected from all participants in this study as specified in the SoA:
   Blood
   Skin
   Faeces
Blood samples may be used to measure circulating levels of cytokines and to assess the responsiveness of the innate and adaptive immune system in an ex vivo antigen stimulation assay. Blood samples may also be used for transcriptome profiling.
Skin samples will be subject to histological analysis and where relevant have IHC and transcription analysis performed on them.
   Other samples may be used for research to identify additional microbes that may have beneficial effects if used as part of a microbiome-based treatment.

### 4.8.1. RNA Transcriptome Research

Transcriptome studies will be conducted for selected blood and skin samples. This will enable the evaluation of changes in transcriptome profiles that may correlate with biological response relating to improvement in psoriasis or atopic dermatitis or the action of Prevotella histicola Strain B.

The same samples may also be used to confirm findings by application of alternative technologies.

### 4.8.2. Microbiome Research

Faeces and fecal fluid analysis may be performed to understand the effects of Prevotella histicola Strain B on the individual's microbiome either during treatment or following cessation of treatment. Associations of specific microbes within the microbiome and drug response may also be investigated if there is marked variability in response. Microbiome analysis will be performed through 16s sequencing and/or whole genome microbial sequencing depending on the question being asked.

### 5. STATISTICAL CONSIDERATIONS

All analyses will be performed using SAS® version 9.3 or later (SAS Institute, Cary, N.C., USA).

Descriptive statistics will be provided to summarize safety and efficacy endpoints by dose cohort. For categorical variables, summary tabulations of frequency and percentage of participants within each category will be presented along with 2-sided 95% exact confidence intervals (Cis) where appropriate. For continuous variables, the number of participants, mean, median, standard deviation (SD), minimum, and maximum values will be presented.

### 5.1. Sample Size Determination

The primary objective of this FIH study is to assess the safety and tolerability of Prevotella histicola Strain B. A minimum number of participants to be recruited (Cohorts 1 to 7) is 120 in total and the maximum number is 132 participants in total, although additional replacements may be enrolled if necessary. Any participant who withdraws from the study may be replaced so as to achieve a minimum of 120 evaluable participants.

The sample size has been chosen to explore the tolerability and safety of this new treatment, while limiting exposure to a minimum number of participants. A larger sample size has been determined for Cohorts 4 to 7 to allow useful conclusions to be drawn about the disease-related efficacy endpoints, although no formal power calculations have been performed.

### 5.2. Populations for Analyses

For purposes of analysis, the following populations are defined:

| Population | Description |
|---|---|
| Enrolled | All participants who sign the ICF. |
| Safety | All participants randomly assigned to study intervention |
| | and who take at least 1 dose of study intervention. |
| | Participants will be analysed according to the |
| | intervention they actually received. |
| Evaluable- | All participants with psoriasis randomly assigned to study |
| psoriasis | intervention and who had no important protocol deviations |
| | affecting psoriasis-related efficacy variables. Participants |
| | will be analysed according to the intervention |
| | they actually received. |
| Evaluable- | All participants with atopic dermatitis randomly assigned |
| atopic | to study intervention and who had no important protocol |
| dermatitis | deviations affecting atopic dermatitis-related efficacy |
| | variables. Participants will be analysed according to |
| | the intervention they actually received. |

In all populations, treatment will be assigned based upon the treatment that the participants actually received, regardless of the treatment to which they were randomized.

### 5.3. Statistical Analyses

The Statistical Analysis Plan (SAP) will be developed and finalized before database lock and will describe the participant populations to be included in the analyses, and procedures for accounting for missing, unused, and spurious data. This section is a summary of the planned statistical analyses of the primary and secondary endpoints.

### 5.3.1. Efficacy Analyses

The efficacy analyses will be performed on the Safety or the Evaluable Populations (more detail will be specified in the SAP).

| Endpoint | Statistical Analysis Methods |
|---|---|
| Primary | The primary endpoint is safety so all efficacy endpoints |
| | are classified as either secondary or exploratory. |
| Secondary | PASI and EASI: |
| | Change from baseline in PASI and EASI will be listed |
| | and summarised for the corresponding evaluable |
| | population by treatment group. |
| | Change from baseline in PASI and EASI on Day 30 |
| | will be analysed using analysis of covariance |
| | (ANCOVA) with the baseline value as a covariate and |
| | treatment group as a fixed effect. |
| | SCORAD will be listed and summarised by treatment group. |
| | LSS will be listed and summarised by participant |
| | population and treatment group. |
| | Percentage of BSA affected by disease will be |
| | listed and summarised by |
| | participant population and treatment group. |
| | TGA will be listed and summarised by participant |
| | population and treatment group. |
| Exploratory | Analysis methods for all other efficacy endpoints will |
| | be described in the SAP finalised before database lock. |

### 5.3.2. Safety Analyses

All safety analyses will be performed on the Safety Population.

| Endpoint | Statistical Analysis Methods |
|---|---|
| Primary | Incidence of SAEs and AEs: AEs will be coded using |
| | the latest available version of the Medical Dictionary |
| | for Regulatory Activities (MedDRA) and categorised by |
| | intensity (mild/moderate/severe). Treatment-emergent AEs |
| | and SAEs will be listed and summarised by treatment |
| | group and participant population, system organ |
| | class and preferred term. |
| | Safety laboratory measurements: These variables will be |
| | listed and summarised by treatment group and |
| | participant population. |
| | Analysis methods for all other safety endpoints will be |
| | described in the SAP finalised before database lock. |
| Secondary | Not applicable |
| Exploratory | Immune Biomarkers: |
| | These variables will be listed and summarised by |
| | treatment group and participant population. |
| | Analysis methods for all other safety endpoints will be |
| | described in the SAP finalised before database lock. |

### 5.3.3. Other Analyses

Pharmacodynamic and biomarker exploratory analyses will be described in the SAP finalized before database lock. The population pharmacodynamic analyses will be presented separately from the main clinical study report (CSR).

### 5.3.4. Safety Review Committee (SRC)

An SRC consisting of the Principal Investigator (or delegate), Medical Monitor, Statistician and the Sponsor's Clinical Lead will review blinded safety data and provide governance over the study and dose escalation steps. The SRC will decide whether to proceed to the next dosing level at the end of each cohort for Cohorts 1 to 3 and they can decide to omit a cohort or dose escalation step if warranted. The Principal Investigator (or delegate) and Medical Monitor will review the transition within each cohort, from single dose to multiple dose for each participant. Ad hoc SRC meetings may be convened if deemed necessary by the Sponsor or the Principal Investigator (or delegate). A detailed description of the procedures will be outlined in a separate SRC charter. Following the successful completion of Cohort 3 and the SRC decision to continue, then Cohorts 4 to 7 can be run in parallel or in an order which optimizes the use of available drug supply. A review of the safety data will be performed after each cohort is finished, but it is not requirement to move from one cohort to the next in Cohorts 4 to 7.

Documented reviews will be conducted at the times specified herein.

### 6. SUPPORTING DOCUMENTATION AND OPERATIONAL CONSIDERATIONS

### 6.1. Appendix 1: Regulatory, Ethical, and Study Oversight Considerations

### 6.1.1. Regulatory and Ethical Considerations

This study will be conducted in accordance with the protocol and with the following:
   Consensus ethical principles derived from international guidelines including the Declaration of Helsinki and Council for International Organizations of Medical Sciences (CIOMS) International Ethical Guidelines
   Applicable International Council for Harmonization (ICH) Good Clinical Practice (GCP) Guidelines
   Applicable laws and regulations
The protocol, protocol amendments, ICF, IB, and other relevant documents (e.g. advertisements) must be submitted to an IRB/IEC by the investigator and reviewed and approved by the IRB/IEC before the study is initiated.
Any amendments to the protocol will require IRB/IEC approval before implementation of changes made to the study design, except for changes necessary to eliminate an immediate hazard to study participants.
The investigator is responsible for the following:
   Providing written summaries of the status of the study to the IRB/IEC annually or more frequently in accordance with the requirements, policies, and procedures established by the IRB/IEC
   Notifying the IRB/IEC of SAEs or other significant safety findings as required by IRB/IEC procedures
   Providing oversight of the conduct of the study at the site and adherence to requirements of 21 CFR, ICH guidelines, the IRB/IEC, European regulation 536/2014 for clinical studies (if applicable), and all other applicable local regulations

### 6.1.2. Informed Consent Process

The investigator or his/her representative will explain the nature of the study to the participant or his/her legally authorized representative and answer all questions regarding the study.
Participants must be informed that their participation is voluntary. Participants or their legally authorized representative will be required to sign a statement of informed consent that meets the requirements of 21 CFR 50, local regulations, ICH guidelines, Health Insurance Portability and Accountability Act (HIPAA) requirements, where applicable, and the IRB/IEC or study site.
The medical record must include a statement that written informed consent was obtained before the participant was enrolled in the study and the date the written consent was obtained. The authorized person obtaining the informed consent must also sign the ICF.
Participants must be reconsented to the most current version of the ICF(s) during their participation in the study.
A copy of the ICF(s) must be provided to the participant or the participant's legally authorized representative.

Participants who are rescreened are required to sign a new ICF unless they are rescreened only because they have exceeded the 28-day screening period time limit.

The ICF will contain a section that addresses the use of samples for focused genetic and biomarker research (e.g. HLA sample). The investigator or authorized designee will explain to each participant the objectives of the research.

### 6.1.3. Data Protection

Participants will be assigned a unique identifier by the Sponsor. Any participant records or datasets that are transferred to the Sponsor will contain the identifier only; participant names or any information which would make the participant identifiable will not be transferred.
The participant must be informed that his/her personal study-related data will be used by the Sponsor in accordance with applicable data protection laws. The level of disclosure must also be explained to the participant.
The participant must be informed that his/her medical records may be examined by Clinical Quality Assurance auditors or other authorized personnel appointed by the Sponsor, by appropriate IRB/IEC members, and by inspectors from regulatory authorities.

### 6.1.4. Committees Structure

The only committee set up for this study is the SRC and a description is listed herein.

### 6.1.5. Dissemination of Clinical Study Data

### 6.1.6. Data Quality Assurance

All participant data relating to the study will be recorded on electronic CRF (eCRF) unless transmitted to the Sponsor or designee electronically (e.g. laboratory data). The investigator is responsible for verifying that data entries are accurate and correct by physically or electronically signing the CRF.
The investigator must maintain accurate documentation (source data) that supports the information entered in the CRF.
The investigator must permit study-related monitoring, audits, IRB/IEC review, and regulatory agency inspections and provide direct access to source data documents.
The Sponsor or designee is responsible for the data management of this study including quality checking of the data.
Study monitors will perform ongoing source data verification to confirm that data entered into the CRF by authorized site personnel are accurate, complete, and verifiable from source documents; that the safety and rights of participants are being protected; and that the study is being conducted in accordance with the currently approved protocol and any other study agreements, ICH GCP, and all applicable regulatory requirements.
Records and documents, including signed ICFs, pertaining to the conduct of this study must be retained by the investigator for 15 years after study completion unless local regulations or institutional policies require a longer retention period. No records may be destroyed during the retention period without the written approval of the Sponsor. No records may be transferred to another location or party without written notification to the Sponsor.

### 6.1.7. Source Documents

Source documents provide evidence for the existence of the participant and substantiate the integrity of the data collected. Source documents are filed at the investigator's site.
Data entered in the eCRF that are transcribed from source documents must be consistent with the source documents or the discrepancies must be explained. The investigator may need to request previous medical records or transfer records, depending on the study. Also, current medical records must be available.
Definition of what constitutes source data can be found in the source data agreement form.

### 6.1.8. Study and Site Closure

The Sponsor reserves the right to close the study site or terminate the study at any time for any reason at the sole discretion of the Sponsor. Study sites will be closed upon study completion. A study site is considered closed when all required documents and study supplies have been collected and a study site closure visit has been performed.

The investigator may initiate study site closure at any time, provided there is reasonable cause and sufficient notice is given in advance of the intended termination.

Reasons for the early closure of a study site by the Sponsor or investigator may include but are not limited to:
Failure of the investigator to comply with the protocol, the requirements of the IRB/IEC or local health authorities, the Sponsor's procedures, or GCP guidelines
Inadequate enrolment of participants by the investigator
Discontinuation of further study intervention development

### 6.1.9. Publication Policy

Full details on the publication policy are provided in the contract between the Sponsor and the investigator. In summary: the results of this study may be published or presented at scientific meetings. If this is foreseen, the investigator agrees to submit all manuscripts or abstracts to the Sponsor at least 30 days before submission. This allows the Sponsor to protect proprietary information, delay the publication if necessary to protect its patent rights, and to provide comments.
The Sponsor will comply with the requirements for publication of study results as detailed herein. In accordance with standard editorial and ethical practice, the Sponsor will generally support publication of multisite studies only in their entirety and not as individual site data. In this case, a coordinating investigator will be designated by mutual agreement.
Authorship will be determined by mutual agreement and in line with International Committee of Medical Journal Editors authorship requirements

### 6.2. Appendix 2: Clinical Laboratory Tests

The majority of the tests detailed in Table 7 will be performed by the study site or by their designated vendor. Additional tests may also be conducted.
Protocol-specific requirements for inclusion or exclusion of participants are detailed in the protocol.
Additional tests may be performed at any time during the study as deemed necessary by the investigator or required by local regulations.

TABLE 7
Protocol-Required Safety Laboratory Assessments

| Laboratory | | | | |
|---|---|---|---|---|
| Assessments | Parameters | | | |
| Haematology | Platelet | RBC | White blood cell | |
| | Count | Indices: | (WBC) count with | |
| | Red blood | Mean cell | Differential: | |
| | cell | volume | Neutrophils | |
| | (RBC) | (MCV) | Lymphocytes | |
| | Count | Mean | Monocytes | |
| | Hemo- | corpuscular | Eosinophils | |
| | globin | haemo- | Basophils | |
| | Hema- | globin | | |
| | tocrit | (MCH) | | |
| | | % | | |
| | | Reticulo- | | |
| | | cytes | | |
| Clinical | Blood | Potassium | AST/Serum Glutamic- | Total and |
| Chemistry¹ | urea | | Oxaloacetic | direct |
| | nitrogen | | Transaminase (SGOT) | bilirubin |
| | (BUN) | | | |
| | Creatinine | Sodium | ALT/Serum Glutamic- | Total |
| | | | Pyruvic Transaminase | Protein |
| | | | (SGPT) | |
| | Fasting | Calcium | Alkaline phosphatase | CRP and |
| | glucose at | | | faecal |
| | baseline | | | cal- |
| | and end | | | protectin |
| | of dosing | | | |

| | | |
|---|---|---|
| Urinalysis | Dipstick: protein, blood, | Microscopy: only if dipstick test |
| | ketones, glucose, | for protein, blood, leukocyte |
| | bilirubin, | esterase or nitrites is abnormal |
| | urobilinogen, leukocyte | |
| | esterase, specific | |
| | gravity, nitrites, pH | |

| | |
|---|---|
| Other | • Drug screen (to include at minimum: |
| Screening | amphetamines, barbiturates, cocaine, |
| Tests | opiates, cannabinoids and benzodiazepines) |
| | • Serum human chorionic gonadotropin |
| | (HCG) pregnancy test for WOCBP |
| | • Serology (HIV antibody, HBsAg, and HCV antibody) |

NOTES:
¹Details of liver chemistry stopping criteria and required actions and follow-up assessments after liver stopping or monitoring event are given in herein and Appendix 6. All events of ALT ≥ 3 × ULN and bilirubin ≥ 2 × ULN (>35% direct bilirubin) or ALT ≥ 3 × ULN and international normalised ratio (INR) > 1.5, if INR measured which may indicate severe liver injury (possible Hy's Law), must be reported as an SAE (excluding studies of hepatic impairment or cirrhosis).

Investigators must document their review of each laboratory safety report.

### 6.3. Appendix 3: Adverse Events: Definitions and Procedures for Recording, Evaluating, Follow-Up, and Reporting

### Definition of AE

AE Definition

An AE is any untoward medical occurrence in a patient or clinical study
participant, temporally associated with the use of study intervention,
whether or not considered related to the study intervention.
NOTE: An AE can therefore be any unfavourable and unintended sign
(including an abnormal laboratory finding), symptom, or disease (new
or exacerbated) temporally associated with the use of study intervention.

Events Meeting the AE Definition

Any abnormal laboratory test results (haematology, clinical chemistry, or
urinalysis) or other safety assessments (e.g. ECG, radiological scans,
vital signs measurements), including those that worsen from baseline,
considered clinically significant in the medical and scientific judgment
of the investigator (i.e. not related to progression of underlying disease).
Exacerbation of a chronic or intermittent pre-existing condition including
either an increase in frequency and/or intensity of the condition.
New conditions detected or diagnosed after study intervention
administration even though it may have been present before the start of
the study.
Signs, symptoms, or the clinical sequelae of a suspected drug-drug
interaction.
Signs, symptoms, or the clinical sequelae of a suspected overdose of
either study intervention or a concomitant medication. Overdose per se
will not be reported as an AE/SAE unless it is an intentional overdose
taken with possible suicidal/self-harming intent. Such overdoses should
be reported regardless of sequelae.
“Lack of efficacy” or “failure of expected pharmacological action” per se
will not be reported as an AE or SAE. Such instances will be captured in
the efficacy assessments. However, the signs, symptoms, and/or clinical
sequelae resulting from lack of efficacy will be reported as an AE or SAE
if they fulfil the definition of an AE or SAE.

Events NOT Meeting the AE Definition

Any clinically significant abnormal laboratory findings or other
abnormal safety assessments which are associated with the underlying
disease, unless judged by the investigator to be more severe than
expected for the participant's condition.
The disease/disorder being studied or expected progression, signs, or
symptoms of the disease/disorder being studied, unless more severe
than expected for the participant's condition.
Medical or surgical procedure (e.g. endoscopy, appendectomy): the
condition that leads to the procedure is the AE.
Situations in which an untoward medical occurrence did not occur
(social and/or convenience admission to a hospital).
Anticipated day-to-day fluctuations of pre-existing disease(s) or
condition(s) present or detected at the start of the study that do not
worsen.

### Definition of SAE

An SAE is defined as any untoward medical occurrence that, at any dose:

a. Results in death
b. Is life-threatening
The term ‘life-threatening’ in the definition of ‘serious’ refers to an event
in which the participant was at risk of death at the time of the event. It
does not refer to an event, which hypothetically might have caused death,
if it were more severe.
c. Requires inpatient hospitalisation or prolongation of existing
hospitalisation
In general, hospitalization signifies that the participant has been detained
(usually involving at least an overnight stay) at the hospital or emergency
ward for observation and/or treatment that would not have been
appropriate in the physician's office or outpatient setting. Complications
that occur during hospitalization are AEs. If a complication prolongs
hospitalization or fulfils any other serious criteria, the event is serious.
When in doubt as to whether ‘hospitalization’ occurred or was necessary,
the AE should be considered serious.
Hospitalization for elective treatment of a pre-existing condition that did
not worsen from baseline is not considered an AE.
d. Results in persistent disability/incapacity
The term disability means a substantial disruption of a person's ability to
conduct normal life functions.
This definition is not intended to include experiences of relatively minor
medical significance such as uncomplicated headache, nausea, vomiting,
diarrhoea, influenza, and accidental trauma (e.g. sprained ankle) which
may interfere with or prevent everyday life functions but do not constitute
a substantial disruption.
e. Is a congenital anomaly/birth defect
f. Other situations:
Medical or scientific judgment should be exercised in deciding whether
SAE reporting is appropriate in other situations such as important medical
events that may not be immediately life-threatening or result in death or
hospitalisation but may jeopardise the participant or may require medical
or surgical intervention to prevent one of the other outcomes listed in the
above definition. These events should usually be considered serious.
Examples of such events include invasive or malignant cancers, intensive
treatment in an emergency room or at home for allergic bronchospasm,
blood dyscrasias or convulsions that do not result in hospitalisation, or
development of drug dependency or drug abuse.

Recording and Follow-Up of AE and/or SAE

AE and SAE Recording

When an AE/SAE occurs, it is the responsibility of the investigator
to review all documentation (e.g. hospital progress notes, laboratory
reports, and diagnostics reports) related to the event.
The investigator will then record all relevant AE/SAE information in
the CRF.
It is not acceptable for the investigator to send photocopies of the
participant's medical records to the Sponsor in lieu of completion of
the AE/SAE CRF page.
There may be instances when copies of medical records for certain
cases are requested by the Sponsor. In this case, all participant
identifiers, with the exception of the participant number, will be
redacted on the copies of the medical records before submission to
the Sponsor.
The investigator will attempt to establish a diagnosis of the event
based on signs, symptoms, and/or other clinical information.
Whenever possible, the diagnosis (not the individual signs/symptoms)
will be documented as the AE/SAE.

Assessment of Intensity

The investigator will make an assessment of intensity for each AE and
SAE reported during the study and assign it to 1 of the following
categories:
Mild: An event that is easily tolerated by the participant, causing
minimal discomfort and not interfering with everyday activities.
Moderate: An event that causes sufficient discomfort and interferes
with normal everyday activities.
Severe: An event that prevents normal everyday activities. An AE that
is assessed as severe should not be confused with a SAE. Severe is a
category utilised for rating the intensity of an event; and both AEs and
SAEs can be assessed as severe.
An event is defined as ‘serious’ when it meets at least 1 of the predefined
outcomes as described in the definition of an SAE, NOT when it is rated
as severe.

Assessment of Causality

The investigator is obligated to assess the relationship between study
intervention and each occurrence of each AE/SAE.
A “reasonable possibility” of a relationship conveys that there are facts,
evidence, and/or arguments to suggest a causal relationship, rather than
that a relationship cannot be ruled out.
The investigator will use clinical judgment to determine the relationship.
Alternative causes, such as underlying disease(s), concomitant therapy,
and other risk factors, as well as the temporal relationship of the event
to study intervention administration will be considered and investigated.
The investigator will also consult the IB and/or Product Information,
for marketed products, in his/her assessment.
For each AE/SAE, the investigator must document in the medical notes
that he/she has reviewed the AE/SAE and has provided an assessment
of causality.
There may be situations in which an SAE has occurred and the
investigator has minimal information to include in the initial report to
the Sponsor. However, it is very important that the investigator always
make an assessment of causality for every event before the initial
transmission of the SAE data to the Sponsor.
The investigator may change his/her opinion of causality in light of
follow-up information and send an SAE follow-up report with the
updated causality assessment.
The causality assessment is one of the criteria used when determining
regulatory reporting requirements.

Follow-up of AEs and SAEs

The investigator is obligated to perform or arrange for the conduct of
supplemental measurements and/or evaluations as medically indicated
or as requested by the Sponsor to elucidate the nature and/or causality
of the AE or SAE as fully as possible. This may include additional
laboratory tests or investigations, histopathological examinations, or
consultation with other health care professionals.
If a participant dies during participation in the study or during a
recognised follow-up period, the investigator will provide the Sponsor
with a copy of any post-mortem findings including histopathology.
New or updated information will be recorded in the originally
completed CRF.
The investigator will submit any updated SAE data to the Sponsor
within 24 hours of receipt of the information.

Reporting of SAEs

SAE Reporting to the Sponsor via an Electronic Data Collection Tool

The primary mechanism for reporting an SAE to the Sponsor will be
directly by telephone and email.
The site will enter the SAE data into the electronic system as soon
as it becomes available.
After the study is completed at a given site, the electronic data
collection tool will be taken off-line to prevent the entry of new data
or changes to existing data.
If a site receives a report of a new SAE from a study participant or
receives updated data on a previously reported SAE after the
electronic data collection tool has been taken offline, then the site
can report this information on a paper SAE form (see next section)
or to the Medical Monitor by telephone.
Contact details for the Medical Monitor for SAE reporting can be
found at the beginning of the protocol.

### 6.4. Appendix 4: Contraceptive Guidance and Collection of Pregnancy Information

Definitions:

Woman of Child-Bearing Potential (WOCBP)

A woman is considered fertile following menarche and until becoming postmenopausal unless permanently sterile (see below).

Women in the following categories are not considered WOCBP:
1. Premenarchal
2. Premenopausal female with 1 of the following:
   Documented hysterectomy
   Documented bilateral salpingectomy
   Documented bilateral oophorectomy
   Note: Documentation can come from the site personnel's review of the participant's medical records, medical examination, or medical history interview.
3. Postmenopausal female
   A postmenopausal state is defined as no menses for 12 months without an alternative medical cause. A high follicle stimulating hormone (FSH) level in the postmenopausal range may be used to confirm a postmenopausal state in women not using hormonal contraception or hormonal replacement therapy (HRT). However, in the absence of 12 months of amenorrhea, a single FSH measurement is insufficient.
   Females on HRT and whose menopausal status is in doubt will be required to use one of the non-estrogen hormonal highly effective contraception methods if they wish to continue their HRT during the study.

Contraception Guidance:

Male participants must either
Be abstinent from penile-vaginal intercourse as their usual and preferred lifestyle (abstinent on a long term and persistent basis) and agree to remain abstinent
Use a male condom during each episode of penile penetration during their participation in the study and for 90 days after the last dose of study drug. In addition, male participants must refrain from donating sperm for the duration of the study and for at least 90 days following their final visit.

Female Participants

Female participants of child-bearing potential are eligible to participate if they agree to use a highly effective method of contraception consistently and correctly as described in Table 8.

TABLE 8
Highly Effective Contraceptive Methods

Highly Effective Contraceptive Methods That Are User Dependent^{a}
Failure rate of <1% per year when used consistently and correctly.
Combined (estrogen and progestogen containing) hormonal
contraception associated with inhibition of ovulation^{b}
Oral
Intravaginal
Transdermal
Progestogen only hormonal contraception associated with inhibition
of ovulation
Oral
Injectable
Highly Effective Methods That Are User Independent^{a}
Implantable progestogen only hormonal contraception associated with
inhibition of ovulation^{b}
Intrauterine device (IUD)
Intrauterine hormone-releasing system (IUS)
Bilateral tubal occlusion
Vasectomized partner
A vasectomized partner is a highly effective contraception method
provided that the partner is the sole male sexual partner of the WOCBP
and the absence of sperm has been confirmed. If not, an additional
highly effective method of contraception should be used.
Sexual abstinence
Sexual abstinence is considered a highly effective method only if
defined as refraining from heterosexual intercourse during the entire
period of risk associated with the study intervention.
The reliability of sexual abstinence needs to be evaluated in relation to
the duration of the study and the preferred and usual lifestyle of the
participant.
NOTES:
^{a}Typical use failure rates may differ from those when used consistently and correctly. Use should be consistent with local regulations regarding the use of contraceptive methods for participants participating in clinical studies.
^{b}Hormonal contraception may be susceptible to interaction with the study intervention, which may reduce the efficacy of the contraceptive method. In this case, a highly effective method of contraception plus condoms should be utilized during their participation in the study up to and including at least 1 complete menstrual cycle (≥30 days) for women and 90 days for men post last dose.

Pregnancy Testing:
WOCBP should only be included after a confirmed menstrual period and a negative serum HCG pregnancy test.
Pregnancy testing is required at screening, randomisation, and 14 days after the last dose at the follow-up visit.
Pregnancy testing will be performed whenever a menstrual cycle is missed or when pregnancy is otherwise suspected.

Collection of Pregnancy Information:

Male participants with partners who become pregnant
The investigator will attempt to collect pregnancy information on any male participant's female partner who becomes pregnant while the male participant is in this study. This applies to all male participants who receive Prevotella histicola Strain B.
After obtaining the necessary signed informed consent from the pregnant female partner directly, the investigator will record pregnancy information on the appropriate form and submit it to the Sponsor within 24 hours of learning of the partner's pregnancy. The female partner will also be followed to determine the outcome of the pregnancy. Information on the status of the mother and child will be forwarded to the Sponsor. Generally, the follow-up will be no longer than 8 weeks following the estimated delivery date. Any termination of the pregnancy will be reported regardless of foetal status (presence or absence of anomalies) or indication for the procedure.

Female participants who become pregnant
The investigator will collect pregnancy information on any female participant who becomes pregnant while participating in this study. Information will be recorded on the appropriate form and submitted to the Sponsor within 24 hours of learning of a participant's pregnancy. The participant will be followed to determine the outcome of the pregnancy. The investigator will collect follow-up information on the participant and the neonate and the information will be forwarded to the Sponsor. Generally, follow-up will not be required for longer than 6 to 8 weeks beyond the estimated delivery date. Any termination of pregnancy will be reported, regardless of foetal status (presence or absence of anomalies) or indication for the procedure.
While pregnancy itself is not considered to be an AE or SAE, any pregnancy complication or elective termination of a pregnancy will be reported as an AE or SAE. A spontaneous abortion is always considered to be an SAE and will be reported as such. Any post-study pregnancy-related SAE considered reasonably related to the study intervention by the investigator will be reported to the Sponsor as herein. While the investigator is not obligated to actively seek this information in former study participants, he or she may learn of an SAE through spontaneous reporting.
Any female participant who becomes pregnant while participating in the study will discontinue study intervention or be withdrawn from the study.

### 6.5. Appendix 5: HLA Testing

Use/Analysis of DNA
Genetic variation may impact a participant's response to study intervention, susceptibility to, and severity and progression of disease. Variable response to study intervention may be due to genetic determinants that impact drug absorption, distribution, metabolism, and excretion; mechanism of action of the drug; disease aetiology; and/or molecular subtype of the disease being treated. Therefore, where local regulations and IRB/IEC allow, blood samples will be collected for DNA analysis from consenting participants.
One sample will be analysed for HLA testing. A second sample will be taken for additional analyses that may be conducted if it is hypothesised that this may help further understand the clinical data.
The samples may be analysed as part of a multi-study assessment of genetic factors involved in the response to Prevotella histicola Strain B or study interventions of this class to understand study disease or related conditions.
The results of genetic analyses may be reported in the CSR.
The Sponsor or its agents will store the DNA samples in a secure storage space with adequate measures to protect confidentiality.
The samples will be retained while research on Prevotella histicola Strain B or study interventions of this class or indication continues but no longer than 12 months or other period as per local requirements.

### 6.6. Appendix 6: Liver Safety: Suggested Actions and Follow-Up Assessments

### 6.6.1. Healthy Volunteers

Healthy volunteers should stop dosing if ALT or AST is >3×ULN and/or bilirubin is >2×ULN. Liver function test (LFT) monitoring should be carried out until abnormal LFTs are back to within the normal range. Routine investigations should be performed to exclude viral/infectious causes of liver abnormalities.

6.6.2. Participants with Mild to Moderate Psoriasis or Atopic Dermatitis

Participants with either condition should stop dosing if ALT or AST is >3×ULN and/or bilirubin is >2×ULN. LFT monitoring should be carried out until abnormal LFTs are back to within the normal range. Routine investigations should be performed to exclude viral/infectious causes of liver abnormalities.

### 6.7. Appendix 8: Abbreviations

AE Adverse Event
ALP Alkaline Phosphatase
ALT Alanine Aminotransferase
ANCOVA Analysis of Covariance
AST Aspartate Aminotransferase
BSA Body Surface Area
BUN Blood Urea Nitrogen
CI Confidence Interval
CIA Collagen-Induced Arthritis
CIOMS Council for International Organizations of Medical Sciences
CNS Central Nervous System
CONSORT Consolidated Standards of Reporting Trials
CRF Case Report Form
CRP C-reactive Protein
CSR Clinical Study Report
DSS Dextran Sulphate Sodium
DTH Delayed-Type Hypersensitivity
EAE Experimental Allergic Encephalomyelitis
EASI Eczema Area and Severity Index
ECG Electrocardiogram
eCRF Electronic Case Report Form
EMA European Medicines Agency
FDA Food and Drug Administration
FIH First-in-Human
FITC Fluorescein Isothyocyanate
FSH Follicle Stimulating Hormone
GCP Good Clinical Practice
GI Gastrointestinal
GM-CSF Granulocyte-Macrophage Colony-Stimulating Factor
HBsAg Hepatitis B surface antigen
HBV Hepatitis B
HCG Human Chorionic Gonadotropin
HCV Hepatitis C
HED Human equivalent dose
HIPAA Health Insurance Portability and Accountability Act
HIV Human Immunodeficiency Virus
HLA Human Leukocyte Antigen
HAMR Hammersmith Medicines Research
HRT Hormonal Replacement Therapy
IB Investigator's Brochure
IBS Irritable Bowel Syndrome
ICF Informed Consent Form
ICH International Council for Harmonization
IEC Independent Ethics Committee
IF Immunofluorescence
IGA Investigator's Global Assessment
IHC Immunohistochemistry
IL Interleukin
INR International Normalized Ratio
IRB Institutional Review Board
IUD Intrauterine Device
IUS Intrauterine Hormone-Releasing System
LFT Liver Function Test
LSS Lesion Severity Score
MCV Mean Cell Volume
MCH Mean Corpuscular Hemoglobin
MedDRA Medical Dictionary for Regulatory Activities
MHRA Medicines and Healthcare Products Regulatory Agency
mRNA Messenger Ribonucleic Acid
MTX Methotrexate
NYHA New York Heart Association
OTC Over-The-Counter
PASI Psoriasis Area and Severity Index
PCR Polymerase Chain Reaction
PK Pharmacokinetic
PUVA Psoralen Plus Ultraviolet A
QC Quality Control
QTc Corrected QT Interval
QTcF QT Interval Corrected using Fridericia's Formula
RBC Red Blood Cell
RNA Ribonucleic Acid
SAE Serious Adverse Event
SAP Statistical Analysis Plan
SCORAD SCORing Atopic Dermatitis
SD Standard Deviation
SGOT Serum Glutamic-Oxaloacetic Transaminase
SGPT Serum Glutamic-Pyruvic Transaminase
SoA Schedule of Activities
SRC Safety Review Committee
SUSAR Suspected Unexpected Serious Adverse Reactions
ULN Upper Limit of Normal
UVA Ultraviolet A
WBC White Blood Cell
WOCBP Woman of Child-Bearing Potential

### 7. REFERENCES

Cosorich I, Dalla-Costa G, Sorini C, Ferrarese R, Messina M J, Dolpady J, et al. High frequency of intestinal TH17 cells correlates with microbiota alterations and disease activity in multiple sclerosis. Sci Adv. 2017; 3(7):e1700492.
De Filippo C, Cavalieri D, Di Paola M, Ramazzotti M, Poullet J B, Massart S, et al. Impact of diet in shaping gut microbiota revealed by a comparative study in children from Europe and rural Africa. Proc Natl Acad Sci USA. 2010; 107(33):14691-14696.
EMA CHMP Guideline on strategies to identify and mitigate risks for first-in-human and early clinical trials with investigational medicinal products. EMEA/CHMP/SWP/28367/07 Rev. 1. 20 Jul. 2017.
FDA Guidance for Industry. Drug-induced liver injury: premarketing clinical evaluation. US Department of Health and Human Services. FDA Center for Drug Evaluation and Research (CDER) Center for Biologics Evaluation and Research (CBER). July 2009.
Hjorth M F, Roager H M, Larsen™, Poulsen S K, Licht T R, Bahl M I, et al. Pre-treatment microbial Prevotella-to-Bacteroides ratio, determines body fat loss success during a 6-month randomized controlled diet intervention. Int J Obes (Lond). 2018; 42(3):580-583. Jangi S, Gandhi R, Cox L M, Li N, von Glehn F, Yan R, et al. Alterations of the human gut microbiome in multiple sclerosis. Nat Commun. 2016; 7:12015.
Karasov W H. Terrestrial vertebrates. In: Sibly R M, Brown J H, Kodric-Brown A, editors. Metabolic Ecology: A Scaling Approach. Hoboken, N.J.: Wiley-Blackwell; 2012. p. 212-224.
Mangalam A, Shahi S K, Luckey D, Karau M, Marietta E, Luo N, et al. Human gut-derived commensal bacteria suppress CNS inflammatory and demyelinating disease. Cell Rep. 2017; 20(6):1269-1277.
Marietta E V, Murray J A, Luckey D H, Jeraldo P R, Lamba A, Patel R, et al. Suppression of inflammatory arthritis by human gut-derived Prevotella histicola in humanized mice. Arthritis Rheumatol. 2016; 68(12):2878-2888.
Miyake S, Kim S, Suda W, Oshima K, Nakamura M, Matsuoka T, et al. Dysbiosis in the gut microbiota of patients with multiple sclerosis, with a striking depletion of species belonging to Clostridia XIVa and IV clusters. PLoS One. 2015; 10(9):e0137429.
Petersen L M, Bautista E J, Nguyen H, Hanson B M, Chen L, Lek S H, et al. Community characteristics of the gut microbiomes of competitive cyclists. Microbiome. 2017; 5(1):98.
Tagliabue A, Elli M. The role of gut microbiota in human obesity: Recent findings and future perspectives. Nutr Metab Cardiovasc Dis. 2013; 23(3):160-168.
Wu G D, Chen J, Hoffmann C, Bittinger K, Chen Y Y, Keilbaugh S A, et al. Linking long-term dietary patterns with gut microbial enterotypes. Science. 2011; 334(6052):105-108.

### Preliminary Findings:

Prevotella histicola Strain B was Well Tolerated with No Overall Difference Reported from Placebo
Patients Dosed with Prevotella histicola Strain B Showed a Reduction in Mean Lesion Severity Score vs. Placebo
Reductions Observed in Cellular Histological and Blood Immune Cell Biomarkers Consistent with Clinical Response

12 patients with mild to moderate psoriasis were randomized 2:1 to receive daily, oral administration of 550 mg (1× dose) of Prevotella histicola Strain B, or placebo, for 28 days. The primary endpoint was safety and tolerability. Secondary and exploratory endpoints included lesion severity score (LSS), a measure of clinical activity, cellular histological biomarkers and blood immune cell biomarkers taken from biopsies and blood samples, respectively, at the start and end of the 28-day dosing period.

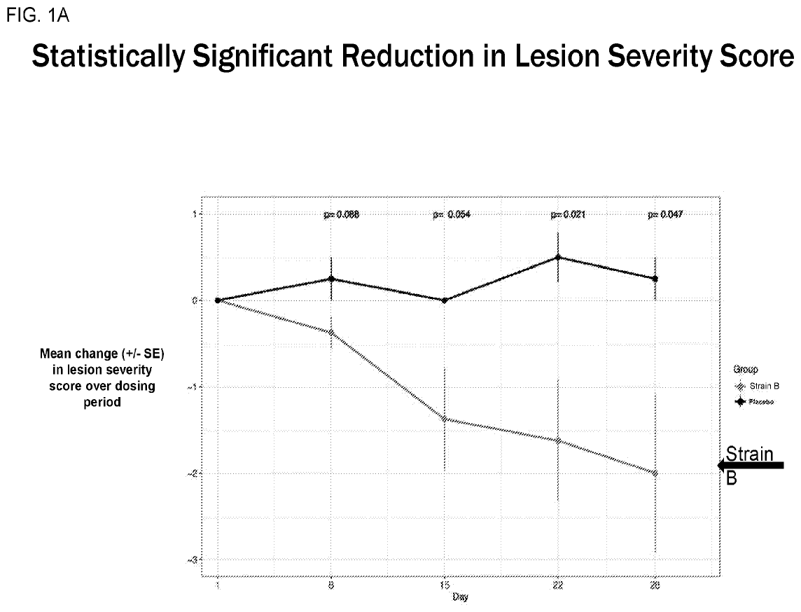

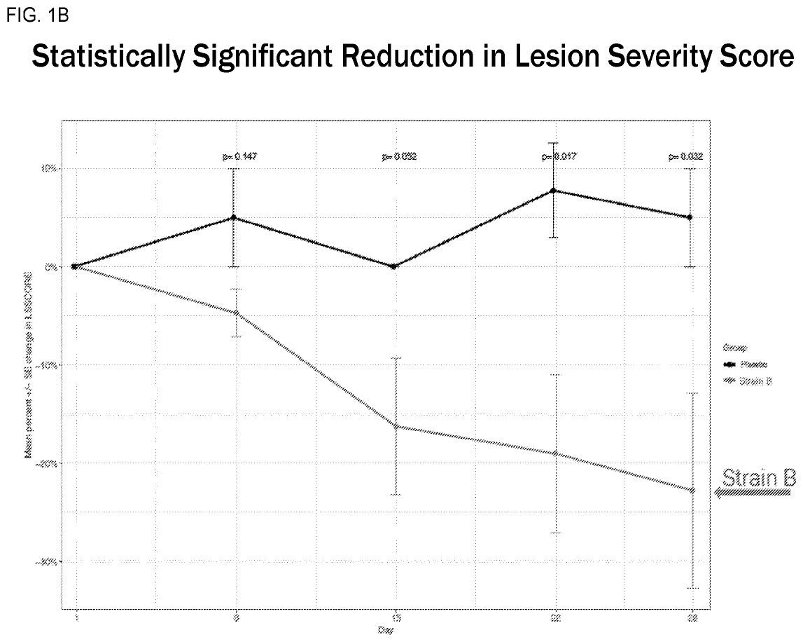

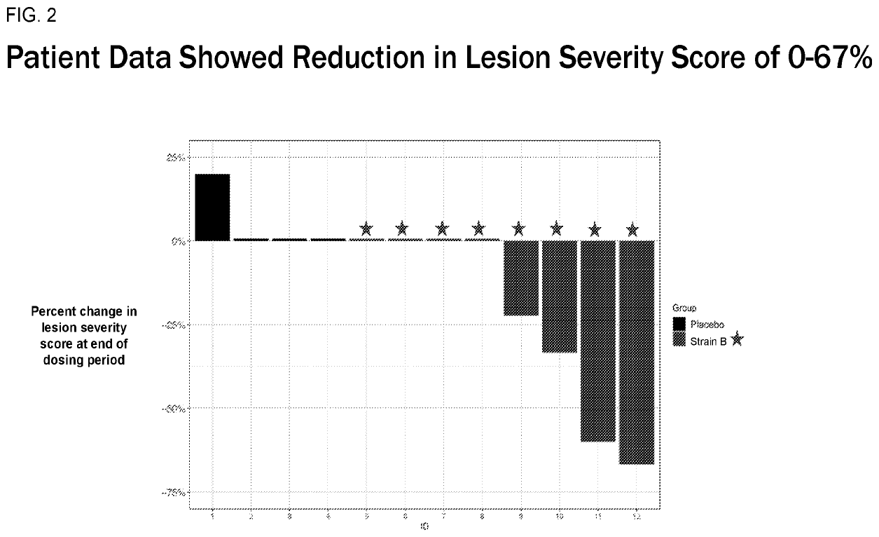

Patients dosed daily for 28 days with 550 mg of the enteric capsule formulation of Prevotella histicola Strain B showed a statistically significant (p≤0.05) reduction in mean LSS at 28 days of 2 points, compared to a mean increase of 0.25 points in patients who received placebo (FIG. 1A). FIG. 1B shows mean percent changes in Lesion Severity Scores (LSS) over the course of the study. Data from patients dosed with Prevotella histicola Strain B showed a reduction in LSS over the dosing period ranging from 0 to 67 percent (FIG. 2). LSS, a secondary endpoint, is a component of the Psoriasis Area and Severity Index (PASI) score and measures redness, thickness, and scaling of an individual psoriatic lesion across the dosing period and is a sensitive clinical measure for patients with mild to moderate disease.

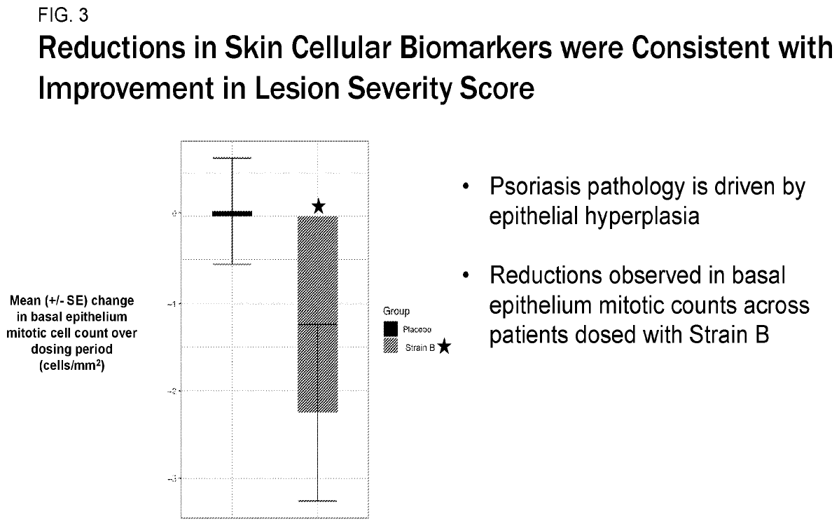

Analysis of the change over the dosing period of the basal epithelium mitotic count, a secondary endpoint and a cellular driver of psoriasis pathology, showed a mean reduction of 2.25 cells/mm² in patients who received Prevotella histicola Strain B compared to no change in patients receiving placebo (FIG. 3). Lower basal epithelium mitotic counts indicate a reduction of psoriasis pathology.

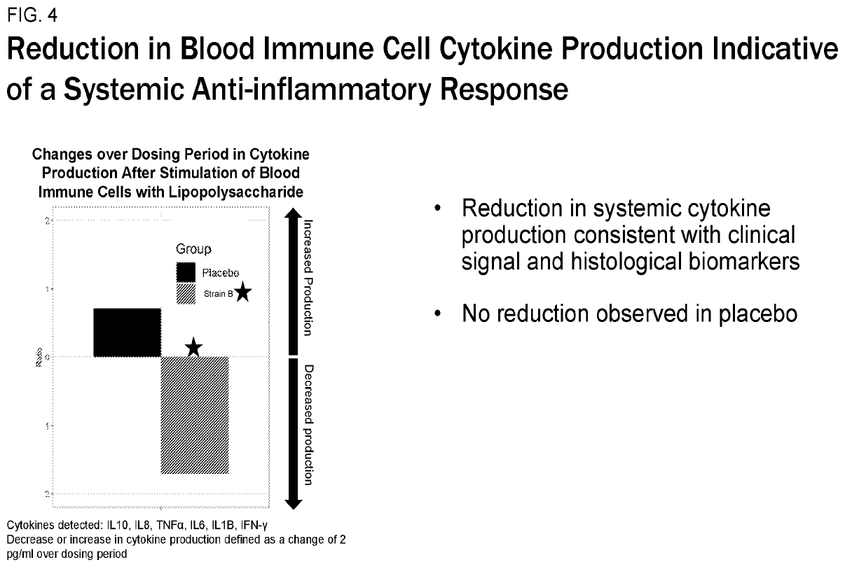

In an analysis of blood immune cell cytokine production following stimulation with lipopolysaccharide, an exploratory endpoint, the Prevotella histicola Strain B dosed patient group showed a reduction in cytokine production indicative of a systemic anti-inflammatory response, compared to no reduction in the placebo group (FIG. 4). Cytokines detected: IL10, IL8, TNFα, IL6, IL1B, IFN-γ.

### Phase 1 Study:

This study is a double-blind placebo-controlled Phase 1b trial designed to evaluate the safety and tolerability of Prevotella histicola Strain B in approximately 108 healthy volunteers and patients with mild or moderate psoriasis or atopic dermatitis. Prospectively defined secondary and exploratory endpoints include the effect of Prevotella histicola Strain B on clinical measures of disease and a range of biomarkers. Enrollment is underway in a cohort of mild to moderate psoriasis patients to be dosed with 2.76 g (5× dose) of the enteric capsule formulation. One further cohort of psoriasis patients and one cohort of atopic dermatitis patients are planned to be dosed with a new formulation of Prevotella histicola Strain B.

### Phase 2 Clinical Trial:

Sponsor plans to advance Prevotella histicola Strain B into Phase 2. This trial is designed to investigate daily dosing of Prevotella histicola Strain B in mild to moderate psoriasis. The primary endpoint of the trial is expected to be reduction in the PASI score over 24 weeks, with an interim analysis at 12 weeks or over 16 weeks, with an interim analysis. Multiple doses and formulations of Prevotella histicola Strain B will be investigated. Part A of the trial is designed to select the optimal formulation and will test the enteric capsule formulation and the new formulation of Prevotella histicola Strain B versus placebo in approximately 180 patients. Part B of the study will test multiple doses of the optimal formulation against placebo for 24 weeks or 16 weeks in approximately 250 patients.

Further Positive Interim Clinical Data in Patients with Psoriasis at High Dose in Phase Lb Trial

Eighteen patients (e.g., subjects) with mild to moderate psoriasis were randomized 2:1 to receive a daily oral administration of 2.76 g (5× or high dose) of Prevotella histicola Strain B or placebo for 28 days. The primary endpoint was safety and tolerability. Secondary and exploratory endpoints included lesion severity score (LSS), Psoriasis Area and Severity Index (PASI), both measures of clinical activity, as well as cellular histological biomarkers and blood immune cell biomarkers taken from biopsies and blood samples at the start and end of the dosing period, respectively. Safety and tolerability and secondary clinical endpoints were also measured at day 42, two weeks after completion of dosing.

Lesion Severity Score (LSS) is a sensitive clinical measure of disease change in psoriasis. LSS is a fundamental component of the PASI scoring system and measures the underlying changes in lesion severity from a single psoriatic lesion. The LSS measures redness, thickness and scaling on a 12-point scale for the same individual lesion. LSS is generally considered a more sensitive measure for patients with mild-to-moderate disease, for which individual lesions may be quite severe but overall affected area compared to body surface may be small. It is also more sensitive when the dosing period is short as it can detect smaller changes from baseline.

PASI (Psoriasis Area and Severity Index) measures the same underlying changes in a psoriasis skin lesion as the LSS but captures those changes across all the skin lesions and weights the score by body surface area affected for each region of the body. PASI is a quantitative rating score for measuring the severity of psoriatic lesions based on area coverage and plaque appearance. PASI combines this assessment into a single score in the range of 0 (no disease) to 72 (maximal disease). The body is divided into four sections (head, arms, trunk, and legs). The average lesion severity score and area affected by lesions is assessed for each of these areas individually, and then the four scores are weighted and combined into a final PASI score.

LSS and PASI are strongly correlated and would be anticipated to move in tandem.

Results:

Prevotella histicola Strain B continued to be well tolerated in this cohort, with no overall difference reported from placebo. At the end of the 28-day dosing period, the high dose cohort showed a mean reduction in LSS consistent with previously reported data from a low dose cohort.

Two weeks following the completion of the dosing period, at day 42, the high dose cohort showed continued reductions from baseline in both mean LSS and PASI, which may be indicative of a sustained clinical effect and dose response.

A summary of the LSS and PASI results are shown in Tables 9 and 10 below.

TABLE 9
Mean (+/−SE) Percentage Change in LSS vs. Start of Dosing Period⁽¹⁾

| | | At end of 28-day | |
|---|---|---|---|
| | n | dosing period | At day 42 |
| Placebo (2) | 10 | 0.6% (9.0%) | −7.2% (6.2%) |
| Prevotella histicola | 12 | −15.1% (6.4%) | −24.1% (7.1%) |
| Strain B (high dose) | | | |
| Prevotella histicola | 8 | −22.8% (9.9%) | −9.0% (12.7%) |
| Strain B (low dose) | | | |

TABLE 10
Mean (+/−SE) Percentage Change in PASI vs. Start of Dosing Period⁽¹⁾

| | | At end of 28-day | |
|---|---|---|---|
| | n | dosing period | At day 42 |
| Placebo⁽²⁾ | 10 | −1.0% (13.2%) | −3.3% (14.8%) |
| Prevotella histicola | 12 | −16.0% (8.1%) | −20.7% (8.2%) |
| Strain B (high dose) | | | |

Note: ⁽¹⁾This study was not sufficiently powered to detect statistical significance between treatment groups.⁽²⁾Represents the combination of placebo arms for the low dose (n=4) and high dose (n=6) cohorts.

A range of histological and molecular biomarkers were measured in the high dose cohort, with trends in line with the clinical effects of Prevotella histicola Strain B at the cohort level.

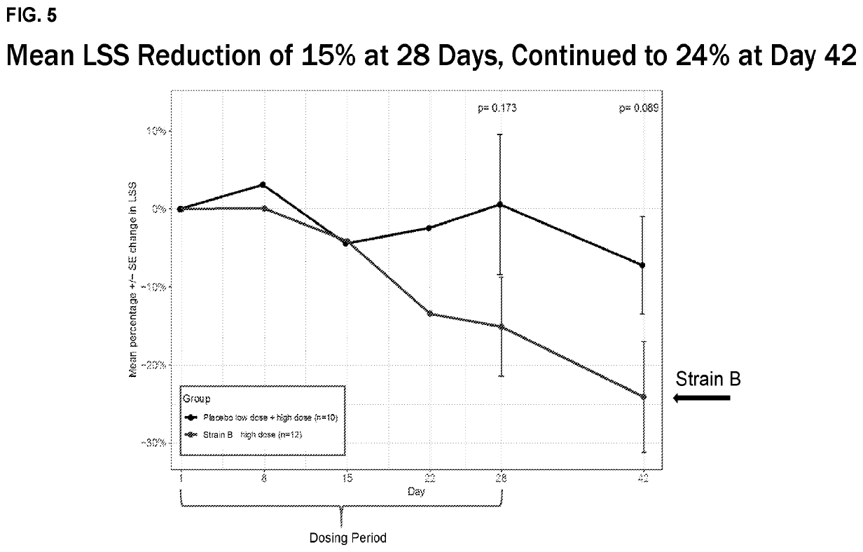

FIG. 5 shows the LSS data from the high dose cohort. The graph plots the change in LSS observed over the 28-day dosing period and the subsequent 2-week follow-up at day 42. The placebo arms from both the low and high dose cohort were pooled. This allows a correction for the asymmetric 2:1 randomization and improves the robustness of the placebo data.

A mean LSS reduction was seen from baseline of 15% in the patients taking the high dose of Prevotella histicola Strain B at day 28. Two weeks post completion of dosing, at day 42, mean LSS reduction continued to 24%, suggesting a sustained clinical effect and dose response.

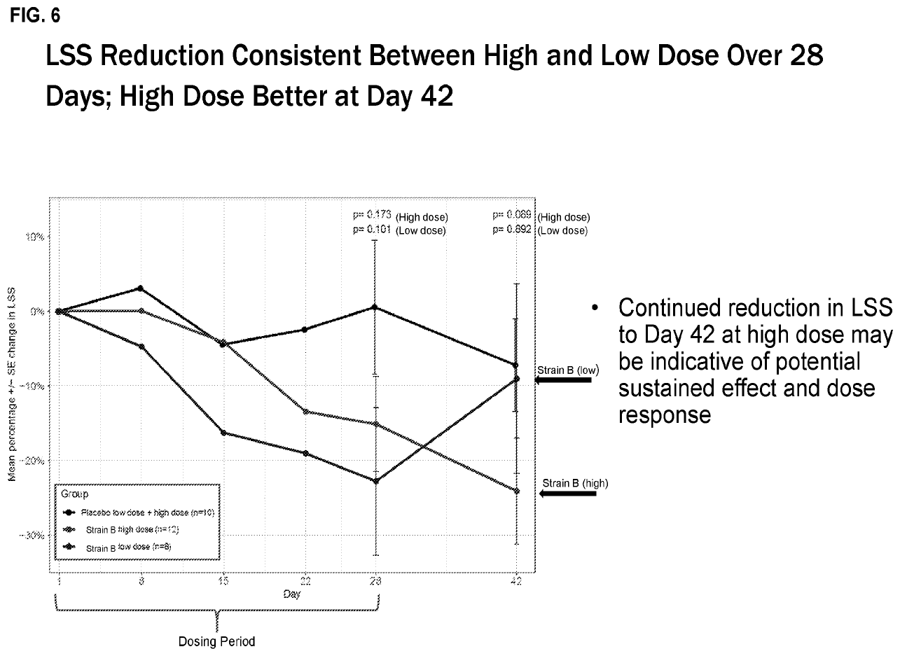

FIG. 6 shows that the observed LSS changes were consistent between the high (2.76 g) and low (550 mg) dose cohorts over the 28-day dosing period. At day 42, however, the low dose cohort returned almost to baseline, while reduction continued in the high dose. Again, this suggests a sustained clinical effect and possible dose response.

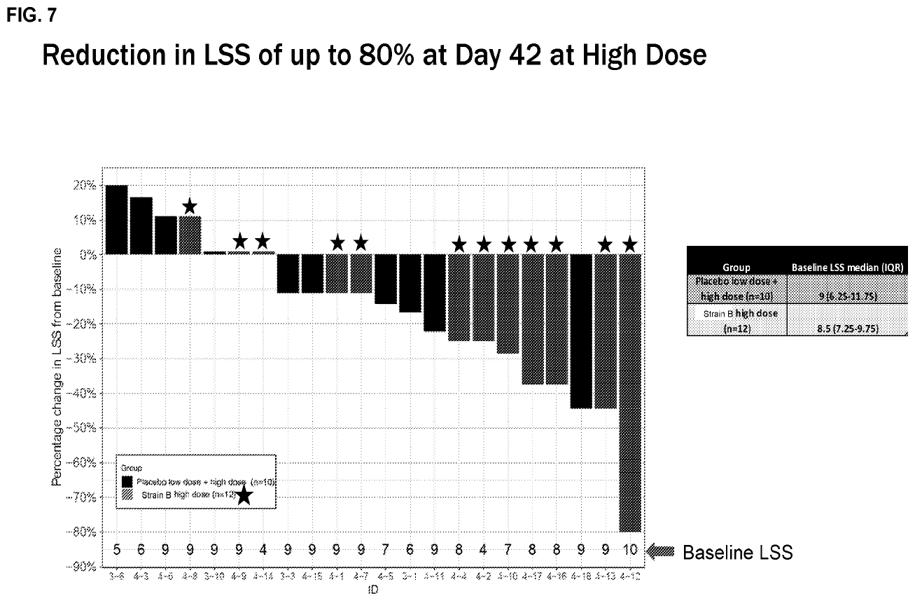

FIG. 7 shows the individual changes from baseline in LSS at day 42 for each of the patients in the high dose cohort.

On the bottom there is numeric score, this is the baseline LSS for the lesion that was tracked. A few things to note about that score:
There is no baseline difference between the patients who were dosed with placebo or dosed with the Prevotella histicola Strain B.
Although the patients were classified as having mild disease in terms of their body surface area, the actual lesions that were tracked had quite active disease.
Most of these patients have scores of between 8 and 10 out of 12.

Nine out of the 12 patients receiving Prevotella histicola Strain B showed a reduction in LSS and in 7 of these 9 patients the reduction was 25% or greater. The maximum observed response in the Prevotella histicola Strain B dosed group was an 80% reduction.

The magnitude of this effect at 42 days is remarkable, given both the short duration period, and the fact that these measurements were taken two weeks after patients discontinued therapy.

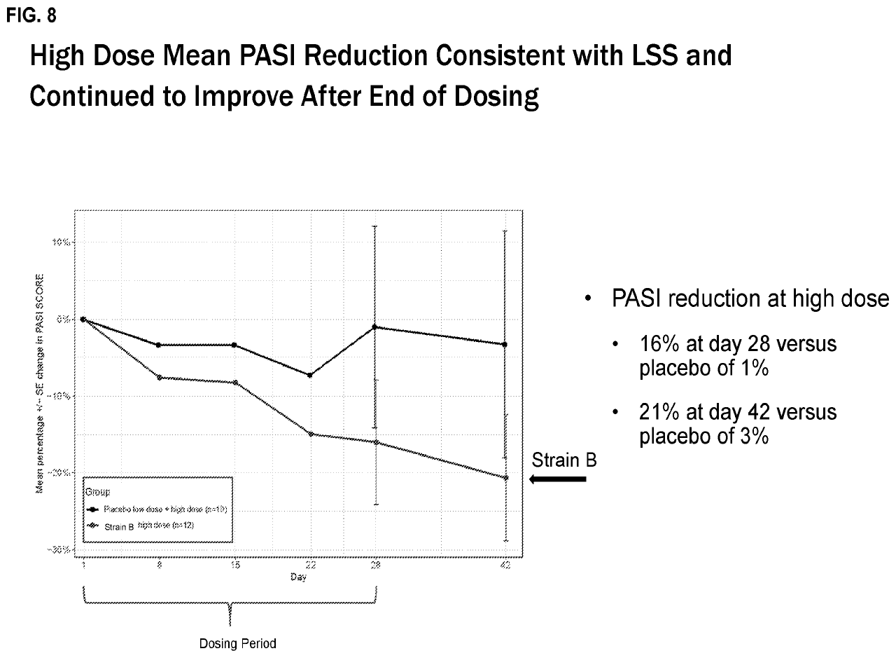

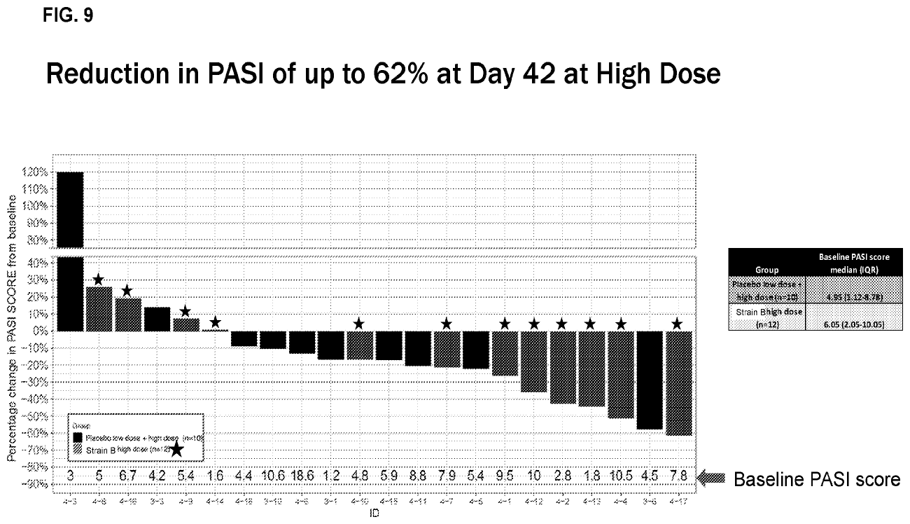

FIG. 8 and FIG. 9 show an analysis of the PASI data over the same dosing period.

FIG. 8 shows the population PASI results:
The mean reduction at the high dose is 16% at 28-days. The PASI reduction continued to improve over the next 2 weeks despite stopping dosing and the reduction at day 42 was 21%.
The observed effects on the PASI score were very consistent with the trends observed in LSS.

FIG. 9 shows individual changes from baseline in PASI at day 42 for each of the patients in the high dose cohort.

On the bottom the baseline PASI score is shown which ranged from as low as 1.2 to 18 reflecting the mild level disease, as measured by PASI, present in these patients.

PASI reductions of up to 61% were observed in the Prevotella histicola Strain B treated patients.

This interim data strongly supports the potential of Prevotella histicola Strain B as a new therapy for patients with mild to moderate psoriasis.

Prevotella histicola Strain B continued to be well-tolerated.

At two weeks post-dosing, the mean reduction in LSS was 23 percent, and the mean reduction in PASI was 21 percent with maximal observed reductions of 80% and 61% respectively.

Safety and Efficacy of an Orally Administered, Single Strain Commensal Microbe in Psoriasis after 28 Days of Therapy: Prevotella histicola Strain B

### Introduction and Objectives:

Prevotella histicola Strain B was prepared in a pharmaceutical preparation of a single strain of Prevotella histicola isolated from the duodenum of a human donor. It has potent anti-inflammatory effects on human immune cells in vitro and mouse models in vivo. Preclinically, Prevotella histicola Strain B systemically suppresses multiple cytokines including TNF, IL-6 and IL-17. These effects are dependent on IL-10 signaling and are associated with increased epithelial expression of FoxP3. Prevotella histicola Strain B acts on the small intestinal axis, the network of connections between the small intestine and the rest of the body. It elicits systemic therapeutic effects without systemic absorption. Epithelial and dendritic cells in the small intestinal mucosa continuously sample the contents of the lumen. Once exposed to Prevotella histicola Strain B these cells modulate inflammation systemically via cytokine signaling and T-cell trafficking. Prevotella histicola Strain B significantly reduced types 1 and 3 inflammation in psoriasis-relevant preclinical mouse models including keyhole limpet haemocyanin delayed-type hypersensitivity, imiquimod-induced skin inflammation, and experimental autoimmune encephalomyelitis.

### Materials and Methods:

Prevotella histicola Strain B was evaluated in a phase 1b clinical study comprising 2 dose cohorts of 12 and 18 patients with mild to moderate psoriasis randomized 2:1 active:placebo. Doses were 1.6×10¹¹ bacterial cells (cohort L) or 8.0×10¹¹ cells (cohort H) of freeze-dried powder in enteric capsules for 28 days, with follow-up off drug through 42 days. Recoverable cell viability was >1%. The percentage change in the Lesional Severity Score (LSS) and the PASI score were measured at baseline, Day 28, and Day 42. Placebo subjects were pooled across both cohorts. This phase 1b study was not powered for statistical significance.

### Results:

Prevotella histicola Strain B was well tolerated at daily doses of up to 8.0×10¹¹ cells administered for up to 28 days, with a tolerability profile comparable to placebo. There were no serious adverse effects. Baseline mean PASI scores were 9.5 (cohort L), 6.2 (cohort H), and 6.7 (pooled placebo cohorts). Mean LSS scores were 8.1 (cohort L), 7.8 (cohort H), and 7.8 (pooled placebo cohorts).

At day 28, the percentage reduction in PASI for both Prevotella histicola Strain B cohorts was 16%, compared to 1% for placebo. At day 42, the percentage improvement from baseline increased to 21% in the high dose cohort, but not in the low dose cohort (10%) or placebo cohorts (3%).

The percentage reduction in LSS scores at 28 days were 15% (cohort H) and 23% (cohort L), compared to a 1% increase from baseline in the placebo group. At day 42, the percentage reduction in LSS in the high dose group continued to improve (24% reduction) but not in the lower dose group (9%) or pooled placebo cohorts (7%).

### Conclusions:

These data provide the first clinical evidence of modulation of systemic inflammation by an oral, safe, luminally-restricted microbial therapeutic. Currently no licensed drugs are known to treat human disease by this mechanism of action. Both doses performed similarly with respect to PASI and LSS score % change. At day 42 the higher dose of Prevotella histicola Strain B continued to show clinical improvement. These data support further clinical development of Prevotella histicola Strain B.

### Additional Data

Table 11 shows representation of biomarker response to Prevotella histicola Strain B treatment in skin and blood. Percent changes in LSS (DLSS %) are shown in the first column as per results in FIG. 2. Skin biopsies were collected at baseline (pre-treatment) and on last day of dosing (post-treatment), and processed to FFPE blocks, then sectioned and stained with H&E. The percent change in the number of mitotic cells in the basal epithelium post- vs pre-treatment is shown in the second column (DBEMC %). Whole blood was collected at pre- and post-treatment and stimulated ex vivo with LPS for 24 hrs. Cytokine levels were assessed by Luminex. Post- vs pre-treatment fold change ratios are shown for each cytokine.

TABLE 11
shows representation of biomarker response to Prevotella histicola
Strain B treatment in skin and blood.

| | Skin | | | | | | | |
|---|---|---|---|---|---|---|---|---|
| | | DBEMC | Blood | | | | | |
| Subject ID | DLSS % | % | IL-1b | IL-6 | IL-8 | IL-10 | IFNg | TNFa |
| 1 | 20.00 | −33.33 | −1.18 | 1.05 | −1.25 | −1.18 | −1.12 | 1.09 |
| 3 | 0.00 | 100.00 | −1.82 | −1.01 | 3.31 | 1.67 | −8.77 | 1.04 |
| 4 | 0.00 | 50.00 | 2.97 | 1.92 | 10.06 | 2.22 | 6.43 | 2.42 |
| 2 | 0.00 | −50.00 | 1.04 | −1.00 | −1.21 | −1.18 | 1.22 | 1.00 |
| 5 | 0.00 | 200.00 | 1.26 | 1.14 | −1.48 | −1.07 | 1.92 | 1.43 |
| 7 | 0.00 | 0.00 | −1.08 | −1.17 | 1.06 | 1.17 | −1.56 | −1.49 |
| 8 | 0.00 | −85.71 | 1.30 | 1.14 | −1.34 | −1.40 | 1.87 | 1.89 |
| 6 | 0.00 | −100.00 | −1.21 | −1.29 | −1.39 | −1.16 | −1.45 | −1.00 |
| 9 | −22.22 | −42.86 | −1.28 | −1.13 | 1.02 | −1.09 | 1.04 | 1.13 |
| 10 | −33.33 | −100.00 | 1.10 | 1.08 | 1.05 | −1.12 | 2.89 | 1.10 |
| 11 | −60.00 | −80.00 | −2.66 | −2.34 | −14.94 | −2.52 | −1.34 | −1.61 |
| 12 | −66.67 | 50.00 | −2.26 | −1.66 | −2.88 | −1.38 | 3.55 | −2.10 |

### Example 4: Effect of Prevotella histicola Strain B on Cytokine Production

Primary Human Cell Assay.

Human CD14+ PBMCs were grown in GM-CSF to induce an M1-type pro-inflammatory phenotype. Cells were then activated for 24 hrs with LPS+IFNg. Cells were incubated with individual strains of microbes for 24 hrs, after which cytokines in the supernatant were measured.

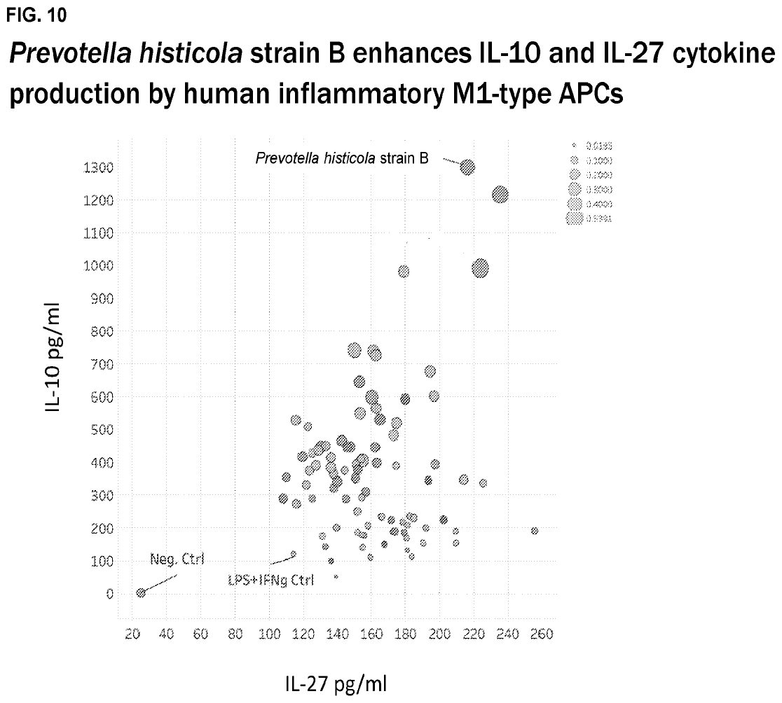

Eighty-eight obligate anaerobes were tested in this screen (FIG. 10). Each point represents the average value from 3 individual healthy donors. The size of the circle represents the IL-10/TNFa ratio. Prevotella histicola Strain B induced high amounts of anti-inflammatory cytokine IL-10 and IL-27 from M1-type skewed macrophages (FIG. 10). These data also demonstrate that each strain has a unique cytokine profile and that taxonomy is not a guide to function.

### Example 5: Prevotella histicola Strain B in a Mouse Model of Delayed-Type Hypersensitivity (DTH)

Delayed-type hypersensitivity (DTH) is an animal model of atopic dermatitis (or allergic contact dermatitis), as reviewed by Petersen et al. (In vivo pharmacological disease models for psoriasis and atopic dermatitis in drug discovery. Basic & Clinical Pharm & Toxicology. 2006. 99(2): 104-115; see also Irving C. Allen (ed.) Mouse Models of Innate Immunity: Methods and Protocols, Methods in Molecular Biology, 2013. vol. 1031, DOT 10.1007/978-1-62703-481-4_13).

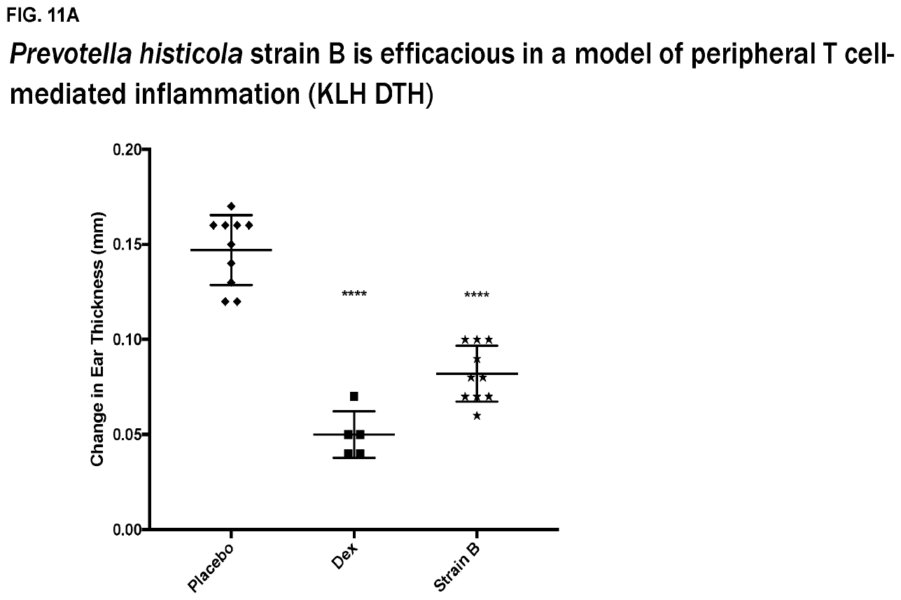

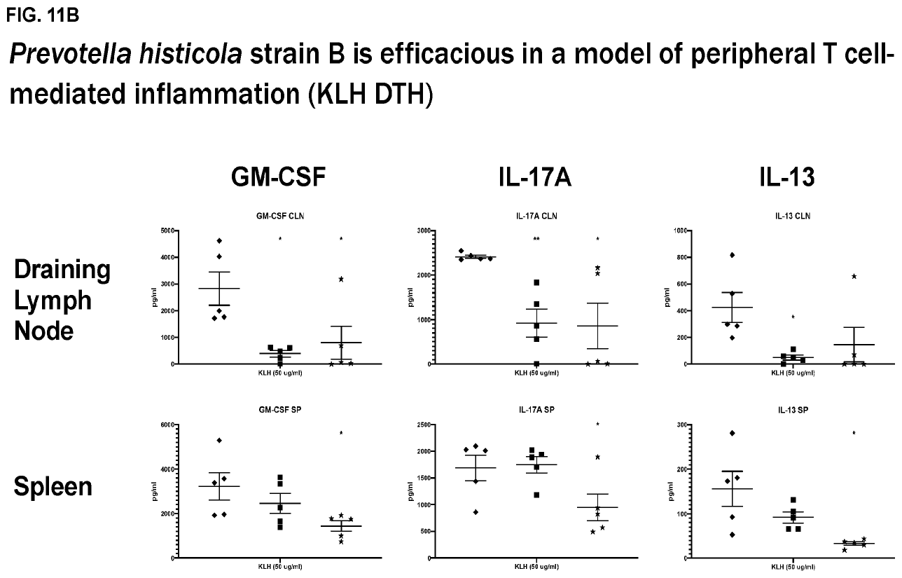

Mice were injected with KLH and CFA i.d. at 4 locations along the back (50 ug per mouse of KLH prepared in a 1:1 ratio with CFA in a total volume of 50 ul per site). Mice were dosed for 8 days as follows; 1) oral administration of anaerobic PBS (vehicle); 2) oral administration of 1.8 mg Prevotella histicola Strain B in an uncoated solid dosage form; 3) i.p. administration of Dexamethasone (Dex) (positive control). At day 9 post-challenge with 10 ug of KLH (10 ul volume), the group receiving Prevotella histicola Strain B had lower changes in ear thickness scores (FIG. 11A). At the end of the DTH study, mice were sacrificed and total cells from ear draining lymph nodes and spleens were incubated with KLH for 2 days. Cytokines from supernatants were measured by MSD (FIG. JIB).

### Example 6: Prevotella histicola Strain B in a Mouse Model of Psoriasis

Psoriasis is a T-cell-mediated chronic inflammatory skin disease. Several animal models have contributed to the understanding of this disease, as reviewed by Gudjonsson et al. (Mouse models of psoriasis. J Invest Derm. 2007. 127: 1292-1308; see also van der Fits et al. Imiquimod-induced psoriasis-like skin inflammation in mice is mediated via the IL-23/IL-17 axis. J. Immunol. 2009 May 1. 182(9): 5836-45).

Day −2 prior to start of experiment, the backs of BALB/c mice were shaved and then depilated with Nair (˜25 sec). The Nair was wiped off and backs of mice washed with warm water (2×).

On Day 0, using calipers, baseline ear measurements were taken.

On Days 1-7, 5% Imiquimod (a TLR7 and TLR8 agonist) (62.5 mg—back, 20 mg—ear per mouse) or control cream was applied on the backs and ears of mice. The cream was re-spread to ensure uniform application. Mice were dosed with Prevotella histicola strain B powder (10 mg) by oral gavage every day in 100 ul volume and the positive control group received dexamethasone (Dex) (1 mg/kg IP) i.p. A negative control group received vehicle.

400-500 ul 0.9% saline was injected s.c. daily to counteract any dehydration due to imiquimod application.

On Day 8, mice were euthanized, and tissues were harvested for downstream analyses.

Preparation of Dexamethasone (positive control): Dexamethasone stock solution was prepared by resuspending 25 mg of dexamethasone (Sigma) in 1.6 ml of 96% ethanol.

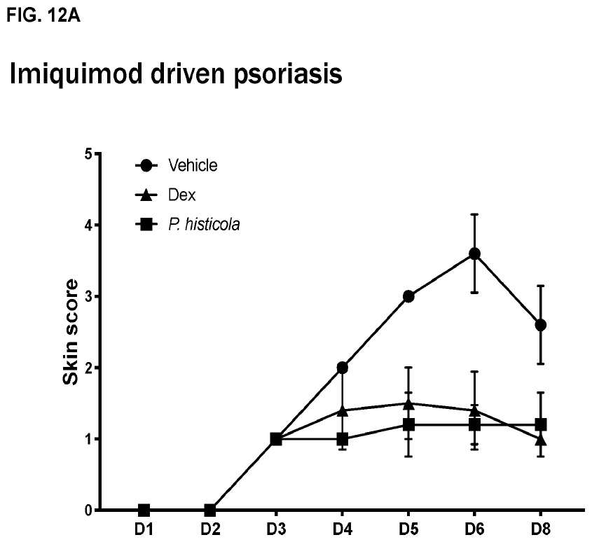

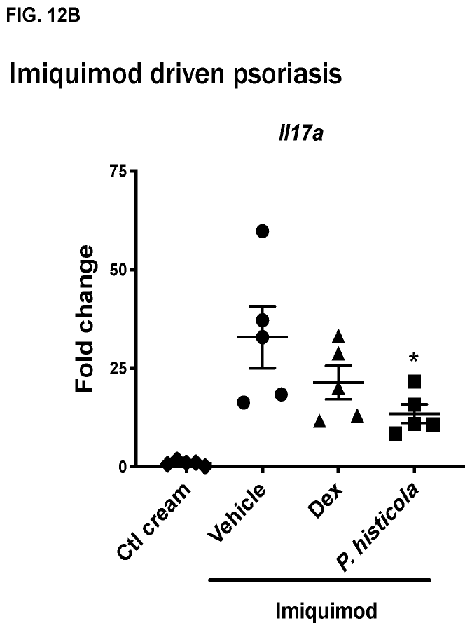

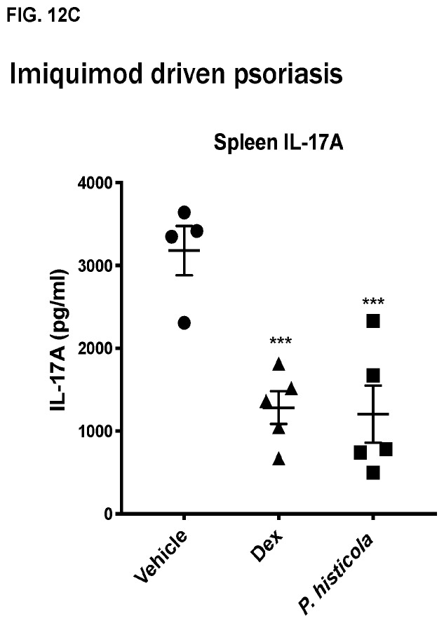

FIG. 12A shows results from the imiquimod driven psoriasis mouse model. Back scores were recorded daily to measure erythema and scaling associated with psoriasis. FIG. 12B shows that Il17a mRNA transcripts from the psoriatic skin of the mice were measured by RT-qPCR. FIG. 12C shows that ex vivo stimulation of splenocytes. At termination of the study, mice were sacrificed and splenocytes were stimulated with PMA/Ionomycin for 48 hrs. IL-17A was measured from supernatants by MSD.

### Example 7: Prevotella histicola Strain B Modulating the Small Intestinal Axis

Resolution of multiple pathways of systemic inflammation is induced by modulation of the small intestinal axis by an orally administered single strain of Prevotella histicola.

### Materials and Methods

Mice. Female BALB/c, C57BL/6 mice (6-8 weeks old) were purchased from Taconic Farms or Jackson Labs. Female DO11.10 TCR Tg and SJL mice (8-10 weeks old) were purchased from Jackson Labs. Animals were housed in specific pathogen-free conditions in a vivarium (5 mice per cage), and all experiments were performed under Institutional Animal Care and Use Committee (IACUC) approved protocols and guidelines at Avastus Preclinical Services facility in Cambridge, Mass. EAE experiments were performed under IACUC approved protocols at Hooke Laboratories (Lawrence, Mass.). Mice were allowed to acclimate in the vivarium for 1-2 weeks prior to the start of experiments. PicoLab Rodent Diet 20 was provided and autoclaved water via sipper bottle, given ad libitum and checked daily.

Bacterial strains. Four individual strains of Prevotella species were obtained for this study. All strains were purified via single colony isolation method. Strain identity was confirmed by 16S rDNA and whole genome sequencing. Prevotella histicola and P. jejuni were isolated from human duodenal biopsy (Marietta et al., 2016) and obtained from Mayo Clinic. P. melaninogenica was isolated from a fresh human subgingival plaque sample of a healthy volunteer. Informed consent was obtained from the volunteers.

Microbial biomass. All strains were grown in commercial Tryptic Soy Broth (TSB, Corning #61-411-RO) medium, or in in-house developed Soy Peptone-Yeast Extract-Glucose medium with L-cysteine-HCl as reducing agent. To support growth of the microorganisms the medium was supplemented with 5 mg/L hemin and 0.05 mg/L vitamin K, or with 20 mg/L hemoglobin. Microbial cultures were incubated anaerobically at 37° C. for 12-18 hours before harvesting. Bacterial biomass was concentrated by centrifugation at 7000 g for 20 min at 10° C., resuspended in anaerobic yeast extract-sucrose solution and distributed into cryovials under anaerobic conditions. Cryovials were immediately frozen in liquid N₂ and stored at −80° C. Bacterial total cell count (TCC) was measured by Coulter Counter Multisizer4e. Biomass TCC varied from 8.2e+10 to 9.4e+10 cells/ml. Bacterial identity was confirmed by 16S rDNA sequencing.

Bacterial biomass was thawed at room temperature. 100 μl of suspension was administered orally to each mouse daily for 4-9 days. For in vitro assays thawed bacterial biomass was serially diluted in RPMI degassed medium inside an anaerobic chamber (Coy Lab Products, USA) to reach approximately 2E+6 bacterial cells/ml. 100000 bacterial cells were added to 200000 purified human immune cells per each 96-well manually or by using automated Liquid handler Biomek 4000 (Beckman Coulter) inside a custom-built Coy Anaerobic chamber. The co-cultures were incubated for 24 hrs under micro-oxic conditions (1% O₂, 5% CO₂, balanced by N₂). After incubation cell supernatants where collected and Luminex technology was used to measure cytokine production.

Lyophilized powders. Prevotella histicola strain B lyophilized powders were produced from Prevotella histicola either by externally contract manufacturers or internally. Powders were stored in sealed mylar bags inside a desiccator at 4° C. Prevotella histicola strain B powders were characterized by TCC. Test aliquots of Prevotella histicola strain B powder were distributed into plastic test tubes with caps and stored at 4° C. For administration to mice the powder was resuspended in anaerobic yeast extract-sucrose solution at room temperature. 100 μl of suspension was administered orally to each mouse daily. The daily dose was calculated based on TCC. On average 10 mg/dose corresponded 4.1e+9 TCC/dose and 1.0 e+7 CFU/dose.

For non-viable lyophilized powders, aliquots were subjected to 25 kGy Gamma Irradiation treatment at Sterigenics U.S., LLC. Treated powders were characterized by TCC and VCC methods. Total cell number did not change. There were no viable cells left after irradiation.

Dosing with Prevotella histicola strain B and controls in vivo. Mice were treated orally with Prevotella histicola strain B (4.1E+9 TCC/0.1 mL/day PO) or vehicle control (anaerobic sucrose, PO) for duration of different models. Prevotella histicola strain B was dosed in a range of forms: biomass, resuspended powder or a compressed tablet formulation (Total cell count (TCC)). Dexamethasone (1 mg/kg, i.p., Sigma) was used as a positive control unless otherwise specified.

For anti-IL10R blockade, anti-IL10R (BioXCell Clone IB13.A) and Rat IgG1 HRPN isotype control (BioXCell) were diluted in corresponding dilution buffers, InVivoPure pH 6.0T Dilution Buffer (BioXCell) and InVivoPure pH 7.0 Dilution Buffer (BioXCell), respectively. Mice were dosed i.p. with 100 μl of solution at a concentration of 200 μg per mouse on days 0, 3 and 7.

For imiquimod driven psoriasis, anti-IL-17A (Bio X Cell Clone C17.8) was dosed at 200 μg per mouse i.p. on days 2, 4 and 6. For EAE studies, fingolimod (1 mg/kg, PO, Tocris Biosciences) was dosed daily.

Delayed Type Hypersensitivity mouse model. Mice were immunized with 50 μl of emulsion of keyhole limpet hemocyanin (KLH) in Complete Freund's Adjuvant (CFA) on four sites on the back. 8 days later, recipient mice were challenged with KLH (10 μg/10 μl) intradermally in the ear. Ear measurements were recorded 24 hours post ear challenge using digital calipers. Change in ear thickness was expressed as ear thickness at day 7 minus ear thickness at baseline.

For the adoptive transfer DTH, cells were isolated from spleens and all lymph nodes of DO11.10 TCR Tg mice. 4-5×10⁷ cells resuspended in 200 μl of PBS were injected into naive BALB/c recipient mice. Mice were then immunized with 200 μl of ovalbumin-CFA emulsion on four sites on the back. 8 days later, recipient mice were challenged with ovalbumin (20 μg/20 μl) intradermally in the ear. Ear measurements were recorded 24 hours post ear challenge. Change in ear thickness was expressed as ear thickness at day 7 minus ear thickness at baseline.

Imiquimod-induced psoriasis-like skin inflammation protocol. Mice were sensitized topically with 62.5 mg imiquimod cream (Aldara; 3M Pharmaceuticals, St Paul, Minn., USA) on shaved backs daily for 7 consecutive days. The severity of inflammation of the back skin was evaluated using a lesion psoriasis severity scoring system. Mice were monitored and graded daily on the scale: 0 (no alteration), 1 (mild erythema), 2 (moderate to severe erythema and some plaques), 3 (marked erythema and plaques) and 4 (very marked erythema and plaques). The same mice were also sensitized with 20 mg imiquimod on the ear. Ear measurements were taken daily using digital calipers and scores were reported as change in ear thickness calculated as ear score on day 8 minus baseline ear score on day 1. On day 8 study termination, skin samples from back lesions of mice were fixed in 10% formalin and embedded in paraffin. Deparaffinized sections were stained with hematoxylin and eosin to study their microarchitecture and scored for disease parameters by a pathologist.

Experimental Autoimmune Encephalomyelitis. Female SJL mice (8-10 weeks old) were subcutaneously injected at four sites with myelin proteolipid protein (PLP)139-151 in CFA emulsion (0.05 mL/injection site; ˜0.5 mg PLP PLP139-151/mL; Hooke Laboratories; EK-2120). Following immunization, EAE induction was completed by intraperitoneal injections of pertussis toxin (6 μg/mL; 0.1 mL/mouse) within 2 hours of immunization. Mice were randomized into groups and monitored for EAE clinical score over the course of 42 days. Disease progression was scored blinded of treatment or prior measurements. Disease severity was scored using standard EAE criteria: 0 (normal); 1 (loss of tail tone); 2 (hind limb weakness); 3 (hind limb paralysis); 4 (hind limb paralysis and forelimb paralysis or weakness); 5 (morbidity/death). Mice were observed daily for clinical symptoms. Mice were euthanized if they had a score of 4 for 2 days, and a score of 5 was recorded for remainder of the study for these animals.

End point tissue collection and histology. After euthanasia at the end of the study, EAE mice were perfused with 5-10 mL PBS and the spinal column was extracted from the base of the skull to the beginning of the pelvic bone. Spinal columns were then drop-fixed in 10% neutral buffered formalin and stored horizontally for 48 hours. After fixation, spinal columns were treated in mild formic acid decalcification solution (Immunocal-Statlab, Fisher Scientific, #141432) overnight (12-24 hours) at room temperature. Spinal columns were then trimmed into 4 mm-thick cervical, thoracic, and lumbar segments and processed using a Sakura Tissue Tek VIP 5 by graded alcohol dehydration, cleared in xylene, and finally infiltrated with paraffin. After processing, spinal column segments were embedded into paraffin blocks. Paraffin blocks were then sectioned at 4 μm on charged slides, air-dried overnight and stained with Hematoxylin and Eosin according to standard automated H&E protocol (Tissue-Tek Prisma) and then cover slipped (Tissue-Tek Glass). Prepared tissue sections were then imaged using a NanoZoomer 2.0 HT (Hamamatsu) at 20× magnification.

FITC-induced allergic inflammation. Backs of female BALB/c mice were shaved and on days 1 and 2 400 μl of 0.5% FITC solution (dissolved in acetone: dibutyl phthalate, 1:1, v/v) was painted on the shaved skin. On day 6, baseline ear measurements were taken and then mice were challenged with 20 μl 0.5% FITC on the right ear. On day 7, ear thickness was measured 24 hours post FITC challenge using digital calipers (Fowler). Change in ear thickness was expressed as ear thickness at day 7 minus ear thickness at baseline.

MC903 driven atopic dermatitis. Mice were sensitized daily for 14 consecutive days with 2 nmol of MC903 (calcipotriol; Tocris Bioscience) in 20 uL of 100% EtOH on ears. Baseline ear measurements were taken prior to the first ear sensitization on day 1 using Digital Calipers (Fowler). On day 14, ear thickness was measured. Delta change in ear thickness was expressed as ear thickness at day 14 minus ear thickness at baseline.

Ex vivo re-stimulation assays. Ear-draining cervical lymph nodes (CLNs), gut draining mesenteric lymph nodes (MLNs) and spleens were harvested at terminal time points from various studies and collected into 0.5 ml of cold, complete-RPMI (10% FBS, 1× Glutamax, 1 mM sodium pyruvate, 100 mM HEPES, 1× non-essential amino acids, 1× beta-mercaptoethanol, 1× antibiotic-antimycotic) (all reagents from Gibco). Single cell suspensions were prepared (spleens were RBC lysed with ACK lysing buffer) and 200,000 cells/well were plated. Cells were stimulated ex vivo with either LPS (200 ng/ml, Invivogen) or PMA (eBioscience) for 48 hours, or KLH (50 μg/ml, Sigma) or OVA (50 μg/ml) for 72 hours at 37° C. and 5% CO₂. Supernatants were collected at the end of stimulations and used for multiplex ELISAs of cytokine levels using Meso Scale Discovery kits. Ear tissues were dissociated in 250 μl T-PER buffer (Thermo Scientific) containing Halt Protease (Thermo Scientific) and protein was quantified with BCA kit (Thermo Scientific). 100 μg of protein was used to measure cytokine levels using MSD kits.

Human Macrophage assay. Frozen PBMCs from 3 different human donors were used for isolation of CD14+ macrophages. PBMCs were washed in 10 ml MACS buffer, spun down and resuspended at a concentration of 10⁷ total cells per 80 μl. Anti-CD14+ beads were added (20 μl per 10⁷ cells) and the cell suspension was incubated at 4° C. for 15 minutes. Following incubation, cells were washed and resuspended in MACS buffer and CD14+ cells were isolated using magnetic separation as per manufacturer's protocol (Miltenyi). Isolated cells were further cultured at 100,000 CD14+ cells/well in 100 μl and incubated at 37° C. overnight. 100 μl of GMCSF was added for final concentration of 50 ng/ml. Every other day 100 μl of the supernatant was removed and replaced with 100 μl of fresh GMCSF (100 ng/ml). Cells were cultured for 7 days. On Day 7, GMCSF was washed out and 100 ng/ml of LPS and IFNγ were added in antibiotic free medium. The culture was incubated for 18 hours prior to addition of microbes. Microbes were added in anaerobic conditions and flushed with 1% oxygen. Plates were incubated for 24 hrs in an anaerobic box at 37° C.+5% CO₂. After 24 hours, plated were centrifuged and supernatants collected to assay cytokine levels using MSD assays.

Human Cell Culture. Human Caco-2 (ATCC, HTB-37) and HT-29 MTX (Sigma, 12040401-1VL) colon epithelial cells were cultured and maintained in tissue culture treated T-175 flasks at 37° C. and 5% CO2. Medium (recipe in supplemental methods) was changed every 2-3 days by aspirating old medium and replaced with 30 ml of fresh, pre-warmed medium. At 90% confluency, cells were passaged after washes with 10 ml warm PBS, followed by 5 ml of 0.25% trypsin-EDTA. After incubation at 37° C. for 5-10 min, 30 ml of complete medium was added to the flask to inhibit the trypsin and single-cell suspension were counted. A co-culture suspension with 75,000 cells/ml comprising 60% Caco-2 and 40% HT-29 was prepared. 200 μl of this co-culture was added to the apical side of the membrane of a 24-well Transwell plate and 600 μl medium was added to the basal side of the membrane. Cells were cultured for 28 days with medium changes every 2-3 days to allow for epithelial barrier formation and cell polarization.

Human Epithelial Cell Line Microbe Stimulations. Stimulation of the transwells with microbes was performed 28 days after epithelial cells were plated. Epithelial barrier integrity was measured via transepithelial electrical resistance (TEER) using Millicell ERS-2 Voltohmmeter (Millipore Sigma, #MERS00002). Cells were washed with 600 μl basally and 200 μl apically of PBS. The same volumes of fresh PBS were then added. A basal resistance measurement was obtained prior to start of assay. On the day of the assay, TEER measurements were recorded for each well. PBS was then replaced with 600 μl of fresh antibiotic-containing medium basally and 150 μL of antibiotic-free media apically. 50 μL of a microbe suspension (at 1×10⁷ TCC/well or at the indicated concentration) or controls was added apically to each well. Cells were incubated for 24 h at 37° C. The plates were placed into flush boxes and flushed with 1% oxygen for 5 min. After 24 h, supernatants were collected for cytokine measurements by MSD U-plex assay. TEER was recorded for each well and the change in TEER was calculated from time zero to 24 h and reported as percent change compared to the sucrose vehicle control.

Statistical analysis. The data were expressed as mean±standard deviation. Statistical significance between groups was compared using the one-way ANOVA compared with sucrose-treated control. For statistical analyses of EAE data, the following tests were used for each readout: EAE incidence, Chi-square test; Mean day of EAE onset, 2-tailed Student's t-test; Median day of EAE onset, Wilcoxon's survival test; Average clinical score, 2-tailed Student's t-test; Average end clinical score, Wilcoxon's non-parametric test; Mean maximum score (MMS), Wilcoxon's non-parametric test; Average weight gain/loss, 2-tailed Student's t-test; End weight gain/loss, 2-tailed Student's t-test; Incidence of EAE relapse, Chi-square test; MMS of relapses, Wilcoxon's non-parametric test; MMS of relapse period, Wilcoxon's non-parametric test. Significance was assigned at p≤0.05, All statistical tests were performed using Prism 8 (GraphPad Software, San Diego, Calif., USA).

### Results

Prevotella histicola strain B treatment reduces type-1 inflammation and pro-inflammatory cytokines production in vivo and is dependent on IL-10

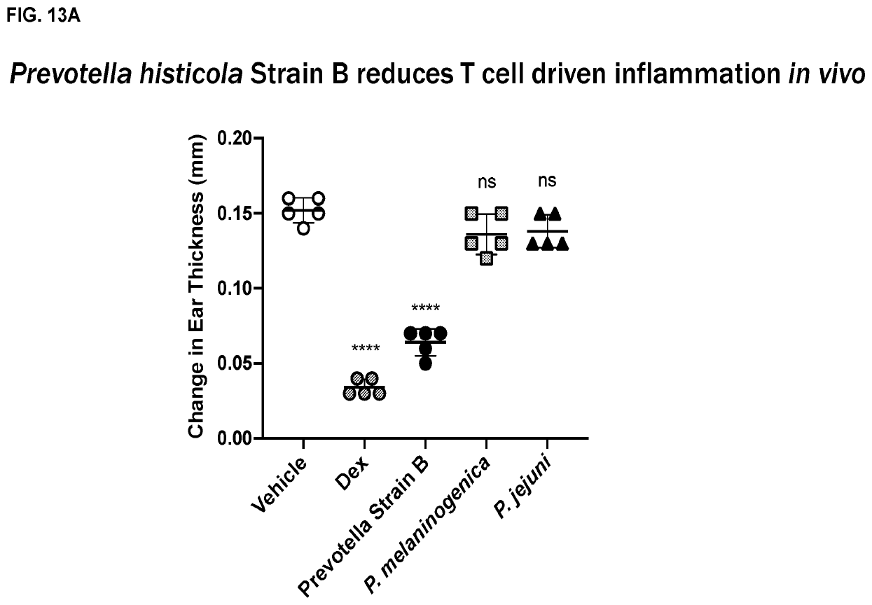

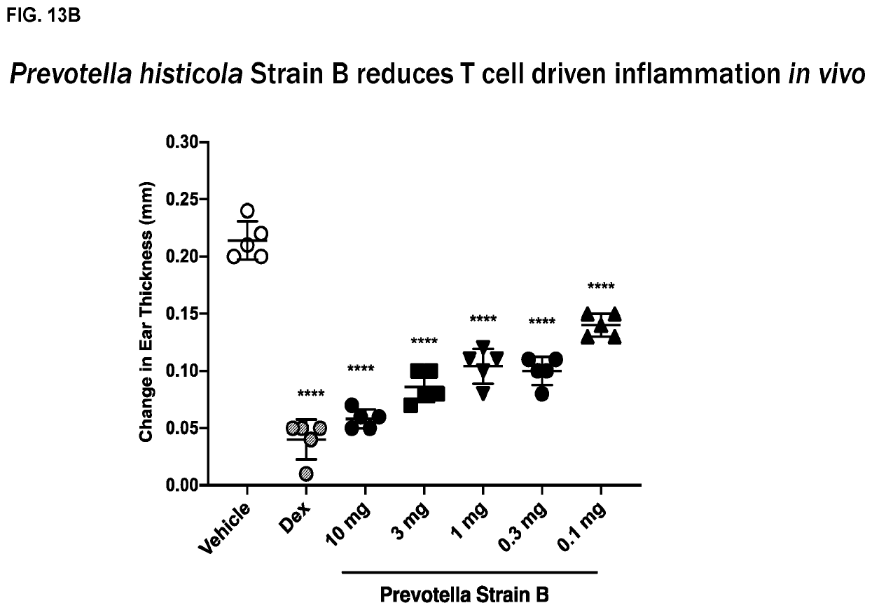

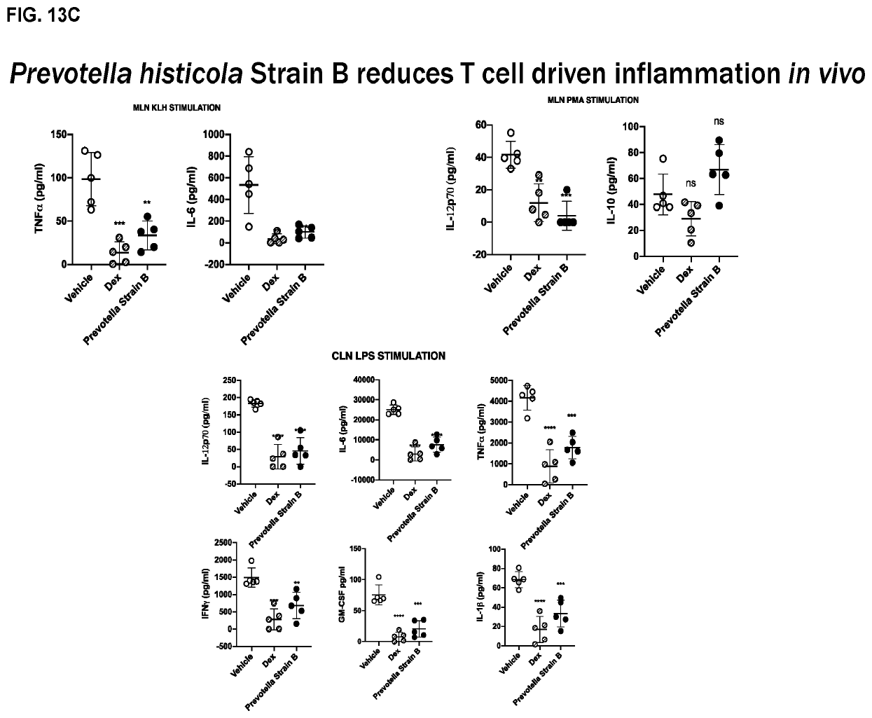

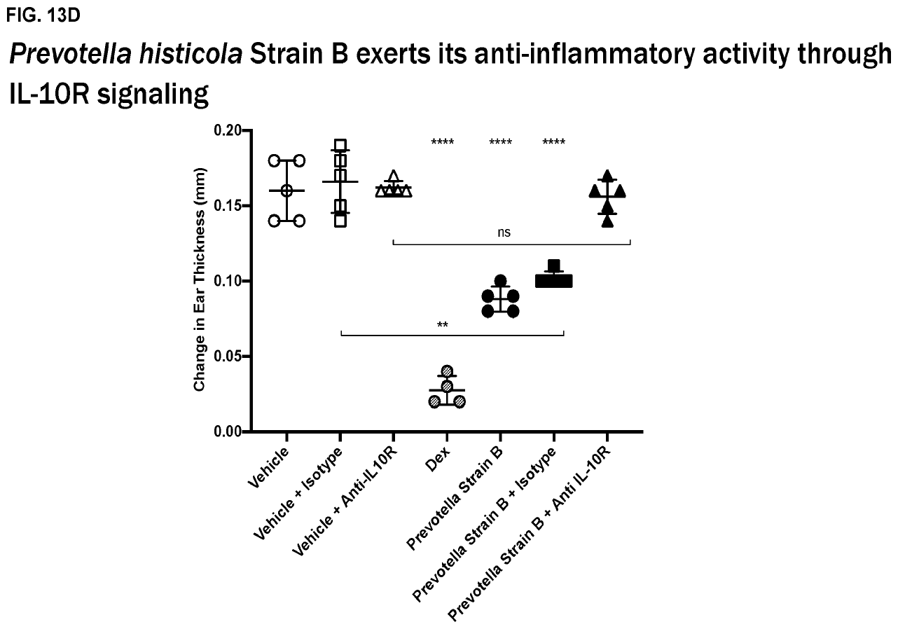

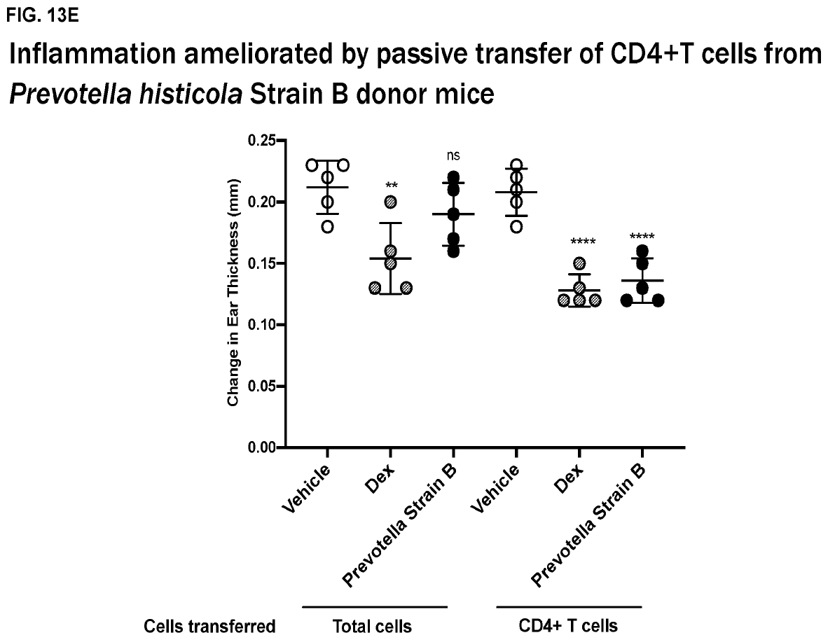

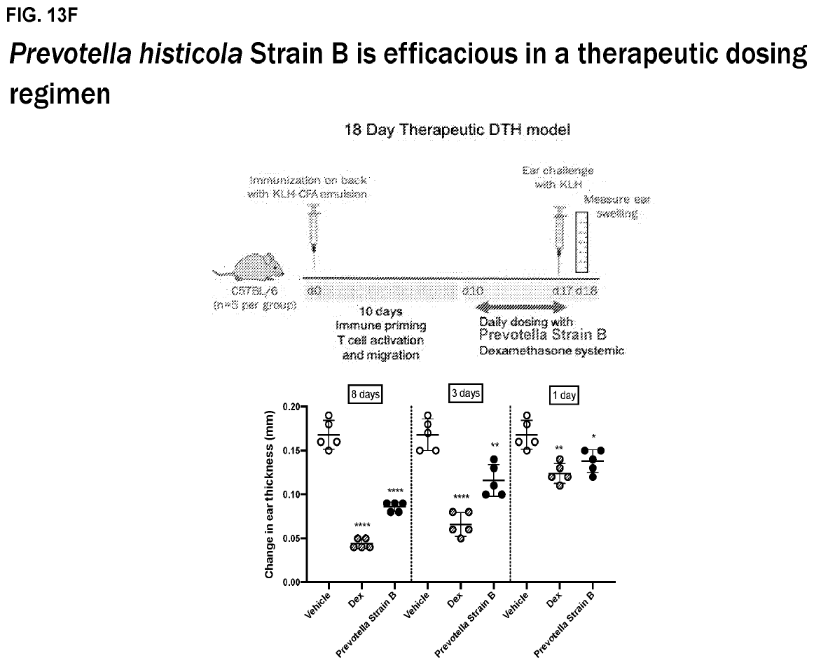

In a delayed type hypersensitivity (DTH) mouse model driven by keyhole limpet hemocyanin (KLH) protein, Prevotella histicola strain B dosed orally once a day and caused a significant suppression of ear inflammation 24 h after ear challenge on day 9. Prevotella histicola strain B was the most efficacious treatment in comparison to other species of Prevotella, P. jejuni and P. melaninogenica (FIG. 13A). Inhibition of inflammation was dose-dependent, across a range of doses from 10 mg to 0.1 mg demonstrating pharmacological effects (FIG. 13B).

Prevotella histicola strain B treatment modulated cellular production of cytokines. Cells from the spleen, gut-draining mesenteric lymph nodes (mLN) and ear-draining cervical lymph nodes (cLN) were taken from mice 24 h post-ear challenge and restimulated with lipopolysaccharide (LPS-to mimic microbial stimulation) or KLH (to represent antigenic stimulation) ex vivo. LPS-stimulated cells from mLN and spleen of vehicle treated mice produced significant amounts of proinflammatory cytokines, including TNFα and IL-6. In contrast, cells from mice treated with Prevotella histicola strain B had significantly reduced levels of pro-inflammatory cytokines and trends of increased IL-10 production suggesting that oral treatment with Prevotella histicola strain B had an adjacent anti-inflammatory effect on cells in the gut-draining mLN (FIG. 13C). In the cLN, treatment with Prevotella histicola strain B resulted in a similar trend of reduced pro-inflammatory cytokines TNFα, IL-6, GMCSF and IFNγ produced by LPS or KLH stimulation, compared to elevated levels of these cytokines in mice treated with vehicle (FIG. 13C).

The role of IL-10 in the anti-inflammatory effect of Prevotella histicola strain B was evaluated by blocking the IL-10 signaling pathway during the DTH. Based on reported efficacy in previous studies, an anti-IL-10 receptor antibody was used to block IL-10 signaling. Of the mice treated with Prevotella histicola strain B, the animals co-administered the anti-IL-10R antibody had significantly high ear inflammation in comparison with mice given the isotype antibody. The finding suggests that IL-10 is required for the modulation of ear inflammation in DTH by Prevotella histicola strain B (FIG. 13D).

Prevotella histicola strain B mediates its anti-inflammatory activity through CD4+T cells. Although Prevotella histicola strain B did not cause changes in frequency of cellular subsets, passive transfer of lymphocytes from Prevotella histicola strain B-treated donor mice induced with DTH-KLH into a second set of immunized but untreated recipient mice suppressed ear inflammation in the latter group of mice (FIG. 13E). This indicated that sustained dosing with Prevotella histicola strain B was not required for its effect. These data suggest that Prevotella histicola strain B could be altering functional responses in the CD4+ T cell which are retained such that these cells are sufficient to drive an anti-inflammatory response in the recipient mice.

Prevotella histicola strain B is effective in a therapeutic dosing regimen. Mice immunized with KLH-CFA were left in an immune priming phase without any therapeutic intervention for 10 days. Following this, they were dosed for either 1, 3 or 8 days prior to ear challenge and ear inflammation measured 24 h post challenge. As little as 1 day of dosing led to some reduction in inflammation. A more robust anti-inflammatory response was seen with 3 or 8 days of dosing (FIG. 13F). This suggested that Prevotella histicola strain B was equally effective in a prophylactic and therapeutic setting even with limited number of doses.

Altogether, these data indicate that Prevotella histicola strain B is efficacious lowering inflammation by inhibiting cytokine production from immune cells both locally in the gut as well as systemically by modulating CD4+ T cell responses in delayed type hypersensitivity model of inflammation.

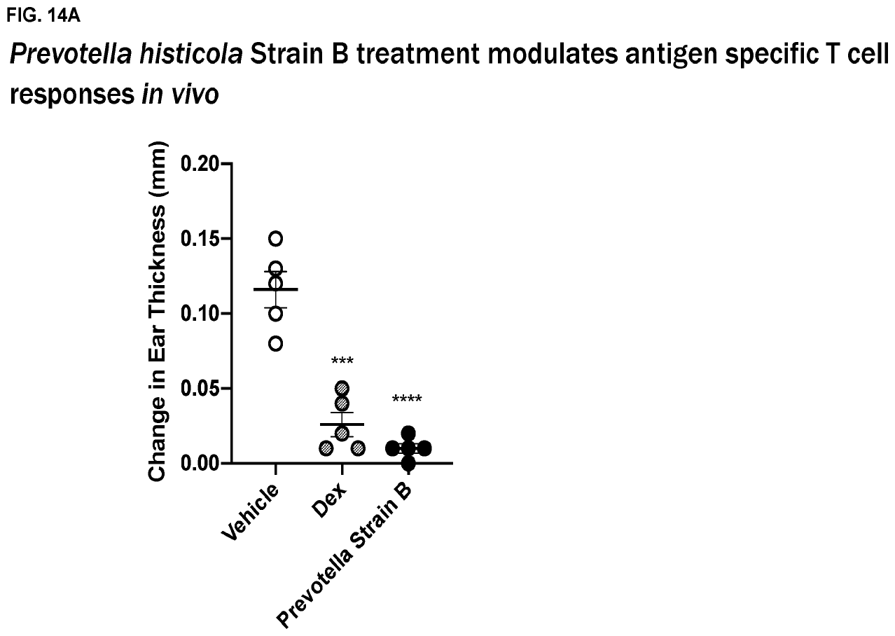

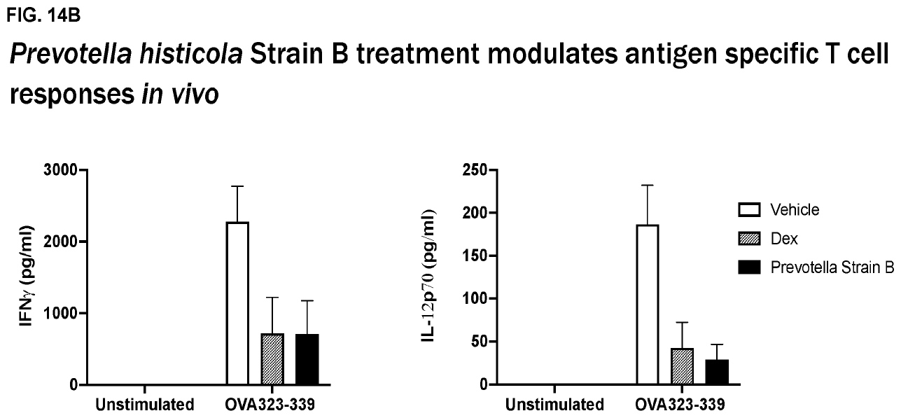

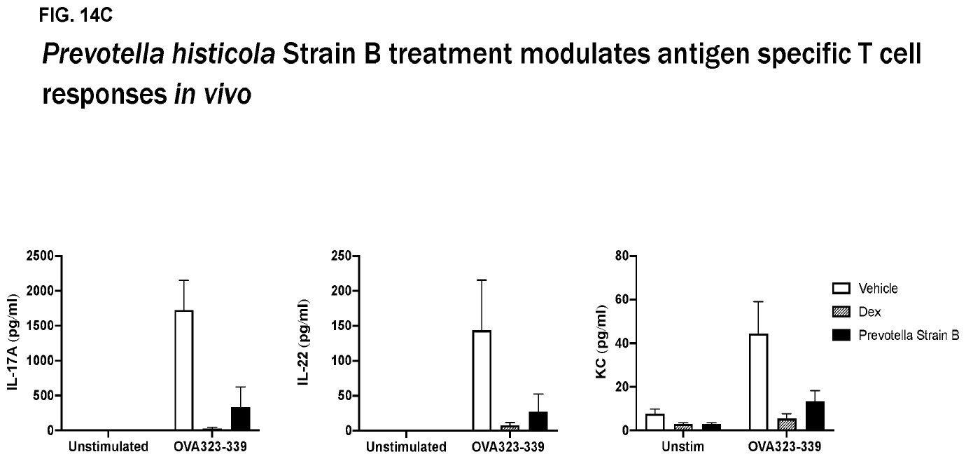

Prevotella histicola strain B treatment inhibits antigen specific T cell responses in vivo. Prevotella histicola strain B modulated antigen specific T cell mediated inflammation. An adoptive transfer model was used with DO11.10 TCR-Tg mice that express ovalbumin (OVA) peptide specific ab-TCR on their CD4+ T cells. Cells from donor DO11.10 TcR Tg mice were transferred into recipient BALB/c mice that were then immunized with OVA in CFA and challenged with OVA intradermally in the ear 9 days later to elicit local inflammation. Mice treated with Prevotella histicola strain B had significantly reduced ear inflammation in comparison with vehicle treated mice (FIG. 14A). To determine functional alteration in T cells in mice that received Prevotella histicola strain B treatment, cells from the ear draining cervical lymph nodes were re-stimulated ex vivo with the OVA 323-339 peptide that is recognized by the CD4+ T cell subset. This reduced levels pro-inflammatory cytokines IL-12p70 and IFNγ (FIG. 14B).

While the immunopathology of DTH is mainly attributed to Th1 cells, the role for Th17 cells has also been established in driving inflammation. Cytokines associated with the Th17 pathway such as IL-17A, IL-22 and KC (mouse homolog of IL-8), a chemokine involved in recruitment of neutrophils that contribute to ear inflammation, were also significantly reduced in the draining cLN by Prevotella histicola strain B treatment (FIG. 14C).

Prevotella histicola Strain B Alleviates Skin Pathology in Imiquimod-Induced Psoriasis

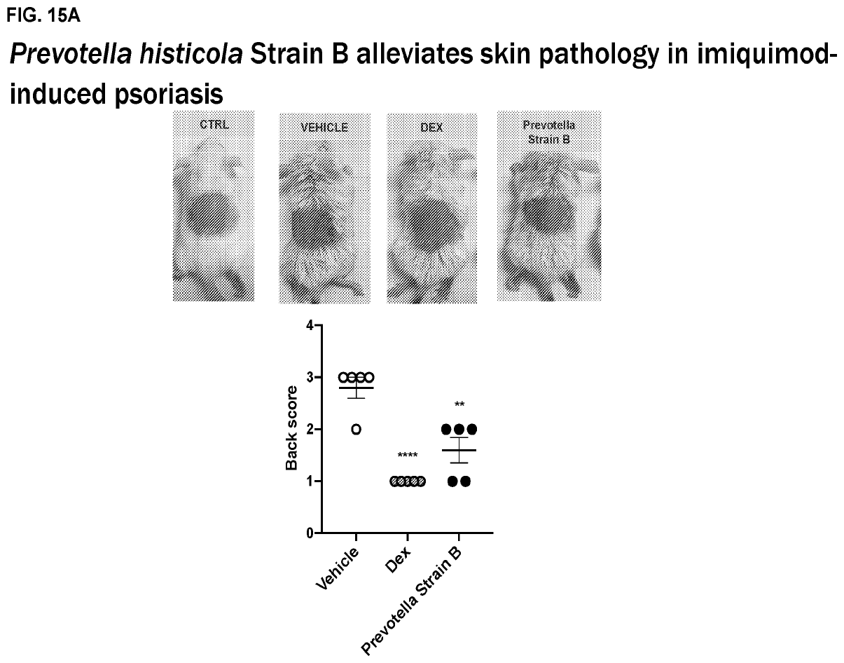

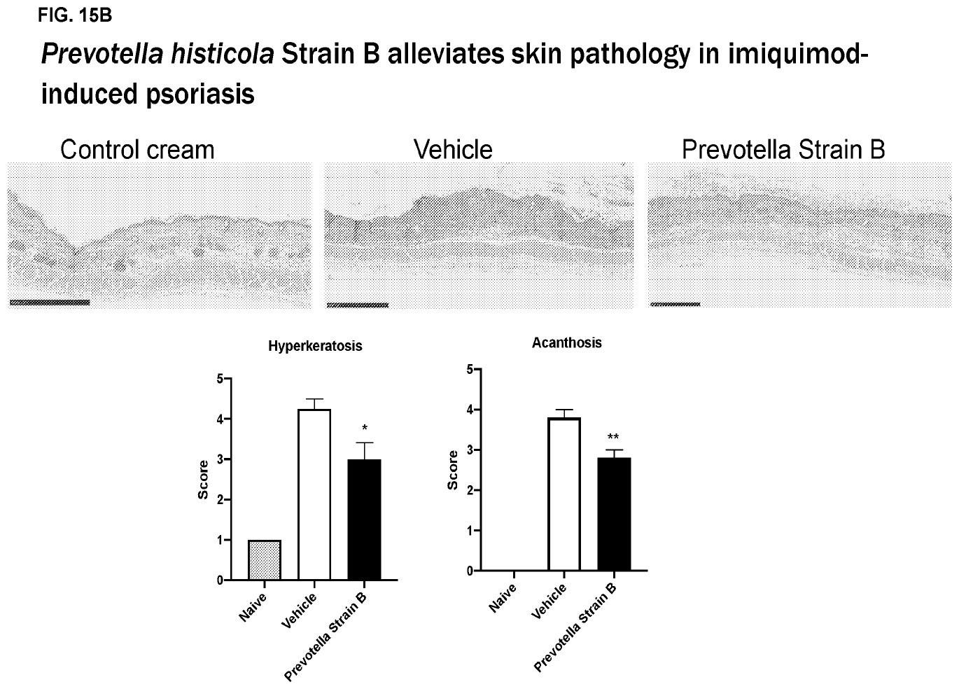

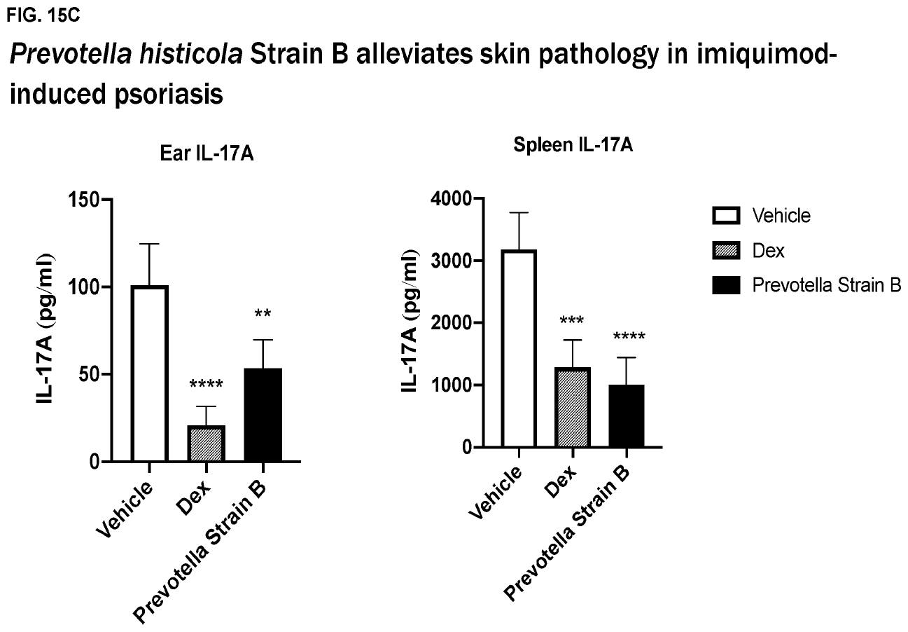

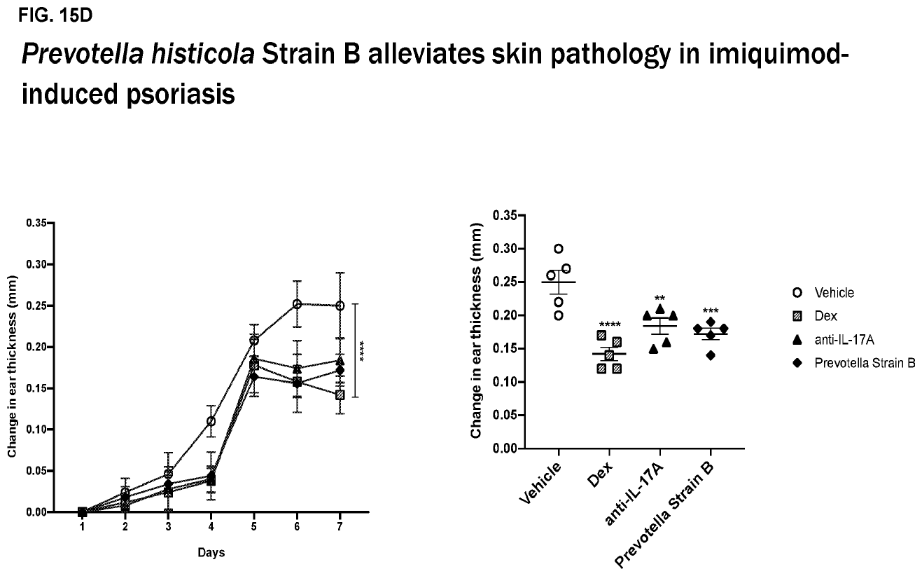

The diminished levels of Th17 pathway cytokines with Prevotella histicola strain B treatment suggested an anti-inflammatory role for Prevotella histicola strain B in diseases with a strong Th17 component. The immunopathology of psoriasis is driven by type 3 inflammatory pathways, particularly IL-23/IL-17A. Imiquimod (IMQ)-induced psoriasis is a well-established mouse model with clinical and histological characteristics similar to human psoriasis, such as epidermal thickening, scaling and erythema, infiltrates of T cells, neutrophils and dendritic cells. IMQ was applied daily for 7 days on the back skin and ears of BALB/c mice. On day 8, mice were scored as per the scoring scheme described in the methods. Prevotella histicola strain B treated mice showed visibly substantial suppression of erythema, scaling and thickening associated with IMQ-induced skin inflammation (FIG. 15A). H&E stained sections from IMQ-treated back skin showed decreased epidermal thickening or hyperkeratosis, and acanthosis, a sign of altered epidermal differentiation typical of psoriatic skin lesions, in the Prevotella histicola strain B treated mice compared to vehicle (FIG. 15B).

Protein levels in the ear tissue revealed a reduction in IL-17A levels upon treatment with Prevotella histicola strain B in comparison to vehicle. IMQ is known to also induce splenomegaly and concomitant increase in IL-17 production in splenocytes. Ex vivo re-stimulation of splenocytes with PMA/Ionomycin showed a decreased production of IL-17A (FIG. 15C).

Current treatments for psoriasis include anti-IL-17A biologics and corticosteroids. While these agents are effective in patients with moderate to severe psoriasis there is an unmet need for safe and effective anti-inflammatory options for patients with mild to moderate disease. Prevotella histicola strain B was compared with these agents. Efficacy was seen as early as 4 days after the start of IMQ application and was equally efficacious as dexamethasone and slightly more than anti-IL-17A in reducing ear as well as back inflammation. (FIG. 15D).

Collectively, these data demonstrate the efficacy of Prevotella histicola strain B in reducing type 3 skin inflammation and pathology in the IMQ-induced psoriasis model with effects that include reducing the levels of cytokines in the IL-23/IL-17A axis.

Treatment with Prevotella histicola Strain B Ameliorates Neuroinflammation in Murine Model of Relapsing-Remitting Multiple Sclerosis

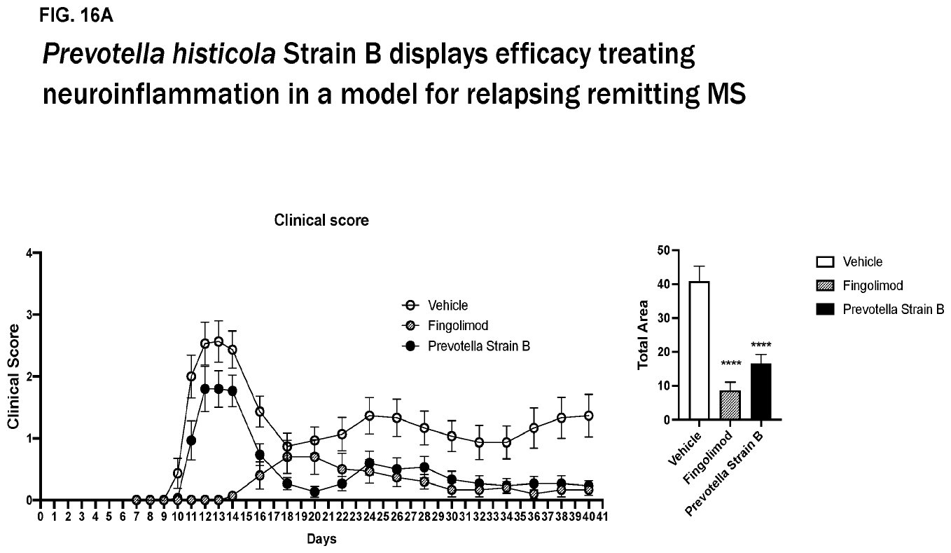

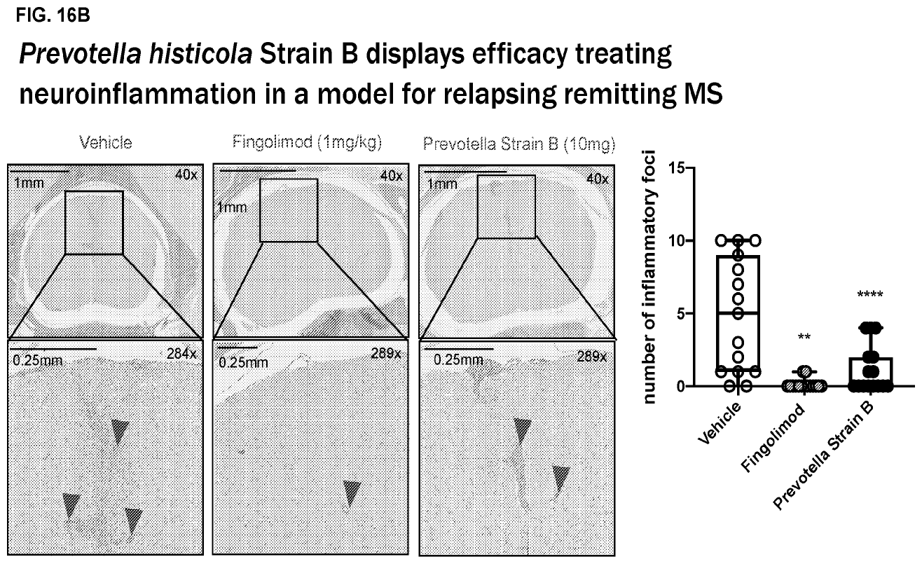

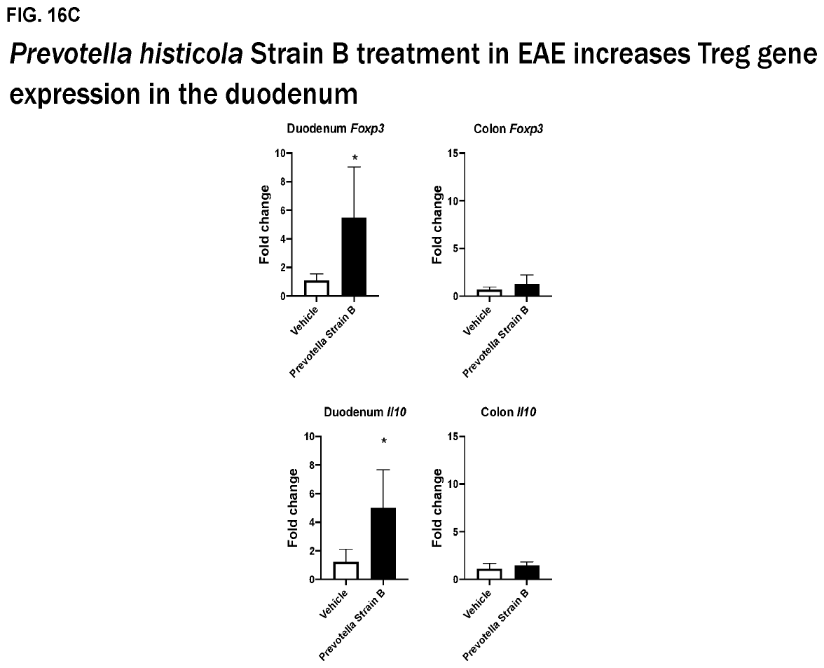

Building further on the role for Prevotella histicola strain B in controlling Type 3/Th17 driven pathology, Prevotella histicola strain B was tested in an experimental autoimmune encephalomyelitis (EAE) model of relapsing remitting multiple sclerosis. In a prophylactic setting, mice treated with Prevotella histicola strain B showed a reduced average clinical score compared to vehicle treated animals over the course of the disease (FIG. 16A). The effect was most pronounced in the relapsing phase of the disease. Prevotella histicola strain B treated mice also showed a lower cumulative EAE score compared to the control group as seen from the area under the curve graph (FIG. 16A).

Treatment with Prevotella histicola strain B was associated with reduced CNS pathology. Sections of spinal cord tissue were analyzed from EAE mice that received treatment with Prevotella histicola strain B, a positive control drug fingolimod and a vehicle treated group. Mice treated with Prevotella histicola strain B showed reduced neuroinflammation as well as significantly reduced frequency of infiltrating inflammatory cells in the spinal cord compared to vehicle treated animals (FIG. 16B). These data indicate that Prevotella histicola strain B is effective in suppressing disease in a relapsing remitting form of EAE, consistent with previously reported results in a non-relapsing EAE model.

Prevotella histicola Strain B Modulates Treg and IL10 Gene Expression in the Murine Small Intestine

Extended treatment for 41 days with Prevotella histicola strain B in the EAE model presented the opportunity to study changes in the gut immune environment. Intestinal transcriptional differences were determined by qPCR analysis from different segments of the intestine from mice treated with Prevotella histicola strain B or vehicle. Prevotella histicola strain B treatment increased the expression of Treg specific genes Foxp3 and Il10 in the duodenal sections compared to vehicle treated mice. In contrast, no differential Foxp3 and Il10 gene expression was observed in the colon (FIG. 16C). These data demonstrate that Prevotella histicola strain B modulates effects in the small intestine to drive responses that alleviate inflammatory pathology in the CNS. This is a remarkable observation that the small intestine is an immune portal to the CNS.

Prevotella histicola Strain B Drives IL-10 Production in Primary Human Macrophages and Improves Epithelial Barrier Integrity

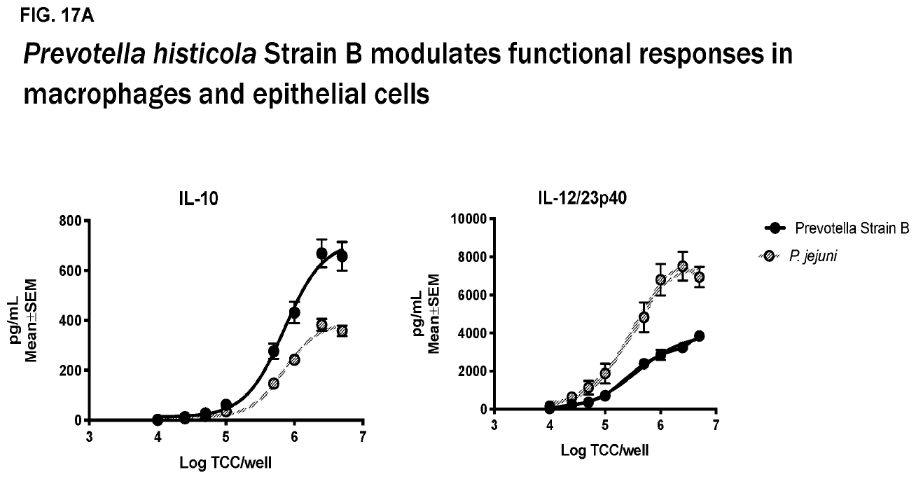

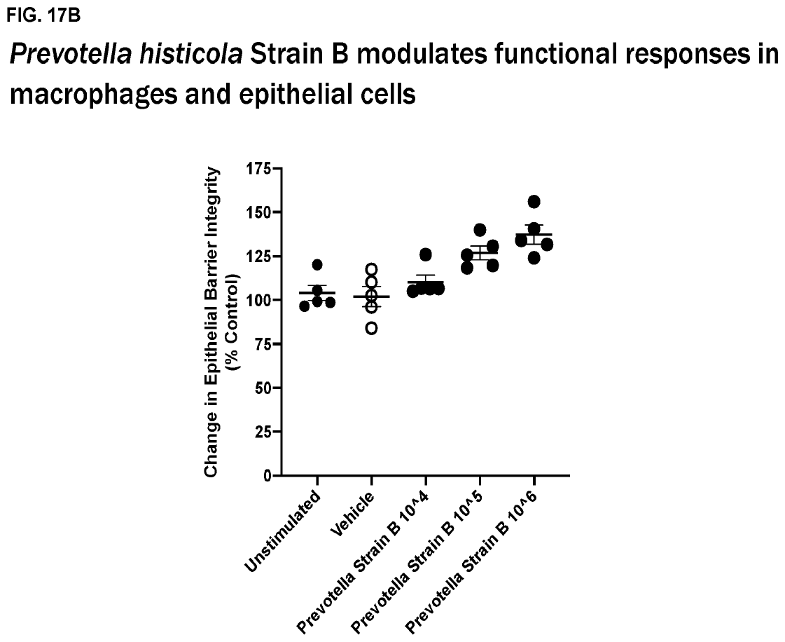

Given the transcriptional changes observed in the duodenal and colonic sections, Prevotella histicola strain B was tested in in vitro to delineate its role in driving functional responses in immune and epithelial cells. In primary human macrophages (HuMACs), Prevotella histicola strain B induced the production of IL-10 while inhibiting IL12p70 secretion in a dose dependent manner. In accordance with the in vivo data (FIG. 13B), cytokine profiles displayed a microbial strain and species specificity, with Prevotella histicola strain B more anti-inflammatory than another species from the same genus, P. jejuni had an inverse cytokine secretion profile (FIG. 17A).

As Prevotella histicola strain B is an oral therapy, a predominant cell type encountered in the gut are epithelial cells. An in vitro polarized human epithelial CaCO2-HT29 Transwell coculture system was used to determine the effect of Prevotella histicola strain B on epithelial barrier integrity prior to and after incubation with Prevotella histicola strain B for 24 hours. Epithelial barrier was assessed via measurement of transepithelial electrical resistance (TEER). Prevotella histicola strain B treated cells induce around a 50% increase in TEER values in comparison to vehicle treated which displayed the fortification of barrier integrity, by Prevotella histicola strain B (FIG. 17B).

Prevotella histicola Strain B Reduces Cutaneous Inflammation in Murine Models of Type 2 Inflammation (Atopic Dermatitis)

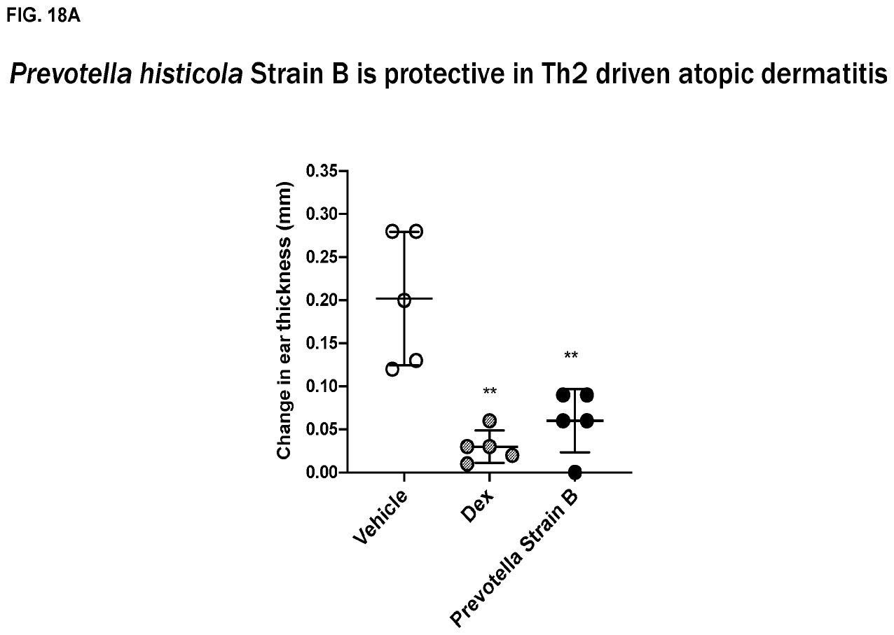

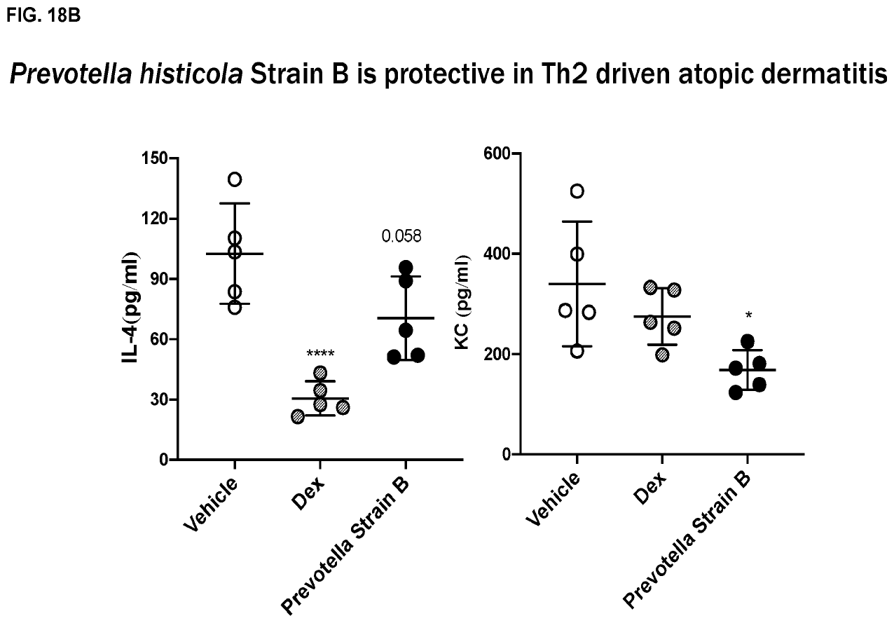

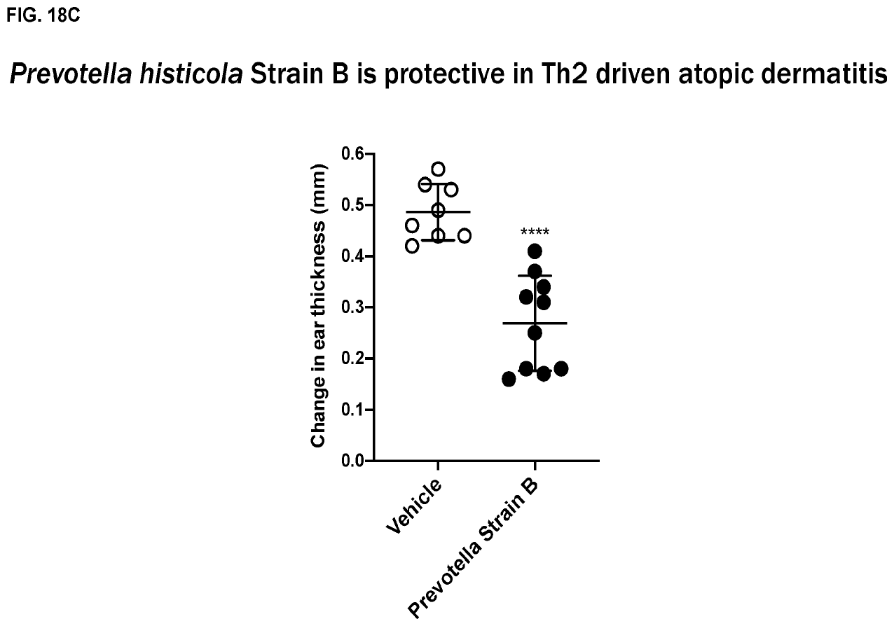

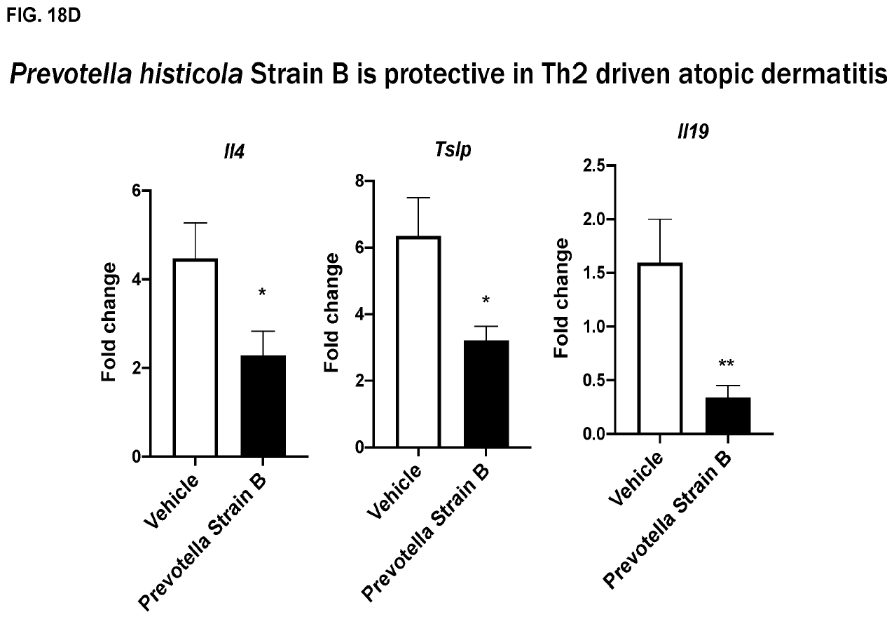

The efficacy of Prevotella histicola strain B in type 1 and type 3 inflammatory pathway suggested that it may also be effective in type 2 inflammatory allergic or atopic diseases. These encompass a family of conditions that include atopic dermatitis (AD), allergic rhinitis, asthma and food allergy that are driven by the Th2 and ILC2 cells among others, as well as cytokines including IL-4, IL-5, IL-13, and alarmins such as IL-33 and TSLP. Prevotella histicola strain B was tested in a murine model of atopic inflammation, using contact hypersensitivity to the hapten fluorescein isothiocyanate (FITC). In this model, mice are sensitized with FITC on days 1 and 2 and receive an ear challenge 6 days post sensitization on the ear. This model has similarities to human AD. It is dependent on CD4+T helper cells and the pathology associated with the disease recapitulates features of acute AD lesions. Oral treatment with Prevotella histicola strain B inhibited ear inflammation 24 hours post FITC challenge when compared to vehicle treatment (FIG. 18A). Cytokine levels were measured in tissue homogenates from ears harvested 24 h post-FITC challenge. Levels of IL-4, an essential Th2 cytokine and KC, a neutrophil attracting chemokine that drives edema associated with inflammation, were inhibited in Prevotella histicola strain B treated animals (FIG. 18B).

An important cytokine alarmin responsible for triggering type 2 inflammation biology is thymic stromal lymphopoietin (TSLP) which is produced by damaged keratinocytes in AD. TSLP induces the expression of type 2 inflammatory cytokines, such as IL-4, IL-5 and IL-13. It has been reported that TSLP can act directly on naïve CD4+ T cells to promote Th2 differentiation during allergic inflammation in the skin. An experimental model for AD induced with topical application of a vitamin D3 analog, MC903, can induce TSLP with changes in skin morphology and inflammation resembling immune features observed in lesions of patients with AD. This model was used to study the effect of Prevotella histicola strain B in lowering cutaneous inflammation associated with AD. Mice with MC903-driven AD treated with Prevotella histicola strain B exhibited decreased ear inflammation (FIG. 18C). There was also a substantial decrease in RNA levels compared with the vehicle-treated cohort of Tslp, Il4 and Il19 (FIG. 18D). These observations underscore that Prevotella histicola strain B can also play an immunomodulatory role in regulating allergen-induced type 2 cutaneous inflammation.

A Non-Replicating Form of Prevotella histicola Strain B has Pharmacological Activity and is Efficacious in Modulating Inflammation

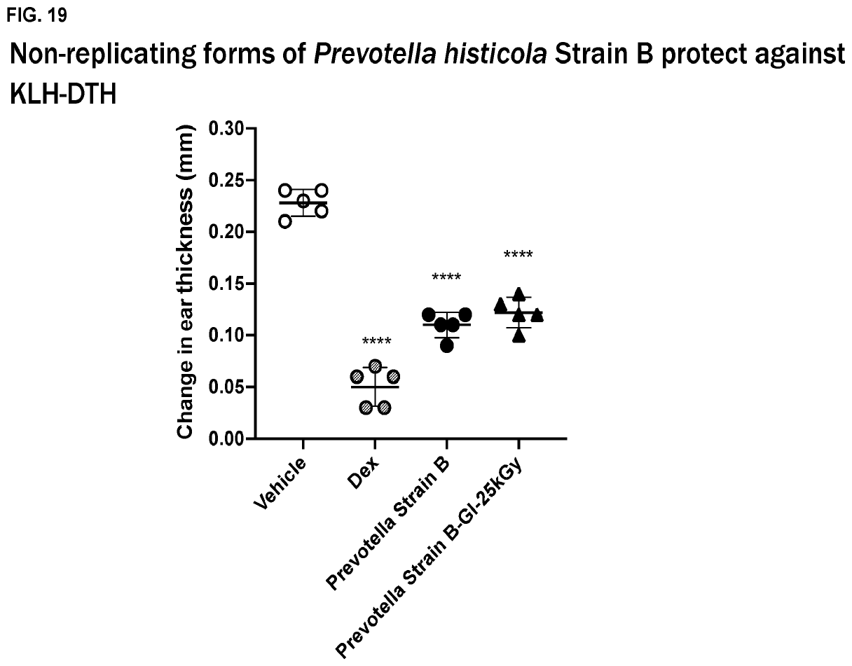

To determine if the therapeutic benefit required microbial colonization of the gut a non-replicating form of Prevotella histicola strain B was tested as an oral therapy. DTH induced mice were treated with a gamma-irradiated form of Prevotella histicola strain B. Treatment with this non replicating form resulted in significant efficacy in lowering ear inflammation upon challenge with the KLH antigen (FIG. 19). Ex vivo analysis of after treatment with Prevotella histicola strain B. GI yielded variable effects in the lymph nodes and spleen.

### Example 8: TLR2 Signaling is Involved in Prevotella histicola Strain B Mechanism of Action

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with Prevotella histicola strain B (10 mg powder) or dosed intraperitoneally with dexamethasone (positive control) at 1 mg/kg from days 5-8. Treatments of anti-TLR2 antibody and isotype control (IgG1) were given at 200 ug/mouse on days 0, 3 and 6. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours.

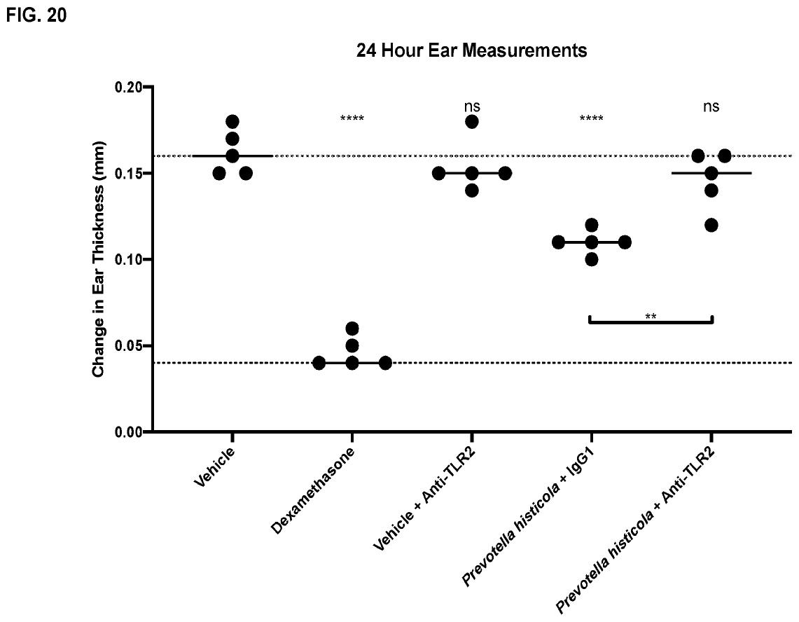

The 24-hour ear measurement results are shown in FIG. 20 (Prevotella histicola strain B is labeled as “Prevotella histicola” in the figure). Anti-TLR2 antibody treatment resulted in a reduction in efficacy for Prevotella histicola strain B, demonstrating that TLR2 is an important factor in the MOA for Prevotella histicola strain B in its reduction of inflammation in the DTH model.

### Further Example

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with Prevotella histicola strain B (10 mg powder) or dosed intraperitoneally with dexamethasone (positive control) at 1 mg/kg from days 5-8. Treatments of anti-TLR2 antibody and isotype controls (IgG1) were given at 200 ug/mouse on days 0, 3 and 6. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours.

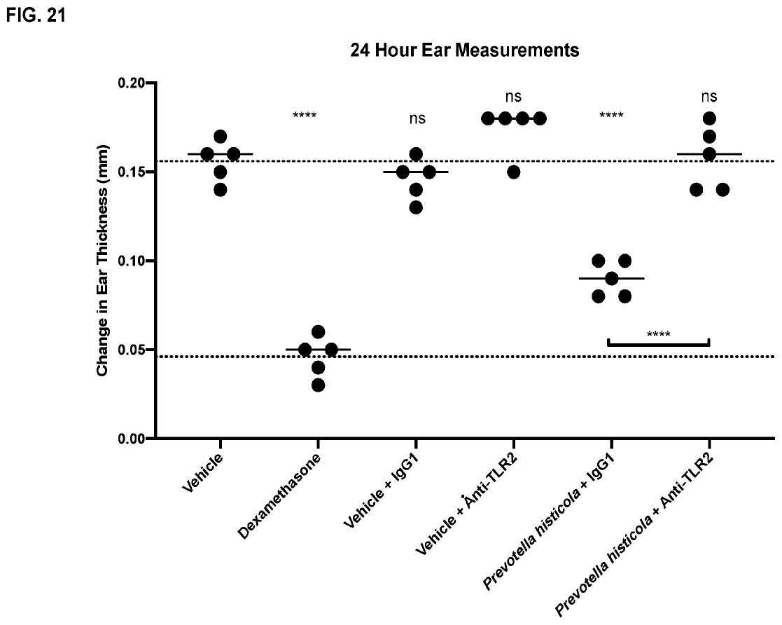

The 24 hour ear measurement results are shown in FIG. 21 (Prevotella histicola strain B is labeled as “Prevotella histicola” in the figure). Consistent with the results shown in FIG. 20, anti-TLR2 antibody treatment resulted in a reduction in efficacy for Prevotella histicola strain B, demonstrating that TLR2 is an important factor in the MOA for Prevotella histicola strain B in its reduction of inflammation in the DTH model.

### Example 9: Prevotella histicola Strain B Shows Increased Efficacy after 30 Days of Dosing

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with 10 mg Prevotella histicola strain B powder or dosed intraperitoneally with dexamethasone (positive control) at 1 mg/kg from days 1-30 on weekdays only. After dosing on day 15 and day 30, mice were anaesthetized with isoflurane, left ears were measured for baseline measurements with Fowler calipers and the mice were challenged intradermally with KLH in saline (10 μl) in the left ear and ear thickness measurements were taken at 24 hours.

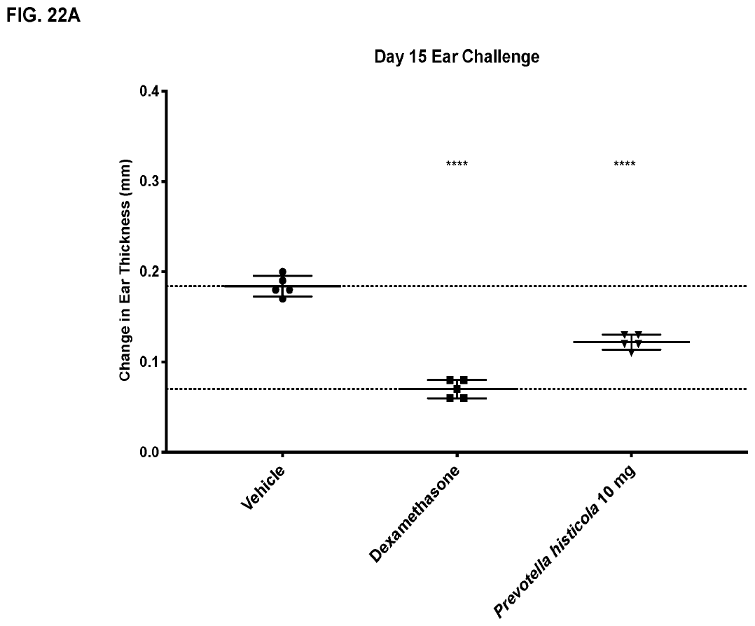

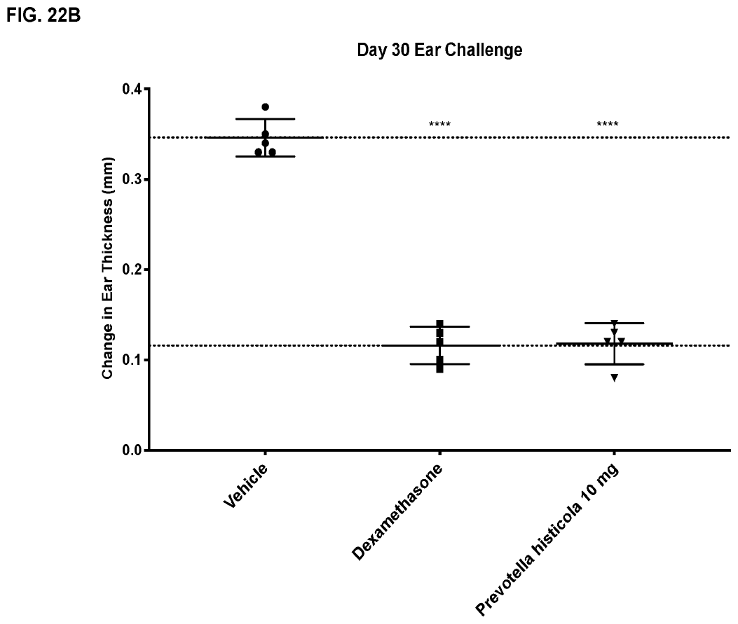

The 24 hour ear measurement results for a challenge on day 15 and a challenge on day 30 are shown in FIGS. 22A and 22B (Prevotella histicola strain B is labeled as “Prevotella histicola” in the figures). Prevotella histicola strain B showed increased efficacy after 30 days of dosing.

### Further Example

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with Prevotella histicola strain B (10 mg powder) or dosed intraperitoneally with dexamethasone (positive control) at 1 mg/kg from days 1-30 on weekdays only. Five separate powder preparations of Prevotella histicola strain B were tested. The same dose was tested for each powder preparation. After dosing on day 15 and day 30, mice were anaesthetized with isoflurane, left ears were measured for baseline measurements with Fowler calipers and the mice were challenged intradermally with KLH in saline (10 μl) in the left ear and ear thickness measurements were taken at 24 hours.

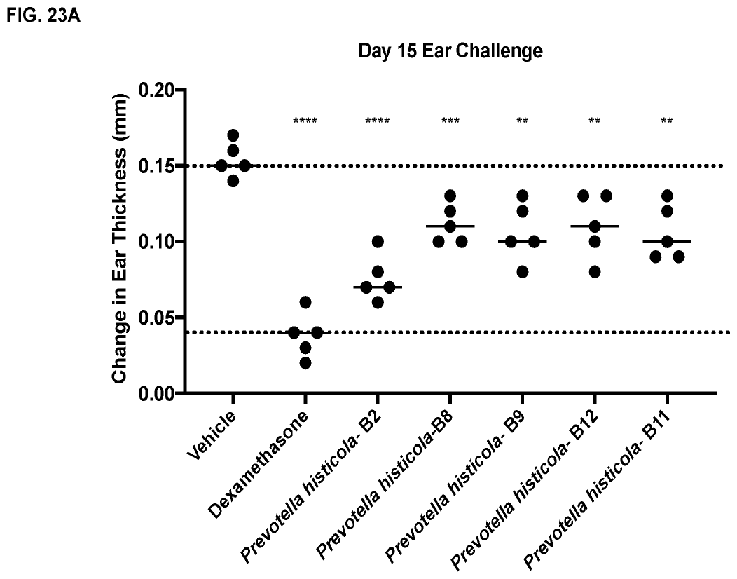

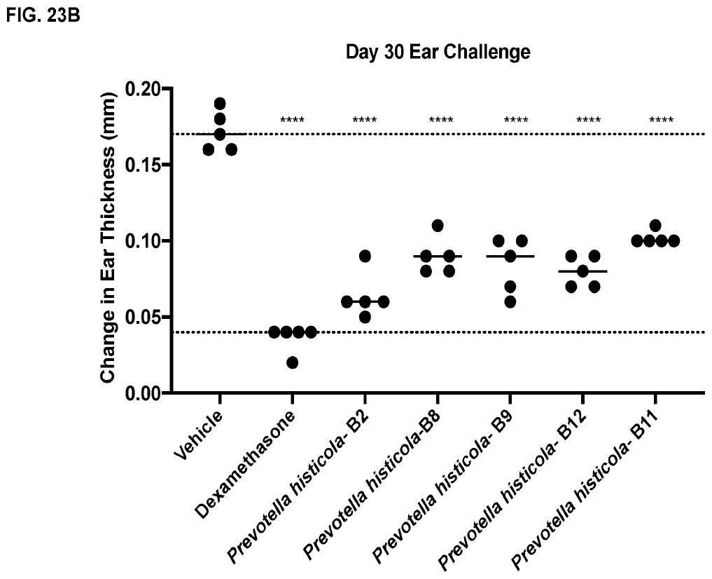

The 24 hour ear measurement results for a challenge on day 15 and a challenge on day 30 are shown in FIGS. 23A and 23B (Prevotella histicola strain B is labeled as “Prevotella histicola” in the figure). Prevotella histicola strain B showed increased efficacy after 30 days of dosing for all batches of Prevotella histicola powders.

### Example 10: Persistence of Efficacy of Prevotella histicola Strain B

Female 9 week old C57BL/6 mice were purchased from Taconic Biosciences and acclimated at a vivarium for one week. Mice were primed with an emulsion of KLH and CFA (1:1) by subcutaneous immunization on day 0. Mice were anesthetized daily from days 1-8 and orally gavaged with Prevotella histicola strain B powder at 10 mg or 1.82 mg, dosed intraperitoneally with dexamethasone (positive control) at 1 mg/kg, or dosed with a 1.82 mg uncoated solid dose form (MMT) of Prevotella histicola strain B (by placing an MMT on a rat feeding needle attached to a syringe filled with 50 ul of diH20 at pH 4.2, ejecting directly into the esophagus of the anesthetized mouse and then tamping down with a stainless steel disposable mouse feeding needle). After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours. Then the mice were left alone for 2 weeks and ear challenged and measured 24 hours after that.

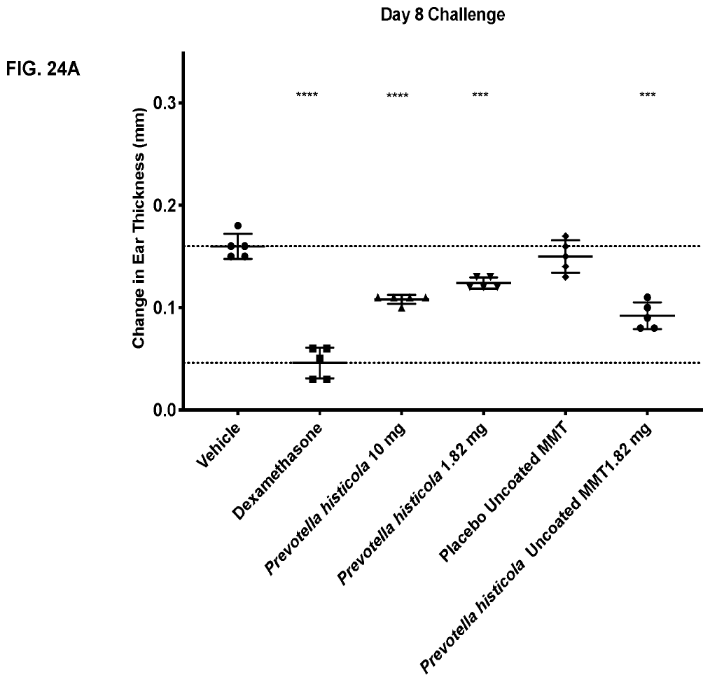

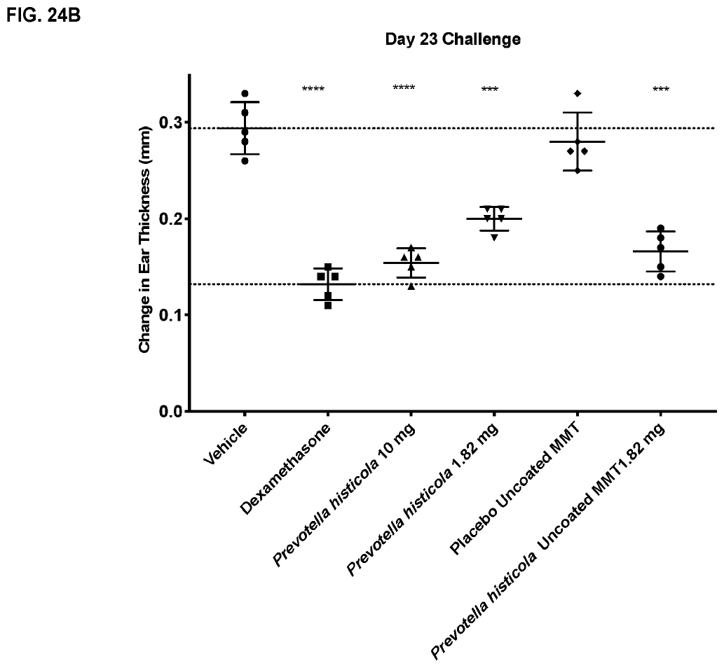

The 24 hour ear measurements from the challenge on day 8 and the challenge on day 23 are shown in FIGS. 24A and 24B. Oral dosing with Prevotella histicola in powder and MMT form show efficacy after 8 days of dosing (FIG. 24A) and this efficacy persists for at least 2 more weeks without any additional doses of the microbe (FIG. 24B).

### Example 11: A Phase 2, Multicenter, Randomized, Double-Blind, Placebo-Controlled, Parallel-Cohort, Dose-Ranging Study Investigating the Effect of Prevotella Strain B 50329 in the Treatment of Mild to Moderate Plaque Psoriasis Example

Rationale:

A therapeutic agent that offers the potential of systemic immune system modulation following oral administration, without systemic exposure is being developed. Prevotella Strain B 50329 is a pharmaceutical preparation of a strain of Prevotella histicola isolated from a human duodenal biopsy: it has not been genetically modified.

Studies of Prevotella Strain B 50329 in vitro in a range of human and mouse assays and studies in vivo in model symptoms support the use of Prevotella Strain B 50329 in the treatment of inflammatory diseases including psoriasis.

Oral administration of Prevotella Strain B 50329 to mice results in striking pharmacodynamic effects on animal models of delayed-type hypersensitivity, fluorescein isothiocyanate cutaneous hypersensitivity, collagen-induced arthritis and experimental acute encephalomyelitis. The high degree of consistency of both effect and dose suggests the potential for clinical benefit across multiple type 1, type 2, and type 3 inflammatory conditions. No potentially related adverse effects were seen in the animals used in these experiments with daily dosing for up to 3 weeks or with alternate day dosing for over 7 weeks. Immunophenotyping ex vivo in these models shows increased regulatory T cell numbers and regulatory dendritic cells in spleen and mesenteric lymph nodes, as well as decreases in pro-inflammatory cytokines such as IL-23p40, IL-17, TNF, IL-6, and IL-13. Treatment also led to enhancement of gut intestinal barrier integrity, which is often disrupted in patients with inflammatory diseases. These effects on immune parameters have been observed both within and outside the GI tract, which demonstrates that host-microbe interactions in the gut can affect the immune response in peripheral tissues.

Psoriasis is a chronic immune-mediated type 1/3 inflammatory skin disease in which hyperactive T cells trigger excessive keratinocyte proliferation. This results in the formation of raised erythematous plaques with scaling. Psoriatic lesions can appear anywhere on the body but are most often seen on the knees, elbows, scalp, and lumbar area. Critical events in the inflammatory process include activation of Langerhans cells and T cells, selective trafficking of activated T cells to the skin, and induction of an inflammatory cytokine and chemokine cascade in skin lesions. Clinical data have validated the role of anti-TNFα, anti-IL-17, and anti-IL-23 therapy in moderate to severe psoriasis. For patients with mild to moderate psoriasis, therapy usually involves topical agents (topical corticosteroids, vitamin D3 analogs), with topical corticosteroids providing the greatest range of efficacy and a wide range of formulations. More recently, physicians are prescribing apremilast, a first-in-class oral PDE4 inhibitor, ahead of biological therapy, which includes etanercept, infliximab, adalimumab, ustekinumab, and secukinumab.

In another study, a total of 56 participants have participated in Cohorts 1-4, with 36 of these participants receiving Prevotella Strain B 50329 once daily; 16 of these were treated for 14 days (healthy volunteers) and 20 participants with psoriasis were treated for 28 days. Clinical responses, similar to apremilast and tofacitinib at the same time point, have been observed on the (psoriatic) LSS and PASI at Day 28. Furthermore, at the Day 42 follow-up visit, when participants were “off treatment” for 14 days, there was continued improvement in the pharmacodynamic response as measured by both LSS and PASI for participants administered the higher dose, but not the lower dose, suggesting a dose relationship on the durability of effect. The safety profile of Prevotella Strain B 50329 was similar to placebo, with no SAEs or AEs of severe intensity.

The evidence available so far suggests Prevotella Strain B 50329 is very well tolerated and it continues to undergo clinical development in mild to moderate psoriasis. A well-tolerated oral therapy could offer significant benefit in the treatment of psoriasis and it is presently anticipated that Prevotella Strain B 50329 would be used in established but early disease, before the use of biologic therapies.

This Phase 2 study has been designed to investigate the clinical safety and efficacy of Prevotella Strain B 50329 and to identify an optimal dose.

Objectives:

Primary Objective:

The primary objective of this study is to evaluate the safety and efficacy of 3 different doses of Prevotella Strain B 50329 for the treatment of psoriasis following daily dosing for 16 weeks.

### Secondary Objectives:

The secondary objectives of this study are the following:
To evaluate the efficacy dose response of Prevotella Strain B 50329 at Week 16
To evaluate the maximal clinical benefit of Prevotella Strain B 50329 at Week 16
To evaluate the optimal dose of Prevotella Strain B 50329 based on efficacy and safety up to Week 16
To evaluate the safety and tolerability of Prevotella Strain B 50329 (all dose levels) throughout the study

Exploratory Objectives:

The exploratory objectives of this study are the following:
To evaluate the time to onset of clinical response to Prevotella Strain B 50329
To evaluate the effect of Prevotella Strain B 50329 treatment on patient-reported outcomes including quality of life and pain
To evaluate the effect of Prevotella Strain B 50329 treatment on biomarkers in blood
To evaluate the effect of Prevotella Strain B 50329 treatment on biomarkers in skin plaques
To evaluate the effect of Prevotella Strain B 50329 treatment on fecal microbiome composition

Estimands:

Primary Estimands

The primary estimand will be the effect of Prevotella Strain B 50329 on the percent change in PASI score from baseline to Week 16 in the modified intent-to-treat (mITT) set of all treated participants, regardless of deviations from the protocol. Only data collected while on treatment will be used to account for the intercurrent event of treatment discontinuation (hypothetical strategy based on adherence to treatment only). The posterior mean difference between each active dose and the pooled placebo will be estimated.

For the primary analysis, 2 supportive estimands will also be considered:
To assess the impact of intercurrent events related to protocol deviations believed to impact efficacy, a supportive analysis will be performed where all data collected after any such identified protocol deviations will be excluded. Participants who had a protocol deviation believed to impact efficacy prior to the first dose of treatment (hypothetical strategy based on adherence to treatment and the aspects of the protocol which impact efficacy) will be completely excluded from this analysis.
   To assess the impact of treatment discontinuation, a supportive analysis will be performed in which all data collected during the study, including any data collected after treatment discontinuation will be included (treatment policy strategy).

Summary of Secondary Estimands:

For all secondary estimands, the population of interest will be the MITT set.

| | Consideration | |
|---|---|---|
| | of intercurrent | Summary |
| Endpoint | events | measure |
| Mean percentage | To include all data | Posterior mean |
| change from | collected prior to | difference between |
| baseline in | treatment | each active |
| PASI Score at | discontinuation, | treatment group |
| Weeks 4, 8, | regardless of | and pooled placebo |
| and 12 | protocol deviations | at each visit |
| Mean absolute | To include all data | Posterior mean |
| change from | collected prior to | difference between |
| baseline in | treatment | each active |
| PASI Score at | discontinuation, | treatment group |
| Weeks 4, 8, | regardless of | and pooled placebo |
| 12, and 16 | protocol deviations | at each visit |
| Achievement of | To include all data | Posterior odds |
| PASI-50 at | collected prior to | ratio for |
| Weeks 4, 8, | treatment | response between |
| 12, and 16 | discontinuation, | each active |
| | regardless of | treatment group |
| | protocol deviations | and pooled placebo |
| | | at each visit |
| Time to first | To include all data | Hazard ratio |
| achievement of | collected prior to | for each |
| PASI-50 | treatment | active group |
| | discontinuation, | versus placebo |
| | regardless of | |
| | protocol deviations | |
| Achievement of | To include all data | Proportion |
| PASI-75, | collected prior to | of response |
| PASI-90 and | treatment | for each |
| PASI-100 at | discontinuation, | endpoint in each |
| Week 16 | regardless of | treatment group |
| | protocol deviations | at each visit |
| Achievement of | To include all data | Posterior odds |
| PGA of 0 or 1 | collected prior to | ratio for response |
| with a ≥2-point | treatment | between each |
| improvement | discontinuation, | active treatment |
| from baseline at | regardless of | group and |
| Week 16 | protocol deviations | pooled placebo |
| Achievement of | To include all data | Posterior odds |
| PGA of 0 at | collected prior to | ratio for response |
| Week 16 | treatment | between each |
| | discontinuation, | active treatment |
| | regardless of | group and |
| | protocol deviations | pooled placebo |
| Mean percentage | To include all data | Posterior mean |
| change from | collected prior to | difference |
| baseline in | treatment | between each |
| PGA × BSA at | discontinuation, | active treatment |
| Weeks 4, 8, | regardless of | group and |
| 12, and 16 | protocol deviations | pooled placebo |
| | | at each visit |
| Mean absolute | To include all data | Posterior mean |
| change from | collected prior to | difference |
| baseline in | treatment | between each |
| PGA × BSA at | discontinuation, | active treatment |
| Weeks 4, 8, | regardless of | group and |
| 12, and 16 | protocol deviations | pooled placebo |
| | | at each visit |
| Mean percentage | To include all data | Posterior mean |
| change from | collected prior to | difference |
| baseline | treatment | between each |
| in LSS at | discontinuation, | active dose and |
| Weeks 4, 8, | regardless of | pooled placebo |
| 12, and 16 | protocol deviations | at each visit |
| Mean absolute | To include all data | Posterior mean |
| change from | collected prior to | difference |
| baseline | treatment | between each |
| in LSS at | discontinuation, | active dose and |
| Weeks 4, 8, | regardless of | pooled placebo |
| 12, and 16 | protocol deviations | at each visit |
| Mean percentage | To include all data | Posterior mean |
| change from | collected prior to | difference |
| baseline in | treatment | between each |
| DLQI score at | discontinuation, | active dose and |
| Weeks 4, 8, | regardless of | pooled placebo |
| 12, and 16 | protocol deviations | at each visit |
| Mean absolute | To include all data | Posterior mean |
| change from | collected prior to | difference |
| baseline in | treatment | between each |
| DLQI score at | discontinuation, | active dose and |
| Weeks 4, 8, | regardless of | pooled placebo |
| 12, and 16 | protocol deviations | at each visit |
| Mean percentage | To include all data | Posterior mean |
| change from | collected prior to | difference |
| baseline in | treatment | between each |
| mNAPSI | discontinuation, | active dose and |
| total score at | regardless of | pooled placebo |
| Weeks 4, 8, | protocol deviations | at each visit |
| 12, and 16 | | |
| Mean absolute | To include all data | Posterior mean |
| change from | collected prior to | difference |
| baseline in | treatment | between each |
| mNAPSI | discontinuation, | active dose and |
| total score at | regardless of | pooled placebo |
| Weeks 4, 8, | protocol deviations | at each visit |
| 12, and 16 | | |

Study Population:

Inclusion Criteria

Each participant must meet all the following criteria to be enrolled in this study:
1. Give written informed consent (signed and dated) and any authorizations required by local law and be able to comply with all study requirements.
2. Males or females≥18 and ≤70 years old at the time of informed consent.
3. A documented diagnosis of plaque psoriasis for ≥6 months.
4. Have mild to moderate plaque psoriasis with plaque covering BSA of 3% and 10% and meet both of the following additional criteria:
   a. PASI score of ≥6 and ≤15, and
   b. PGA score of 2 or 3.
   All parameters in this criterion should be reconfirmed at baseline visit prior to randomization.
5. Meet the following contraception criteria:
   a. Male participants:
      i. A male participant must agree to use contraception as detailed in Appendix Error! Reference source not found. of this protocol during their participation in this study and for a period of 90 days after the last dose and refrain from donating sperm during this period.
   b. Female participants:
      i. A female participant is eligible to participate if she is not pregnant (Appendix Error! Reference source not found.), not breastfeeding, and at least 1 of the following conditions applies:
         1. Not a WOCBP as defined in Appendix Error! Reference source not found., OR
         2. A WOCBP who agrees to follow the contraceptive guidance in Appendix Error! Reference source not found. during their participation in this study, 28 days prior to the first dose and for at least 1 complete menstrual cycle (≥28 days) after the last dose.
6. Agrees to not increase their usual sun exposure during the study.

Exclusion Criteria

Participants meeting any of the following criteria will be excluded from the study:
1. Have received Prevotella Strain B 50329 within the 3 months prior to screening.
2. Have a diagnosis of non-plaque psoriasis.
3. Plaque psoriasis restricted to scalp, palms and soles only.
4. Evidence of skin conditions that would interfere with psoriasis evaluation or treatment response (eg, atopic dermatitis, fungal or bacterial superinfection).
5. Having received systemic immunosuppressive therapy (MTX, apremilast, azathioprine, cyclosporine, 6-thioguanine, mercaptopurine, mycophenolate mofetil, hydroxyurea, and tacrolimus) within 4 weeks of first administration of study drug.
6. Unresponsive to prior use of biologics (including, but not limited to, TNFα inhibitors, natalizumab, efalizumab, anakinra or agents that modulate B cells or T cells).
7. If prior biologic therapy and responsive, participants must have been off therapy for at least 12 months prior to first administration of study drug.
8. Has received phototherapy or any systemic medications/treatments that could affect psoriasis or PGA evaluation (including, but not limited to oral or injectable corticosteroids, retinoids, 1,25-dihydroxy vitamin D3 and analogues, psoralens, sulfasalazine, hydroxyurea, or fumaric acid derivatives) within 4 weeks first administration of study drug.
9. Currently receiving lithium, antimalarials, leflunomide, or IM gold, or have received lithium, antimalarials, IM gold, or leflunomide within 4 weeks first administration of study drug.
10. Have used topical medications/treatments that could affect psoriasis or PGA evaluation (including [but not limited to] high- and mid-potency corticosteroids, anthralin, calcipotriene, topical vitamin D derivatives, retinoids, tazarotene, methoxsalen, trimethylpsoralens, picrolimus, and tacrolimus) within 2 weeks of the first administration of study drug. Topical unmedicated emollients and low-potency topical corticosteroids are not excluded.
11. Gastrointestinal tract disease (eg, short-bowel syndrome, diarrhea-predominant irritable bowel syndrome) that could interfere with GI delivery and transit time.
12. Active inflammatory bowel disease.
13. Active infection requiring systemic antiviral or antimicrobial therapy that will not be completed prior to Day 1 (Visit 2).
14. Has received live or live-attenuated vaccination within 6 weeks prior to screening or intends to have such a vaccination during the study.
15. Clinically significant abnormalities in screening laboratory values that would render a participant unsuitable for inclusion (per investigator judgment).
16. For women, serum creatinine≥125 μmol/L (1.414 mg/dL); for men, serum creatinine
≥135 μmol/L (1.527 mg/dL).
   17. ALT and AST>2×ULN.
   18. Known history of or positive test for HIV, or active infection with hepatitis C or chronic hepatitis B.
   19. History of clinically significant acute cardiac or cerebrovascular event within 6 months before screening (includes stroke, transient ischemic attack, and coronary heart disease [angina pectoris, myocardial infarction, heart failure, revascularization procedures]).
   20. In the opinion of the investigator, evidence of clinically important cardiac conduction abnormalities at screening as judged by ECG.
   21. Current acute or chronic inflammatory disease other than psoriasis or psoriatic arthritis (eg, inflammatory bowel disease, rheumatoid arthritis, systemic lupus erythematosus).
   22. Hypersensitivity to P histicola or to any of the excipients.
   23. Active untreated mental or psychiatric disorder. Participants who are on stable dosing of medication for a mental or psychiatric disorder for at least 6 months before screening and whose treating physicians consider them to be mentally stable may be enrolled.
   24. Any major or minor GI surgery within 6 months of screening.
   25. Any major surgery within 6 months of screening.
   26. Malignancy within 5 years, except for basal or squamous cell carcinoma of the skin or carcinoma in situ of the cervix that has been successfully treated.
   27. Treatment with another investigational drug, biological agent, or device within 1 month of screening, or 5 half-lives of investigational agent, whichever is longer.
   28. Initiating any OTC or prescription medication including vitamins, herbal supplements and nutraceuticals (eg, supplements including high doses of probiotics and prebiotics as usually found in capsules/tablets/powders), except acetaminophen/paracetamol and anti-histamines, within 14 days prior to baseline or anticipates change in dosage for the duration of the study period. Note that probiotic and prebiotic foods that contain low doses are allowed (eg, yoghurt, kefir, kombucha).
   29. Blood donation of >100 mL within 30 days of screening or of >499 mL within 12 weeks of screening.
   30. Unwillingness to comply with study procedures, including follow-up, as specified by this protocol, or unwillingness to cooperate fully with the investigator.
   31. Have any other conditions, which, in the opinion of the investigator or sponsor, would make the participant unsuitable for inclusion or could interfere with the participant participating in or completing the study.

Study Design:

This is a multicenter, randomized, double-blind, placebo-controlled, parallel-cohort, dose-ranging study of participants with mild to moderate plaque psoriasis, comprising a screening period of up to 4 weeks, a baseline visit, a treatment period of 16 weeks (8 planned study site visits), and a follow-up period of 4 weeks (1 planned study site visit at the end of study).

After eligibility is confirmed during the screening period, participants will be randomly assigned in a 1:1:1 ratio to 1 of the following 3 parallel cohorts:
Cohort 1: 0.8×10¹¹ cells of Prevotella Strain B 50329 or matching placebo administered as 1 PIC, once daily.
Cohort 2: 3.2×10¹¹ cells of Prevotella Strain B 50329 or matching placebo administered as 4 PICs, once daily.
Cohort 3: 8.0×10¹¹ cells of Prevotella Strain B 50329 or matching placebo administered as 10 PICs, once daily.

In each cohort, approximately 75 participants will be randomly assigned in a 2:1 ratio to receive either Prevotella Strain B 50329 or matching placebo once daily for 16 weeks.

An interim analysis may be performed after at least 50% of participants have completed at least 12 weeks of treatment.

After the planned 16 weeks of treatment, all participants will enter a 4-week post-treatment follow-up period and undergo end of treatment evaluations.

Estimated Study Duration:

The maximum planned duration for each participant will be 24 weeks (including 11 scheduled study visits), and the duration of the study is defined for each participant as the date signed written informed consent is provided through the last follow-up visit.

Efficacy Assessments:

The efficacy assessments will include the PAST score, the LSS, the National Psoriasis Foundation Psoriasis Score version of a static PGA, the percent of BSA involvement, the mNAPSI, the DLQI, the PSI, the SF-36 Bodily Pain Scale, the VAS Pain assessment, the vitality subscale of the SF-36 (to assess fatigue), and a fatigue VAS.

Pharmacokinetic or Pharmacodynamic Assessments:

Pharmacokinetic assessments will be limited to a predose blood sample at baseline and another sample at the Week 16 visit (end of treatment).

Pharmacodynamic and biomarker assessments are exploratory endpoints and analytical results for biomarkers will not be included in the CSR. They will be reported separately from the CSR.

Pharmacodynamic and biomarker assessments include digital photography of up to 6 lesion sites, standard histologic assessments of skin plaque biopsies, mRNA transcription analysis of skin plaque biopsies, blood cytokine and chemokine analyses, and microbiome composition of the fecal microbiome.

Safety and Tolerability Assessments:

Safety and tolerability assessments include monitoring AEs (including SAEs), monitoring concomitant medications, BSFS categorization (recorded in a stool diary), physical examinations, vital sign measurements, and ECGs.

Study drug, Dosage, and Route of Administration:

The study drug will be capsules containing Prevotella Strain B 50329 or matching capsules containing placebo.

There will be 3 dosing cohorts, with dosages of 1 capsule, 4 capsules, or 10 capsules; capsules of Prevotella Strain B 50329 each contain 8.0×10¹⁰ cells of Prevotella Strain B 50329, while placebo capsules contain no bacteria.

Participants will self-administer their doses of study drug orally in the morning with water.

Sample Size:

The sample size of 225 participants in total, has been chosen to explore the tolerability and safety of Prevotella Strain B 50329. Although the study will use a model-based probability inference approach in a Bayesian framework, the following power calculation was also performed (using a basic frequentist approach) in order to give confidence that enough participants are available to find a clinically meaningful difference between active dose and placebo if the below assumptions are met.

The primary efficacy endpoint is the percent change from baseline in the PASI score at Week 16. Percent change from baseline relative to placebo will be estimated within the model as (percent change in active)−(percent change in placebo), with a negative value indicating a greater improvement for active than placebo. A percent change from baseline relative to placebo of at least 20% will be considered clinically meaningful. The pooled standard deviation across all doses is assumed to be 25%.

Participants in the placebo arm of each cohort will be pooled for the statistical analysis in order to compare active and control arms resulting in 75 participants randomized to the pooled placebo group and 50 participants randomized to each active treatment group (Prevotella Strain B 50329 0.8×10¹¹ cells, Prevotella Strain B 50329 3.2×10¹¹ cells, and Prevotella Strain B 50329 8.0×10¹¹ cells). Assuming that no more than 15% of participants will discontinue treatment before the Week 16 visit, at least 42 active and 21 placebo participants in each of the 3 cohorts are expected to provide data through the Week 16 visit.

Each pairwise comparison between pooled placebo and active dose would be expected to have more than 95% power to detect a difference between the treatment groups at the 5% significance level under the assumption that pooling the placebo groups is a valid strategy. If the 3 placebo cohorts are considered to be too heterogeneous for pooling into a single reference group, the power to detect a difference in each within-cohort pairwise comparison between active and placebo doses would be greater than 80%.

As the statistical inference for this study will focus on estimation rather than testing a formal hypothesis, no multiplicity adjustments of the different comparisons between groups in order to control the study-wise type I error rate will be performed.

Similarly, as there is no intention to use any interim analyses to stop the study early for efficacy, no adjustments for multiplicity will be made to account for any analyses performed as part of the interim analyses.

Statistical Methods:

Analysis methods for key endpoints are briefly described below. Further details on all analyses will be described in the SAP.

No formal hypothesis will be tested. A model-based probability inference approach in a Bayesian framework will be used to guide decision-making around dose selection. Posterior estimates and 95% credible intervals (CrI) for the difference between each active dose and placebo will be produced for relevant primary and secondary endpoints.

Unless otherwise specified, missing data will be considered as missing at random and will be accounted for using mixed models for repeated measures, with all time points collected for the relevant endpoint included in the model. This includes data which is excluded due to collection after treatment discontinuation or after a protocol deviation as applicable to the definition of the estimands used in the analysis.

### Analysis Sets:

The mITT set will consist of all participants who were randomized to treatment and who received at least one dose of study treatment.

The PPS will consist of all mITT participants who were not replaced (following study withdrawal before the end of Week 4) and who do not have a protocol deviation that may impact efficacy with a start date for the deviation before initiation of study treatment.

The safety set will consist of all participants who received any study drug.

The mITT set will be the primary population of interest for the efficacy section, with some supportive analyses performed using the PPS. The safety set will be used for all safety summaries.

### Statistical Analysis Methodology:

Statistical analysis will be performed using SAS software Version 9.3 or later. Continuous variables will be summarized using the mean, standard deviation, median, minimum value, and maximum value. Categorical variables will be summarized using frequency counts and percentages. Time-to-event variables will be summarized using Kaplan-Meier estimates of the proportion of participants with the event at each visit. Data will be listed in data listings.

### Analysis of Primary Efficacy Endpoint:

The primary analysis will be performed using a Bayesian MMRM. The model will include parameters for treatment*visit and baseline PASI score*visit interactions. Body mass index, gender, and other baseline covariates will also be considered and included as parameters if found to be significant (p≤0.05). The model will not include an intercept. The priors for all parameters in the model will be non-informative and follow a normal distribution with mean 0 and SD 1000. The prior for the variance-covariance matrix will follow an inverted Wishart distribution with degrees of freedom equal to the number of visits and an identity scale matrix.

The adjusted posterior mean percentage change from baseline and the associated 95% HDP CrI for each treatment at Week 16 will be reported, together with the adjusted mean difference from placebo and the associated 95% HDP CrI for each active dose at each visit and the probability that each treatment difference is less than 0%, −20%, −30%, and −50%.

### Analysis of Secondary Efficacy Endpoints:

All secondary analyses will be performed on the mITT set, excluding data collected after treatment discontinuation, without consideration of any protocol deviations. Dose will be treated as a categorical variable and no dose response modelling will be done. Comparisons of interest will be between individual Prevotella Strain B 50329 doses and placebo. All posterior probabilities and CrI calculated will be considered as descriptive with no further adjustments for multiplicity performed.

### Analyses of Exploratory Efficacy Endpoints

Exploratory endpoints will be summarized using the mITT population, with data collected after discontinuation of treatment excluded but without consideration of any protocol deviations.

Analyses of biomarkers will be addressed in a data analysis plan outside the study SAP.

### Pharmacokinetic Analyses:

The number and percentage of participants who have a quantifiable concentration of Prevotella Strain B 50329 in their blood sample will be summarized using the safety set by visit. Placebo participants will be pooled into a single treatment group. If at least 20% of participants within a treatment group are found to have a quantifiable level at one of the visits, then the concentration will be summarized as a continuous variable for the relevant treatment group at that visit.

### Safety Analyses:

All safety endpoints will be tabulated or plotted by treatment group using the safety set. All safety analyses will use the pooled placebo. Further details will be described in the SAP.

List of Abbreviations and Definition of Terms

| Abbreviation | Definition |
|---|---|
| AE | adverse event |
| ALT | alanine aminotransferase |
| AST | aspartate aminotransferase |
| BSA | body surface area |
| BMI | body mass index |
| BSFS | Bristol Stool Form Scale |
| CFR | Code of Federal Regulations |
| CrI | credible interval(s) |
| CRP | C-reactive protein |
| CSR | clinical study report |
| CTCAE | Common Terminology Criteria for Adverse Events |
| DIC | deviance information criterion |
| DLQI | Dermatology Life Quality Index |
| ECG | electrocardiogram |
| eCRF | electronic case report form |
| EDC | electronic data capture |
| Prevotella | investigational study drug |
| Strain | |
| B 50329 | |
| EOS | end of study |
| FDA | US Food and Drug Administration |
| FSH | follicle-stimulating hormone |
| FUP | functional uniform prior |
| GCP | Good Clinical Practice |
| GI | gastrointestinal |
| HbsAg | hepatitis B surface antigen |
| HCG | human chorionic gonadotropin |
| HCV | hepatitis C virus |
| HDP | high-density probability |
| HIV | human immunodeficiency virus |
| HPMC | hydroxypropyl methylcellulose |
| HRT | hormone replacement therapy |
| IB | investigator's brochure |
| ICF | informed consent form |
| ICH | International Council for Harmonisation |
| IEC | independent ethics committee |
| IL | interleukin |
| IM | intramuscular |
| IFNγ | interferon gamma |
| IRB | institutional review board |
| IRE | Ireland |
| IRT | interactive response technology |
| LSS | lesion severity score |
| MedDRA | Medical Dictionary for Regulatory Activities |
| mITT | modified intent-to-treat |
| MMRM | mixed effects model with repeated measures |
| MTX | methotrexate |
| mNAPSI | modified Nail Psoriasis Severity Index |
| OTC | over-the-counter |
| PASI | Psoriasis Area and Severity Index |
| PCR | polymerase chain reaction |
| PDE4 | phosphodiesterase type 4 |
| PGA | Physician's Global Assessment |
| PIC | powder in capsule |
| PPS | per-protocol set |
| PSI | Psoriasis Symptom Inventory |
| QTcF | QT interval corrected using Fridericia's formula |
| SAE | serious adverse event |
| SAP | statistical analysis plan |
| SoA | schedule of study site activities |
| SUSAR | suspected unexpected serious adverse reaction |
| TEAE | treatment-emergent adverse event |
| TNFα | tumor necrosis factor alpha |
| ULN | upper limit of normal |
| USA | United States of America |
| VAS | visual analog scale |
| WHO | World Health Organization |
| WOCBP | woman/women of child-bearing potential |

Introduction

A therapeutic agent that offers the potential of systemic immune system modulation following oral administration, without systemic exposure, is being developed. Prevotella Strain B 50329 is a pharmaceutical preparation of a strain of Prevotella histicola isolated from a human duodenal biopsy: it has not been genetically modified. Strains of the Prevotella genus of microbes have been found in all human populations tested to date, at abundances ranging from less than 1% to nearly 50% of total fecal microbial load (Vandeputte 2017). Prevotella are gram-negative, obligate anaerobes that are natural human commensals in the oral cavity and GI tract. Prevotella Strain B 50329 is a gram-negative bacterium sensitive to the major classes of antibiotics, eg, penicillins and cephalosporins. In non-clinical and clinical studies, its therapeutic effects have been dose-dependent.

Several studies (de Groot et al 2017; Hindson et al 2017; Yan et al 2017; Felix et al 2018) suggest that host-microbe interactions in the gut, and particularly in the small intestine, can influence systemic inflammation. Preclinical data confirms that individual strains of microbes exhibit unique pharmacological profiles. This is thought to be based on multiple distinct microbial structural pattern motifs interacting with varying combinations of host pattern recognition receptors in small intestinal epithelium.

Studies of Prevotella Strain B 50329 in vitro in a range of human and mouse assays and studies in vivo in model symptoms support the use of Prevotella Strain B 50329 in the treatment of inflammatory diseases including psoriasis. Prevotella Strain B 50329 increases secretion of anti-inflammatory cytokines such as IL-10, IL1RA, and IL-27 from human immune cells, while inducing minimal production of pro-inflammatory cytokines such as IL-6, TNFα, and IFNγ.

Oral administration of Prevotella Strain B 50329 to mice results in striking pharmacodynamic effects on animal models of delayed-type hypersensitivity, fluorescein isothiocyanate cutaneous hypersensitivity, collagen-induced arthritis (Marietta et al 2016) and experimental acute encephalomyelitis (Mangalam et al 2017). The high degree of consistency of both effect and dose suggests the potential for clinical benefit across multiple type 1, type 2, and type 3 inflammatory conditions. No potentially related adverse effects were seen in the animals used in these experiments with daily dosing for up to 3 weeks or with alternate day dosing for over 7 weeks. Immunophenotyping ex vivo in these models shows increased regulatory T cell numbers and regulatory dendritic cells in spleen and mesenteric lymph nodes, as well as decreases in pro-inflammatory cytokines such as IL-23p40, IL-17, TNF, IL-6, and IL-13. Treatment also led to enhancement of gut intestinal barrier integrity, which is often disrupted in patients with inflammatory diseases. These effects on immune parameters have been observed both within and outside the GI tract, which demonstrates that host-microbe interactions in the gut can affect the immune response in peripheral tissues.

Psoriasis is a chronic immune-mediated type 1/3 inflammatory skin disease in which hyperactive T cells trigger excessive keratinocyte proliferation. This results in the formation of raised erythematous plaques with scaling. Psoriatic lesions can appear anywhere on the body but are most often seen on the knees, elbows, scalp, and lumbar area. Critical events in the inflammatory process include activation of Langerhans cells and T cells, selective trafficking of activated T cells to the skin, and induction of an inflammatory cytokine and chemokine cascade in skin lesions. Clinical data have validated the role of anti-TNFα, anti-IL-17, and anti-IL-23 therapy in moderate to severe psoriasis. For patients with mild to moderate psoriasis, therapy usually involves topical agents (topical corticosteroids, vitamin D3 analogs), with topical corticosteroids providing the greatest range of efficacy and a wide range of formulations. More recently, physicians are prescribing apremilast, a first-in-class oral PDE4 inhibitor, ahead of biological therapy, which includes etanercept, infliximab, adalimumab, ustekinumab, and secukinumab.

In another study, a total of 56 participants have participated in Cohorts 1-4, with 36 of these participants receiving Prevotella Strain B 50329 once daily; 16 of these were treated for 14 days (healthy volunteers) and 20 participants with psoriasis were treated for 28 days. Clinical responses, similar to apremilast and tofacitinib at the same time point, have been observed on the (psoriatic) LSS and PASI at Day 28. Furthermore, at the Day 42 follow-up visit, when participants were “off treatment” for 14 days, there was continued improvement in the pharmacodynamic response as measured by both LSS and PASI for participants administered the higher dose, but not the lower dose, suggesting a dose relationship on the durability of effect. The safety profile of Prevotella Strain B 50329 was similar to placebo, with no SAEs or AEs of severe intensity.

The evidence available so far suggests Prevotella Strain B 50329 is very well tolerated and it continues to undergo clinical development in mild to moderate psoriasis. A well-tolerated oral therapy could offer significant benefit in the treatment of psoriasis and it is presently anticipated that Prevotella Strain B 50329 would be used in established but early disease, before the use of biologic therapies.

This Phase 2 study has been designed to investigate the clinical safety and efficacy of Prevotella Strain B 50329 and to identify an optimal dose.

Study Objectives

All objectives are related to understanding the safety, efficacy, and dose effects of Prevotella Strain B 50329 treatment of mild to moderate plaque psoriasis in adult participants.

Primary Objective

The primary objective of this study is to evaluate the safety and efficacy of 3 different doses of Prevotella Strain B 50329 for the treatment of psoriasis following daily dosing for 16 weeks.

Secondary Objectives

The secondary objectives of this study are the following:
To evaluate the efficacy dose response of Prevotella Strain B 50329 at Week 16
To evaluate the maximal clinical benefit of Prevotella Strain B 50329 at Week 16
To evaluate the optimal dose of Prevotella Strain B 50329 based on efficacy and safety up to Week 16
To evaluate the safety and tolerability of Prevotella Strain B 50329 (all dose levels) throughout the study

Exploratory Objectives

The exploratory objectives of this study are the following:
To evaluate the time to onset of clinical response to Prevotella Strain B 50329
To evaluate the effect of Prevotella Strain B 50329 treatment on patient-reported outcomes including quality of life and pain
To evaluate the effect of Prevotella Strain B 50329 treatment on biomarkers in blood
To evaluate the effect of Prevotella Strain B 50329 treatment on biomarkers in skin plaques
To evaluate the effect of Prevotella Strain B 50329 treatment on fecal microbiome composition

Investigational Plan

### Study Design

This is a multicenter, randomized, double-blind, placebo-controlled, parallel-cohort, dose-ranging study of participants with mild to moderate plaque psoriasis, comprising a screening period of up to 4 weeks, a baseline visit, a treatment period of 16 weeks (8 planned study site visits), and a follow-up period of 4 weeks (1 planned study site visit at EOS). There are a total of 11 scheduled study visits.

After eligibility is confirmed during the screening period (as described herein), participants will be randomly assigned in a 1:1:1 ratio to 1 of the following 3 parallel cohorts:
Cohort 1: 0.8×10¹¹ cells of Prevotella Strain B 50329 or matching placebo administered as 1 PIC, once daily.
Cohort 2: 3.2×10¹¹ cells of Prevotella Strain B 50329 or matching placebo administered as 4 PICs, once daily.
Cohort 3: 8.0×10¹¹ cells of Prevotella Strain B 50329 or matching placebo administered as 10 PICs, once daily.

In each cohort, approximately 75 participants will be randomly assigned in a 2:1 ratio to receive either Prevotella Strain B 50329 or matching placebo once daily for 16 weeks.

An interim analysis may be performed after at least 50% of participants have completed at least 12 weeks of treatment.

After the planned 16 weeks of treatment, all participants will enter a 4-week post-treatment follow-up period and undergo end of treatment evaluations. The maximum planned duration for each participant will be 24 weeks, and the duration of the study is defined for each participant as the date signed written informed consent is provided through the last follow-up visit. Participants will be considered to have completed the study with the completion of all phases of the study, culminating with their EOS follow-up visit.

### Rationale for Study Design

The Prevotella Strain B 50329 Phase 1 program evaluated doses of 1.6×10¹⁰ cells to 8.0×10¹¹ cells given daily for 2 weeks in healthy volunteers and doses of 1.6×10¹¹ cells and 8.0×10¹¹ cells given daily for 4 weeks to participants with mild to moderate psoriasis. All doses were found to be well tolerated and doses of both 1.6×10¹¹ and 8.0×10¹¹ cells induced clinically relevant reductions in signs and symptoms of plaque psoriasis and psoriasis lesion severity.

The doses tested in the program are based on predictions from the preclinical data and the clinical and biomarker data obtained in the Phase 1 study. All doses tested up to 8.0×10¹¹ cells have been equally well tolerated. No clear difference in efficacy was observed between the 1.6×10¹¹ cells and the 8.0×10¹¹ cells in the previous study over the 28-day dosing period, but at the 14-day follow up (Day 42) the participants (e.g., subjects) receiving the higher dose had a continued improvement in their psoriasis compared to participants who had received the lower dose. This suggests a more sustained and potentially deeper response in the high dose group. It is therefore proposed to include the lowest and highest feasible doses (based on capsule load) in this study to establish the dose response, the maximum clinical benefit, and to assess participant tolerability and acceptability of the doses tested.

The clinical response to Prevotella Strain B 50329 treatment will be evaluated using multiple assessments, facilitating appropriate selection of efficacy measures for future studies.

The use of a placebo comparator is appropriate for this participant population of individuals with mild to moderate plaque psoriasis for the following reasons:
The limited proven efficacy of other treatments (topical corticosteroids, vitamin D3 analogs, and apremilast) in patients with mild to moderate plaque psoriasis that could potentially serve as an active comparator
The limited duration of the study (maximum of 16 weeks of treatment) for each participant
A randomization ratio of 2:1 for Prevotella Strain B 50329 treatment to placebo treatment in each cohort

Participant Selection and Withdrawal Criteria

### Selection of Study Population

Approximately 225 participants will be enrolled (randomly assigned to treatment) in multiple countries, including (but not limited to) sites in the United States, the United Kingdom, and Poland. Participants will be assigned to study treatment only if they meet all inclusion criteria and no exclusion criteria during screening.

Deviations from the inclusion and exclusion criteria are not allowed: adherence to the eligibility criteria as specified in the protocol is essential.

Screen failures are defined as participants who consent to participate in the clinical study but are not subsequently randomly assigned to study drug (Prevotella Strain B 50329 or placebo). A minimal set of screen failure information is required to be entered in the eCRF to ensure transparent reporting of screen failure participants to meet the Consolidated Standards of Reporting Trials publishing requirements and to respond to queries from regulatory authorities. Minimal information includes demography, screen failure details, eligibility criteria, and any SAEs.

Individuals who fail to satisfy inclusion and exclusion criteria at screening may be rescreened 1 additional time with the agreement of the medical monitor before rescreening. Participants may also be rescreened if they initially pass the screening assessments but go beyond the screening period time limit. In exceptional circumstances, the screening window can be extended on a case-by-case basis after consultation with the sponsor: such an exceptional extension will not be considered a protocol deviation.

### Inclusion Criteria

Each participant must meet all the following criteria to be enrolled in this study:
1. Give written informed consent (signed and dated) and any authorizations required by local law and be able to comply with all study requirements.
2. Males or females≥18 and ≤70 years old at the time of informed consent.
3. A documented diagnosis of plaque psoriasis for ≥6 months.
4. Have mild to moderate plaque psoriasis with plaque covering body surface area (BSA) of ≥3% and ≤□□% and meet both of the following additional criteria:
   a. PASI score of ≥6 and ≤15, and
   b. PGA score of 2 or 3.
   All parameters in this criterion should be reconfirmed at baseline visit prior to randomization.
5. Meet the following contraception criteria:
   a. Male participants:
      i. A male participant must agree to use contraception as detailed in Appendix Error! Reference source not found. of this protocol during their participation in this study and for a period of 90 days after the last dose and refrain from donating sperm during this period.
   b. Female participants:
      i. A female participant is eligible to participate if she is not pregnant (Appendix Error! Reference source not found.), not breastfeeding, and at least 1 of the following conditions applies:
         1. Not a WOCBP as defined in Appendix Error! Reference source not found., OR
         2. A WOCBP who agrees to follow the contraceptive guidance in Appendix Error! Reference source not found. during their participation in this study, 28 days prior to the first dose and for at least 1 complete menstrual cycle (≥28 days) after the last dose.
6. Agrees to not increase their usual sun exposure during the study.

### Exclusion Criteria

Participants meeting any of the following criteria will be excluded from the study:
1. Have received Prevotella Strain B 50329 within the 3 months prior to screening.
2. Have a diagnosis of non-plaque psoriasis.
3. Plaque psoriasis restricted to scalp, palms, and soles only.
4. Evidence of skin conditions that would interfere with psoriasis evaluation or treatment response (eg, atopic dermatitis, fungal or bacterial superinfection).
5. Have received systemic immunosuppressive therapy (MTX, apremilast, azathioprine, cyclosporine, 6-thioguanine, mercaptopurine, mycophenolate mofetil, hydroxyurea, and tacrolimus) within 4 weeks of first administration of study drug.
6. Unresponsive to prior use of biologics (including, but not limited to, TNFα inhibitors, natalizumab, efalizumab, anakinra or agents that modulate B cells or T cells).
7. If prior biologic therapy and responsive, participants must have been off therapy for at least 12 months prior to first administration of study drug.
8. Have received phototherapy or any systemic medications/treatments that could affect psoriasis or PGA evaluation (including, but not limited to oral or injectable corticosteroids, retinoids, 1,25-dihydroxy vitamin D3 and analogues, psoralens, sulfasalazine, hydroxyurea, or fumaric acid derivatives) within 4 weeks of first administration of study drug.
9. Currently receiving lithium, antimalarials, leflunomide, or IM gold, or have received lithium, antimalarials, IM gold, or leflunomide within 4 weeks of first administration of study drug.
10. Have used topical medications/treatments that could affect psoriasis or PGA evaluation (including [but not limited to] high- and mid-potency corticosteroids [Appendix Error! Reference source not found.], anthralin, calcipotriene, topical vitamin D derivatives, retinoids, tazarotene, methoxsalen, trimethylpsoralens, picrolimus, and tacrolimus) within 2 weeks of the first administration of study drug. Topical unmedicated emollients and low-potency topical corticosteroids are not excluded.
11. Gastrointestinal tract disease (eg, short-bowel syndrome, diarrhea-predominant irritable bowel syndrome) that could interfere with GI delivery and transit time.
12. Active inflammatory bowel disease.
13. Active infection requiring systemic antiviral or antimicrobial therapy that will not be completed prior to Day 1 (Visit 2).
14. Have received live or live-attenuated vaccination within 6 weeks prior to screening or intend to have such a vaccination during the study.
15. Clinically significant abnormalities in screening laboratory values that would render a participant unsuitable for inclusion (per investigator judgment).
16. For women, serum creatinine≥125 μmol/L (1.414 mg/dL); for men, serum creatinine≥135 μmol/L (1.527 mg/dL).
17. ALT and AST>2×ULN.
18. Known history of or positive test for HIV, or active infection with hepatitis C or chronic hepatitis B.
19. History of clinically significant acute cardiac or cerebrovascular event within 6 months before screening (includes stroke, transient ischemic attack, and coronary heart disease [angina pectoris, myocardial infarction, heart failure, revascularization procedures]).
20. In the opinion of the investigator, evidence of clinically important cardiac conduction abnormalities at screening as judged by ECG.
21. Current acute or chronic inflammatory disease other than psoriasis or psoriatic arthritis (eg, inflammatory bowel disease, rheumatoid arthritis, systemic lupus erythematosus).
22. Hypersensitivity to P histicola or to any of the excipients.
23. Active untreated mental or psychiatric disorder. Participants who are on stable dosing of medication for a mental or psychiatric disorder for at least 6 months before screening and whose treating physicians consider them to be mentally stable may be enrolled.
24. Any major or minor GI surgery within 6 months of screening.
25. Any major surgery within 6 months of screening.
26. Malignancy within 5 years, except for basal or squamous cell carcinoma of the skin or carcinoma in situ of the cervix that has been successfully treated.
27. Treatment with another investigational drug, biological agent, or device within 1 month of screening, or 5 half-lives of investigational agent, whichever is longer.
28. Initiating any OTC or prescription medication including vitamins, herbal supplements and nutraceuticals (eg, supplements including high doses of probiotics and prebiotics as usually found in capsules/tablets/powders), except acetaminophen/paracetamol and anti-histamines, within 14 days prior to baseline or anticipates change in dosage for the duration of the study period. Note that probiotic and prebiotic foods that contain low doses are allowed (eg, yoghurt, kefir, kombucha).
29. Blood donation of >100 mL within 30 days of screening or >499 mL within 12 weeks of screening.
30. Unwillingness to comply with study procedures, including follow-up, as specified by this protocol or unwillingness to cooperate fully with the investigator.
31. Have any other conditions, which, in the opinion of the investigator or sponsor, would make the participant unsuitable for inclusion or could interfere with the participant participating in or completing the study.

### Study Treatments

### Method of Assigning Participants to Treatment Groups

Participants will be randomly assigned at the baseline visit (Visit 2) to 1 of 3 cohorts (in a 1:1:1 allocation ratio) that are distinguishable to participants and study staff by the number of capsules administered per once-daily dose. Within the cohort, participants will be randomly assigned in a 2:1 allocation ratio to receive either Prevotella Strain B 50329 or matching placebo treatment (as described herein). Interactive response technology (IRT) will be used to administer the randomization schedule.

### Treatments Administered

Participants in each cohort (as described herein) will self-administer study drug doses orally in the morning with water, refraining from consuming acidic drinks 1 hour either side of dosing and from eating 2 hours before dosing and 1 hour after dosing. The composition of capsules is described herein. Strategy to improve compliance is presented herein.

### Identity of Study Drug

The Prevotella Strain B 50329 drug product is available as enteric-coated HPMC hard capsules in Swedish-Orange color. The Prevotella Strain B 50329 PIC consists of freeze-dried powder of P histicola, mannitol, magnesium stearate, and colloidal silicon dioxide. Each Prevotella Strain B 50329 PIC contains 8.0×10¹⁰ cells of P histicola. The matching placebo is identical in appearance but do not contain P histicola or any other bacteria. The placebo excipients include microcrystalline cellulose and magnesium stearate.

### Management of Clinical Supplies

### Study Drug Packaging and Storage

Prevotella Strain B 50329 PICs and matching placebo will be prepared in blister wallets of 10 capsules. Blister wallets will be packaged in packs that contain approximately 1 week's supply of study drug for 1 randomized participant, identified by a numeric code. When appropriate for the interval between study visits, multiple packs will be assigned and dispensed for each participant throughout the treatment period.

Study drug (Prevotella Strain B 50329 and placebo) must be stored in a secure area (eg, a locked refrigerator) and kept at a controlled temperature of 2° C. to 8° C. The investigator or designee must confirm appropriate temperature conditions have been maintained during transit and during storage at each site for all study drug received and any discrepancies are reported and resolved before use of the study drug.

### Concomitant Therapy

Anti-histamines and acetaminophen/paracetamol following labeled dosing instructions are permitted for use at any time during the study. Topical unmedicated emollients and low-potency topical steroids are also permitted if participants were already using them as part of their care prior to study entry (exclusion criterion #10). Participants will be advised to continue to use these therapies as they were prior to study entry.

Non-live vaccines are permitted in this study.

Other concomitant medication may be considered on a case-by-case basis by the investigator in consultation with the medical monitor if required.

### Prohibited Concomitant Therapy

Prior therapies restricted for participants eligible for this study as detailed in the exclusion criteria (as described herein) are prohibited concomitant therapy during the study.

Live or live-attenuated vaccines are contra-indicated in this study.

### Efficacy Assessments

### Psoriasis Area and Severity Index Score

The PASI score will be assessed as described by Langley and Ellis (2004). The PASI is a physician assessment that combines the assessment of the severity of and area affected by psoriasis into a single score in the range 0 (no disease) to 72 (maximal disease). The absolute PASI score in this study is used as part of inclusion criterion #4. The PASI percentage response rates are efficacy endpoints (ie, PASI-50, PASI-75, PASI-90, and PASI-100). For example, the percentage of participants who achieve a 75% or greater reduction in PASI score from baseline is represented by the PASI-75 value. Details of the PASI assessment will be provided in the study manual.

### Lesion Severity Score

The LSS is used to score the severity of psoriasis plaques (Patel and Tsui 2011). The dimensions of scaling, erythema, and plaque elevation are each scored on a scale from 0 to 4, and the total LSS is the numerical sum of the 3-dimensional scores observed at a single study visit.

### Physician's Global Assessment

The National Psoriasis Foundation Psoriasis Score version of a static PGA is calculated by averaging the total body erythema, induration, and desquamation scores (Feldman and Krueger 2005). Erythema, induration, and desquamation will be scored on a 6-point scale, ranging from 0 (clear) to 5 (severe): the total PGA score is defined as the average of the erythema, induration, and desquamation scores. Details of the PGA assessment will be provided in the study manual.

### Percent of Body Surface Area Involvement

The percent of BSA involvement will be estimated for each participant, where 1% is approximately the area of the participant's handprint (Walsh et al 2013). Details of the BSA assessment will be provided in the study manual.

Walsh and colleagues proposed the product of the PGA and the BSA involvement as a simple and effective alternative for measuring severity of psoriasis in clinical trials (Walsh et al 2013).

### Modified Nail Psoriasis Severity Index

The mNAPSI is a numeric, reproducible, objective, and simple tool for physicians to evaluate the severity of nail bed psoriasis and nail matrix psoriasis by area of involvement in the nail unit (Cassell et al 2007). Details of conducting the mNAPSI will be provided in the study manual.

### Dermatology Life Quality Index

The DLQI is a patient reported outcomes instrument for assessing the impact of dermatologic conditions on patients' quality of life (Finlay and Khan 1994). Details of administering the DLQI will be provided in the study manual.

### Psoriasis Symptom Inventory

The PSI is a patient reported outcomes instrument that is used to assess the severity of plaque psoriasis symptoms (Bushnell et al 2013). All symptoms (itch, redness, scaling, burning, cracking, stinging, flaking, and pain) are rated on a 5-point severity scale. The PSI demonstrated good construct validity and was sensitive to within-subject change (p≤0.0001). Details of administering the PSI will be provided in the study manual.

### Pain

Pain will be assessed by the SF-36 Bodily Pain Scale (SF-36 BPS) and the VAS Pain (Hawker et al 2011). Details of administering the pain assessments will be provided in the study manual.

### Fatigue

Consistent with a recent study of fatigue in psoriasis (Skoie et al 2017), fatigue will be assessed by the vitality subscale of the SF-36 (van der Heijden et al 2003) and a fatigue VAS (Wolfe 2004). Details of administering the fatigue assessments will be provided in the study manual.

### Histologic Assessment

Standard histology will be performed on skin plaque biopsies (including epidermal thickness, basal mitotic counts and immune cell infiltrates, immunohistochemistry) from approximately 15 participants in each cohort. Details will be provided in the study manual. The histologic evaluations are exploratory and are outside the scope of the CSR.

mRNA Transcription Analysis

An mRNA transcription analysis will be performed on the skin plaque biopsies.

### Blood Cytokine and Chemokine Analysis

Blood samples will be stimulated ex vivo and analyzed for levels of cytokines and chemokines, including IL-1 beta, IL-2, IL-4, IL-6, IL-8, IL-10, IL-12p40, IL-17A, TNFα, and IFNγ.

### Statistical Considerations

Analysis methods for key endpoints are described below. Further details on all analyses will be described in the SAP.

No formal hypothesis will be tested. A model-based probability inference approach in a Bayesian framework will be used to guide decision-making around dose selection. Posterior estimates and 95% CrI for the difference between each active dose and placebo will be produced for relevant primary and secondary endpoints.

Unless otherwise specified, missing data will be considered as missing at random and will be accounted for using mixed models for repeated measures, with all time points collected for the relevant endpoint included in the model. This includes data which is excluded due to collection after treatment discontinuation or after a protocol deviation as applicable to the definition of the estimands used in the analysis.

### Estimands and Intercurrent Events

### Primary Efficacy Estimand

The primary estimand will be the effect of Prevotella Strain B 50329 on the percent change in PASI score from baseline to Week 16 in the mITT set of all treated participants, regardless of deviations from the protocol. Only data collected while on treatment will be used to account for the intercurrent event of treatment discontinuation (hypothetical strategy based on adherence to treatment only). The posterior mean difference between each active dose and the pooled placebo will be estimated.

Percent change from baseline in PASI score at each visit will be calculated as:

100*(PASI score at Visit−baseline PASI score)/baseline PASI score.

A negative percentage change from baseline will indicate an improvement.

For the primary analysis, 2 supportive estimands will also be considered.
To assess the impact of intercurrent events related to protocol deviations believed to impact efficacy, a supportive analysis will be performed where all data collected after any such identified protocol deviations will be excluded. Participants who had a protocol deviation believed to impact efficacy prior to the first dose of treatment (hypothetical strategy based on adherence to treatment and the aspects of the protocol which impact efficacy) will be completely excluded from this analysis.
To assess the impact of treatment discontinuation, a supportive analysis will be performed in which all data collected during the study, including any data collected after treatment discontinuation will be included (treatment policy strategy).

The primary analysis will be performed using a Bayesian MMRM as fully described herein. Data from visits prior to Week 16 will be included in the model and missing data will not be explicitly imputed.

Supportive analyses will also be performed in the same manner, using the 2 alternative estimands as defined above. These will explore the possible impact of the intercurrent events of treatment discontinuation and events relating to protocol deviations that may have an impact on efficacy.

### Secondary Efficacy Estimands

Estimands for the analyses of all secondary endpoints are shown in the following table.

Table: Summary of Secondary Estimands

| Population | | Consideration of | Summary |
|---|---|---|---|
| of interest | Endpoint | intercurrent events | measure |
| mITT set | Mean percentage | To include all data | Posterior mean |
| | change from | collected prior to | difference between |
| | baseline in | treatment | each active |
| | PASI Score at | discontinuation, | treatment group |
| | Weeks 4, 8, | regardless of | and pooled placebo |
| | and 12 | protocol deviations | at each visit |
| mITT set | Mean absolute | To include all data | Posterior mean |
| | change from | collected prior to | difference between |
| | baseline in | treatment | each active |
| | PASI Score at | discontinuation, | treatment group |
| | Weeks 4, 8, | regardless of | and pooled placebo |
| | 12, and 16 | protocol deviations | at each visit |
| mITT set | Achievement of | To include all data | Posterior odds |
| | PASI-50 at | collected prior to | ratio for |
| | Weeks 4, 8, | treatment | response between |
| | 12, and 16 | discontinuation, | each active |
| | | regardless of | treatment group |
| | | protocol deviations | and pooled placebo |
| | | | at each visit |
| mITT set | Time to first | To include all data | Hazard ratio |
| | achievement of | collected prior to | for each |
| | PASI-50 | treatment | active group |
| | | discontinuation, | versus placebo |
| | | regardless of | |
| | | protocol deviations | |
| mITT set | Achievement of | To include all data | Proportion |
| | PASI-75, | collected prior to | of response |
| | PASI-90 and | treatment | for each |
| | PASI-100 at | discontinuation, | endpoint in each |
| | Week 16 | regardless of | treatment group |
| | | protocol deviations | at each visit |
| mITT set | Achievement of | To include all data | Posterior odds |
| | PGA of 0 or 1 | collected prior to | ratio for response |
| | with a ≥2-point | treatment | between each |
| | improvement | discontinuation, | active treatment |
| | from baseline at | regardless of | group and |
| | Week 16 | protocol deviations | pooled placebo |
| mITT set | Achievement of | To include all data | Posterior odds |
| | PGA of 0 at | collected prior to | ratio for response |
| | Week 16 | treatment | between each |
| | | discontinuation, | active treatment |
| | | regardless of | group and |
| | | protocol deviations | pooled placebo |
| mITT set | Mean percentage | To include all data | Posterior mean |
| | change from | collected prior to | difference |
| | baseline in | treatment | between each |
| | PGA × BSA at | discontinuation, | active treatment |
| | Weeks 4, 8, | regardless of | group and |
| | 12, and 16 | protocol deviations | pooled placebo |
| | | | at each visit |
| mITT set | Mean absolute | To include all data | Posterior mean |
| | change from | collected prior to | difference |
| | baseline in | treatment | between each |
| | PGA × BSA at | discontinuation, | active treatment |
| | Weeks 4, 8, | regardless of | group and |
| | 12, and 16 | protocol deviations | pooled placebo |
| | | | at each visit |
| mITT set | Mean percentage | To include all data | Posterior mean |
| | change from | collected prior to | difference |
| | baseline | treatment | between each |
| | in LSS at | discontinuation, | active dose and |
| | Weeks 4, 8, | regardless of | pooled placebo |
| | 12, and 16 | protocol deviations | at each visit |
| mITT set | Mean absolute | To include all data | Posterior mean |
| | change from | collected prior to | difference |
| | baseline | treatment | between each |
| | in LSS at | discontinuation, | active dose and |
| | Weeks 4, 8, | regardless of | pooled placebo |
| | 12, and 16 | protocol deviations | at each visit |
| mITT set | Mean percentage | To include all data | Posterior mean |
| | change from | collected prior to | difference |
| | baseline in | treatment | between each |
| | DLQI score at | discontinuation, | active dose and |
| | Weeks 4, 8, | regardless of | pooled placebo |
| | 12, and 16 | protocol deviations | at each visit |
| mITT set | Mean absolute | To include all data | Posterior mean |
| | change from | collected prior to | difference |
| | baseline in | treatment | between each |
| | DLQI score at | discontinuation, | active dose and |
| | Weeks 4, 8, | regardless of | pooled placebo |
| | 12, and 16 | protocol deviations | at each visit |
| mITT set | Mean percentage | To include all data | Posterior mean |
| | change from | collected prior to | difference |
| | baseline in | treatment | between each |
| | mNAPSI | discontinuation, | active dose and |
| | total score at | regardless of | pooled placebo |
| | Weeks 4, 8, | protocol deviations | at each visit |
| | 12, and 16 | | |
| mITT set | Mean absolute | To include all data | Posterior mean |
| | change from | collected prior to | difference |
| | baseline in | treatment | between each |
| | mNAPSI | discontinuation, | active dose and |
| | total score at | regardless of | pooled placebo |
| | Weeks 4, 8, | protocol deviations | at each visit |
| | 12, and 16 | | |

Summaries and analyses of the secondary endpoints are detailed in full herein.

### Exploratory Endpoints

The exploratory endpoints include the following:
Percentage of participants achieving PASI-50, PASI-75, PASI-90, and PASI-100 at Weeks 4, 8, and 12
Percentage of participants achieving PGA of 0 or 1 with a ≥2-point improvement at Weeks 4, 8, and 12

Percentage of participants achieving PGA of 0 at Weeks 4, 8, and 12

Mean change from baseline in PSI quality of life scores at Weeks 12 and 16
Mean percentage change from baseline in PSI quality of life scores at Weeks 12 and 16

Mean change from baseline in Pain and Fatigue scores at Weeks 4, 8, 12, and 16

Mean percentage change from baseline in Pain and Fatigue scores at Weeks 4, 8, 12, and 16
Mean change from baseline in fasting blood glucose and fasting lipid panel at Weeks 8 and 16

Biomarker endpoints (statistical analysis to appear separately from the CSR) include the following:
Histological assessment of skin plaque biopsies (including epidermal thickness, basal mitotic counts and immune cell infiltrates) at Week 16 versus baseline
mRNA transcription analysis on skin plaque biopsies at Week 16 versus baseline
Blood cytokine and chemokine levels at Week 16 versus baseline
Microbiome composition (in feces) at Week 16 and Week 20 versus baseline

Exploratory endpoints will be summarized using the mITT set, with data collected after discontinuation of treatment excluded but without consideration of any protocol deviations.

### Sample Size Determination

The sample size of 225 participants in total, has been chosen to explore the tolerability and safety of Prevotella Strain B 50329. Although the study will use a model-based probability inference approach in a Bayesian framework, the following power calculation was also performed (using a basic frequentist approach) in order to give confidence that enough participants are available to find a clinically meaningful difference between active dose and placebo if the below assumptions are met.

The primary efficacy endpoint is the percent change from baseline in the PASI score at Week 16. Percent change from baseline relative to placebo will be estimated within the model (as described herein) as (percent change in active)−(percent change in placebo), with a negative value indicating a greater improvement for active than placebo. A percent change from baseline relative to placebo of at least 20% will be considered clinically meaningful. The pooled standard deviation across all doses is assumed to be 25%.

Participants in the placebo arm of each cohort will be pooled for the statistical analysis in order to compare active and control arms resulting in 75 participants randomized to the pooled placebo group and 50 participants randomized to each active treatment group (Prevotella Strain B 50329 0.8×10¹¹ cells, Prevotella Strain B 50329 3.2×10¹¹ cells, and Prevotella Strain B 50329 8.0×10¹¹ cells). Assuming that no more than 15% of participants will discontinue treatment before the Week 16 visit, at least 42 active and 21 placebo participants in each of the 3 cohorts are expected to provide data through the Week 16 visit.

Each pairwise comparison between pooled placebo and active dose would be expected to have more than 95% power to detect a difference between the treatment groups at the 5% significance level under the assumption that pooling the placebo groups is a valid strategy. If the 3 placebo cohorts are considered to be too heterogeneous for pooling into a single reference group, the power to detect a difference in each within-cohort pairwise comparison between active and placebo doses would be greater than 80%.

As the statistical inference for this study will focus on estimation rather than testing a formal hypothesis, no multiplicity adjustments of the different comparisons between groups in order to control the study-wise type I error rate will be performed.

Similarly, as there is no intention to use any interim analyses to stop the study early for efficacy (as described herein), no adjustments for multiplicity will be made to account for any analyses performed as part of the interim analyses.

### Analysis Sets

The following analysis sets will be used in the statistical analyses.

Enrolled set: The enrolled set will consist of all participants who sign the ICF.

mITT set: The mITT set will consist of all participants who were randomized to treatment and who received at least one dose of study treatment. Participants who withdraw from the study before the end of Week 4 and are replaced will be included in this analysis set. All analyses using the mITT will group participants according to randomized treatment.

PPS: The PPS will consist of all mITT participants who were not replaced (following study withdrawal before the end of Week 4) and who do not have a protocol deviation that may impact efficacy with a start date for the deviation before initiation of study treatment. Note that in the case of participants who have a protocol deviation with a potential impact on efficacy after initiation of treatment, the participant will remain in the PPS but all data collected after the protocol deviation occurred will be excluded from any analyses performed using the PPS. All analyses using the PPS will group participants according to treatment received at the start of the study.

Safety set: The safety set will consist of all participants who received any study drug. All analyses using the safety set will group participants according to treatment received. If participants received multiple treatments during the study, they will be assigned to treatment group in the following manner:
If participant received both active and placebo treatments, they will be assigned to the active treatment group.
If participant received 2 or more different active dose levels, they will be assigned to the treatment they received for the longest period.

The mITT set will be the primary population of interest for the efficacy section, with some supportive analyses performed using the PPS. The safety set will be used for all safety summaries.

### Analysis of Primary Efficacy Endpoint

The assumption that the 3 cohorts of placebo participants can be pooled into a single placebo group to be used as a control for all active doses will be examined using mean (+SD) plots of percent change in PASI score against time.

The primary analysis will be performed using a Bayesian MMRM. The model will include parameters for treatment*visit and baseline PASI score*visit interactions. Body mass index, gender, and other baseline covariates will also be considered and included as parameters if found to be significant (p≤0.05). The model will not include an intercept. Visit will consist of 6 levels (Weeks 1, 2, 4, 8, 12, and 16) and treatment will consist of 4 levels (pooled placebo, Prevotella Strain B 50329 0.8×10¹¹ cells, Prevotella Strain B 50329 3.2×10¹¹ cells, and Prevotella Strain B 50329 8.0×10¹¹ cells) if the placebo pooling strategy is considered appropriate or 6 levels (Placebo matching Prevotella Strain B 50329 0.8×10¹¹ cells, Placebo matching Prevotella Strain B 50329 3.2×10¹¹ cells, Placebo matching Prevotella Strain B 50329 8.0×10¹¹ cells, Prevotella Strain B 50329 0.8×10¹¹ cells, Prevotella Strain B 50329 3.2×10¹¹ cells, and Prevotella Strain B 50329 8.0×10¹¹ cells) if the placebo pooling strategy is not considered appropriate.

The priors for all parameters in the model will be non-informative and follow a normal distribution with mean 0 and SD 1000. The prior for the variance-covariance matrix will follow an inverted Wishart distribution with degrees of freedom equal to the number of visits and an identity scale matrix. The choice of Wishart distribution is based on it being the conjugate prior of the inverse-covariance matrix of a multivariate-normal random vector.

If the assumption of similarity between the 3 placebo cohorts is considered appropriate, the placebo cohorts will be pooled, and a single placebo control group will be used for the pairwise differences for each active dose to placebo. If the assumption of similarity is considered inappropriate, each placebo dose will be included in the model as a separate dose level and pairwise comparisons between each active dose and placebo will be performed using only the matching placebo dose data for the relevant active dose.

The adjusted posterior mean percentage change from baseline and the associated 95% HDP CrI for each treatment at Week 16 will be reported, together with the adjusted mean difference from placebo and the associated 95% HDP CrI for each active dose at each visit and the probability that each treatment difference is less than 0%, −20%, −30%, and −50%.

Model checking and diagnostic plots, including posterior density plots of the posterior samples for all parameters in the model and residual plots to evaluate the distributional assumptions underlying the model, will be produced. The assumption that data are missing at random will be evaluated by plotting the mean percentage change in PASI score against visit, by treatment group, for the subgroups of participants who completed 16 weeks of study drug compared with those who discontinued study drug before the Week 16 visit.

If model checking and diagnostic plots show a violation of the assumptions underlying the analysis, alternative statistical methods will be considered, appropriate to the type of violation observed.

This primary analysis will be repeated using the 2 supportive estimands defined herein.

A further sensitivity analysis will be performed on the model with the primary estimand, in which participants who withdrew from study drug due to treatment failure (demonstrated by the participant commencing an oral agent, biological, or intermediate or high-potency topical therapy for plaque psoriasis) will have their percentage change from PASI imputed at all visits after study drug was discontinued as the maximum on-treatment value reached (ie, worst score carried forward).

If the assumption of similarity between the placebo cohorts is supported, a supplementary analysis will be performed on the percent change from baseline to Week 16 in PASI score using a dose-response model on the pooled cohorts. The log-linear, 3-parameter, and 4-parameter Eₘₐₓ models will be fitted and compared, with the best fitting model (lowest DIC) selected for use in the outputs.

The dose-response model will be fitted to the data using Bayesian techniques with noninformative priors for E₀ and Eₘₐₓ and an FUP for ED50 (3- and 4-parameter models only) and the slope parameter m (4-parameter model only). The rationale for this choice of inference is that the FUP shrinks the dose response towards a flat line throughout the dose range, therefore providing more conservative estimates of the dose-response relationship compared to maximum likelihood (Bornkamp 2014). The models will be fully described in the SAP.

Based on the selected model, the posterior mean with associated 95% HDP CrI, for the difference from placebo for each active dose will be produced for the pairwise differences between each active dose and placebo, together with the posterior mean and 95% HDP CrI of the treatment difference from placebo for each active dose and posterior probabilities that difference from placebo is less than 0, −20%, −30%, and −50%. A further sensitivity analysis will be performed on the dose response model, in which participants who withdrew from study drug due to treatment will have their Week 16 percentage change from PASI imputed as 100% after study drug was discontinued.

Percent change from baseline in PASI score will be summarized by visit.

Analysis of Secondary Efficacy Endpoints

All secondary analyses will be performed either using the pooled placebo group if the assumption of similarity for the placebo cohorts is considered appropriate or using the 3 cohort-level placebo groups if it is not considered appropriate.

Unless otherwise specified, all secondary analyses will be performed on the mITT set, excluding data collected after treatment discontinuation, without consideration of any protocol deviations. Dose will be treated as a categorical variable and no dose response modelling will be done. Comparisons of interest will be between individual Prevotella Strain B 50329 doses and placebo. All posterior probabilities and CrI calculated will be considered as descriptive with no further adjustments for multiplicity performed.

Data will be analyzed as collected and no imputation of missing data will be performed.

Mean percentage change from baseline in PASI score at Weeks 4, 8, and 12 will be analyzed as part of the MMRM for the primary estimand. The same statistics produced for the Week 16 time point will also be produced at Weeks 4, 8, and 12.

The following secondary endpoints will be analyzed in the same manner as described for the primary analysis:
Mean absolute change from baseline in PASI score at Weeks 4, 8, 12, and 16
Mean percentage change from baseline in LSS at Weeks 4, 8, 12, and 16
Mean absolute change from baseline in LSS at Weeks 4, 8, 12, and 16
Mean percentage change from baseline in PGA×BSA at Weeks 4, 8, 12, and 16
Mean absolute change from baseline in PGA×BSA at Weeks 4, 8, 12, and 16
Mean percentage change from baseline in DLQI score at Weeks 4, 8, 12, and 16
Mean absolute change from baseline in DLQI score at Weeks 4, 8, 12, and 16
Mean percentage change from baseline in mNAPSI total score at Weeks 4, 8, 12, and 16
Mean absolute change from baseline in mNAPSI total score at Weeks 4, 8, 12, and 16

For the PASI-50, a Bayesian generalized linear mixed effects model with a logit link function will be fitted using data from all visits. Treatment*visit and baseline PASI score*visit interactions will be included in the model as fixed effects. Body mass index, gender, and other baseline covariates will also be considered and fitted as fixed effects if found to be significant (p≤0.05). Odds ratios and 95% HDP CrI for each active dose compared to placebo at each visit will be presented.

A sensitivity analysis for the PASI-50 will also be performed, in the same manner as described above, in which participants who withdraw from study drug before Week 16 due to treatment failure will be included in the model with the PASI-50 endpoint imputed as ‘not achieved’ at all visits after study drug withdrawal.

Percentage of participants achieving PGA of 0 or 1 with a ≥2-point improvement and percentage of participants achieving a PGA of 0 will be analyzed in the same manner as described above for PAST-50.

For the time to first achievement of PASI-50, a Bayesian Cox proportional hazards model will be fitted with treatment and baseline PASI score as covariates. Hazard ratios and 95% HDP CrI for each active dose compared to placebo will be presented.

Analyses of Exploratory Efficacy Endpoints

Exploratory endpoints will be summarized using the mITT population, with data collected after discontinuation of treatment excluded but without consideration of any protocol deviations. Details of all analyses to be performed on the exploratory endpoints will be detailed in the SAP.

Analyses of biomarkers will be addressed in a data analysis plan outside the study SAP.

Pharmacokinetic Analyses

The number and percentage of participants who have a quantifiable concentration of Prevotella Strain B 50329 in their blood sample will be summarized using the safety set by visit. Placebo participants will be pooled into a single treatment group. If at least 20% of participants within a treatment group are found to have a quantifiable level at one of the visits, then the concentration will be summarized as a continuous variable for the relevant treatment group at that visit.

Interim Analyses

An interim analysis may be undertaken during the conduct of the study after at least 50% of participants have completed at least 12 weeks of treatment or withdrawn from treatment. The purpose of this analyses will be to aid in the planning of future studies and for a better understanding of the benefit/risk profile of Prevotella Strain B 50329.

For the interim analysis, unblinded aggregate results will be produced by an unblinded team for strategic planning use. These will not be shared with any study site staff, participants, or clinical monitors who will be involved in the collection and review of individual study data.

The interim analysis will look at the primary endpoint of percentage change from baseline in PASI score, secondary, and safety endpoints. The posterior predictive probability (Spiegelhalter et al 2004) of the percent change from baseline in PASI score being at least 20% lower in each active dose compared to the pooled placebo will also be calculated, using the estimates of treatment difference found at Week 12 using the Bayesian MMRM described for the primary analysis. If the posterior predictive probabilities for all active doses are found to be ≤30%, then the study may be stopped for futility.

No decisions regarding study conduct, other than the potential to stop the study early for futility, will be made based on these assessments and the study will not be stopped if superior efficacy is found. Outputs featuring unblinded treatment assignments will be created by the unblinded analysis group (to be included in the data dissemination plan).

### REFERENCES

Blake M R, Raker J M, Whelan K. Validity and reliability of the Bristol Stool Form Scale in healthy adults and patients with diarrhoea-predominant irritable bowel syndrome. Aliment Pharmacol Ther. 2016; 44:693-703.
Bornkamp B. Practical considerations for using functional uniform prior distributions for dose-response estimation in clinical trials. Biom J. 2014; 56(6):947-62.
Bushnell D M, Martin M L, McCarrier K, et al. Validation of the Psoriasis Symptom Inventory (PSI), a patient-reported outcome measure to assess psoriasis symptom severity. J Dermatolog Treat. 2013; 24(5):356-60.
Cassell S E, Bieber J D, Rich P, et al. The modified Nail Psoriasis Severity Index: validation of an instrument to assess psoriatic nail involvement in patients with psoriatic arthritis. J Rheumatol. 2007 January; 34(1):123-9.
de Groot P F, Belzer C, Aydin 0, et al. Distinct fecal and oral microbiota composition in human type 1 diabetes, an observational study. PLoS One. 2017; 12(12):e0188475.
Feldman S R, Krueger G G. Psoriasis assessment tools in clinical trials. Ann Rheum Dis. 2005; 64(Suppl 2):ii65-8; discussion ii69-73.
Felix K M, Tahsin S, Wu H J. Host-microbiota interplay in mediating immune disorders. Ann N Y Acad Sci. 2018; 1417(1):57-70.
Finlay A Y, Khan G K. Dermatology Life Quality Index (DLQI): a simple practical measure for routine clinical use. Clin Exp Dermatol. 1994; 19:210-6.
Hawker G A, Mian S, Kendzerska T, et al. Measures of adult pain: Visual Analog Scale for Pain (VAS Pain), Numeric Rating Scale for Pain (NRS Pain), McGill Pain Questionnaire (MPQ), Short□Form McGill Pain Questionnaire (SF□MPQ), Chronic Pain Grade Scale (CPGS), Short Form□36 Bodily Pain Scale (SF□36 BPS), and Measure of Intermittent and Constant Osteoarthritis Pain (ICOAP). Arthritis Care Res (Hoboken). 2011; 63 Suppl 11:S240-52.
Hindson J. Multiple sclerosis: A possible link between multiple sclerosis and gut microbiota. Nat Rev Neurol. 2017; 13(12):705.
Human Microbiome Project Consortium. A framework for human microbiome research. Nature. 2012; 486:215-21.
Langley R G, Ellis C N. Evaluating psoriasis with Psoriasis Area and Severity Index, Psoriasis Global Assessment, and Lattice System Physician's Global Assessment. J Am Acad Dermatol. 2004; 51(4):563-9.
Mangalam A, Shahi S K, Luckey D, et al. Human gut-derived commensal bacteria suppress CNS inflammatory and demyelinating disease. Cell Rep. 2017; 20(6):1269-77.
Marietta E V, Murray J A, Luckey D H, et al. Human gut-derived Prevotella histicola suppresses inflammatory arthritis in humanized mice. Arthritis Rheumatol. 2016; 68(12):2878-88.
O'Donnell L J D, Virjee J, Heaton K W. Detection of pseudodiarrhoea by simple clinical assessment of intestinal transit rate. BMJ. 1990; 300:439-40.
Patel R V, Tsui C L. Evaluating psoriasis: a review of the assessments most commonly used in clinical trials. Psoriasis Forum. 2011; 17(4):259-66. doi: 10.1177/247553031117a00403.
Skoie I M, Dalen I, Ternowitz T, et al. Fatigue in psoriasis: a controlled study. Br J Dermatol. 2017; 177(2):505-12.
Spiegelhalter D J, Abrams K R, Myles J P. Bayesian approaches to clinical trials and health-care evaluation. Chichester: John Wiley and Sons Ltd; 2004. 408 p.
van der Heijden P G, van Buuren S, Fekkes M, et al. Unidimensionality and reliability under Mokken scaling of the Dutch language version of the SF-36. Qual Life Res. 2003; 12:189-98.
Vandeputte D, Falony G, Vieira-Silva S, et al. Stool consistency is strongly associated with gut microbiota richness and composition, enterotypes and bacterial growth rates. Gut. 2016; 65:57-62.
Vandeputte D, Kathagen G, D'hoe K, et al. Quantitative microbiome profiling links gut community variation to microbial load. Nature. 2017; 551:507-11.
Walsh J A, McFadden M, Woodcock J, et al. Product of the Physician Global Assessment and body surface area: a simple static measure of psoriasis severity in a longitudinal cohort. J Am Acad Dermatol. 2013; 69(6):931-7.
Wolfe F. Fatigue assessments in rheumatoid arthritis: comparative performance of visual analog scales and longer fatigue questionnaires in 7760 patients. J Rheumatol. 2004; 31(10):1896-902.
Yan D, Issa N, Afifi L, et al. The role of the skin and gut microbiome in psoriatic disease. Curr Dermatol Rep. 2017; 6:94-103.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of treating psoriasis in a human subject comprising orally administering to the human subject a dose of 8×10¹⁰ to 8×10¹¹ total cells of Prevotella histicola Strain B 50329 (NRRL accession number B 50329) formulated in one or more enteric coated capsules or tablets.

2. A method of decreasing Lesion Severity Score (LSS) (e.g., mean LSS) (e.g., as compared to baseline or placebo control) in a human subject (e.g., a subject with psoriasis) comprising orally administering to the human subject a dose of 8×10¹⁰ to 8×10¹¹ total cells of Prevotella histicola Strain B 50329 (NRRL accession number B 50329) formulated in one or more enteric coated capsules or tablets.

3. The method of claim 2, wherein the mean LSS is decreased in the subject.

4. The method of claim 2, wherein the LSS is reduced as compared to baseline or placebo control.

5. A method of decreasing Psoriasis Area and Severity Index (PASI) score (e.g., mean PASI score) (e.g., as compared to baseline or placebo control) in a human subject (e.g., a subject with psoriasis) comprising orally administering to the human subject a dose of 8×10¹⁰ to 8×10¹¹ total cells of Prevotella histicola Strain B 50329 (NRRL accession number B 50329) formulated in one or more enteric coated capsules or tablets.

6. The method of claim 5, wherein the mean PASI score is decreased in the subject.

7. The method of claim 5, wherein the PASI score is reduced as compared to baseline or placebo control.

8. A method of increasing a sustained clinical effect (e.g., continued reductions from baseline (or placebo) in mean LSS and/or PASI, e.g., two weeks after completion of dosing) in a human subject (e.g., a subject with psoriasis) comprising orally administering to the human subject a dose of 8×10¹⁰ to 8×10¹¹ total cells of Prevotella histicola Strain B 50329 (NRRL accession number B 50329) formulated in one or more enteric coated capsules or tablets.

9. The method of claim 8, wherein the sustained clinical effect comprises continued reductions from baseline or placebo in mean LSS and/or PASI after completion of dosing.

10. The method of claim 9, wherein the reductions from baseline or placebo in mean LSS and/or PASI are continued for at least 2 weeks after dosing.

11. The method of claim 2, wherein the LSS and/or PASI score are reduced by at least 10%, 15%, 20%, 25%, 30%, 35%, 40% 50%, 60%, 70%, 80%, or 90% compared to baseline or placebo.

12. The method of claim 1, wherein the bacterial composition comprises about 0.8×10¹¹ total cells of Prevotella histicola.

13. The method of claim 1, wherein the bacterial composition comprises about 3.2×10¹¹ total cells of Prevotella histicola.

14. The method of claim 1, wherein the bacterial composition comprises about 8.0×10¹¹ total cells of Prevotella histicola.

15. The method of claim 1, wherein the bacterial composition is administered at least once daily.

16. The method of claim 1, wherein the bacterial composition is administered once daily.

17. The method of claim 1, wherein the bacterial composition is administered once daily for 15 continuous days.

18-19. (canceled)

20. The method of claim 1, wherein the psoriasis is mild to moderate psoriasis.

21. A method of treating atopic dermatitis in a human subject comprising orally administering to the human subject a dose of 8×10¹⁰ to 8×10¹¹ total cells of Prevotella histicola Strain B 50329 (NRRL accession number B 50329) formulated in one or more enteric coated capsules or tablets.

22-30. (canceled)

31. A method of enhancing anti-inflammatory cytokine production in a human subject comprising orally administering to the human subject a dose of 8×10¹⁰ to 8×10¹¹ total cells of Prevotella histicola Strain B 50329 (NRRL accession number B 50329), wherein the bacterial composition comprises about 0.8×10¹¹ to about 8×10¹¹ total cells of Prevotella histicola.

32-41. (canceled)


SEQUENCE LISTING (69 sequences)

SEQ ID NO: 1 - protein, 770 aa, Prevotella histicola
MNLKTFTKTVLCFALFAVSAITAKAADHLAIVGEAVWGGWDLVKATAMVKSPNNPDVFMATVHLNAGKGFKFLTEREWGKLEYRSGASDVVLKSGIRYKLYASIGASEDGKFKVSESANYEIICDLARKTVEVKKVAYQAKEIRYAALWMIGDATAGDWDYNNGVLLSQDSGNPTCYTATVELKEGEFKFTTNKQWGYDHSVYIFRDVNDQNKIVFGGEDNKWRITEDGMYNVTVDVPTKTISIKQIDDPAGHKPQFGNDVILVGDATIAGWNLDNAIYLEHTGQAGRVFKTTTYLEAGKGFKFLSMLSYDDIDYRPANNTVLNPGVPGTFVPSLPSSTDTKFSVERSGNYDIVCNMNNRTVVVTLSENQVLVNYPALWLIGSATSAGWNPGKAVELKRSEADPAVYTARVQLKKGEFKILTSKNVGFDQPTYYRDSTNEHRIVFGVDGDEVAKKDCKWTLSENAEGTYDVTVDIEAMTIFCDKVNMDEPSVESTDKELILIGDATYSAWDLPKSIVMTPVGPTTFKAVTHLEAGKEFKFLTELAWKRYEYRAESLRKELQEGSMSMLVPYRYTNDKDDKDHDFKFVVKESGNYEIVCDLYIPALIIRKVRYQDTPVTYSSLWIVGSATPGGWTIERGIKMTQDENYPTKFTAKANLVPGELKFATNKFADFTQDFFFRGKDDYTAVLGGNDNKWNITEAGTYSVTIDVASKRVTITKPARNAPTGISTVDSSDEAPAEYFTLNGIKVTTPSSGIYIKRQGGRTTKVVMK

SEQ ID NO: 2 - protein, 193 aa, Salmonella typhimurium
MDTYQILDIIGCIVGLIYIYQEYKASIWLWMTGIIMPVIYMFVYYEAGLYADFGMQIYYTLAAIYGYLYWKLGKKKGTEDKEIPITHFPRRYIIPAIIVFFVLWIALYYILICFTNSTVPVLDSFGNALSFIGLWALAKKYLEQWWIWIVVDAELSALYIYKGIPFTAMLYALYTVIAVAGYFKWRRYIKQQK

SEQ ID NO: 3 - protein, 544 aa, Bacillus licheniformis
MRVRLYKNILLFLFLWVNTLACVSADTSRTVESQPIENGLIITESKGWLETIYAKWKPVAEADGYYVYVKGGQYADYSKVDSELIRVYNGYVRVDIPGLKAGTYSLKIVAVKGGKETQSSEVTGLKVLNYVREGFAHKNYSGVGAYNDDGTLKSGAVVIYVNKDNAKTVSAHLGKTTFIGLQAILNAYQKGNITTPLSVRILGLLRNGDTDTFGSSTEGIQIKGKQADSEMNITIEGIGEDASIYGFGFLVRNAKSVEFRNLGIMRAMDDGVSLDTNNSNIWIHHMDLFYGKASGGDHIKGDGSIDVKTDSKYVTIDNCHFWDTGKTSMCGMKKETGPNYITYHHNWFDHSDSRHARVRTMSVHLWNNYYDGCAKYGIGATMGCSVFSENNYFRATKNPILISKQGSDAKGTGKFSGEPGGMVKEYGSLFTEKGAESTYTPISYADNNSSFDFYHAISRNEKVPASVKTLNGGNIYNNFDTDAALMYSYTPDATALVPSQVTGFYGAGRLNHGSLQFKFNNAVEDTNSTPIPALEALIDAYSGK

SEQ ID NO: 4 - protein, 366 aa, Streptococcus gordonii
MKYNIAYCIEGFYNHGGMERILSVCANLLSDIYSITIIVANQRGREHAYNLAQNVNVVDLGVSCKNYKEEYKKSLTRYLQDHQFSVVISLAGLELFFLPQIKDGSKKVMWFHFAFDVSKMFLSERFHGWKLNLLYYIHTIRRIYFAKKFDTIVVLSKSDCDSWSRFCNNVKYIYNPITIDRKVISNLSEESVIAVGRLGWQKGFDFLIDSWVLVDDKHPDWHLDIFGEGPDRLELQHQIDRKGLHDKVRLCGVTKQIEEEYGKHSIYVMSSRAEGFPLALLEASSCGLPMISFNCHQGPNEIIQEGENGFLVDKVGDIYTLSDRICKLIEDNNLRNMMGKKALDSSFRFEGEVIKKDWISLLKQLI

SEQ ID NO: 5 - protein, 158 aa, Prevotella histicola
MKRLFFMFLFLGTITMNSLAQEEKPIKYETKNFSLPDKMPLYPGGDGALRAFLSLNLHYPEKAQAFGVEGRSLMKFCVSSDGSIKDISAVDCKITNYNRTEFNKLPLSKQESLKKECAKAFAKEAARVIRLMPKWEPAELNGKKMNVYYSLPFTFKLR

SEQ ID NO: 6 - protein, 415 aa, Prevotella histicola
MNYPLFIARKIYNGGDRTRKVSKPAIRIATIGVAIGLAVMIISVGVVLGFKHTIRNKVVGFGSDITVANFLTLQSSEQYPIQITDSLVKSLQITPGIKHVQRYDYTQGILKTDNDFLGVLLKGVGPDFDSTFIHENMVEGSLPHFHDNESQQKIVISKTIADKLNLKVGQRIFAYFINKQGVRTRKFTITGIYATNMKQFDSQICFTDIYTTNKLNGWEPDQYSGAELQVDNFSQLTPISMRVLNKVKNTVDHYGGTYSSENIIEQNPQIFSWLDLMDMNVWIILALMISVAGVTMISGLLIIILERTQMIGILKALGSRNRQIRHIFLWFATFIIGKGLLWGNIIGLGCILFQSWTGLVKLDPQTYYVNTVPVEINIPLIIALNMVTMLVCLVILIAPSYLISHIHPAKSMHYE

SEQ ID NO: 7 - protein, 736 aa, Mycobacterium tuberculosis
MEDKFIYTDKERKLSYQILDELKDTLDKSFLENDLPMLQVQLKDSVAKNTIHRNVFGLNPILCSLQTAAIAVKDIGLKRDSVIAILLHQSVQDGYITLEDIDNRFGKSVAKIIHGLIRIQTLYQKNPIIESENFRNLLLSFAEDMRVILIMIADRVNLMRQIRDAEDKEAQHKVAEEASYLYAPLAHKLGLYQLKRELEDLSLKYLEHDAYYLIKDKLNATKASRDAYINQFIAPVRERLTAGGLRFHIKGRTKSIHSIWQKMKKQKCGFEGIYDLFAIRIILDAPLEKEKIQCWQAYSIVTDMYQPNPKRLRDWLSVPKSNGYECLHITVLGPEKKWVEVQIRTERMDEIAEHGLAAHWRYKGIKEEGGLDDWLASIRAALEAGDNLEVMDQFKSDLYEKEIYVFTPKGDLLKFPKGATILDFAYHIHSKVGNQCVGGKINAKNVSLRTELHSGDTVEILTSATQKPKAEWLKIVKSSRAKAKIRLALKETQIKDGLYAKELLERRFKNKKIEIEESTMGHLLRKLGFKEVSEFYKQVADEKLDPNYIIEEYQKVYNHDHNLNQPKETESAENFEFENPTNEFLKKNDDVLVIDKNLKGLDFSLAKCCHPIYGDPVFGFVTVNGGIKIHRTDCPNAPEMRKRFGYRIVKARWSGKGSSQYAITLRVIGNDDIGIVSNITNVISKDEKIVMRSINIDSHDGLFSGNLVVLLDDNSKLNMLIKKLRTVKGVKQVTRI

SEQ ID NO: 8 - protein, 336 aa, Escherichia coli
MKRRIFLFVALSVSIVILFGLNLIIGSVHIPLSDILTILSGSFTGKESWRFIIWDSRLPQALTAMLCGSSLAVCGLMLQTAFRNPLAGPDVFGISSGASLGVALVMLLLGGTVETSMFTASGFLAILIVAFAGAILVTAFILFLSSVVRNSVLLLIVGIMVGYVASSAVTLLNFFSSEDGVKGYIVWGMGNFGGVSMSHIPLFAFLCLAGIIASFLLVKPLNILLLGPQYAESLGISIRRIRNILLVVVGILTAVTTAFCGPISFIGLAAPHVARLLFRTENHQKLLPGTLLVGTVVALLCNLICFLPRESGMIPLNAVTPLIGAPIIIYVIMKRH

SEQ ID NO: 9 - protein, 524 aa, Escherichia coli
MKLENKEFGFDSFATEMARLKNEKHFDYLVTVVGEDFGTEEGLGCIYILENTSTHERCSVKQLAKKVGEEFVIPSVIKLWADADLLEREVYDFYGIKFLGHPDMRRLFLRNDFKGYPLRKDYDMDPAKNMYTTEDDVELDTTTEWNLDKNGELVGTQHALFTDDNFVVNIGPQHPSTHGVLRLQTVLDGETVTNIYPHLGYIHRGIEKLCEQFTYPQTLALTDRMNYLSAMMNRHALVGVIEEGMGIELSERILYIRTIMDELQRIDNHLLYTACCAQDLGALTAFLYGMRDREHVLNVMEETTGGRLIQNYYRIGGLQADIDPNFVSNVKELCKYLRPMIQEYVDVFGDNVITHQRFEGVGVMDEKDCISYGVTGPAGRASGWKNDVRKYHPYAMYDKVNFEEITLTNGDSMDRYFCHIKEIYQSLNIIEQLIDNIPEGEFYIKQKPIIKVPEGQWYFSVEGASGEFGAYLDSRGDKTAYRLKFRPMGLTLVGAMDKMLRGQKIADLVTTGAALDFVIPDIDR

SEQ ID NO: 10 - protein, 142 aa, Escherichia coli
MRTSTQSKDMGKKQEYKLRNEEFLHNISKKDSIKTLPHGIFYEIIKEGSGEGTVQPRSIVICNYRGSLISGQVFDDSWQKPTPEAFRLNELITGLQIALCAMHKGDSWRIYIPYQEGYGSKRNADIPAFSTLIFDIELINIA

SEQ ID NO: 11 - protein, 196 aa, Mycobacterium tuberculosis
MADNKIAKESVKREVIAGERLYTLLVYSENVAGVLNQIAAVFTRRQVNIESLNVSASSIEGIHKYTITAWSDAATIEKITKQVEKKIDVIKADYYEDSDLFIHEVGLYKIATPILLENAEVSRAIRKRNARMMEVNPTYSTVLLAGMTDEVTALYHDLKNFDCLLQYSRSGRVAVTRGFSEPVSDFLKSEEESSVL

SEQ ID NO: 12 - protein, 390 aa, Escherichia coli
MKKKVKIGLLPRVIIAILLGIFFGYFMPTPLARVFLTFNGIFSQFLGFMIPLIIIGLVTPAIADIGKGAGKLLLVTVIIAYVDTVVAGGLAYGTGLCLFPSMIASTGGAMPHIDKATELAPYFSINIPAMADVMSGLVFSFMLGLGIAYGGLTATKNIFNEFKYVIEKVIAKAIIPLLPLYIFGVFLNMAHNGQAQQILLVFSQIIIVILVLHVFILVYQFCIAGAIIRRNPFRLLWNMMPAYLTALGTSSSAATIPVTLEQTMKNGVGKEIAGFVVPLCATIHLSGSAMKITACALTICLLVGLPHDPALFIYFILMLSIIMVAAPGVPGGAIMAALAPLASILGFNSEAQALMIALYIAMDSFGTACNVTGDGAIALVVNKMFGKKER

SEQ ID NO: 13 - protein, 162 aa, Prevotella histicola
MKKLLLLVCAAVMSLSASAQAGDKALGAQLVFGSETNSLGFGVKGQYYFTDHIRGEGSFDYFLKNKGISMWDINANVHYLFDVADKFKVYPLAGLGYTNWSYKYEYAGAPVVEGSDGRLAVNLGGGVEYELTKNLNVNAEAKYQIISNYNQLVLGVGVAYKF

SEQ ID NO: 14 - protein, 84 aa, Nostoc sp.
MHFYCTKSSLDTMSERYVKRMIAKLASQGKTVISIAHRFSTIMDAKHIILLAKGKVVAEGTHQELLKTSEDYRKLWSDQNDEID

SEQ ID NO: 15 - protein, 254 aa, Pseudomonas aeruginosa
MKNVYFLSDAHLGSLAIAHRRTQERRLVRFLDSIKHKASAVYLLGDMFDFWDEYKYVVPKGFTRFLGKVSELTDMGVEVHFFTGNHDLWTYGYLEEECGVILHRKPVTMEIYGKVFYLAHGDGLGDPDPMFQFLRKVFHNRVCQRLLNFFHPWWGMQLGLNWAKKSRLKRADGKEMPYLGEDKEYLVRYTKDYMRSHKDIDYYIYGHRHIELDLTLSGKVRMLILGDWIWQFTYAVFDGEHMFLEEYIEGESKP

SEQ ID NO: 16 - protein, 532 aa, Escherichia coli
MNSKQNDNYDVIIIGGGITGAGTARDCALRGLKVLLVEKFDFTNGATGRNHGLLHSGARYAVTDPESATECIKENMVLRRIAKHCIEETDGLFITLPEDDINYQKTFVEACARAGISANIISPEEALRLDPSVNPDLLGAVRVPDASVDPFHLTTANVLDARQHGADVLTYHEVVAILTSNGRVEGVRLRNNHTGEEIEKHAVLVINAAGIWGHDIAKMADIKINMFPAKGTLLVFGHRVNKMVINRCRKPANADILVPDDAVCVIGTTSDRVPYDTVDNLKITSEEVDTLIREGEKLAPSLATTRILRAYAGVRPLVAADNDPTGRSISRGIVCLDHEKRDGLTGMITITGGKMMTYRLMAEQATDLACKKLGINKTCETATTPLPGTAGKDSDNPHHTYSTAHKAAKGRQGNRVKEIDERTEDDRALICECEEVSVGEAKYAIEELHVHDLLNLRRRTRVGMGTCQGELCACRAAGVMCENGVKVDKAMTDLTKFINERWKGMRPVAWGSTLDEAQLTTIIYQGLCGLGI

SEQ ID NO: 17 - protein, 416 aa, Escherichia coli
MRYDTIIIGGGLSGLTAGITLAKAGQKVCIVSAGQSSLHFHSGSFDLLGYDADGEVVTHPLQAIADLKAEHPYSKIGISNIEHLASQAKTLLCEAGISVMGNYEQNHYRVTPLGTLKPAWLTTEGYAMIDDPEILPWKKVELLNIQGFMDFPTQFIAENLRMMGVECQIKTFTTDELSTARQSPTEMRATNIAKVLANKDALSKVSERINAISGDPDALLLPAVLGFSNAESLDEMKQWIKKPVQYIATLPPSVSGVRTTILLKRLFAQAGGTLLIGDSATTGQFSGNHLVSITTDHLPDEKLYADHFILASGSFMSHGIRSNYAGVYEPVFKLDVDAAEKRDDWSVTNAFEAQPYMEFGVHTDKDFHATKDGKNIENLYAIGSVLSGHNSIKHADGTGVSLLTALYVAKKITGKG

SEQ ID NO: 18 - protein, 416 aa, Escherichia coli
MAEGIQLKNISGNNLEQCLKCSICTAYCPVSAVEPKYPGPKQSGPDQERYRLKDSKFFDEALKMCLNCKRCEVACPSGVRIADIIQASRITYSTHRPIPRDIMLANTDFVGTMANMVAPIVNATLGLKPVKAVLHGVMGIDKHRTFPAYSSQKFETWYKRMAAKKQDSYSKHVSYFHGCYVNYNFPQLGKDLVKIMNAVGYGVHLLEKEKCCGVALIANGLSGQARRQGKVNIRSIRKAAEQNRIVLTTSSTCTFTMRDEYEHLLDIKTDDVRENITLATRFLYRLIEKGDIKLAFRKDFKMRTAYHSACHMEKMGWIIYSTELLKMIPGLELIMLDSQCCGIAGTYGFKKENYQRSQEIGEGLFKQIKELNPDCVSTDCETCKWQIEMSTGYEVKNPISILADALDVEETIKLNQ

SEQ ID NO: 19 - protein, 270 aa, Bacillus subtilis
MMIKNIVLSIPISLIIYLNHLIMEYSMTTQFLMELIGTLILVLFGDGVCACVTLNKSKGQKAGWVVITIAWGLAVCMGVLVAGPYTGAHLNPAVSIGLAVAGMFPWSSVPYYIVAQMIGGFLGGLLVWFFYKDHYDATDDEAAKLGTFCTSPAIRNYKMNFLSEVIATLVLVFIIISFSVDGNTGDAEHFKFGLAALGPIPVTLLIIALGMSLGGTTGYAMNPARDLSPRLAHAVCMKGDNDWSYSWIPVLGPIIGAIIAGFCGAALLLV

SEQ ID NO: 20 - protein, 398 aa, Streptococcus pneumoniae
MSEKIIPSNEPAQAASEPIKASYTEYTVIPSQGYCQFVKCKKGDQPVVLKGLKEAYRERVLLRNALKREFKQCQRLNHPGIVRYQGLVDVEGYGLCIEEEYVDGRTLQAYLKESHTDDEKITIVNQIADALRYAHQQGVAHRNLKPSNILITKQGDHVKLIDFNVLSLDDVKPTADTTRFMAPELKDETMTADGTADIYSLGTIMKVMGLTLAYSEVIKRCCAFKRSDRYSDIDEFLADFNHDGSSFSMPKIGKGTVVIGFIAVVVIALAALAYNYGGALVDQVGKIDVTSIFKSDAETAPEDSAMVKSVEQNNNDSVADEAPATGKLAFMNTMKPALYKDLDRLFAKHSDDRAKLNRAIKVYYRGLIQANDTLDNEQRAELDRVFGNYVKQKKAALK

SEQ ID NO: 21 - protein, 62 aa, Prevotella histicola
MLVAQLFVGVLQAQKPVQNRRQAVGQSMERQGLVNVKAVVPSIKVALMYARTDNFCHRMALS

SEQ ID NO: 22 - protein, 440 aa, Escherichia coli
MITGLVIIQLLIVLALIFIGARVGGIGLGIYGMIGVFILVYGFGLAPGSAPIDVMMIIVAVITAASALQASGGLEYLVGVAAKFLQKHPDHITYFGPITCWLFCVVAGTAHTSYSLMPIIAEIAQTNKIRPERPLSLSVIAASLGITCSPVSAATAALISQDLLGAKGIELGTVLMICIPTAFISILVAAFVENHIGKELEDDPEYKRRVAAGLINPEAACEEVQKAENEHDPSAKHAVWAFLFGVALVILFGFLPQLRPEGVSMSQTIEMIMMSDAALILLVGKGKVGDAVNGNIFKAGMNAVVAIFGIAWMGNTFYVGNEKILDAALSSMISSTPILFAVALFLLSIMLFSQAATVTTLYPVGIALGINPLLLIAMFPACNGYFFLPNYPTEVAAIDFDRTGTTRVGKYVINHSFQIPGFITTIVSILLGVLMVQFFR

SEQ ID NO: 23 - protein, 351 aa, Escherichia coli
MRILKITFVTVLALVMSTVVFAQKPKIRIIATGGTIAGVSASATSSAYGAGQVGVQTLIDAVPQIKDIADVSGEQLVNIGSQDMNDEVWLKLAKRINDLLNKEGYDGVLITHGTDTMEETAYFLSLTVHTDKPVVMVGSMRPSTAISADGPANLYNGICTLVDPSSKGHGVMVCMNNELFEAKSVIKTHTTDVSTFKGGLYGEMGYVYNGKPYFLHKPVAKQGLTSEFNVDNLTSLPKVGIVYGYANCSPLPIQAFVNAKFDGIVLAGVGDGNFYKDVFDVALKAQNSGIQIVRSSRVPFGPTNLNGEVDDAKYHFVASLNLNPQKARVLLMLALTKTKDWQKIQQYFNEY

SEQ ID NO: 24 - protein, 574 aa, Mycobacterium tuberculosis
MALACAMTMSASAQMGTNPKWLGDAIFYQIYPSSYMDTDGNGIGDLPGITQKLDYIKSLGVNAIWLNPVFESGWFDGGYDVIDFYKIDPRFGTNTDMVNLVKEAHKRGIKVCLDLVAGHTSTKCPWFKESANGDRNSRYSDYFIWTDSISEADKKEIAERHKEANPASSTHGRYVEMNAKRGKYYEKNFFECQPALNYGFAKPDPNQPWEQPVTAPGPQAVRREMRNIMAFWFDKGVDGFRVDMASSLVKNDWGKKEVSKLWNEMREWKDKNYPECVLISEWSDPAVAIPAGFNIDFMIHFGIKGYPSLFFDRNTPWGKPWPGQDISKDYKFCYFDKAGKGEVKEFVDNFSEAYNATKNLGYIAIPSANHDYQRPNIGTRNTPEQLKVAMTFFLTMPGVPFIYYGDEIGMKYQMDLPSKEGSNERAGTRTPMQWTSGPTAGFSTCNPSQLYFPVDTEKGKLTVEAQQNDPRSLLNYTRELTRLRHSQPALRGNGEWILVSKESQPYPMVYKRTSGGETVVVAINPSDKKVSANIAHLGKAKSLIMTGKASYKTGKTEDAVELNGVSAAVFKIAE

SEQ ID NO: 25 - protein, 244 aa, Listeria monocytogenes
MNIAVIFAGGSGLRMHTKSRPKQFLDLNGKPIIIYTLELFDNHPGIDAIVVACIESWIPFLEKQLRKFEINKVVKIVPGGESGQASIYNGLCAAEAYIKSKNVASEDTTVLIHDGVRPLITEETITDNINKVAEVGSCITCIPATETLVVKQHDGSLEIPSRADSLIARAPQSFLLSDILTAHRRAIDEKKNDFIDSCTMMSHYGYRLGTIIGPMENIKITTPTDFFVLRAMVKVHEDQQIFGL

SEQ ID NO: 26 - protein, 327 aa, Escherichia coli
MTEKKSVSIVLCTYNGTKYLQEQLDSILAQTYPLHEIIIQDDGSTDNTWQILEKYEEKYPLIHIYHNEGTHGVNANFLSAMHRTTGDFIAIADQDDIWETDKIANQMTTIGNKLLCSGLTRPFSSDGSFAYFDNRPRNVSIFRMMFLGLPGHTMLFRRELLRMMPPVTHSFFNVSLYDAALSILAASHDSIAFCNKVLVNFRRHADATTYNDYSRSLPSWQNGLYELLWGLRHYHQARSIALPIYRGKLALMEGITTNYHDFIEAKAIMRLETQKGLWAFLRLQYLLTKNHQRLFQTSGGSFIKMIRAWLYPVMQLYMYHHALRRCK

SEQ ID NO: 27 - protein, 437 aa, Enterobacter cloacae
MESFIIEGGHRLSGTIAPQGAKNEALEVICATLLTTEEVIIRNIPNILDVNNLIKLLQDIGVKVKKLGANDFSFQADEVKLDYLESIDFVKKCSSLRGSVLMIGPLLGRFGKATIAKPGGDKIGRRRLDTHFLGFKNLGARFVRIEDRDVYEIQADKLVGDYMLLDEASVTGTANIIMSAVMAEGTTTIYNAACEPYIQQLCHLLNAMGAKITGIASNLITIEGVTSLHGAEHRILPDMIEVGSFIGMAAMVGDGVRIKDVSIPNLGLILDTFRRLGVQIIEDEDDLIIPRQDHYVIDSFIDGTIMTISDAPWPGLTPDLISVLLVVATQAQGSVLFHQKMFESRLFFVDKLIDMGAQIILCDPHRAVVVGHDHAKKLRAGRMSSPDIRAGIALLIAALTAEGTSRIDNIAQIDRGYENIEGRLNALGAKVQRVEIC

SEQ ID NO: 28 - protein, 772 aa, Escherichia coli
MERSGNFYKAIRLGYILISILIGCMAYNSLYEWQEIEALELGNKKIDELRKEINNINIQMIKFSLLGETILEWNDKDIEHYHARRMAMDSMLCRFKATYPAERIDSVRHLLEDKERQMCQIVQILEQQQAINDKITSQVPVIVQKSVQEQPKKSKRKGFLGIFGKKEEAKPTVTTTMHRSFNRNMRTEQQAQSRRLSVHADSLAARNAELNRQLQGLVVQIDGKVQTDLQKREAEITAMRERSFIQIGGLTGFVILLLVISYIIIHRNANRIKRYKQETADLIERLQQMAKRNEALITSRKKAVHTITHELRTPLTAITGYAGLIQKNFNADKTGMYIRNIQQSSDRMREMLNTLLSFFRLDDGKEQPNFSTCRISSIAHTLESEFMPIAINKGLALTVTNHTDAVVLTDKERILQIGNNLLSNAIKFTENGAVSLTMGYDNGMLKLIVKDTGSGMTEEEQQRVFGAFERLSNAAAKDGFGLGLSIVQRIVTMLGGTIQLKSEKGKGSRFTVEIPMQSAEELPERINKTQIHHNRTLHDIVAIDNDKVLLLMLKEMYAQEGIHCDTCTNAAELMEMIRRKEYSLLLTDLNMPDINGFELLELLRTSNVGNSRIIPIIVTTASGSCNREELLERGFSDCLLKPFSISELMEVSDKCAMKGKQNEKPDFSSLLSYGNESVMLDKLIAETEKEMQSVRDGEQRKDFQELDALTHHLRSSWEILRADQPLRELYKQLHGSAVPDYEALNNAVTAVLDKGSEIIRLAKEERRKYENG

SEQ ID NO: 29 - protein, 321 aa, Staphylococcus aureus
MKRSRFYITVGLILSLTLLMSACGQKKAKDGRTDTPTSGTIKFASDESFSPIVEELLQNYQFRYPQAHLLPIYTDDNTGMKLLLDQKVNLFITSHAMTKGEDAILRGKGPIPEVFPIGYDGIAFIVNRSNPDSCITVDDVKKILQGKIAKWNQLNPKNNRGSIEVVFDNKASATLHYVVDSILGGKNIKSENIVAAKNSKSVIDYVNKTPNAIGVIGSNWLNDHRDTTNTTFKKDVTVASISKATVASPSNSWQPYQAYLLDGRYPFVRTIYALLADPHKALPYAFANYIANPIGQMIIFKAGLLPYRGNINIREVEVKNQ

SEQ ID NO: 30 - protein, 198 aa, Mycobacterium tuberculosis
MAGTKRIKTALISVFHKDGLDDLLKKLDEEGVQFLSTGGTQQFIESLGYECQKVEDVTSYPSILGGRVKTLHPKIFGGILARRDNEEDQKQMVEYTIPAIDLVIVDLYPFEQTVASGASAQDIIEKIDIGGISLIRAGAKNFKDVVIVPSKAEYPVLLQLLNTKGAETEIEDRKMFAERAFGVSSHYDTAIHSWFAAE

SEQ ID NO: 31 - protein, 472 aa, Escherichia coli
MEEEKGGRIGQRPYILKIITERNYIIIIDMKKAKILLFVTALVAVLTSCGGGQKGLPTSDEYPVITIGASNAQLKTTYPATIKGVQDVEVRPKVSGFITKLNIHEGEYVHAGQVLFVIDNSTYQAAVRQAQAQVNSAQSAVAQAKANVVQANASLNSANAQAATSRLTYNNSQNLYNNKVIGDYELQSAKNTYETAQASVRQAQSGIASAQAAVKQAEAGVRQAQAMLSTAKDNLGFCYVKSPASGYVGSLPFKEDALVSASSAQPVTTISNTSTIEVYFSMTEADVLKLSRTDDGLSNAIKKFPAVSLLLADGSTYNHEGAIVKTSGMIDATTGTINVIARFPNPEHLLKSGGSGKIVIAKNNNRALLIPQEAVTQVQNKMFVYKVDAKDKVHYSEITVDPQNDGINYIVTSGLKMGERIVSKGVSSLEDGAKIKALTPAEYEEAIKKAEKLGENQSSASGFLKTMKGDSK

SEQ ID NO: 32 - protein, 292 aa, Bacillus anthracis
MAKRRNKARSHHSLQVVTLCISTAMVLILIGMVVLTVFTSRNLSSYVKENLTVTMILQPDMSTEESAALCQRIRSLHYINSLNFISKEQALKEGTRELGANPAEFAGQNPFTGEIELQLKANYANNDSIKNIERELRTYRGVSDITYPQNLVESVNHTLGKISLVLLVIAILLTIVSFSLMNNTIRLSIYARRFSIHTMKLVGASWGFIRAPFLRRAVMEGLVSALLAIAVLGVGLCLLYDYEPDITKVLSWDVLVITAGVMLAFGVLIATFCSWLSVNKFLRMKAGDLYKI

SEQ ID NO: 33 - protein, 828 aa, Campylobacter jejuni
MKLSDLKTGETGVIVKVLGHGGFRKRIIEMGFIQGKQVEVLLNAPLRDPVKYKIMGYEVSLRHSEADQIEVISAEEARQLEQAKADNEPQQGALSNNIPDESDHALTPFELTDAANRKSKVINVALVGNPNCGKTSLFNFASGAHERVGNYSGVTVDAKVGRANYEGYEFHLVDLPGTYSLSAYSPEELYVRKQLVEKTPDVVINVIDASNLERNLYLTTQLIDMHVRMVCALNMFDETEQRGDNIDYQKISELFGIPMVPTVFTNGRGVKELFHQVIAVYEGKEDETSQFRHIHINHGHELEGGIKNIQEHLRAYPDICQRYSTRYLAIKLLEHDKDVEELIKPLKDSDEIFKHRDIAAQRVKEETGNESETAIMDAKYGFIHGALEEADYSTGQKKDTYQTTHFIDQILTNKYFGFPIFFLILFIMFTATFVIGQYPMDWIDGGVSWLGDFISSNMPDGPVKDMLVDGIIGGVGAVIVFLPQILILYFFISYMEDSGYMARAAFIMDKLMHKMGLHGKSFIPLIMGFGCNVPAVMATRTIESRRSRLVTMLILPLMSCSARLPIYVMITGSFFALKYRSLAMLSLYVIGILMSVIMSRVFSRFLVKGEDTPFVMELPPYRFPTWKAIGRHTWEKGKQYLKKMGGIILVASIIVWALGYFPLPDKPDMGQQERQEHSFIGQIGHAVEPVFRPQGFNWKLDVGLLAGVGAKEIVASTMGVLYSNDDSFKDDNSFSSEGGKYVKLHKQITQDVANLHGVSYNEAEPIATLTAFCFLLFVLLYFPCIATIAAIKGETGSWGWALFAAGYTTLLAWVVSAIVFQVGMLFIG

SEQ ID NO: 34 - protein, 566 aa, Streptococcus pneumoniae
MKKNLLKAVLPASLALFAVTFGSCSQDGQLTGTKEDTGERVLDNTREIQNYLRTLPLAPMMSRASDPVPSDDGTTVPVDEGTSKTEEKGVLNGIPGSWVKTTRRYKMTQAFDESFLFDPTSDIVYPGCVLKGGTIANGTYAIITSHETGDVTFSINLSPANPQEARETSATVHNIRKSEYQEVWNKWANMQWKESPITTIESVEKINSQEELATKLGVAVNSPVANGSLNFGFNFNKKKNHILARLIQKYFSVSTDAPKKGNIFESIDKEALDGYQPVYISNINYGRIIYLSVESDEDEKVVDEAINFAMNQIKGVDVSVSADQSLHYRKVLANCDIRITVLGGGQTIQKEVLKGDIDSFQRFLNADIPMEQMSPISFSLRYAVDNSQARVVTSNEFTVTQRDFVPEFKKVRMQLQVLGFSGTNTGPFPNLDREAGLWGSISLSLNGQDNELVKISQSNPFFFNYREKKETMHPIGFGGIVTVEFDKDPNESLEDFVDHQKMTFVSDLHSTRSIYNYNFGRTTFTHTLGTLYTKYKGDDPIFVLESNNKNVKIHTYVKVLDMKFFN

SEQ ID NO: 35 - protein, 922 aa, Prevotella histicola
MTKFIYAMSLFLLAAISIKAQPIQKTSGCLLHGSVVSSTDATAIAGATVRLYQLKKLVGGTVSDASGNFDVKCPSSGSLQLRITAVGFKEVDTTLNVPTVTPLSIYMRAGKHAMDEVTVTASEKRGMTSTTVIGQTAMEHLQPSSFADLLALLPGGMTKIPALGSANVITLREAGPPSSQYATSSLGTKFVIDGQAIGTDANMQYIAGSFQGDADNSRNHVSYGVDMREIPTDNIEKVEVVRGIPSVKYGELTSGLINITRKRSQSPLLLRLKADEYGKLVSVGKGFLLSGKWNLNVDGGLLDARKEPRNRFETYRRLTFSARLRRKWNLGERYVLEWSGATDYSLNIDNVKTDPEIQIHREDSYRSSYLKMGMNHRLLLRRKALVGLQSVSLAYSASLASDRIHQTEAVALQRDYVVPLAYEGGEYDGLFLPMQYLCDYRVEGKPFYSTLRGETEWLARTSFISHHITAGGEFLLNKNYGRGQIFDITKPLHASTARRPRSYKDIPATDILSFYAEDKATMPIGKHQLTVMAGLRTTQMLNIPASYAVHGKLFTDTRVNVQWDFPSFLGFKSFVSGGLGMMTKMPTVLDLYPDYVYKDITEMNYWDIRPAYKRIHIRTYKLNQVNPDLRPARNKKWEIRLGMDKGAHHFSVTYFHEDMKDGFRSTTTMRPFIYKRYDTSVINPSALTGPPSLASLPVVTDTLLDGYGRTENGSRITKQGIEFQYSSPRIPVIQTRITVNGAWFRTLYENSIPLFRSAPNVVVGTVAIADRYAGYYMSTDKYDKQIFTSNFIFDSYVDKLGLILSATAECFWMSNTKRPATSSTPMGYMDITGTVHPYVEADQSDPYLRWLVLTGTAGQDMDYRERSYMLVNFKATKRFGRHLSLSFFADRVFYVAPDYEVNGFIVRRTFSPYFGMEIGLKI

SEQ ID NO: 36 - protein, 257 aa, Escherichia coli
MLIDFKKVNIYQDERLILKDIDFQATEGEFIYLIGRVGSGKSSLLKTFYGELDIDQEDAEKAEVLGESVLDIKQKRIPALRRQMGIIFQDFQLLHDRSVAKNLKFVLQATGWKDKEKIKQRIKEVLEQVGMIDKAAKMPSELSGGEQQRIAIARAFLNNPKIILADEPTGNLDPETASNIVSILKDTCKNGTTVIMSTHNINLLSQFPGKVYRCMEQALVPVTNEAQTKDLEEDSTSVEPLIEPVLEEEAQAEDSKE

SEQ ID NO: 37 - protein, 508 aa, Lactococcus lactis
MFENQPKALYALALANTGERFGYYTMIAVFALFLRANFGLEPGTAGLIYSIFLGLVYFLPLIGGIMADKFGYGKMVTIGIIVMFAGYLFLSVPLGGGTVAFGAMLAALLLISFGTGLFKGNLQVMVGNLYDTPELASKRDSAFSIFYMAINIGALFAPTAAVKIKEWAETSLGYAGNDAYHFSFAVACVSLIVSMGIYYAFRSTFKHVEGGTKKTEKAAAAAVEELTPQQTKERIVALCLVFAVVIFFWMAFHQNGLTLTYFADEFVSPTSTGVQSMAFDVVNLVMIVFIVYSIMALFQSKTTKAKGIACAVILAAIAVLAYKYMNVNGQVEVSAPIFQQFNPFYVVALTPISMAIFGSLAAKGKEPSAPRKIAYGMIVAGCAYLLMVLASQGLLTPHEQKLAKAAGETVPFASANWLIGTYLVLTFGELLLSPMGISFVSKVAPPKYKGAMMGGWFVATAIGNILVSVGGYLWGDLSLTVVWTVFIVLCLVSASFMFLMMKRLEKVA

SEQ ID NO: 38 - protein, 492 aa, Myroides odoratus
MKKILIFVAGLCMSLAASAQIQRPKLVVGLVVDQMRWDYLYYYYNEYGTDGLRRLVDNGFSFENTHINYAPTVTAIGHSSVYTGSVPAITGIAGNYFFQDDKNVYCCEDPNVKSVGSDSKEGQMSPHRLLASTIGDELQISNDFRSKVIGVALKDRASILPAGHAADAAYWWDTSAGHFVTSTFYTDHLPQWVIDFNEKNHTAPNFNIKTSTQGVTMTFKMAEAALKNENLGKGKETDMLAVSISSTDAIGHVYSTRGKENHDVYMQLDKDLAHFLKTLDEQVGKGNYLLFLTADHGAAHNYNYMKEHRIPAGGWDYRQSVKDLNGYLQGKFGIAPVMAEDDYQFFLNDSLIAASGLKKQQIIDESVEYLKKDPRYLYVFDEERISEVTMPQWIKERMINGYFRGRSGEIGVVTRPQVFGAKDSPTYKGTQHGQPFPYDTHIPFLLYGWNVKHGATTQQTYIVDIAPTVCAMLHIQMPNGCIGTARNMALGN

SEQ ID NO: 39 - protein, 1138 aa, Staphylococcus epidermidis
MDRQVFQTDSRQRWNRFKWTLRVLITIAILLGVVFVAMFALEGSPQMPFRHDYRSVVSASEPLLKDNKRAEVYKSFRDFFKEQKMHSNYAKVAARQHRFVGHTDNVTQKYIKEWTDPRMGIRSAWYVNWDKHAYISLKNNLKNLNMVLPEWYFINPKTDRIEARIDQRALKLMRRAHIPVLPMLTNNYNSAFRPEAIGRIMRDSTKRMGMINELVAACKHNGFAGINLDLEELNINDNALLVTLVKDFARVFHANGLYVTQAVAPFNEDYDMQELAKYDDYLFLMAYDEYNAGSQAGPVSSQRWVEKATDWAAKNVPNDKIVLGMATYGYNWAQGQGGTTMSFDQTMATALNAGAKVNFNDDTYNLNFSYQDEDDGTLHQVFFPDAVTTFNIMRFGATYHLAGFGLWRLGTEDSRIWKYYGKDLSWESAARMPIAKIMQLSGTDDVNFVGSGEVLNVTSEPHAGRIGIVLDKDNQLIIEERYLSLPATYTVQRLGKCKEKQLVLTFDDGPDSRWTPKVLSILKHYKVPAAFFMVGLQIEKNIPIVKDVFNQGCTIGNHTFTHHNMIENSDRRSFAELKLTRMLIESITGQSTILFRAPYNADADPTDHEEIWPMIIASRRNYLFVGESIDPNDWQQGVTADQIYKRVLDGVHQEYGHIILLHDAGGDTREPTVTALPRIIETLQREGYQFISLEKYLGMSRQTLMPPIKKGKEYYAMQANLSLAELIYHISDFLTALFLVFLVLGFMRLVFMYVLMIREKRAENRRNYAPIDPLTAPAVSIIVPAYNEEVNIVRTISNLKEQDYPSLKIYLVDDGSKDNTLQRVREVFENDDKVVIISKKNGGKASALNYGIAACSTDYIVCVDADTQLYKDAVSKLMKHFIADKTGKLGAVAGNVKVGNQRNMLTYWQAIEYTTSQNFDRMAYSNINAITVIPGAIGAFRKDVLEAVGGFTTDTLAEDCDLTMSINEHGYLIENENYAVAMTEAPESLRQFIKQRIRWCFGVMQTFWKHRASLFAPSKGGFGMWAMPNMLIFQYIIPTFSPIADVLMLFGLFSGNASQIFIYYLIFLLVDASVSIMAYIFEHESLWVLLWIIPQRFFYRWIMYYVLFKSYLKAIKGELQTWGVLKRTGHVKGAQTIS

SEQ ID NO: 40 - protein, 508 aa, Propionigenium modestum
MSQINGRISQIIGPVIDVYFDTKGENPEKVLPNIYDALRVKKADGQDLIIEVQQQIGEDTVRCVAMDNTDGLQRGLEVVPTGSPIVMPAGEQIKGRMMNVIGQPIDGMSALQMEGAYPIHREAPKFEDLSTHKEMLQTGIKVIDLLEPYMKGGKIGLFGGAGVGKTVLIMELINNIAKGHNGYSVFAGVGERTREGNDLIRDMLESGVIRYGEKFRKAMDEGKWDLSLVDSEELQKSQATLVYGQMNEPPGARASVALSGLTVAEEFRDHGGKNGEAADIMFFIDNIFRFTQAGSEVSALLGRMPSAVGYQPTLASEMGAMQERITSTKHGSITSVQAVYVPADDLTDPAPATTFTHLDATTELSRKITELGIYPAVDPLGSTSRILDPLIVGKEHYDCAQRVKQLLQKYNELQDIIAILGMDELSDDDKLVVNRARRVQRFLSQPFTVAEQFTGVKGVMVPIEETIKGFNAILNGEVDDLPEQAFLNVGTIEDVKEKAKQLLEATKA

SEQ ID NO: 41 - protein, 984 aa, Prevotella histicola
MNPIYKIITSILFCVLSINTMAQDLTGHVTSKADDKPIAYATVTLKENRLYAFTDEKGNYTIKNVPKGKYTVVFSCMGYASQTVVVMVNAGGATQNVRLAEDNLQLDEVQVVAHRKKDEITTSYTIDRKTLDNQQIMTLSDIAQLLPGGKSVNPSLMNDSKLTLRSGTLERGNASFGTAVEVDGIRLSNNAAMGETAGVSTRSVSASNIESVEVVPGIASVEYGDLTNGVVKVKTRRGSSPFIVEGSINQHTRQIALHKGVDLGGNVGLLNFSIEHARSFLDAASPYTAYQRNVLSLRYMNVFMKKSLPLTLEVGLNGSIGGYNSKADPDRSLDDYNKVKDNNVGGNIHLGWLLNKRWITNVDLTAAFTYADRLSESYTNESSNATQPYIHTLTEGYNIAEDYDRNPSANIILGPTGYWYLRGFNDSKPLNYSLKMKANWSKAFGKFRNRLLVGGEWTSSMNRGRGTYYADMRYAPSWREYRYDALPSLNNIAIYAEDKLSMDVNERQNAELTAGIREDITSIPGSEYGSVGSFSPRMNARYVFRFGQNSWLNSMTLHAGWGRSVKIPSFQVLYPSPSYRDMLAFASTSDADNRSYYAYYTYPSMARYNANLKWQRADQWDLGVEWRTKIADVSLSFFRSKVSNPYMATDVYTPFTYKYTSPAMLQRSGIAVADRRFSIDPQTGIVTVSDASGVKSPVTLGYEERNTYVTNTRYVNADALQRYGLEWIVDFKQIKTLRTQVRLDGKYYHYKAQDETLFADVPVGLNTRQSDGRLYQYVGYYRGGAATTTNYTANASASNGSVSGQVDLNATITTHIPKIRLIVALRLESSLYAFSRATSSRGYVVSSGNEYFGVPYDDKTENQTVIVYPEYYSTWDAPDVLIPFAEKLRWAETNDRGLFNDLAQLVVRTNYPYTLNPNRLSAYWSANLSVTKEIGRHVSVSFYANNFFNTLSQVHSTQTGLETSLFGSGYVPSFYYGLSLRLKI

SEQ ID NO: 42 - protein, 273 aa, Escherichia coli
MERIDISVLMAVYKKDNPAFLRESLESIFSQTVEAAEVVLLEDGPLTDALYDVIKSYEAIYSTLKVVSYPENRGLGKTLNDGLLLCKYNLVARMDADDICKPNRLEMEYNWLKSHEDYDVIGSWVDEFTDNKTRVKSIRKVPEAYDEIKNYAQYRCPINHPTAMYRKAAVLAVGGYLTEYFPEDYFLWLRMLNNGSKFYNIQESLLWFRYSEETVAKRGGWAYACDEVRILVRMLKMGYIPFHVFCQSVVIRFTTRVMPLPIRQRLYNLIRKT

SEQ ID NO: 43 - protein, 507 aa, Paracoccus denitrificans
MSQINGRISQIIGPVIDVYFDTKGENPEKVLPKIHDALRVKRANGQDLIIEVQQHIGEDTVRCVAMDNTDGLQRNLEVVPTGSPIVMPAGDQIKGRMMNVIGQPIDGMEALSMEGAYPIHREAPKFEDLSTHKEMLQTGIKVIDLLEPYMKGGKIGLFGGAGVGKTVLIMELINNIAKGHNGYSVFAGVGERTREGNDLIRDMLESGVIRYGEKFRKAMDEGKWDLSLVDQEELQKSQATLVYGQMNEPPGARASVALSGLTVAEEFRDHGGKNGEAADIMFFIDNIFRFTQAGSEVSALLGRMPSAVGYQPTLASEMGTMQERITSTKHGSITSVQAVYVPADDLTDPAPATTFTHLDATTELSRKITELGIYPAVDPLGSTSRILDPLIVGKDHYECAQRVKQLLQHYNELQDIIAILGMDELSDEDKLVVNRARRVQRFLSQPFTVAEQFTGVKGVMVPIEETIKGFNAILNGEVDDLPEQAFLNVGTIEDVKEKAKRLLEATK

SEQ ID NO: 44 - protein, 268 aa, Mycobacterium tuberculosis
MPIGNGQKYQLTIINHTEIIMLIDYKKVNIYQDERLILKDVDFQAETGEFIYLIGRVGSGKSSLLKTIYGELDIDSEDAEKAVVLDESMPNIKRSRIPALRKQMGIIFQDFQLLHDRSVAKNLKFVLQATGWTSKQKIERRIEEVLAQVGMTDKKNKMPSELSGGEQQRIAIARALLNTPKIIIADEPTGNLDPETAANIVSILKDSCQAGTTVIMSTHNINLIDQFPGKVYRCHEGELHQLTDKKEVSELAEETAPVETIDEPEQND

SEQ ID NO: 45 - protein, 336 aa, Yersinia pestis
MKRNILLFICLATSILLLFGLNLTTGSVQIPFADILDILCGRFIGKESWEYIILENRLPQTLTAILCGASLSVCGLMLQTAFRNPLAGPDVFGISSGAGLGVALVMLLLGGTVSTSIFTVSGFLAILTAAFVGAIAVTALILFLSTLVRNSVLLLIVGIMVGYVSSSAVSLLNFFASEEGVKSYMVWGMGNFGAVSMNHIPLFSILCLIGIIASFLLVKPLNILLLGPQYAESLGISTRQIRNILLVVVGLLTAITTAFCGPISFIGLAIPHIARLLFRTENHQILLPGIVLSGAAIALLCNFICYLPGESGIIPLNAVTPLIGAPIIIYVIIQRR

SEQ ID NO: 46 - protein, 408 aa, Bacillus subtilis
MKKYYPWVLVALLWFVALLNYMDRQMLSTMQEAMKVDIAELNHAEAFGALMAVFLWIYGIVSPFAGIIADRVNRKWLVVGSIFVWSAVTYLMGYAESFDQLYWLRAFMGISEALYIPAALSLIADWHEGKSRSLAIGIHMTGLYVGQAVGGFGATLAAMFSWHAAFHWFGIIGIVYSLVLLLFLKENPKHGQKSVLQGETKPSKNPFRGLSIVFSTWAFWVILFYFAVPSLPGWATKNWLPTLFANSLDIPMSSAGPMSTITIAVSSFIGVIMGGVISDRWVQRNLRGRVYTSAIGLGLTVPALMLLGFGHSLVSVVGAGLCFGIGYGMFDANNMPILCQFISSKYRSTAYGIMNMTGVFAGAAVTQVLGKWTDGGNLGNGFAILGGIVVLALVLQLSCLKPTTDNME

SEQ ID NO: 47 - protein, 694 aa, Mycobacterium tuberculosis
MVTKKTTTKKAPVKKTSAKTTKVKEPSHIGLVKNDAYLAPYEDAIRGRHEHALWKMNQLTQNGKLTLSDFANGHNYYGLHQTADGWVFREWAPNATEIYLVGDFNGWNEQEAYQCHRIEGTGNWELTLPHDAMQHGQYYKMRVHWEGGEGERIPAWTQRVVQDEASKIFSAQVWAPAEPYVWEKKTFKPQTSPLLIYECHIGMAQDEEKVGTYNEFREKVLPRIIKDGYNAIQIMAIQEHPYYGSFGYHVSSFFAASSRFGTPEELKALIDEAHKNGIAVIMDIVHSHAVKNEVEGLGNLAGDPNQYFYPGERHEHPAWDSLCFDYGKDEVLHFLLSNCKYWLEEYHFDGFRFDGVTSMLYYSHGLGEAFCNYADYFNGHQDDNAICYLTLANCLIHEVNKNAVTIAEEVSGMPGLAAKFKDGGYGFDYRMAMNIPDYWIKTIKELPDEAWKPSSIFWEIKNRRSDEKTISYCESHDQALVGDKTIIFRLVDADMYWHFRKGDETEMTHRGIALHKMIRLATIAAINGGYLNFMGNEFGHPEWIDFPREGNGWSHKYARRQWNLVDNEELCYHLLGDFDRKMLEVITSEKKFNETPIQEIWHNDGDQILAFSRGELVFVFNFSPSHSYSDYGFLVPEGSYNVVLNTDAREFGGFGFADDTVEHFTNSDPLYEKDHKGWLKLYIPARSAVVLRKK

SEQ ID NO: 48 - protein, 448 aa, Prevotella melaninogenica
MKIDIERIKYFLTVGMFMKTEHSSKRRNMLIRQFQKFYLTVKFFFVRDHAASTAQLSFSTIMAIVPIASMIFAIANGFGFGQFLEKQFREMLSAQPEAATWLLKLTQSYLVHAKTGLFIGIGLMIMLYSVFSLIRTVETTFDNIWQVKDSRPISRIVIDYTALMFLVPISIIILSGLSIYFYSFVENLNGLRFLGTIASFSLRYLVPWAILTLMFIVLYVFMPNAKVKITKTVAPAMIASIAMLCLQAVYIHGQIFLTSYNAIYGSFAALPLFMLWILASWYICLFCAELCYFNQNLEYYECLIDTEDICHNDLLILCATVLSHICQRFANDQKPQTALQIKTETHIPIRVMTDILYRLKEVNLISENFSPTSDEVTYTPTHDTNNITVGEMIARLESTPASDFALLGFSPKKAWNHDIYDRVGSIREIYLNELKSINIKELISYSEN

SEQ ID NO: 49 - protein, 326 aa, Bacillus anthracis
MMKRPSIARVVKVIICLLTPILLSFSGIGDNDIDKKKSTSKEVDDTLRIVITGDLLLDRGVRQKIDMAGVDALFSPTIDSLFHSSNYVIANLECPVTKIRERVFKRFIFRGEPEWLPTLRRHGITHLNLANNHSIDQGRNGLLDTQEQIKKAGMIPIGAGKNMEEAAEPVLISTSPRHVWVISSLRLPLENFLYLPQKPCVSQESIDSLIMRVKRLRATDKNCYILLILHWGWEHHFRATPQQREDAHKLIDAGADAIVGHHSHTLQTIETYRGKPIYYGIGNFIFDQRKPMNSRACLVELSITAEKCKAKALPIEIKNCTPYLSK

SEQ ID NO: 50 - protein, 259 aa, Helicobacter pylori
MILLSFDTEEFDVPREHGVDFSLEEGMKVSIEGTNRILDILKANNVCATFFCTGNFAELAPEVMERIKNEGHEVACHGVDHWQPKPEDVFRSKEIIERVTGVKVAGYRQPRMFPVSDEDIEKAGYLYNSSLNPAFIPGRYMHLTTSRTWFMQGKVMQIPASVSPHLRIPLFWLSMHNFPEWFYLRLVRQVLRHDGYFVTYFHPWEFYDLKSHPEFKMPFIIKNHSGHELEQRLDRFIKAMKADKQEFITYVDFVNRQKK

SEQ ID NO: 51 - protein, 252 aa, Escherichia coli
MAKNISFTIKYWKQNGPQDQGHFDTHEMKNIPDDTSFLEMLDILNEELIAAGDEPFVFDHDCREGICGMCSLYINGTPHGKTERGATTCQLYMRRFNDGDVITVEPWRSAGFPVIKDCMVDRTAFDKIIQAGGYTTIRTGQAQDANAILISKDNADEAMDCATCIGCGACVAACKNGSAMLFVSSKVSQLALLPQGKPEAAKRAKAMVAKMDEVGFGNCTNTRACEAVCPKNEKIANIARLNREFIKAKFAD

SEQ ID NO: 52 - protein, 398 aa, Mycobacterium tuberculosis
MSENKLSTNEQAQTADAPVKASYTEYKVIPSQGYCMIVKCRKGDQTVVLKTLKEEYRERVLLRNALKREFKQCQRLNHSGIVRYQGLVEVDGYGLCIEEEYVEGRTLQAYLKENHTDDEKIAIINQIADALRYAHQQGVIHRNLKPSNVLVTTQGDYVKLIDFSVLSPEDVKPTAETTRFMAPEMKDETLTADATADIYSLGTIMKVMGLTLAYSEVIKRCCAFKRSDRYSNVDELLADLNNEGSSFSMPKIGKGTVVLGLIIAVVIGIGALLYNYGGALIDQVGKIDVSSVFSSDAETAPEDTVKVNTAEQSDSLSTEAEAPAIGKLAFMNRMKPALYKDLDNIFEKNSADKAKLTKAIKTYYRGLIQANDTLDNEQRAEVDRVFGDYVKQKKAALN

SEQ ID NO: 53 - protein, 522 aa, Bacillus subtilis
MRKYICLLLFYLFTFLPLSAQQGNDSPLRKLQLAEMAIKNFYVDSVNEQKLVEDGIRGMLEKLDPHSTYTDAKETKAMNEPLQGDFEGIGVQFNMIEDTLVVIQPVVNGPSQKVGILAGDRIVSVNDSTIAGVKMARIDIMKMLRGKKGTKVKLGVVRRGVKGVLTFVVTRAKIPVHTINASYMIRPNVGYIRIESFGMKTHDEFMSAVDSLKKKGMKTLLLDLQDNGGGYLQSAVQISNEFLKNNDMIVYTEGRRARRQNFKAIGNGRLQDVKVYVLVNELSASAAEIVTGAIQDNDRGTVVGRRTFGKGLVQRPFDLPDGSMIRLTIAHYYTPSGRCIQKPYTKGDLKDYEMDIEKRFKHGELTNPDSIQFSDSLKYYTIRKHRVVYGGGGIMPDNFVPLDTTKFTRYHRMLAAKSIIINAYLKYADANRQALKAQYSSFDAFNKGYVVPQSLLDEIVAEGKKEKIEPKDAAELKATLPNIALQIKALTARDIWDMNEYFRVWNTQSDIVNKAVALATGK

SEQ ID NO: 54 - protein, 348 aa, Prevotella melaninogenica
MKLTEQRSSMLHGVLLITLFACAAFYIGDMGWVKALSLSPMVVGIILGMLYANSLRNNLPDTWVPGIAFCGKRVLRFGIILYGFRLTFQDVVAVGFPAIIVDAIIVSGTILLGVLVGRLLKMDRSIALLTACGSGICGAAAVLGVDGAIRPKPYKTAVAVATVVIFGTLSMFLYPILYRAGIFDLSPDAMGIFAGSTIHEVAHVVGAGNAMGAAVSNSAIIVKMIRVMMLVPVLLVIAFFVAKNVAERDDEAGGSRKINIPWFAILFLVVIGFNSLNLLPKELVDFINTLDTFLLTMAMSALGAETSIDKFKKAGFKPFLLAAILWCWLIGGGYCLAKYLVPVLGVAC

SEQ ID NO: 55 - protein, 833 aa, Prevotella sp.
MNKQFLLAALWLSPLGLYAHKANGIGAVTWKNEAPKERMIRGIDEDKTHQRFTLSGYVKDRNGEPLINATIYDLTTRQGTMTNAYGHFSLTLGEGQHEIRCSYVGYKTLIETIDLSANQNHDIILQNEAQLDEVVVTTDLNSPLLKTQTGKLSLSQKDIKTEYALLSSPDVIKTLQRTSGVADGMELASGLYVHGGNGDENLFLLDGTPLYHTNHSLGLFSSFNADVVKNVDFYKSGFPARYGGRLSSVIDVRTADGDLYKTHGSYRIGLLDGAFHIGGPIRKGKTSYNFGLRRSWMDLLTRPAFAIMNHKSDNEDKLSMSYFFHDLNFKLTNIFNERSRMSLSVYSGEDRLDAKDEWHSNNSSGYNDVDIYVNRFHWGNFNAALDWNYQFSPKLFANFTAVYTHNRSTVSSSDEWRFTRPGEKEQLTLTSHGYRSSIDDIGYRAAFDFRPSPRHHIRFGQDYTYHRFQPQTYNRFDNYQTNSEAKADTIATHSYNKNVAHQLTFYAEDEMTLNEKWSLNGGVNADVFHISGKTFATLSPRLSMKFQPTERLSLKASYTLMSQFVHKIANSFLDLPTDYWVPTTARLHPMRSWQVAAGAYMKPNKHWLLSLEAYYKRSSHILQYSSWAGLEPPAANWDYMVMEGDGRSYGVELDADYNVSNLTLHGSYTLSWTQKKFDDFYDGWYYDKFDNRHKLTLTGRWNITKKIAAFAAWTFRTGNRMTIPTQYIGLPDVPAQEQGGLTFNSSDDNTLNFAYEKPNNVILPAYHRLDIGFDFHHTTKKGHERIWNLSFYNAYCHLNSLWVRVKIDSNNQMKIRNIAFIPVIPSFSYTFKF

SEQ ID NO: 56 - protein, 1133 aa, Escherichia coli
MSKQVFQTDSRQRWSYFKWTLRVILTILSLLGIVFLAMFALEGSPQMPFRHDYRNAVTAASPYTKDNKTAKLYKSFRDFFKEKKMHNNYAKATIKKQRFIGKADSVTQKYFREWDDPRIGVRSAWYVNWDKHAYISLKNNIKHLNMVLPEWFFINPKTDKVEYRIDKQALRLMRRTGIPVLPMLTNNYNSDFHPEAIGRIMRDEKKRMALINEMVRTCRHYGFAGINLDLEELNIQDNDLLVELLKDFSRVFHANGLYVTQAVAPFNEDYNMQELAKYNDYLFLMAYDEHNIESQPGAVSSQRWVEKATDWAAKNVPNDKIVLGMATYGYDWANGEGGTTVSFDQTMAIAQDADAKVKFDDDTYNVNFSYQNTDDGKIHHVFFTDAATTFNIMRFGAEYHLAGYGLWRLGTEDKRIWRFYGKDMSWENVARMSVAKLMQLNGTDDVNFVGSGEVLEVTTEPHPGDISIRIDKDNRLISEEYYRALPSTYTIQRLGKCKDKQLVITFDDGPDSRWTPTVLSTLKKYNVPAAFFMVGLQMEKNLPLVKQVYEDGHTIGNHTFTHHNMIENSDRRSYAELKLTRMLIESVTGHSTILFRAPYNADADPTEHEEIWPMIVASRRNYLFVGESIDPNDWEPNVTSDQIYQRVIDGVHHEDGHIILLHDAGGSSRKPTLDALPRIIETLQHEGYQFISLEQYLGMGKQTLMPEINKGKAYYAMQTNLWLAEMIYHVSDFLTALFLVFLALGMMRLIFMYVLMIREKRAENRRNYAPIDAATAPAVSIIVPGYNEEVNIVRTITTLKQQDYPNLHIYFVDDGSKDHTLERVHEAFDNDDTVTILAKKNGGKASALNYGIAACRSEYVVCIDADTQLKNDAVSRLMKHFIADTEKRVGAVAGNVKVGNQRNMLTYWQAIEYTSSQNFDRMAYSNINAITVVPGAIGAFRKEVIEAVGGFTTDTLAEDCDLTMSINEHGYIIENENYAVALTEAPETLRQFVKQRIRWCFGVMQAFWKHRSSLFAPSKKGFGLWAMPNMLIFQYIIPTFSPLADVLMLIGLFTGNALQIFFYYLIFLVIDASVSIMAYIFEGERLWVLLWVIPQRFFYRWIMYYVLFKSYLKAIKGELQTWGVLKRTGHVKG

SEQ ID NO: 57 - protein, 292 aa, Bacillus subtilis
MAKKRNKARSRHSLQVVTLCISTAMVLMLIGIVVLTGFTSRNLSSYVKENLTITMILQPDMNTEESAALCERIRTLHYINSLNFISKEQALKDGTKELGANPAEFAGENPFTGEIEVQLKANYANNDSIRNIVQQLRTYRGVSDITYPQSLVESVNQTLGKISLVLLVIAVLLTIISFSLINNTIRLSIYAHRFSIHTMKLVGGSWSFIRAPFLRRAVLEGLVSALLAIAVLGIGICLLYEKEPEITKLLSWDALIITAIVMLAFGVIIATFCAWLSVNKFLRMKAGDLYKI

SEQ ID NO: 58 - protein, 254 aa, Haemophilus influenzae
MKNIYFLSDAHLGSLAIDHRRTHERRLVRFLDSIKHKAAAVYLLGDMFDFWNEYKYVVPKGFTRFLGKISELTDMGVEVHFFTGNHDLWTYGYLEKECGVILHRKPITTEIYDKVFYLAHGDGLGDPDPMFRFLRKVFHNRFCQRLLNFFHPWWGMQLGLNWAKRSRLKRKDGKEVPYLGEDKEYLVQYTKEYMSTHKDIDYYIYGHRHIELDLTLSRKARLLILGDWIWQFTYAVFDGEHMFLEEYVEGESKP

SEQ ID NO: 59 - protein, 388 aa, Escherichia coli
MVGLDVLCYFIHAKGREKECYFERIIYQITCHSRTKCYLCNIMKYSIIVPVFNRPDEVEELLESLLSQEEKDFEVVIVEDGSQIPCKEVCDKYADKLDLHYYSKENSGPGQSRNYGAERAKGEYLLILDSDVVLPKGYICAVSEELKREPADAFGGPDCAHESFTDTQKAISYSMTSFFTTGGIRGGKKKLDKFYPRSFNMGIRRDVYQELGGFSKMRFGEDIDFSIRIFKAGKRCRLFPEAWVWHKRRTDFRKFWKQVYNSGIARINLYKKYPESLKLVHLLPMVFTVGTALLVLMILFGLFLQLFPIINVFGSVFIMMGLMPLVLYSVIICVDSTMQNNSLNIGLLSIEAAFIQLTGYGCGFISAWWKRCVCGMDEFAAYEKNFYK

SEQ ID NO: 60 - protein, 435 aa, Streptococcus mutans
MKIEKVHAREIMDSRGNPTVEVEVTLENGVMGRASVPSGASTGENEALELRDGDKNRFLGKGVLKAVENVNNLIAPALKGDCVLNQRAIDYKMLELDGTPTKSKLGANAILGVSLAVAQAAAKALNIPLYRYIGGANTYVLPVPMMNIINGGAHSDAPIAFQEFMIRPVGAPSEKEGIRMGAEVFHALAKLLKKRGLSTAVGDEGGFAPKFDGIEDALDSIIQAIKDAGYEPGKDVKIAMDCAASEFAVCEDGKWFYDYRQLKNGMPKDPNGKKLSADEQIAYLEHLITKYPIDSIEDGLDENDWENWVKLTSAIGDRCQLVGDDLFVTNVKFLEKGIKMGAANSILIKVNQIGSLTETLEAIEMAHRHGYTTVTSHRSGETEDTTIADIAVATNSGQIKTGSMSRTDRMAKYNQLIRIEEELGACAKYGYAKLK

SEQ ID NO: 61 - protein, 501 aa, Brucella suis
MKKLFTIAMLLGVTLGIHAQEVYSLQKCRELALQNNRQLKVSRMTVDVAENTRKAAKTKYLPRVDALAGYQHFSREISLLSDDQKNAFSNLGTNTFGQLGGQIGQNLTSLAQQGILSPQMAQQLGQLFSNVATPLTQVGNNIGQSINDAFRSNTKNVYAGGIVVNQPIYMGGAIKAANDMAAIGEQVAQNNISLKRQLVLYGVDNAYWLAISLKKKEALAIRYRDLAQKLNEDVKKMIREGVATRADGLKVEVAVNTADMQIARIQSGVSLAKMALCELCGLELNGDIPLSDEGDADLPPTPSTQFDNYTVSSSDTTGLNEARPELRLLQNAVDLSIQNTKLIRSLYMPHVLLTAGYSVSNPNLFNGFQKRFTDLWNIGITVQVPVWNWGENKYKVRASKTATTIAQLEMDDVRKKIDLEIEQNRLRLKDANKQLATSQKNMAAAEENLRCANVGFKEGVMTVTEVMAAQTAWQTSRMAIIDAEISVKLAQTGLQKALGGL

SEQ ID NO: 62 - protein, 577 aa, Salmonella typhimurium
MKRTFVTKMVKPIEENSLFFMFMLLVGAFTNVSHRNVFGYIELIADVYIICFLLSLCQRTIRQGLVIMLSSVIYVVAIIDTCCKTLFDTPITPTMLLLAQETTGREATEFFLQYLNLKLFFSAADIILFLAFCHIVMAVKKMKFSTSYLKQPFVAFVLMFTIFVGMALSIYDKVQLYTVKNLSGLEVAVTNGFAHLYHPVERIVYGLYSNHLIAKQVDGVIMANQQIKVDSCSFTSPTIVLVIGESANRHHSQLYGYPLPTTPYQLAMKNGKDSLAVFTNVVSPWNLTSKVFKQIFSLQSVDEKGDWSKYVLFPAVFKKAGYHVSFLSNQFPYGINYTPDWTNNLVGGFFLNHPQLNKQMFDYRNVTIHNYDEDLLNDYKEIISYKKPQLIIFHLLGQHFQYSLRCKSNMKKFGIKDYKRMDLTDKEKQTIADYDNATLYNDFVLNKIVEQFRNKDAIIVYLSDHGEDCYGKDVNMAGRLTEVEQINLKKYHEEFEIPFWIWCSPIYKQRHRKIFTETLMARNNKFMTDDLPHLLLYLAGIKTKDYCEERNVISPSFNNNRRRLVLKTIDYDKALYQ

SEQ ID NO: 63 - protein, 517 aa, Escherichia coli
MFKNHPKGLLQAAFSNMGERFGYYIMNAVLALFLCSKFGLSDETSGLIASLFLAAIYVMSLVGGVIADRTQNYQRTIESGLVVMALGYVALSIPVLATPENNSYLLAFTIFALVLIAVGNGLFKGNLQAIVGQMYDDFETEAAKVSPERLKWAQGQRDAGFQIFYVFINLGALAAPFIAPVLRSWWLGRNGLTYDAALPQLCHKYINGTIGDNLGNLQELATKVGGNSADLASFCPHYLDVFNTGVHYSFIASVVTMLISLIIFMSSKKLFPMPGKKEQIVNVEYTDEEKASMAKEIKQRMYALFAVLGISVFFWFSFHQNGQSLSFFARDFVNTDSVAPEIWQAVNPFFVISLTPLIMWVFAYFTKKGKPISTPRKIAYGMGIAGFAYLFLMGFSLVHNYPSAEQFTSLEPAVRATMKAGPMILILTYFFLTVAELFISPLGLSFVSKVAPKNLQGLCQGLWLGATAVGNGFLWIGPLMYNKWSIWTCWLVFAIVCFISMVVMFGMVKWLERVTKS

SEQ ID NO: 64 - protein, 395 aa, Pseudomonas aeruginosa
MQKKIKIGLLPRVIIAILLGLFLGYYLPDPAVRVFLTFNSIFSQFLGFMIPLIIIGLVTPAIAGIGKGAGKLLLATVAIAYVDTIVAGGLSYGTGTWLFPSMIASTGGAIPHIDKATELTPYFTINIPAMVDVMSSLVFSFIAGLGIAYGGLRTMENLFNEFKTVIEKVIEKAIIPLLPLYIFGVFLSMTHNGQARQVLLVFSQIIIVILVLHVLILIYEFCIAGAIVKHNPFRLLWNMLPAYLTALGTSSSAATIPVTLKQTVKNGVSEEVAGFVVPLCATIHLSGSAMKITACALTICMLTDLPHDPGLFIYFILMLAIIMVAAPGVPGGAIMAALAPLSSILGFNEEAQALMIALYIAMDSFGTACNVTGDGAIALAVNKFFGKKKETSILS

SEQ ID NO: 65 - protein, 208 aa, Escherichia coli
MISVYSIKPQFQRVLTPILELLHRAKVTANQITLWACVLSLVIGILFWFAGDVGTWLYLCLPVGLLIRMALNALDGMMARRYNQITRKGELLNEVGDVVSDTIIYFPLLKYHPESLYFIVAFIALSIINEYAGVMGKVLSAERRYDGPMGKSDRAFVLGLYGVVCLFGINLSGYSVYIFGVIDLLLVLSTWIRIKKTLKVTRNSQTPE

SEQ ID NO: 66 - protein, 582 aa, Escherichia coli
MKLSTILLSIMLGLSSSTMAQQKDVTIKLIETTDVHGSFFPYDFITRKPKSGSMARVYTLVEELRKKDGKDNVYLLDNGDILQGQPISYYYNYVAPEKTNIAASVLNYMGYDVATVGNHDIETGHKVYDKWFKELKFPILGANIIDTKTNKPYILPYYTIKKKNGIKVCVIGMLTPAIPNWLKESIWSGLRFEEMVSCAKRTMAEVKTQEKPDVIVGLFHSGWDGGIKTPEYDEDASKKVAKEVPGFDIVFFGHDHTPHSSIEKNIVGKDVICLDPANNAQRVAIATLTLRPKTVKGKRQYTVTKATGELVDVKELKADDAFIQHFQPEIDAVKAWSDQVIGRFENTIYSKDSYFGNSAFNDLILNLELEITKADIAFNAPLLFNASIKAGPITVADMFNLYKYENNLCTMRLTGKEIRKHLEMSYDLWCNTMKSPEDHLLLLSSTQNDAQRLGFKNFSFNFDSAAGIDYEVDVTKPDGQKVRILRMSNGEPFDENKWYTVAVNSYRANGGGELLTKGAGIPRDSLKSRIIWESPKDQRHYLMEEIKKAGVMNPQPNHNWKFIPETWTVPAAARDRKLLFGE

SEQ ID NO: 67 - protein, 826 aa, Escherichia coli
MKLSELKTGETGVIVKVSGHGGFRKRIIEMGFIKGKTVEVLLNAPLQDPVKYKIMGYEVSLRHSEADQIEVLSDVKTHSVGNEEEQEDNQLEMDSTTYDSTDKELTPEKQSDAVRRKNHTINVALVGNPNCGKTSLFNFASGAHERVGNYSGVTVDAKVGRAEFDGYVFNLVDLPGTYSLSAYSPEELYVRKQLVDKTPDVVINVIDSSNLERNLYLTTQLIDMHIRMVCALNMFDETEQRGDHIDAQKLSELFGVPMIPTVFTNGRGVKELFRQIIAVYEGKEDESLQFRHIHINHGHEIENGIKEMQEHLKKYPELCHRYSTRYLAIKLLEHDKDVEQLVSPLGDSIEIFNHRDTAAARVKEETGNDSETAIMDAKYGFINGALKEANFSTGDKKDTYQTTHVIDHVLTNKYFGFPIFFLVLLVMFTATFVIGQYPMDWIEAGVGWLGEFISKNMPAGPVKDMIVDGIIGGVGAVIVFLPQILILYFFISYMEDCGYMSRAAFIMDRLMHKMGLHGKSFIPLIMGFGCNVPAVMATRTIESRRSRLITMLILPLMSCSARLPIYVMITGSFFALKYRSLAMLSLYIIGVLMAVAMSRLFSAFVVKGEDTPFVMELPPYRFPTWKAIGRHTWEKGKQYLKKMGGIILVASIIVWALGYFPLPDDPNMDNQARQEQSYIGRIGKAVEPVFRPQGFNWKLDVGLLSGMGAKEIVASTMGVLYSNDGSFSDDNGYSSETGKYSKLHNLITKDVATMHHISYEEAEPIATLTAFSFLLFVLLYFPCVATIAAIKGETGSWGWALFAAGYTTALAWIVSAVVFQVGMLFM

SEQ ID NO: 68 - protein, 437 aa, Mycobacterium tuberculosis
MESFIIEGGHQLSGTIAPQGAKNEALEVICATLLTSEEVIIRNVPDILDVNNLIKLLQDIGVKVKKLAPNEFSFQADEVNLDYLESSDFVKKCSSLRGSVLMIGPLLGRFGKATIAKPGGDKIGRRRLDTHFLGFKNLGAHFGRVEDRDVYEIQADKLVGTYMLLDEASITGTANIIMAAVLAEGTTTIYNAACEPYIQQLCKMLNAMGAKISGIASNLITIEGVKELHSADHRILPDMIEVGSFIGIAAMIGDGVRIKDVSVPNLGLILDTFHRLGVQIIVDNDDLIIPRQDHYVIDSFIDGTIMTISDAPWPGLTPDLISVLLVVATQAQGSVLFHQKMFESRLFFVDKLIDMGAQIILCDPHRAVVVGHDNAKKLRAGRMSSPDIRAGIALLIAALTAQGTSRIDNIVQIDRGYENIEGRLNALGAKIQRAEVC

SEQ ID NO: 69 - protein, 244 aa, Bacillus subtilis
MNIAVIFAGGSGLRMHTKSRPKQFLDLNGKPIIIYTLELFDNHPNIDAIVVACIESWIPFLEKQLRKFEINKVVKIIPGGKSGQESIYKGLCAAEEYAQSKGVSNEETTVLIHDGVRPLITEETITDNIKKVEEVGSCITCIPATETLIVKQADDALEIPSRADSFIARAPQSFRLIDIITAHRRSLAEGKADFIDSCTMMSHYGYKLGTIIGPMENIKITTPTDFFVLRAMVKVHEDQQIFGL